SEQUENCE LISTING (65 sequences)

SEQ ID NO: 1 - DNA, 22 bp, synthetic construct
  source 1..22: mol_type = other DNA
ccatgacatccgcagaaggagt

SEQ ID NO: 2 - DNA, 22 bp, synthetic construct
  source 1..22: mol_type = other DNA
gcgttgacagaatgtggtctcg

SEQ ID NO: 3 - DNA, 22 bp, synthetic construct
  source 1..22: mol_type = other DNA
caccgaaggagacagtgaatcc

SEQ ID NO: 4 - DNA, 23 bp, synthetic construct
  source 1..23: mol_type = other DNA
gctgttctggagtaagcttgtgc

SEQ ID NO: 5 - DNA, 22 bp, synthetic construct
  source 1..22: mol_type = other DNA
ctgctctcaaggttgttcggct

SEQ ID NO: 6 - DNA, 22 bp, synthetic construct
  source 1..22: mol_type = other DNA
ccttccgtttctcctccagagt

SEQ ID NO: 7 - DNA, 22 bp, synthetic construct
  source 1..22: mol_type = other DNA
cactctggagatgacacctgag

SEQ ID NO: 8 - DNA, 22 bp, synthetic construct
  source 1..22: mol_type = other DNA
gtgttcctctgtcagcatcacc

SEQ ID NO: 9 - DNA, 23 bp, synthetic construct
  source 1..23: mol_type = other DNA
gaacaggttatgttcgccacgtg

SEQ ID NO: 10 - DNA, 22 bp, synthetic construct
  source 1..22: mol_type = other DNA
ccctctggaaatatccgcagga

SEQ ID NO: 11 - DNA, 22 bp, synthetic construct
  source 1..22: mol_type = other DNA
ccaggcagagaatgctgagttc

SEQ ID NO: 12 - DNA, 22 bp, synthetic construct
  source 1..22: mol_type = other DNA
aagactgggctctccttgttgc

SEQ ID NO: 13 - DNA, 22 bp, synthetic construct
  source 1..22: mol_type = other DNA
atcacccagcactttgccgtct

SEQ ID NO: 14 - DNA, 22 bp, synthetic construct
  source 1..22: mol_type = other DNA
gactccaggaagcatttcagcc

SEQ ID NO: 15 - DNA, 22 bp, synthetic construct
  source 1..22: mol_type = other DNA
gatggctcagtggacttcaacc

SEQ ID NO: 16 - DNA, 22 bp, synthetic construct
  source 1..22: mol_type = other DNA
tgctatgcaccttctccagacc

SEQ ID NO: 17 - DNA, 22 bp, synthetic construct
  source 1..22: mol_type = other DNA
gacatgaagggtgcctctcgaa

SEQ ID NO: 18 - DNA, 23 bp, synthetic construct
  source 1..23: mol_type = other DNA
cctggaagttgttcatgctctgg

SEQ ID NO: 19 - DNA, 22 bp, synthetic construct
  source 1..22: mol_type = other DNA
gccatgaagtaccaccctgaca

SEQ ID NO: 20 - DNA, 24 bp, synthetic construct
  source 1..24: mol_type = other DNA
tcgtctattagcatctgagagtgt

SEQ ID NO: 21 - DNA, 21 bp, synthetic construct
  source 1..21: mol_type = other DNA
gtttgaccgctccacgagcct

SEQ ID NO: 22 - DNA, 23 bp, synthetic construct
  source 1..23: mol_type = other DNA
gcacacaagtgttcatcctcagg

SEQ ID NO: 23 - DNA, 24 bp, synthetic construct
  source 1..24: mol_type = other DNA
tatgagccagaagaacttttaggc

SEQ ID NO: 24 - DNA, 23 bp, synthetic construct
  source 1..23: mol_type = other DNA
cacctcttttggcaagcatcctg

SEQ ID NO: 25 - DNA, 22 bp, synthetic construct
  source 1..22: mol_type = other DNA
tgagcagcatggacgacctgat

SEQ ID NO: 26 - DNA, 23 bp, synthetic construct
  source 1..23: mol_type = other DNA
cgtacataacccgttccattgcc

SEQ ID NO: 27 - DNA, 24 bp, synthetic construct
  source 1..24: mol_type = other DNA
ctgtctggtattttgatgccaagg

SEQ ID NO: 28 - DNA, 22 bp, synthetic construct
  source 1..22: mol_type = other DNA
cggctgtgatacaggtacttgg

SEQ ID NO: 29 - DNA, 19 bp, synthetic construct
  source 1..19: mol_type = other DNA
agtccgccctgagcaaaga

SEQ ID NO: 30 - DNA, 20 bp, synthetic construct
  source 1..20: mol_type = other DNA
tccagcaggaccatgtgatc

SEQ ID NO: 31 - DNA, 22 bp, synthetic construct
  source 1..22: mol_type = other DNA
gtgcctatgagcagttgctgtc

SEQ ID NO: 32 - DNA, 22 bp, synthetic construct
  source 1..22: mol_type = other DNA
aagtagcggctgaagtcagagg

SEQ ID NO: 33 - DNA, 22 bp, synthetic construct
  source 1..22: mol_type = other DNA
tggtggatgactccagagagca

SEQ ID NO: 34 - DNA, 22 bp, synthetic construct
  source 1..22: mol_type = other DNA
actctgccttgtggctcctcat

SEQ ID NO: 35 - DNA, 22 bp, synthetic construct
  source 1..22: mol_type = other DNA
gccaaagctctgttacgtctcc

SEQ ID NO: 36 - DNA, 22 bp, synthetic construct
  source 1..22: mol_type = other DNA
caaagcagtcctcagccgttag

SEQ ID NO: 37 - DNA, 22 bp, synthetic construct
  source 1..22: mol_type = other DNA
gacacacgatgggcttctggtt

SEQ ID NO: 38 - DNA, 22 bp, synthetic construct
  source 1..22: mol_type = other DNA
acaacctggaggcatcgctctt

SEQ ID NO: 39 - DNA, 22 bp, synthetic construct
  source 1..22: mol_type = other DNA
ttcgtgttgagtaacctggcgg

SEQ ID NO: 40 - DNA, 22 bp, synthetic construct
  source 1..22: mol_type = other DNA
gagatccaggttacgctccgat

SEQ ID NO: 41 - DNA, 22 bp, synthetic construct
  source 1..22: mol_type = other DNA
agagactgtggatgtcggatgg

SEQ ID NO: 42 - DNA, 22 bp, synthetic construct
  source 1..22: mol_type = other DNA
agctgttccaccgtcacttcgt

SEQ ID NO: 43 - DNA, 22 bp, synthetic construct
  source 1..22: mol_type = other DNA
gcgagacagatacagagggcga

SEQ ID NO: 44 - DNA, 22 bp, synthetic construct
  source 1..22: mol_type = other DNA
ctgagcggcgatcctgttcttg

SEQ ID NO: 45 - DNA, 22 bp, synthetic construct
  source 1..22: mol_type = other DNA
gctggatgaagcagtggctctt

SEQ ID NO: 46 - DNA, 22 bp, synthetic construct
  source 1..22: mol_type = other DNA
gatgtggctgacttcttcctgg

SEQ ID NO: 47 - DNA, 23 bp, synthetic construct
  source 1..23: mol_type = other DNA
cctcaagtttccatgtccaaggc

SEQ ID NO: 48 - DNA, 22 bp, synthetic construct
  source 1..22: mol_type = other DNA
gaggagagttgtaacggcaagg

SEQ ID NO: 49 - DNA, 24 bp, synthetic construct
  source 1..24: mol_type = other DNA
acgtattggaaggagattacagct

SEQ ID NO: 50 - DNA, 22 bp, synthetic construct
  source 1..22: mol_type = other DNA
tctgtcagcgttactatcccgc

SEQ ID NO: 51 - DNA, 22 bp, synthetic construct
  source 1..22: mol_type = other DNA
aggctcagtgatatggtgccca

SEQ ID NO: 52 - DNA, 22 bp, synthetic construct
  source 1..22: mol_type = other DNA
agccaagtccagcagttcagga

SEQ ID NO: 53 - DNA, 24 bp, synthetic construct
  source 1..24: mol_type = other DNA
ctgtgtctgagaagagtaacttcc

SEQ ID NO: 54 - DNA, 22 bp, synthetic construct
  source 1..22: mol_type = other DNA
ttgccataggctgaggactcct

SEQ ID NO: 55 - DNA, 20 bp, synthetic construct
  source 1..20: mol_type = other DNA
agtcgcgatttcctcatccc

SEQ ID NO: 56 - DNA, 20 bp, synthetic construct
  source 1..20: mol_type = other DNA
ttccctttgtttccgcgagt

SEQ ID NO: 57 - DNA, 20 bp, synthetic construct
  source 1..20: mol_type = other DNA
gctctcgcttcagtcagtcg

SEQ ID NO: 58 - DNA, 19 bp, synthetic construct
  source 1..19: mol_type = other DNA
ttcactggacctgtccgcc

SEQ ID NO: 59 - DNA, 17 bp, synthetic construct
  source 1..17: mol_type = other DNA
ccggtgacagccgatcc

SEQ ID NO: 60 - DNA, 19 bp, synthetic construct
  source 1..19: mol_type = other DNA
agaagccacagccagtacg

SEQ ID NO: 61 - DNA, 21 bp, synthetic construct
  source 1..21: mol_type = other DNA
atgactcagctagttggaccg

SEQ ID NO: 62 - DNA, 20 bp, synthetic construct
  source 1..20: mol_type = other DNA
ctcacatggaccacagcgat

SEQ ID NO: 63 - DNA, 20 bp, synthetic construct
  source 1..20: mol_type = other DNA
gcggttccgagcccaccgct

SEQ ID NO: 64 - DNA, 20 bp, synthetic construct
  source 1..20: mol_type = other DNA
ctcaagaatcgtaggccagg

SEQ ID NO: 65 - DNA, 20 bp, synthetic construct
  source 1..20: mol_type = other DNA
agtgcaccctaacaagccgg

(19) United States
(12) Patent Application Publication
(10) Pub. No.: US 2026/0306455 A1
(43) Pub. Date: Oct. 8, 2026

(54) COMPOSITIONS AND METHODS FOR TREATING CANCER BY TARGETING BACH1

(71) Applicant: THE UNIVERSITY OF CHICAGO, Chicago, IL (US)

(72) Inventors: Marsha Rich ROSNER, Chicago, IL (US); Long Chi NGUYEN, Chicago, IL (US); Emily SHI, New York, NY (US); Margarite MATOSSIAN, Chicago, IL (US); Wenchao LIU, Chicago, IL (US); Madeline BUNGERT, Chicago, IL (US); Kent SCHECHTER, Orinda, CA (US)

(21) Appl. No.: 19/642,558

(22) Filed: Apr. 8, 2026

Related U.S. Application Data
(60) Provisional application No. 63/785,476, filed on Apr. 8, 2025.

(51) Int. Cl. A61K 31/409; A61K 39/395; A61P 35/00; C07K 16/28

(52) U.S. Cl. CPC A61K 31/409; A61K 39/3955; A61P 35/00; C07K 16/2818

(57) ABSTRACT

The present disclosure relates to compositions and methods for treating cancers, such as breast cancer, or enhancing a therapeutic efficacy of an anti-cancer agent. The methods include administering a therapeutically effective amount of a transcription factor BTB and CNC Homology 1 (BACH1) inhibitor to the subject. The methods also further include administering a therapeutically effective amount of an anti-cancer agent. The compositions can include a therapeutically effective amount of a BACH1 inhibitor, or a therapeutically effective amount of a BACH1 inhibitor and a therapeutically effective amount of an anti-cancer agent. Finally, the present disclosure also relates to methods of treating cancer in a subject, including detecting a level of expression and/or activity of BACH1 and/or of an immune checkpoint protein in the subject's cancer tumor; and administering a therapeutically effective amount of the BACH1 inhibitor and an inhibitor of the immune checkpoint protein to the subject.

### CROSS-REFERENCE TO RELATED APPLICATIONS

This application claims priority to U.S. provisional application No. 63/785,476, filed Apr. 8, 2025, the disclosure of which is expressly incorporated by reference herein.

### STATEMENT REGARDING FEDERALLY SPONSORED RESEARCH

This invention was made with government support under W81XWH2110120 awarded by the Department of Defense and CA266643 awarded by the National Institutes of Health. The government has certain rights in the invention.

### REFERENCE TO AN ELECTRONIC SEQUENCE LISTING

This application contains a Sequence Listing that has been submitted electronically and is hereby incorporated by reference in its entirety. The Sequence Listing was created on Apr. 1, 2026, is named “23-1686-US-PRO_SequenceListing.xml”, and is 58,305 bytes in size.

### BACKGROUND OF THE DISCLOSURE

Metastasis is the leading cause of mortality in solid tumors, presenting significant therapeutic challenges. Non-genetic phenotypic plasticity, largely driven by external stress, has been implicated in metastatic progression, immune evasion, and drug resistance. Current treatment options for cancer include chemotherapy, targeted therapy, immunotherapy, radiation therapy, hormone therapy, hematopoietic stem cell transplant, surgery, or a combination thereof. However, these options are not always effective on some patients with aggressive cancer forms such as triple-negative breast cancer (TNBC).

Thus, there remains a need for improved treatments of cancer.

### SUMMARY OF THE DISCLOSURE

As shown and described herein, the present disclosure relates to compositions and methods for treating cancer.

In a first aspect, the present disclosure provides a method of treating breast cancer in a subject that includes administering a therapeutically effective amount of a transcription factor BTB and CNC Homology 1 (BACH1) inhibitor to the subject.

In a second aspect, the present disclosure provides a method of treating breast cancer in a subject that includes administering a therapeutically effective amount of a transcription factor BTB and CNC Homology 1 (BACH1) inhibitor to the subject. In some embodiments, the method further includes administering a therapeutically effective amount of an anti-cancer agent to the subject.

In a third aspect, the present disclosure provides a method of enhancing therapeutic efficacy of an anti-cancer agent in a subject having breast cancer. The method includes:
a) administering a therapeutically effective amount of a BACH1 inhibitor to the subject; and
b) administering a therapeutically effective amount of an anti-cancer agent to the subject.

In a fourth aspect, the present disclosure provides a method of enhancing therapeutic efficacy of an anti-cancer agent in a subject having breast cancer. The method includes:
a) administering a therapeutically effective amount of a BACH1 inhibitor to the subject; and
b) administering a therapeutically effective amount of an anti-cancer agent to the subject.

In one embodiment of any of the preceding aspects or embodiments thereof, the anti-cancer agent is an inhibitor of an immune checkpoint protein. The immune checkpoint protein can be CTLA4, PD-1, PD-L1, LAG-3, TIM-3, TIGIT, PD-1H, CD278, CD39/CD73, or CD276.

In one embodiment of any of the preceding aspects or embodiments thereof, the therapeutically effective amount of the BACH1 inhibitor is administered prior to or concurrent with the therapeutically effective amount of the inhibitor of the immune checkpoint protein.

In a fifth aspect, the present disclosure provides a method of treating breast cancer in a subject, The method includes:
a) administering a therapeutically effective amount of a transcription factor BTB and CNC Homology 1 (BACH1) inhibitor to the subject; and
b) administering a therapeutically effective amount of a cytotoxic T lymphocyte-associated molecule-4 (CTLA4) inhibitor to the subject.

In a sixth aspect, the present disclosure provides a method of enhancing therapeutic efficacy of cytotoxic T lymphocyte-associated molecule-4 (CTLA4) checkpoint blockade therapy in a subject having breast cancer. The method includes:
a) administering a therapeutically effective amount of a BACH1 inhibitor to the subject; and
b) administering a therapeutically effective amount of a CTLA4 inhibitor to the subject.

In one embodiment of any of the preceding aspects or embodiments thereof, the therapeutically effective amount of the BACH1 inhibitor is administered prior to or concurrent with the therapeutically effective amount of the CTLA4 inhibitor. In one embodiment, the CTLA4 inhibitor is an anti-CTLA4 antibody. In one embodiment, the anti-CTLA4 antibody is ipilimumab, tremelimumab, botensilimab, or a combination thereof.

In one embodiment of any of the preceding aspects or embodiments thereof, the breast cancer is triple-negative breast cancer.

In a seventh aspect, the present disclosure provides a method of treating cancer in a subject. The method includes:
a) taking a sample from a cancer tumor from the subject;
b) detecting a level of expression and/or activity of BACH1 in the tumor;
c) detecting a level of expression of an immune checkpoint protein in the tumor; and
d) administering a therapeutically effective amount of the BACH1 inhibitor and a therapeutically effective amount of an inhibitor of the immune checkpoint protein to the subject, if the level of expression and/or activity of BACH1 in the cancer tumor is greater than in cancer subjects that are responsive to the inhibitor of the immune checkpoint protein alone without the BACH1 inhibitor, and
if the level of expression of the immune checkpoint protein in the cancer tumor is scored as high.

In one embodiment of the seventh aspect, the step of detecting the level of expression of BACH1 or immune checkpoint protein includes single cell RNA sequencing, Northern blot, serial analysis of gene expression (SAGE), microarray analysis, quantitative reverse transcription polymerase chain reaction (RT-qPCR), bulk RNA sequencing (bulk RNAseq), Western Blot, immunofluorescence, immunohistochemistry, in situ hybridization, or a combination thereof.

In one embodiment of the seventh aspect, the therapeutically effective amount of the BACH1 inhibitor is administered prior to or concurrent with the therapeutically effective amount of the inhibitor of the immune checkpoint protein.

In one embodiment of the seventh aspect, detecting the level of activity of BACH1 comprises detecting expression of one or more genes that are promoted and/or inhibited by BACH1.

In one embodiment of the seventh aspect, the one or more genes promoted by BACH1 is B4GALNT3, PLXDC2, MAP2, SPOCK1, CMTM8, AC011893.1, AC068985.1, AC090809.1, ADAMTS15, AL590652.1, ARHGAP25, ARHGAP28, ARHGAP40, ARMH1, B4GALNT3, BCAM, C5orf46, CASP1, CCDC149, CD24, CD96, CEMIP, CENPV, CHDH, CHRDL1, CMTM8, COL6A2, COLGALT2, CST5, DNALI1, DSC2, DUOX1, EEF1A2, ENPP4, EWSAT1, EYA4, FIBCD1, GJB2, GNAO1, GPR87, IGFL2-AS1, ITIH4, KLHDC8B, KRT86, LINC00261, LINC00526, LINC01088, LINC02562, LONRF2, LRRC75B, MANEAL, MANSC1, MAOB, MAP2, MPP7, ONECUT2, PDE8B, PDGFD, PLB1, PLCH1, PLCL2, PLD5, PLLP, PLXDC2, PPARGC1A, PRSS2, RARRES2, RASL10A, RAVER2, RBM24, RHPN1, RIMKLA, RNF43, SCARA3, SERPINA1, SH3BP1, SLC38A5, SOD3, SPINT1, SPOCK1, SPOCK3, SSTR2, ST6GAL1, STK32A, SYTL2, TCIM, TET1, TFF2, TIAM1, TIMP4, TINAGL1, TMC5, TMEM38A, TMLHE, TNFSF10, TOGARAM2, TRBV30, TRPV4, TSPAN8, URB1-AS1, VSNL1, WFS1, ZNF467, or ZNF726.

In one embodiment of the seventh aspect, the one or more genes inhibited by BACH1 is MAGEC2, IL13RA2, GCLM, TNFSF15, NUPR1, SERPINE1, FTL, IGFBP3, ABCA3, AC005224.3, AC064869.1, AL590681.1, CREB3L2, CT55, EEF1A1, ESM1, FBXO30, FTH1, FTL, GCLM, GNAII, HMOX1, ICAM5, IGFBP3, IL13RA2, KISS1, LINC02303, MAGEC2, NEDD4, NKX2-1, NUPR1, PCK2, PLCG2, PTPRN2, RGS20, SERPINE1, SLC6A8, SLITRK1, TNFSF15, TPT1, or XAGE1B.

In one embodiment of the seventh aspect, the immune checkpoint protein is CTLA4, PD-1, PD-L1, LAG-3, TIM-3, TIGIT, PD-1H, CD278, CD39/CD73, or CD276. In one embodiment, the inhibitor of the immune checkpoint protein is an anti-CTLA4 antibody. In one embodiment, the anti-CTLA4 antibody is ipilimumab, tremelimumab, botensilimab, or a combination thereof.

In one embodiment of the seventh aspect, the cancer is a breast cancer. In one embodiment, the breast cancer is a triple-negative breast cancer.

In an eighth aspect, the present disclosure provides a pharmaceutical composition. The pharmaceutical composition includes:
a) a therapeutically effective amount of a BACH1 inhibitor;
b) a therapeutically effective amount of an anti-cancer agent; and
c) a pharmaceutically acceptable carrier, solvent, adjuvant, diluent, or a combination thereof.

In a ninth aspect, the present disclosure provides a pharmaceutical composition. The pharmaceutical composition includes:
a) a therapeutically effective amount of a BACH1 inhibitor;
b) a therapeutically effective amount of a CTLA4 inhibitor; and
c) a pharmaceutically acceptable carrier, solvent, adjuvant, diluent, or a combination thereof.

In one embodiment of any of the preceding aspects or embodiments thereof, the anti-cancer agent is an inhibitor of an immune checkpoint protein. In one embodiment, the immune checkpoint protein is CTLA4, PD-1, PD-L1, LAG-3, TIM-3, TIGIT, PD-1H, CD278, CD39/CD73, or CD276.

In one embodiment of any of the preceding aspects or embodiments thereof, the CTLA4 inhibitor is an anti-CTLA4 antibody. In one embodiment, the anti-CTLA4 antibody is ipilimumab, tremelimumab, botensilimab, or a combination thereof.

In one embodiment of any of the preceding aspects or embodiments thereof, the BACH1 inhibitor comprises hemin.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

FIG. 1A illustrates single-cell and spatial RNA-seq analysis of MDA-MB-231 xenograft primary tumors 8 weeks post-inoculation, capturing heterogeneous cell states.

FIG. 1B shows 3D UMAP clustering of MDA-MB-231 tumor scRNA-seq (10,377 cells) that reveals distinct cellular subpopulations (clusters 0-6).

FIG. 1C shows Metascape Gene Ontology (GO) terms for marker genes in each cluster, highlighting key biological processes. Circles in black boxes show—log ₁₀pvalue from 5 to 60, the rest of the circles show-log ₁₀pvalue around 0.

FIG. 1D shows single-cell reference mapping of in vitro BM1 cells (5,347 cells) and published MDA-MB-231 scRNA-seq (805 cells) [6] onto seven distinct cell-state clusters from MDA-MB-231 xenograft tumors (used as the reference for annotations).

FIG. 1E shows counts, read counts, and mitochondrial content before and after filtering of single cell RNA-seq of MDA-MB-231 control tumors for quality control.

FIG. 1F shows slingshot trajectory inference from scRNA-seq, modeling the progression from proliferative states to extreme cellular stress with a 3D breakdown of each cluster along this trajectory.

FIG. 1G shows slingshot trajectory across cell clusters of MDA-MB-231 control tumors with different starting points (clusters).

FIG. 1H shows 3D UMAP visualizations that reveal state transitions, with cluster 0 interacting and connecting with other clusters.

FIG. 1I shows H&E staining and spatial transcriptomic overlay of tumor sections, visualizing the spatial organization of cluster gene signatures identified in single-cell data. Clusters 4 and 1 gene signatures are highly expressed (in light shade) at the periphery. Cluster 3 gene signatures are highly expressed (in light shade) in the center adjacent to the necrotic region which comprises cells under high stress. Cluster 0 gene signatures are highly expressed (in light shade) in the intersection between these two states. The intersection comprises cells associated with the transition state.

FIG. 1J shows a correlation heatmap that highlights gene signature relationships between MDA-MB-231 tumors single-cell RNA-seq clusters and Hallmark Hypoxia/G2M pathways. Circles in boxes show negative correlation values.

FIG. 1K shows Mean Hallmark Hypoxia (M5891) and Hallmark G2M (M5901) (proliferation) scores across clusters.

FIG. 1L shows colocalization analysis of hypoxia hallmark scores with cluster gene signatures, with higher L-index values indicating stronger spatial correlation. Cluster 3 shows highest L-index, cluster 4 shows lowest L-index, and cluster 0 shows intermediate L-index.

FIG. 1M shows EPR oxygen imaging of MDA-MB-231 control tumors in live mice reveals intratumoral hypoxia. The circled region indicates hypoxia (pO₂≤10 torr) or the registered MRI-derived tumor outline accordingly. The hypoxic area with lowest oxygen content is in the center of the tumor which overlaps with stress cluster 3.

FIG. 1N shows differential gene expression analysis of hypoxia-exposed MDA-MB-231 tumor cells labeled with RFP under normoxic conditions or labeled with GFP upon activation of the hypoxia response element (HRE)-linked GFP. Heatmap displays MDA-MB-231 cluster gene signatures between two conditions. This analysis was based on data from Godet et al. [7]. The black box regions show highly differentially expressed scores.

FIG. 1O shows UMAP projection maps bulk RNA-seq from BM1 cells exposed to 1% O₂ for 0, 24 or 72 h onto MDA-MB-231 tumor cell states. Clusters are numbered 0-6.

FIG. 1P shows Palantir trajectory analysis in 3D UMAP that reveals a progressive movement along the trajectory from proliferative to extreme stress clusters.

FIG. 1Q shows slingshot trajectory modeling that reveals a linear trajectory after cluster 0 removal.

FIG. 1R shows slingshot trajectory across cell clusters of MDA-MB-231 control tumor after removing cluster 0 with different starting point (cluster).

FIG. 1S shows upregulated and downregulated genes in transition cluster 0 of MDA-MB-231 xenograft tumor cells, ranked by GSEA based on p-adjust <0.05 and fold change.

FIG. 1T shows DAVID analysis of transcription factor binding sites (TFBS) across clusters, identifying key regulators of transition states (left), along with top predicted transcription factors driving the cluster 0 transition state.

FIG. 1U shows cross-cohort correlation of Cluster 0 gene signatures across patient datasets, including TCGA, a Nigerian cohort, and I-SPY2, highlighting key pathways that positively and negatively correlate with Cluster 0 gene signatures.

FIG. 1V shows reference mapping of clinical breast cancer epithelial cells scRNA-seq to MDA-MB-231 tumor cell states, supporting the clinical relevance of transition states across different models. T=transition state; H=hypoxia; A=metabolic stress/autophagy; I=inflammation; P=proliferation; M=mitosis; R=migration.

FIGS. 1W and 1X show correlation analysis of MDA-MB-231 cell state clusters with hallmark pathway gene signatures across clinical breast cancer scRNA-seq datasets, using only isolated cancer epithelial cells. Circles in boxes show negative correlation values.

FIG. 1Y shows Kaplan-Meier survival analysis of Distant Metastasis-Free Survival or Relapse-Free Survival in TNBC breast cancer patients, stratified by the transition state (cluster 0) gene signature, using KMplot [81, 82] analysis.

FIG. 1Z shows forest plots displaying odds ratios of therapy response in TNBC patients stratified by transition-state gene signatures across I-SPY2 treatment arms.

FIG. 2A shows a schematic representation of in vivo models assessing tumor growth and metastasis.

FIG. 2B shows orthotopic implantation of 5×10⁵ BM1-1uc cells into the mammary fat pad of athymic nude mice that enables longitudinal tumor volume over 7 weeks and weight measurement (n=8 per condition; Control and BACH1 KO).

FIG. 2C shows intracardiac injection of 1×10⁵ luciferase-expressing BM1 cells (BM1-1uc) into the left ventricle that facilitates systemic dissemination and metastatic burden assessment over 4 weeks (n=5 per condition; Control and BACH1 KO).

FIG. 2D shows tumor growth comparison between MDA-MB-231 control and BACH1 KO tumors. ns=no significant difference.

FIG. 2E shows 3D UMAP visualization of MDA-MB-231 tumor clusters, comparing control and BACH1 KO cells, showing comparable cell states/clusters between control and BACH1 KO cells. However, BACH1 KO cells show less connectivity between cell clusters. FIG. 2E shows alongside spatial mapping of gene signatures derived from MDA-MB-231 control tumor clusters. The gene signatures of each cluster are expressed in similar locations in the tumors of control and BACH1 KO. Data were obtained from single-cell and spatial RNA-seq analysis of MDA-MB-231 control and BACH1 KO tumors.

FIG. 2F shows Gene Ontology (GO) enrichment analysis of BACH1 KO gene expression changes across cell clusters, displaying-log 10 (p-value) of differentially regulated pathways. Black box indicates upregulated genes.

FIG. 2G shows differential gene expression analysis between control and BACH1 KO tumors, displaying only significantly adjusted genes across clusters. Data obtained from single-cell and spatial RNA-seq analysis of MDA-MB-231 control and BACH1 KO tumors.

FIG. 2H shows representative spatial visualization of matched tumors, highlighting expression differences of specific up-regulated or down-regulated genes after BACH1 KO. Data obtained from single-cell and spatial RNA-seq analysis of MDA-MB-231 control and BACH1 KO tumors. The light part showed the spatial high expression regions of indicated genes from spatial transcriptomic data. Some of the genes involved in immune evasion like PRSS2, CD24, COL1A2, and COL6A2 are expressed at lower levels in BACH1 KO. HMOX1, FH1, and GCLM are expressed at higher levels in BACH1 KO.

FIG. 2I shows (left) smooth gene expression heatmap along the Palantir trajectory, illustrating the progressive transition from the start (0) to the end (1) of cell trajectory, with cluster 0 positioned near the terminal state of the trajectory. Next, (right, top) 3D UMAP visualization of MDA-MB-231 cell clusters (Control and BACH1 KO) are shown, highlighting interactions between clusters 4, 1, and 5 with cluster 0. The circle in BACH1 KO shows less connectivity than the circle in control. Further, (right, bottom) Heatmaps of cluster 0 gene markers in Control and BACH1 KO cells are shown, demonstrating a reduction in cluster 0 gene expression (and thus, less heterogeneity of genes expression in the transition state) along the trajectory in BACH1 KO tumors, consistent with decreased inter-cluster connectivity and plasticity.

FIG. 2J shows quantification of cluster 0 gene signature scores across different MDA-MB-231 clusters, demonstrating a significant reduction following BACH1 KO.

FIG. 2K shows Palantir trajectory analysis of macro cell states, showing stationary distribution starting from cluster 4-1. Cluster 0 exhibits a high stationary distribution in control tumors but is markedly reduced in BACH1 KO.

FIG. 2L shows single-cell RNA sequencing (scRNA-seq) analysis of BM1 xenograft tumors that overexpress BACH1 (OE), highlighting Gene Ontology (GO) terms associated with significantly upregulated or downregulated genes. Shaded boxes within the black box are upregulated. Others are either unchanged (no shading) or downregulated.

FIG. 2M shows UMAP visualization and proportional distribution of cell clusters between control and BACH1 OE tumors, demonstrating a significant shift in relative BM1-derived cluster populations following BACH1 OE.

FIG. 2N shows tumor mass comparison in BM1 BACH1 overexpression (BACH-OE) xenografts in nude mice.

FIG. 2O shows GO term enrichment of marker genes from BMI control tumor-derived single-cell clusters. Circles in black boxes show-log ₁₀pvalue from 5 to 80, the rest of the circles show-log ₁₀pvalue around 0.

FIG. 2P shows MDA-MB-231-derived cluster 0 gene signature scores across control versus BACH1 OE BMI tumors and within individual BM1-derived clusters.

FIG. 2Q shows a volcano plot of differentially expressed genes between control and BACH1 KO in pseudo-bulk MDA-MB-231 xenograft tumors from scRNA-seq.

FIG. 2R shows a volcano plot of differentially expressed genes between control and BACH1 overexpression (OE) in pseudo-bulk BM1 xenograft tumors from scRNA-seq; Y-axis displays the absolute value of FDR-corrected Z-score.

FIG. 2S shows identification of BACH1-promoted and BACH1-inhibited gene signatures derived from both scRNA-seq and spatial transcriptomics of BACH1 KO and BACH1 OE in BM1 and MDA-MB-231 xenograft tumors.

FIG. 2T shows spatial mapping of BACH1-promoted and BACH1-inhibited gene signatures in MDA-MB-231 xenograft tumors. Expression of BACH1-promoted genes are lower in BACH1 KO (less of the light dots) compared to control and expression of BACH1-inhibited genes are higher in BACH1 KO (more of the light dots) compared to control (more region with darker/lower expression).

FIG. 2U shows UMAP visualization of BACH1-promoted and FIG. 2V shows UMAP visualization of BACH1-inhibited gene signatures in BM1 OE cells. BACH1-promoted genes are expressed in higher level in BACH1 OE (more of the light dots of positive values). BACH1-inhibited genes are expressed in lower level in BACH1 OE (more of the dark dots of negative values)

FIG. 2W shows violin plots illustrating the distribution of BACH1-promoted and BACH1-inhibited gene signatures across MDA-MB-231 xenograft-derived cell state clusters in control and BACH1 KO tumors.

FIG. 2X shows a correlation analysis between BACH1-promoted or inhibited gene signature scores and MDA-MB-231-derived cluster gene signature scores across control MDA-MB-231 xenograft tumors. Color intensity represents the correlation coefficient and size represents the relative proportion. Circles in black boxes have negative correlation coefficients.

FIG. 2Y shows bulk RNA sequencing of BM1 xenograft tumors, displaying a heatmap of MDA-MB-231 state transition cluster 0 gene signature scores, along with representative genes from the cluster 0 signature, comparing control and BACH1 KO conditions. Enclosed black boxes show increasing expression, outside the boxes show decreasing expression. Darker shade shows higher or lower expression, while white color shows no changes (see heatmap bar).

FIG. 2Z shows single-cell RNA-seq of shBACHI BMI tumors, with violin plots comparing the MDA-MB-231-derived state transition cluster 0 gene signature scores between control and shBACH1 tumors, demonstrating a significant reduction upon BACH1 knockdown.

FIG. 2AA shows tumor growth analysis of BM1 xenograft tumors in BACH1 KO conditions, with tumor mass quantification.

FIG. 2AB shows gene Set Enrichment Analysis (GSEA) of differentially expressed genes in BACH1 KO BMI xenograft tumors, along with a heatmap of representative genes, including HMOX1 and inflammatory markers. In the heatmap, enclosed black boxes show decreasing expression, outside the boxes show increasing expression. Darker shade shows higher or lower expression, while white color shows no changes (see heatmap bar).

FIG. 2AC shows Gene Set Enrichment Analysis (GSEA) of bulk RNA-seq data assessing the impact of BACH1 KO on BMI tumors or in vitro under hypoxia.

FIG. 3A shows a schematic representation of multi-omics experiments in BM1 cells under normoxia and hypoxia conditions in vitro.

FIG. 3B shows a heatmap illustrating six distinct gene response patterns to acute (24h) and prolonged (72h) hypoxia, highlighting the modulatory effect of BACH1 on hypoxia-responsive genes. Normalized expression levels of the 5000 most variable genes across all samples were plotted, and K-means clustering was used to divide genes into six clusters based on expression patterns among different treatment conditions. The black boxes show predominantly low expression (negative values).

FIG. 3C shows Gene Set Enrichment Analysis (GSEA) of hypoxia effects in BMI cells at 24 h and 72 h time points. The black boxes show positive normalized enrichment score (NES) values.

FIG. 3D shows principle component analysis (PCA) component of bulk RNA-seq control vs BACH1 KO BMI cells (0, 24, 72 h hours) exposure in hypoxia in FIG. 3B.

FIG. 3E shows metascape analysis of gene groups from hierarchical clustering in FIG. 3B.

FIG. 3F shows representative examples of genes induced or repressed by BACH1 KO with or without hypoxia, across different time points. Enclosed black boxes show increasing expression, outside the enclosure show decreasing expression. Darker shade shows higher or lower expression, while white color shows no changes (see heatmap bar).

FIG. 3G shows Gene Set Enrichment Analysis (GSEA) of bulk RNA-seq data assessing the impact of BACH1 KO on hypoxia response at different time points, demonstrating BACH1's role in modulating hypoxia adaptation. Black box indicates positive NES values.

FIG. 3H shows GSEA of proteomic changes induced by BACH1 KO in BMI cells at 24 h and 72 h hypoxia time points, further illustrating BACH1's role in hypoxia regulation.

FIG. 3I shows schematic representation of BACH1 KO tandem mass tag (TMT) labeling and LC-MS/MS mass spectrometry analysis.

FIG. 3J shows a heatmap displaying representative proteins modulated by BACH1 under normoxia and hypoxia conditions across matched time points. Enclosed black boxes show increasing expression, outside the enclosure show decreasing expression. Darker shade shows higher or lower expression, while white color shows no changes (see heatmap bar).

FIG. 3K shows tumor growth and tumor weight analysis of MDA-MB-436 xenograft tumors in control and BACH1 KO conditions.

FIG. 3L shows qPCR analysis of known hypoxia-responsive genes in MDA-MB-436 tumors, showing that BACH1 promotes the expression of these genes in MDA-MB-436 models without affecting tumor growth or size.

FIG. 3M shows ATAC-seq analysis comparing chromatin accessibility in control, normoxia, and hypoxia conditions with BACH1 KO, showing that BACH1 promotes chromatin accessibility around transcription start sites (TSS) under hypoxia. BACH1 KO has lower signal enrichment around transcription start sites (TSS) under hypoxia compared to control, suggesting that BACH1 promotes chromatin accessibility under hypoxia.

FIG. 3N shows overlapping analysis of peaks with altered chromatin accessibility under normoxia and hypoxia with or without BACH1 KO.

FIG. 3O shows Gene Ontology (GO) analysis of biological functions associated with genes displaying altered chromatin accessibility.

FIG. 3P shows ChIP-seq of BACH1 under normoxia and 24 h hypoxia conditions.

FIG. 3Q shows quantification of the data in FIG. 3P, revealing a significant increase in DNA binding under hypoxia, including gene regions lacking MARE motifs and genes that also express HRE sites. Additionally, BACH1 binds to de novo motifs such as CDX2 (bottom).

FIG. 3R shows ChIP-qPCR or Replicate 2 of ChIP-seq comparing BACH1 DNA binding between normoxia and hypoxia (24h) conditions in BMI cells.

FIG. 3S shows relative fold enrichment of HMOX1 E1 and SERPINE1, confirming increased BACH1 DNA binding under hypoxia. *=p<0.05

FIG. 3T shows quantification of the data in FIG. 3R, including other motifs such as CDX2 in gene regions lacking MARE motifs.

FIG. 3U shows overlapping analysis (top) and Gene Ontology (GO) enrichment (bottom) of BACH1 DNA-binding targets under hypoxia and low glucose conditions, genes with reduced chromatin accessibility and downregulated mRNA expression under hypoxia. This analysis includes gene markers for cluster 0, further evidence of BACH1's role in regulating state transition cluster 0 under hypoxic conditions. The dark lines indicate genes with shared regulation, while gray lines connect genes with similar GO biological functions.

FIG. 3V shows representative ChIP-seq tracks of cluster 0 signature genes, including RFX2, NEAT1, and EDIL3, highlighting BACH1 occupancy at these loci under hypoxia.

FIG. 3W shows heatmap demonstrating that the MDA-MB-231-derived cluster 0 gene signature is regulated by BACH1 under hypoxia and correlates with the BACH1-promoted and BACH1 distance scores. The below schematic illustrates situations with high or low BACH1 distance scores that reflect the difference in BACH1-promoted and BACH1-inhibited gene scores. Representative heatmap of cluster 0 gene expression in response to hypoxia with or without BACH1. In both heatmaps, enclosed black boxes show increasing expression, outside the enclosure show decreasing expression.

FIG. 4A shows a schematic representation of murine TNBC 4T1 syngeneic models in BALB/c mice.

FIG. 4B shows tumor volume and flow cytometry analyses from the tumor at 6 weeks post-inoculation of 4T1 cells (late-stage) from control and BACH1 KO mice.

FIG. 4C shows Western blot confirmation of BACH1 KO and subsequent re-expression of mBACH1 in knockout cells in 4T1 cells (top). Tumor mass after 6 weeks of growth in BALB/c mice implanted with control, BACH1 knockout, or mBACH1 re-expressing 4T1 cells in the mammary fat pad (bottom). *=p<0.05.

FIG. 4D shows Western blot confirmation of BACH1 KO in LMB cells, a significantly lower BACH1 KO LMB tumor weight compared to control, and a significantly fewer number of metastatic lesions for BACH1 KO LMB tumors compared to control. *=p<0.05; ****=p<0.0001.

FIG. 4E shows comparison of control and BACH1 KO 4T1 tumors to monitor lung metastasis. *=p<0.05.

FIG. 4F shows comparison of tumor growth between control and BACH1 KO 4T1 tumors in athymic nude mice.

FIG. 4G shows tumor mass and number of spontaneous lung metastatic lesions in BALB/c mice, comparing control and BACH1 KO 4T1 tumors. ns=no significant difference.

FIG. 4H shows projected Tumor-Infiltrating Lymphocyte (TIL) states in BACH1 KO 4T1 tumors.

FIG. 4I shows projected Tumor-Infiltrating Lymphocyte (TIL) states in BACH1 KO 4T1 tumors stratified using projected TIL proportions and absolute cell counts.

FIG. 4J shows immunohistochemistry (IHC) staining of CD3+, CD4+, and CD8+ markers (dark dots), with quantification of tumor-infiltrating T cells in control, BACH1 KO 4T1 tumors, and BACH1 KO 4T1 tumors stably expressing murine WT BACH1 or the double mutant (P182A/P530A) for rescue experiment, and hypoxia-inducible factor 1 alpha subunit (HIF1A) KO and its corresponding control. *=p<0.05; **=p<0.01; ***=p<0.001.

FIG. 4K shows immunohistochemistry (IHC) staining CD4+ (dark dots) in LMB syngeneic TNBC tumor model in control and BACH1 KO tumors; and quantification of CD4⁺ tumor infiltration in control and BACH1 KO LMB tumors. *=p<0.05.

FIG. 4L shows tumor weight in HIF1A KO 4T1 tumors compared to control. *=p<0.05.

FIG. 4M shows spontaneous lung metastatic burden (internal and surface metastasis) in HIF1A KO 4T1 tumors compared to control. ns=no significant difference.

FIG. 4N shows cytokine profiling (interferon alpha protein (IFN-alpha), IFN-gamma, and granulocyte-macrophage colony-stimulating factor (GM-CSF)) in 4T1 tumors. IFN-alpha expression was significantly increased in BACH1 KO 4T1 tumors compared to control.

FIG. 4O shows GO analysis (from sc-RNA seq data) of genes up- and downregulated in a specific T cell state from control or BACH1 KO 4T1 tumors.

FIG. 4P shows Gene Set Enrichment Analysis (GSEA) of BACH1 KO (RNA-seq) in BM1 cells with or without hypoxia, across different time points; and GSEA results of the Hallmark Interferon Alpha Response pathway.

FIG. 4Q shows GSEA of the Hallmark Interferon Alpha Response pathway comparing BACH11 KO and control in proteomic data.

FIG. 4R shows a slingshot pseudotime trajectory analysis showing a transition from the proliferative cluster to stress clusters in control 4T1 tumor cells.

FIG. 4S shows UMAP and spatial mapping of MDA-MB-231 xenograft-derived tumor cell states and Hallmark hypoxia gene signatures onto 4T1 tumors reveal a comparable transition from proliferative states at the tumor periphery to stress and extreme hypoxic states near the necrotic core in control 4T1 tumors.

FIG. 4T shows UMAP visualization of 4T1 control tumors, showing distinct cell states comparable to MDA-MB-231 tumors, including clusters ranging from proliferative to stress-related, as well as additional epithelial-like and MHC class II-high clusters and Gene Ontology (GO) enrichment analysis of biological processes across different cell clusters in 4T1 tumors.

FIG. 4U shows a dot plot demonstrating that MHC class II genes (H2-Eb1, H2-Ab1, and H2-Aa) are expressed in 4T1 cancer cells but not key costimulatory molecules (Cd80, Cd86), unlike antigen-presenting cells (APCs) such as macrophages and dendritic cells.

FIG. 4V shows UMAP visualization of 4T1 control tumors, showing the expression of the same genes, with Cd86 and Cd80 being expressed at lower level (darker dots) than Cd74 and H2-Ab1.

FIG. 4W shows qPCR time-course analysis of MHC Class II-related genes (Cd74, H2Ab1) and co-stimulatory molecule (Cd80; Cd86) expression in 4T1 cells treated with 10 ng/mL IFN-γ under normoxia.

FIG. 4X shows violin plots which reveal significant downregulation of BACH1-promoted gene signatures (derived from MDA-MB-231 xenograft tumors) and MHC class II gene signatures in BACH1 KO 4T1 tumors.

FIG. 4Y shows UMAP visualization displaying MHC class II gene signatures in 4T1 cancer cells, comparing control and BACH1 KO conditions. Darker region (indicating lower expression score of MHC class II genes) is more dominant in BACH1 KO.

FIG. 4Z shows a volcano plot highlighting top differentially expressed genes in 4T1 cancer cells from control vs. BACH1 KO tumors.

FIG. 4AA shows pseudo-bulk RNA-seq analysis of BACH1 KO effects, displaying Gene Ontology (GO) enrichment of biological processes impacted by BACH1 KO.

FIG. 4AB shows cell-cell interaction analysis using CellChat which predicts a reduction in MHC class II interactions with CD4⁺ and BACH1 low 4T1 tumors utilizing MDA-MB-231 derived BACH1-promoted signatures. The black boxes indicate high communication probability towards maximum probability.

FIG. 4AC cell-cell interaction analysis using CellChat which predicts a reduction in MHC class II interactions with CD4⁺ T cells and an increase in MHC class II interactions with dendritic cells in BACH1 KO 4T1 tumors. The black boxes indicate high communication probability towards maximum probability.

FIG. 4AD shows dot plot analysis of cancer epithelial cells from scRNA-seq of breast cancer patients, revealing frequent expression of MHC class II genes but not costimulatory molecules (CD80, CD86) in tumor epithelial cells.

FIGS. 4AE and 4AF show volcano plots of differentially expressed genes between control and BACH1 KO single-cell suspensions from lymph nodes collected at 5 days (4AE) and 20 days (4AF) post-inoculation respectively in scRNA-seq.

FIG. 4AG shows flow cytometry analysis of single-cell suspensions from lymph nodes collected at 28 days post-inoculation. All graphs show significant differences between BACH1 KO and control.

FIG. 4AH shows differential expression analysis identifying transcriptional changes between control and BACH1 KO dendritic cells collected a 6 weeks post-inoculation in scRNA-seq, and volcano plot highlighting annotated gene function categories among differentially expressed genes.

FIG. 4AI depicts an experiment to test the role of IFN-alpha in mediating BACH1 KO effects in lymph nodes and flow cytometry analysis from the experiment showing the frequencies of the indicated immune cells. Balb/c mice were pretreated with control IgG or anti-IFNAR1 antibody (200 μg per mouse) on days-7 and -3, followed by injection of 4T1 tumor cells on day 0. Antibody treatments continued on days 3, 7, 10, and 14. On day 24 post-tumor inoculation, tumors were harvested and analyzed by flow cytometry to measure PD-1+CD44+ CD T cells, M1 macrophages, and CD103+ dendritic cells (DCs). *=p<0.05; **=p<0.01, or significant differences shown numerically.

FIG. 4AJ depicts an experiment to test the effect of short-course hemin treatment and shows a volcano plot of differentially expressed genes between control and heme-treated mice bearing 4T1 tumors that were associated with T cell quiescence and activation at Day 5.

FIG. 5A shows ChIP-seq showing direct BACH1 binding to CD274 (PD-L1) gene region.

FIG. 5B shows violin plots of checkpoint molecule expression in T cells from 4T1 tumors, showing higher CTLA-4 expression compared to LAG3 and PD-1 in BACH1 KO tumors.

FIG. 5C shows immunofluorescence validation of CTLA-4 expression (arrows) in 4T1 tumor models.

FIGS. 5D and 5E show checkpoint blockade therapies (anti-CTLA4 or PD-L1) in BACH1 KO 4T1 tumors, assessing primary tumor-free survival over time, tumor growth (volume) and weight, and spontaneous lung metastasis at the treatment endpoint. *=p<0.05, or significant differences shown numerically.

FIG. 5F shows checkpoint blockade therapy (anti-PD-1) in BACH1 KO 4T1 tumors, evaluating: primary tumor growth, tumor weight, and spontaneous lung metastasis at the treatment endpoint. *=p<0.05.

FIG. 5G shows cell-cell interaction analysis (CellChat) that reveals altered CD80-CTLA4 interactions between control and BACH1 KO 4T1 tumors, highlighting increased Treg interactions in BACH1 KO tumors collected at the 6-week endpoint.

FIG. 5H shows evaluation on primary tumor growth, mouse weight, and T cell infiltration of BALB/c mice bearing 4T1 tumors on continuous hemin treatment (50 mg/kg). *=p<0.05; **=p<0.01.

FIG. 5I show that mice bearing 4T1 tumors were first treated with three doses of hemin (50 mg/kg), followed by anti-CTLA-4 checkpoint blockade therapy. Tumor volumes were measured over time to assess the effect of sequential therapy on primary tumor growth. Significant differences shown numerically.

FIG. 5J shows a schematic illustration of BACH1 inhibition restoring T cell infiltration and enhancing checkpoint blockade efficacy.

FIG. 6A is a heatmap showing the Z-score distribution of BACH1-promoted and BACH1-inhibited gene signatures, TIL (tumor-infiltrating lymphocyte) score, and BACH1 activity “distance” score, comparing TNBC vs. non-TNBC high-risk breast cancer patients from the I-SPY2 clinical trial dataset. Boxed region shows predominantly positive Z-scores.

FIG. 6B shows correlation between the Tumor-Infiltrating Lymphocyte (TIL) gene signature and BACH1 signatures or BACH1 mRNA among high-risk breast cancer patients in the I-SPY2 trials.

FIG. 6C shows negative correlation between the Tumor-Infiltrating Lymphocyte (TIL) related genes and BACH1 promoted score among basal patients in TCGA data set.

FIG. 6D shows correlation between the Tumor-Infiltrating Lymphocyte (TIL) gene signature and BACH1 signatures among high-risk breast cancer patients in the I-SPY2 trials.

FIG. 6E show Gene Ontology (GO) analyses of biological processes enriched in genes that positively or negatively correlate with BACH1 activity distance scores across different datasets and breast cancer subtypes, including TCGA, I-SPY2, and the Nigerian breast cancer cohort.

FIG. 6F shows Gene Ontology (GO) analysis of biological processes enriched in genes that positively or negatively correlate with BACH1 promoted scores across different datasets and breast cancer subtypes, including TCGA, I-SPY2, and the Nigerian breast cancer cohort.

FIG. 6G shows dot plots showing a strong positive association between Cluster 0 and BACH1-promoted gene signatures in I-SPY2 high-risk breast cancer patients.

FIG. 6H shows correlation analysis of scRNA-seq data from isolated cancer epithelial cells of breast cancer patients, demonstrating a strong positive relationship between the MDA-MB-231 cluster 0 signature and BACH1-promoted gene signatures, compared to other cluster signatures. Enclosed black boxes show decreasing expression, outside the enclosure show increasing expression. Darker shade shows higher or lower expression, while white color shows no changes (see heatmap bar).

FIG. 6I shows Kaplan-Meier survival analysis of overall or distant metastasis-free survival (DMFS) in basal-like or all breast cancer patients undergoing chemotherapy, stratified by BACH1-promoted gene signature levels, using KMplot analysis.

FIG. 6J shows a violin plot comparing BACH1-promoted signature z-scores between responders and non-responders in TNBC patients from the I-SPY2 trial arm treated with pembrolizumab and paclitaxel.

FIG. 6K show (Top) forest plot of odds ratios (ORs) for pathological complete response (pCR) responders vs. non-responders among high-risk TNBC patients, stratified by BACH1 mRNA expression, BACH1-promoted and BACH1-inhibited gene signatures, and their combinations (Z-score>0=high). (Bottom) Z-score distribution of BACH1-promoted gene signatures across four stratified patient categories.

FIG. 6L depicts a framework to generate AI-trained histology-predicted gene signatures for patient stratification and response prediction.

FIG. 6M show correlation between histology-predicted and true gene expression signatures. Histology-based predictions of gene expression signatures-including six single-cell derived proliferative clusters, TIL score, and four BACH1-related signatures-were evaluated against ground-truth signatures using five-fold cross validation in TCGA. Error bars denote 95% confidence intervals.

FIG. 6N shows predictive correlation of histology-inferred gene signatures with TIL annotations from pathologists on patient tumor tissues.

FIG. 6O shows correlation between histology-predicted gene expression signatures and pathologist-annotated TILs from tissue.

FIG. 6P shows predictive performance of histology-derived gene expression signatures for pathologic complete response (pCR). Area under the receiver operating characteristic curve (AUROC) values are shown for histology-predicted gene expression signatures across Yale and UChicago patients (Supplementary Table 8).

FIG. 6Q shows that an elastic net model trained on histology-derived gene expression signatures improves prediction of pathologic complete response. Receiver operating characteristic (ROC) curves compare the performance of an elastic net logistic regression model trained on histology-predicted signatures in the UChicago neoadjuvant cohort and validated in the Yale durvalumab plus chemotherapy cohort.

### DETAILED DESCRIPTION

It is to be understood that the particular aspects of the specification described herein are not limited to the specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

All publications, including but not limited to patents and patent applications, cited in this specification are herein incorporated by reference as though set forth in their entirety in the present application.

### Definitions

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indicates otherwise.

In some embodiments, percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the term “level of expression” or “expression level” refers to a measurable level of expression of the products of biomarkers, such as, without limitation, the level of messenger RNA transcript expressed or of a specific exon or other portion of a transcript, the level of proteins or portions thereof expressed of the biomarkers, the number or presence of DNA polymorphisms of the biomarkers, the enzymatic or other activities of the biomarkers, and the level of specific metabolites.

As used herein, the term “differentially expressed” or “differential expression” refers to a difference in the level of expression of the biomarkers that can be assayed by measuring the level of expression of the products of the biomarkers, such as the difference in level of messenger RNA transcript or a portion thereof expressed or of proteins expressed of the biomarkers. In a preferred embodiment, the difference is statistically significant. The term “difference in the level of expression” refers to an increase or decrease in the measurable expression level of a given biomarker, for example, as measured by the amount of messenger RNA transcript and/or the amount of protein in a sample as compared with the measurable expression level of a given biomarker in a control.

As used herein, “sample” or “biological sample” is referred to in its broadest sense, and includes solid and liquid or any biological sample obtained from nature, including an individual, body fluid, cell line, tissue culture, or any other source, which may contain genetic material.

As utilized in accordance with the present disclosure, unless otherwise indicated, all technical and scientific terms shall be understood to have the meaning commonly understood by one of ordinary skill in the art. Unless otherwise required by context, singular terms shall include the plural and plural terms shall include the singular.

Methods of Treatment with BACH1 Inhibitors

The present disclosure provides a method of treating breast cancer in a subject that includes administering a therapeutically effective amount of a transcription factor BTB and CNC Homology 1 (BACH1) inhibitor to the subject. In some embodiments, the method further includes administering a therapeutically effective amount of an anti-cancer agent.

Also provided herein is a method of enhancing therapeutic efficacy of an anti-cancer agent in a subject having breast cancer that includes: a) administering a therapeutically effective amount of a BACH1 inhibitor to the subject, and b) administering a therapeutically effective amount of an anti-cancer agent to the subject.

Inhibition of BACH1 expression and/or activity has therapeutic benefits for non-responders because the Examples have demonstrated that BACH1 suppresses the adaptive immune response and contributes to an immune “cold” phenotype (low immune response) in patients that do not respond to cancer treatments and have low survival outcome. The Examples further demonstrate that a combined treatment of a BACH1 inhibitor with an immune checkpoint CTLA-4 inhibitor produces remarkable therapeutic efficacy compared to a CTLA-4 inhibitor alone. The Examples also provide evidence that breast cancer patients can benefit from the combination of targeting BACH1 expression and/or activity and chemotherapy.

As used herein, an “anti-cancer agent” refers to a compound that is used to kill tumors or inhibit tumor growth in cancer treatment. For example, an anti-cancer agent includes a cytotoxic drug in chemotherapy or an inhibitor of an immune checkpoint protein to increase immune response to kill cancer cells in immunotherapy. In the disclosed methods, a BACH1 inhibitor can enhance the therapeutic efficacy of an anti-cancer agent. In some embodiments, the anti-cancer agent is an inhibitor of an immune checkpoint protein. The immune checkpoint proteins that can be targeted for inhibition include, for example, CTLA4, PD-1, PD-L1, LAG-3, TIM-3, TIGIT, PD-1H, CD278, CD39/CD73, or CD276. In some embodiments, the immune checkpoint protein is CTLA-4. In some embodiments, the inhibitor of CTLA-4 is ipilimumab, tremelimumab, botensilimab, or a combination thereof. In some embodiments, the immune checkpoint protein is PD-1. In some embodiments, the inhibitor of PD-1 is nivolumab, pembrolizumab, cemiplimab, or a combination thereof. In some embodiments, the anti-cancer agent is a cytotoxic drug including, but not limited to, cyclophosphamide, doxorubicin, fluorouracil, epirubicin, paclitaxel, irinotecan, vinorelbine, alkylating agents, topoisomerase inhibitors, anti-metabolites, taxanes, or a combination thereof.

In certain embodiments, the therapeutically effective amount of the BACH1 inhibitor can be administered prior to or concurrent with the therapeutically effective amount of the inhibitor of the immune checkpoint protein.

The present disclosure also provides a method of treating cancer in a subject that includes:
a) taking a sample from a cancer tumor from the subject;
b) detecting a level of expression and/or activity of BACH1 in the tumor; and
c) administering a therapeutically effective amount of the BACH1 inhibitor, if the level of expression and/or activity of BACH1 in the cancer tumor is greater than in cancer subjects that are responsive to an anti-cancer agent.

In some embodiments, a therapeutically effective amount of the BACH1 inhibitor is administered to the subject, if the level of expression and/or activity of BACH1 in the cancer tumor is greater than in cancer subjects that are responsive to an anti-cancer agent.

The present disclosure also provides a method of treating cancer in a subject that includes:
a) taking a sample from a cancer tumor from the subject;
b) detecting a level of expression and/or activity of BACH1 in the tumor;
c) detecting a level of expression of an immune checkpoint protein in the tumor; and
d) administering a therapeutically effective amount of the BACH1 inhibitor and a therapeutically effective amount of an inhibitor of the immune checkpoint protein to the subject.

In some embodiments, a therapeutically effective amount of the BACH1 inhibitor and a therapeutically effective amount of an inhibitor of the immune checkpoint protein are administered to the subject, if a level of expression and/or activity of BACH1 in the cancer tumor is greater than in cancer subjects that are responsive to the inhibitor of the immune checkpoint protein alone without the BACH1 inhibitor, and/or if the level of expression of the immune checkpoint protein in the cancer tumor is scored as high. The described methods above provide a personalized treatment to cancer patients that do not respond to the immune checkpoint therapy. In the patients that are receiving a BACH1 inhibitor, the level of expression and/or activity of BACH1 in the cancer tumor is greater than in cancer subjects that are responsive to an anti-cancer agent that is not a BACH1 inhibitor. In some embodiments, the anti-cancer agent is an inhibitor of the immune checkpoint protein. The Examples show that strikingly, high risk TNBC patients have highest score for BACH1-promoted genes and BACH1-inhibited genes, indicating high BACH1 activity. These patients also do not respond well to cancer treatments and have worse survival outcomes. BACH1-promoted genes refer to genes that are upregulated in expression as a result of BACH1 activity. BACH1-inhibited genes refer to genes that are downregulated in expression as a result of BACH1 activity.

BACH1 activity refers the biological activity of the BACH1 protein that encompasses several functions. BACH1 is a transcription factor that regulates the rate of the transcription of genes involved in reactive oxygen species (ROS) production, the cell cycle, heme homeostasis, hematopoiesis, and immunity [1]. Detection of BACH1-promoted genes and BACH1-inhibited genes provides a comprehensive assessment of BACH1 transcriptional activity in patient's tumor. In some embodiments, a BACH1 distance score is used to quantify BACH1 transcriptional activity by evaluating the differential expression of BACH1-promoted and BACH1-inhibited genes:

$$BACH 1 Distance Score = BACH {1}_{promoted} - BACH {1}_{inhibited}$$

wherein a positive score indicates higher BACH1 transcriptional activity while a negative score suggests reduced BACH1 activity.

A list of BACH1-promoted genes and BACH1-inhibited genes is provided in Table 6 and Table 7. The combination of the genes described in these Tables has never been described elsewhere for the prognosis of cancer. In some embodiments, expression of one or more of BACH1-promoted genes or BACH1-inhibited genes is used to determine BACH1 activity. In some embodiments, expression of at least 2, at least 5, at least 10, at least 15, at least 20, at least 25, at least 30, at least 35, at least 40, at least 45, at least 50, or at least 100 of BACH1-promoted genes and/or BACH1-inhibited genes are used to determine BACH1 activity.

Methods of detecting gene or protein expression of BACH1 or an immune checkpoint protein include, but are not limited to, single cell RNA sequencing, Northern blot, serial analysis of gene expression (SAGE), microarray analysis, quantitative reverse transcription polymerase chain reaction (RT-qPCR), bulk RNA sequencing (bulk RNAseq), Western Blot, immunofluorescence, immunohistochemistry, in situ hybridization, or a combination thereof.

In some embodiments, the level of expression of the immune checkpoint protein is scored as high based on scoring systems that are well known in the art. In some embodiments, the scoring system is histoscore (H-score), tumor proportion score (TPS), combined positive score (CPS), or immunoreactive score (IRS). These scoring systems are designed to categorize the level of expression based on the intensity of staining and/or the number of cells expressing the protein of interest (H-score, TPS, and CPS), or the number of tumor cells and immune cells that express the immune checkpoint protein (CPS). The threshold for which the level of expression of the immune checkpoint protein is considered high is well described in the art [2, 3].

There are currently no drugs targeting BACH1 that have been described. However, it has been reported that heme binding to BACH1 triggers its degradation and thus, inhibits its activity. As such, heme-containing compounds, can be used to inhibit BACH1 [4]. For example, hemin can be used to inhibit BACH1. Other ways of inhibiting BACH1 include gene-editing methods using gene-editing system such as CRISPR/Cas9 that can be used to knock out BACH1 or introduce loss-of-function BACH1 mutations.

In some embodiments, the cancer to be treated is a breast cancer. In some embodiments, the cancer is a triple-negative breast cancer (TNBC). TNBC is an aggressive type of invasive breast cancer that does not respond well to cancer treatments such as immunotherapy, hormone therapy, and/or chemotherapy. It is further contemplated that inhibiting BACH1 alone or in combination with application of one or more other anti-cancer agents might also be beneficial in other types of non-responsive cancer that expresses a high level of BACH1 compared to cancer that is responsive to treatment. Cancers that can be treated include pancreatic cancer, liver cancer, metastatic lung cancer, ovarian cancer, glioblastoma, kidney cancer, esophageal cancer, bile duct cancer, gallbladder cancer, hematologic cancer, acute myeloid leukemia, chronic myeloid leukemia in blast phase, multiple myeloma, non-Hodgkin's lymphoma, mantle cell lymphoma, T-cell lymphoma, Burkitt lymphoma, skin and soft tissue cancers, late-stage melanoma, Merkel cell carcinoma, sarcomas, angiosarcoma, rhabdomyosarcoma, genitourinary cancer, prostate cancer, bladder cancer, testicular cancer, gynecological cancers, advanced stage cervical cancer, endometrial cancer, vaginal cancer, vulvar cancer, head and neck cancer, nasopharyngeal carcinoma, oral cavity cancers, laryngeal cancers, rare and aggressive cancers, neuroendocrine tumors, adrenocortical carcinoma, thymic carcinoma, small cell carcinoma, hereditary or molecularly driven cancers, triple-negative breast cancer, HER2-positive breast cancer, RET-mutated thyroid cancer, pediatric and adolescent cancers, medulloblastoma, neuroblastoma, Wilms tumor, retinoblastoma, digestive system cancers, metastatic colorectal cancer, anal cancer, or small intestine adenocarcinoma. In one embodiment, the cancer is at advanced stage and resistant to cancer therapies. In one embodiment, the cancer is partially responsive cancer therapies. In one embodiment, any cancer can be treated with BACH1 inhibitor alone or in combination with application of one or more other anti-cancer agents.

### Pharmaceutical Compositions

The present disclosure provides pharmaceutical compositions comprising a therapeutically effective amount of a BACH1 inhibitor and a pharmaceutically acceptable carrier, solvent, adjuvant, diluent, or a combination thereof. Also provided herein are pharmaceutical compositions comprising a) a therapeutically effective amount of a BACH1 inhibitor; b) a therapeutically effective amount of an anti-cancer agent; and c) a pharmaceutically acceptable carrier, solvent, adjuvant, diluent, or a combination thereof.

The pharmaceutical compositions can be administered to the subject by intravenous injection to the subject in the methods described herein. “Pharmaceutical composition” as used herein refers to a composition that includes a therapeutically effective amount of a BACH1 inhibitor alone or with a therapeutically effective amount of an anti-cancer agent; and a pharmaceutically acceptable salt, carrier, a solvent, an adjuvant, and/or a diluent, or any combination thereof.

As used herein, the terms “patient,” “subject,” and “individual” can be used interchangeably and refer to an animal. For example, the patient, subject, or individual can be a mammal, such as a human to be treated for a disorder, condition, or a disease.

The term “cancer” as used herein refers to an abnormal growth of cells which proliferate in an uncontrolled way and, in some cases, to metastasize (spread).

“Treating” or “treatment,” as used herein, covers the treatment of a disorder, condition, or a disease described herein, in a subject, preferably a human, and includes:
i. inhibiting a disease or disorder, i.e., arresting its development;
ii. relieving a disease or disorder, i.e., causing regression of the disorder;
iii. slowing progression of the disorder; and/or
iv. inhibiting, relieving, ameliorating, or slowing progression of one or more symptoms of the disease or disorder. For example, the terms “treating,” “treat,” or “treatment” refer to either preventing development or exacerbation of, providing symptomatic relief for, or curing a patient's disorder, condition, or disease.

As used herein, the term “therapeutically effective amount” means at least the minimum dose of a BACH1 inhibitor or an anti-cancer agent, or a pharmaceutical composition comprising the BACH1 inhibitor and/or the anti-cancer agent disclosed herein, necessary to achieve a desired therapeutic or prophylactic effect. Some embodiments include an amount sufficient to prevent or disrupt cancer progression, kill tumor cells, or reduce the extent or duration of cancer disease. Some embodiments include a dose sufficient to reduce a symptom associated with the cancer being treated. An effective dosage or amount of the BACH1 inhibitor or the anti-cancer agent or a pharmaceutical composition comprising the BACH1 inhibitor and/or the anti-cancer agent disclosed herein can readily be determined by the person of ordinary skill in the art considering all criteria.

For purposes of this invention, treating cancer includes, without limitation, alleviating one or more clinical indications, decreasing tumor growth or tumor cell proliferation, reducing the severity of one or more clinical indications of cancer condition, diminishing the extent of the condition, stabilizing the subject's disease state (i.e., not worsening), delaying or slowing, halting, or reversing cancer progression, and bringing about partial or complete remission. Treating cancer also includes prolonging survival by days, weeks, months, or years as compared to prognosis if treated according to standard medical practice not incorporating or administering a BACH1 inhibitor, or a BACH1 inhibitor and an anti-cancer agent, according to a method provided herein.

Unless otherwise defined, all technical terms used herein have the same meaning as commonly understood by a person of ordinary skill in the art to which this disclosure belongs.

For the purposes of promoting an understanding of the principles of the disclosure, reference will now be made to embodiments and specific language will be used to describe the same. It will nevertheless be understood that no limitation of the scope of the disclosure was thereby intended, such alteration and further modifications of the disclosure as illustrated herein, being contemplated as would normally occur to one skilled in the art to which the disclosure relates.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### Examples

The Examples that follow are further illustrations of specific embodiments of the disclosure, and various uses thereof. They are set forth for explanatory purposes only and should not be construed as limiting the scope of the disclosure in any way.

In the Examples, analysis of tumors obtained from xenograft TNBC mouse models reveals distinct gene clusters that are associated with different states of cancer progression. Furthermore, the gene clusters associated with aggressive cancer states, called high stress cluster signatures, comprise the most hypoxia-induced genes compared to clusters associated with milder states. The analysis also identifies cluster 0 as a central module that interfaces with other clusters and most likely is a transition cluster from mild state to aggressive state. Strikingly, the gene expression profile of tumor samples obtained from clinical breast cancer patients also show correlation with these gene clusters, with patients having worse survival and treatment outcome displaying genes in high stress cluster signatures. The Examples further demonstrate that BACH1 regulates expression of several genes in cluster 0 and acts as a regulator of phenotypic states in cancer. BACH1 activation is regulated by hypoxia, PHD1 inhibition, and gain-of-function proline mutations. Significantly, high risk TNBC patients have the highest score for BACH1-promoted genes and BACH1-inhibited genes, indicating high BACH1 activity. These patients are also associated with reduced Tumor-infiltrating Lymphocyte (TIL) gene signatures, indicating low immune response (an “immune cold” phenotype). This finding lead to the discovery that BACH1 activity worsens treatment outcomes for cancer patients by impacting the responsiveness of the immune system. Finally, the Examples demonstrate that a combination of a BACH1 inhibitor with an immune checkpoint CTLA-4 inhibitor significantly improves therapeutic outcomes compared to treatment with a CTLA-4 inhibitor alone.

### Methods and Materials

Cell culture: several human triple-negative breast cancer (TNBC) cell lines were used: MDA-MB-231 (ATCC), MDA-MB-436 (ATCC), BM154 (also known as MDA-MB-231-BM1, BoM1, 1833, MSK), SUM159PT (ATCC), TU-BcX-4IC (Tulane University School of Medicine), the mouse TNBC cell lines 4T1 (ATCC) and LMB (also known as E0771-LMB), and the human 293T cell line (ATCC). The E0771-LMB (LMB) cells were generated by Robin Anderson [5], BM1 cells by Massagué and colleagues [54], and TU-BcX-4IC cells by M.D. Matossian and colleagues [55]. MDA-MB-231, MDA-MB-436, TU-BcX-4IC, SUM159PT, LMB, and 293T cells were cultured in DMEM, while 4T1 cells were cultured in RPMI-1640. Media were supplemented with 10% fetal bovine serum (FBS), 50 U/mL penicillin, and 50 μg/mL streptomycin. All cell lines were authenticated by short tandem repeat analysis and used within 15 passages after arrival in the laboratory. Mycoplasma detection was routinely performed using the MycoAlert Detection kit (Lonza, LT07-218) to ensure cells were mycoplasma-free. All cell cultures were maintained at 37° C. with 5% CO₂, unless otherwise indicated.

Patient-Derived Organoid (PDO) Establishment and Maintenance: the procurement of biospecimens for the generation of breast cancer patient-derived organoids (PDOs) was conducted in accordance with the approved institutional review board protocol (16352A) and with signed patient consent. All patient samples and associated clinical information were deidentified to protect patient confidentiality. Fresh core needle biopsy samples or cells from pleural effusions were obtained as remnant specimens without compromising necessary diagnostic procedures. PDOs were established as previously described [56]. Briefly, tissue from core biopsies was enzymatically digested for 1-2 hours to generate a cell suspension. Cells from pleural effusions were collected by centrifugation at 450 g for 10 minutes and washed in Advanced DMEM-F12 (ThermoFisher: 12634010) supplemented with 10 mM HEPES (ThermoFisher: 15630080), 1×GlutaMAX (ThermoFisher: 35050061), and 1×Antibiotic-Antimycotic (ThermoFisher: 5240062) (AdvM+). The resulting cell pellets were mixed with Matrigel (Corning: 356237), plated into prewarmed 24-well plates (Greiner Bio-One: 662102) to allow Matrigel to polymerize at 37° C., and cultured in breast cancer PDO media to support organoid growth. Breast cancer PDO media are AdvM+ medium supplemented with Y27632 (5 μM; AbMole: M20999), R-Spondin-3 (250 ng/ml; ThermoFisher: 120-44), Noggin (100 ng/ml; ThermoFisher: 120-10C), B27 (1X; ThermoFisher: 17504044), NIC (5 mM; Sigma: N0636-100G), NAC (1.25 mM; Sigma: A9165-5G), Primocin (100 μg/mL; Invitrogen: ant-pm-1), EGF (5 ng/mL; ThermoFisher: AF-100-15), FGF-10 (20 ng/mL; ThermoFisher: 100-26), A83-01 (500 nM; Tocris: 2939), FGF-7 (5 ng/ml; ThermoFisher: 100-19), SB202190 (500 nM; Sigma: S7067), and Heregulin beta-1 (5 nM; ThermoFisher: 100-03). To prevent misidentification and cross-contamination, each PDO sample was cultured in a separate, uniquely labeled dish. Identity of PDOs was confirmed by DNA fingerprinting using the AmpFLSTR® Identifiler® PCR Amplification Kit (ThermoFisher: 4322288). All PDOs were tested for mycoplasma contamination. Once organoids reached a size of 50 to 100 μm, they were harvested for subsequent experimental applications. To dissociate from Matrigel, PDOs were incubated with Dispase (STEMCELL Technologies: 07913) at 37° C. for 20 min, washed twice with AdvM+, and centrifuged at 450 g for 5 min. To obtain a single-cell suspension, the PDO pellet was incubated with TrypLE (ThermoFisher; 12563011) at 37° C. for 5 to 10 min. Trypsinization was stopped with 10% FBS, and cells were washed twice with AdvM+ and centrifuged at 450 g for 5 min. All PDO cultures were maintained at 37° C. with 5% CO₂, unless otherwise indicated.

Generation of Stable Knockout Cell Lines: to generate knockout (KO) cells, lentivirus stocks were produced using the lentiCRISPR v2 plasmid (Addgene) containing a single guide RNA (Table 4). Lentiviral supernatants were harvested at 72 hours post-transfection, spun at 2000 g for 10 minutes at 4° C., and filtered through a 0.45 μm PES filter. The viral media was then transferred to the target cells for knockout, supplemented with 8 μg/mL polybrene (Millipore Sigma, TR1003G), and incubated for 24-48 hours. The infected cells were polyclonally selected and validated by western blot and maintained in medium supplemented with 4 g/mL puromycin for 4-10 days (duration dependent on cell line) until all non-transfected cells died.

Generation of Stable Cell Lines with BACH1 expression manipulation: BM1 Control and BM1 stably overexpressing mBACH1 cell lines were generated through lentiviral transduction using the lentiviral vectors generated by Lee and colleagues [23]. Mutant mBACH1 plasmids (P182A, P530A, or P182A/P530A) were generated using Q5 Site-Directed Mutagenesis (New England Biolabs). The mutations were introduced into Wild-Type (WT) mBACH1 (optimized codons) cloned into the pLV-EF1a-IRES-Hygro backbone (Addgene 85134). WT or mutant mBACH1 lentiviral vectors were transduced to re-express mBACH1 in 4T1 or BM1 BACH1 KO cells. Hygromycin selection (300 μg ml⁻¹) was applied for 2 weeks. To generate ectopically inducible BACH1 cells: the MDA-MB-231 BACH1 KO cells were subsequently co-transfected with the LPutopia-7 donor vector and AAVS1 sgRNA to establish the stable MDA-MB-231 BACH1-KO-LPutopia (BK-LP) parental cell line as previously described [57]. To generate monoclonal populations for circuit reintroduction, single-cell sorting was performed. To reintroduce the ectopic WT and double-mutant BACH1 P182A and P527A (BACH1DM) constructs, site-directed mutagenesis was applied at nucleotide 177, converting C to T, to disable the protospacer adjacent motif (PAM) recognition by Cas9 and prevent off-target cleavage of the ectopic BACH1 copy. The silent-mutated BACH1 and BACH1DM sequences were then cloned into the negative-feedback circuit frame (pUt-mNF) and inserted individually into the AAVS1 locus of MDA-MB-231 BK-LP cells via a recombinase-mediated cassette exchange (RMCE) process. Cells were selected using 5 μg/mL blasticidin as described [57]. Enriched recombinants were further processed through fluorescence-activated cell sorting (FACS) to isolate properly engineered cells for subsequent experiments.

Chromatin Immunoprecipitation (ChIP) Single-cell RNA sequencing: BM1, MDA-436, or BM1-BACH1 KO cells overexpressing BACH1 (WT or mutants) (10×10⁶ cells) were treated under either normoxic conditions, hypoxic conditions at 1% O₂ for 24 hours, or a combined treatment of 24 hours of hypoxia (1% O₂) with glucose-limited conditions (1 mM glucose). Cells were crosslinked with 1% formaldehyde for 10 minutes at 37° C. and immediately quenched with a final concentration of 125 mM glycine for 1 minute. Cells were then lysed and sheared in a buffer containing 0.1% SDS, 50 mM Tris-HCl (pH 7.6), 1 mM EDTA (pH 8.0), 0.2% Triton X-100, PMSF, and a protease inhibitor (Roche 04693159001). Lysates were sonicated using a Bioruptor in four cycles (30 seconds on/30 seconds off per cycle) for ChIP-qPCR. For ChIP-seq preparation, the sonication step was optimized to 30 cycles, resulting in chromatin fragments between 150 and 300 bp. The sonicated chromatin was vortexed and incubated on ice for 10 minutes, then centrifuged at 16,000 g for 10 minutes at 4° C. Ten percent of the supernatant was saved as input. The remaining supernatant was incubated with an antibody (rabbit polyclonal anti-Bach1 (A1-6) [24]) or normal rabbit serum conjugated with Dynabeads Protein A/G for 150 minutes at 4° C. Beads were washed twice for five minutes each with RIPA buffer (10 mM Tris-HCl, pH 7.6, 1 mM EDTA, 0.1% SDS, 0.1% NaDOC, 1% Triton X-100), RIPA buffer with 0.3 M NaCl, LiCl buffer (0.21 M LiCl, 0.5% NP-40, 0.5% NaDOC), and TE buffer with 0.2% Triton X-100. Finally, the beads were washed once with TE buffer for five minutes. Beads were resuspended in 100 μL of TE (pH 8.0) with 0.3% SDS, supplemented with 1 mg/mL Proteinase K (Invitrogen 25530049), and incubated for 4 hours at 65° C. with agitation at around 1000 rpm. ChIP-DNA was purified using AMPure XP beads (Beckman Coulter A63881). ChIP-DNA samples were prepared for library construction and sequencing, either as described at Tohoku University School of Medicine or at the University of Chicago sequencing core facility or subjected to qPCR analysis. The list of human primers used for the ChIP assay quantitative RT-PCR is provided in the Table 3.

ChIP-seq and analysis: the DNA sample quality check, library construction and sequencing were performed by the University of Chicago Genomics Facility following standard protocols. All samples were sequenced across four runs using a NovaSeq 6000 platform, generating paired-end 100-base pair reads. For each sample, raw FASTQ files from four flow cells were merged prior to downstream analysis. ChIP-seq data processing and analyses were performed as described [42]. ChIP-seq data processing was conducted using a local Galaxy 20.05 instance [59]. Quality and adapter trimming of raw reads were performed with Trim Galore! (v0.6.3). Trimmed reads were aligned to the human reference genome (UCSC hg19 with GENCODE annotation) using BWA-MEM (version 0.7.17.1) [60]. To visualize ChIP-seq results, the mapped reads files were counted and the resulting TDF files were graphed using Integrative Genomics Viewer (version 2.9.4) [61]. The mapped reads were converted to a SAM file format using SAMtools (version 1.2) [62]. Peak calling, motif analysis and annotations were performed by HOMER 4.11.1 using the IP and input SAM files for each sample [63]. Unique and overlapping peaks between the hypoxia and normoxia samples were determined on the basis of whether peak centers were within a 100-bp distance. HOMER was also used to detect the presence of the MARE motif TGCTGASTCA, AP-1 motif TGASTCA, and HRE motif CACGT within the #150-bp region surrounding the peak center for the sample peaks. deepTools 3.3.2 was used to compare differences in BACH1 binding with or without hypoxia [64]. Specifically, each sample's IP reads were compared to its input reads and then normalized to total read count. Signals at each binding peak were calculated and then plotted as a heat map for each sample. The average BACH1 signal for the normoxia and hypoxia samples were determined by calculating the average across all peaks for each sample using the peak coordinates of the hypoxia sample. The signals of normoxia and hypoxia samples were compared for differential binding using GraphPad Prism 10 (GraphPad Software, San Diego, CA).

Western Blot: cultured cells were washed with cold PBS and lysed in RIPA buffer containing protease (Millipore Sigma, 539134) and phosphatase inhibitors (GoldBio, GB-450). Tumor samples were snap-frozen in liquid nitrogen, pulverized, and lysed in the same buffer. All samples were sonicated (3×10 s at 35% power) and centrifuged at max speed for 15 min at 4° C. Protein concentration was measured using the Bradford assay, and lysates were boiled in Laemmli buffer immediately after measurement. Equal amounts of protein (10-50 μg) were loaded onto SDS-PAGE gels (Bio-Rad), transferred to PVDF membranes at 300 mA for 2 hours, and blocked for 1 hour at room temperature using Odyssey Blocking Buffer (LI-COR, 927-40010, diluted 1:1 with PBS) or 5% Skim milk in TBS-T. Membranes were incubated overnight with primary antibodies at 4° C. and with secondary antibodies for 1 hour at room temperature. Blots treated with ECL reagent (Thermo Scientific, 32106) were imaged on the Chemiluminescence channel, while fluorescent blots were imaged at 700 nm or 800 nm using the LI-COR Fc system. Signal intensity was quantified with Image Studio Lite software (LI-COR). Details about antibodies, their concentrations, blocking conditions, and additional notes are provided in the accompanying Table 1.

RNA Isolation and quantitative real-time polymerase chain reaction (qRT-PCR): tumor RNA was extracted using the Direct-zol™ RNA MiniPrep Kit (Zymo Research, R2052) following lysis in TRI Reagent (Zymo Research, R2050-1-200), while in vitro RNA was isolated using the Qiagen RNeasy Kit (74124). RNA concentration and purity were measured with a NanoDrop spectrophotometer. A total of 2 μg RNA per sample was reverse-transcribed into complementary DNA (cDNA) using the High-Capacity cDNA Reverse Transcription Kit (Applied Biosystems, 4368813). cDNA was diluted 1:20 in molecular biology-grade water and stored at −30° C. qRT-PCR was performed on a Roche LightCycler® 96 Instrument using the PowerUp™ SYBR™ Green Master Mix (Applied Biosystems, A25776). Each reaction consisted of 16 μL master mix (10 μL SYBR™ Green, 4 μL H₂O, and 2 μL 5 μM primer mix) and 4 μL of 1:20 diluted cDNA, in a total volume of 20 μL. Amplification and detection were conducted according to the manufacturer's protocols, and data were analyzed using the Roche LightCycler® 96 software. Primer details are provided in Table 2.

Mouse studies: all animal experiments were conducted in accordance with the University of Chicago Institutional Animal Care and Use Committee (IACUC; protocol no. 72439). Mice were housed in specific pathogen-free conditions, maintained at a controlled temperature (20-25° C.), humidity (30-70%), and a 12-hour light-dark cycle. For Primary Tumor Growth and Metastasis studies, BM1 (5×105), LMB (3×10⁵), 4T1 (2×10⁴), or MDA-MB-231 (2×10⁶) cells were injected orthotopically into the mammary fat pad of athymic nude, C57Bl/6, or BALB/c mice, as described for each experiment. MDA-MB-231 cells were mixed 1:1 with Matrigel (Cultrex Basement Membrane Extract, Type 3, PathClear; R&D Systems, Cat. #3632-010-02) in a total volume of 100 μL prior to injection. For immune checkpoint blockade experiment, BALB/c mice were orthotopically implanted with 20,000 4T1 mouse breast cancer cells into the mammary fat pad. Beginning three days post-inoculation, mice were treated with immune checkpoint blockade antibodies. Anti-mouse PD-1 (clone RMP1-14, Catalog #BE0146), anti-mouse PD-L1 (clone 10F.9G2, Catalog #BE0101), and anti-mouse CTLA-4 (clone 9H10, Catalog #BE0131) were administered intraperitoneally at 100 μg per dose as [65], twice weekly, for a total of six doses. Tumor growth was monitored twice weekly using digital calipers, measuring two perpendicular dimensions. Tumor volume was calculated using the formula:

$$Volume = \frac{π}{6} \times {width}^{2} \times length$$

Tumor volumes were reported as mean±s.e.m., and statistical significance was determined using multiple unpaired t-tests. Tumors were harvested for downstream analyses, including spatial transcriptomics, single-cell RNA-seq, bulk RNA-seq, or fixation for histological assessment. For lung metastasis efficacy studies, lungs were excised, fixed in Bouin's solution, washed with 70% ethanol, and processed at the University of Chicago histology core. Lung sections were imaged with a 40× objective on an Olympus VS200 microscope and analyzed with QuPath software in a blinded manner. Metastatic tumors were identified and scored by an experienced pathologist in a blinded manner. Tumors were fixed in 10% formalin for 24 hours, transferred to 70% ethanol, and processed at the University of Chicago Human Tissue Resource Center.

Electron Paramagnetic Resonance (EPR) Oxygen Imaging: EPR oxygen imaging (EPROI) offers images with spatial resolution of ~1 mm and oxygen resolution ~1 torr [66, 67]. For images oxygen spin probes with oxygen sensitive relaxation times are used. Preparation of spin probe: Trityl OX071 (methyl-tris[8-carboxy-2,2,6,6-tetrakis [(2-hydroxyethyl]benzo[1,2-d:4,5-d′]bis[1,3]dithiol-4-yl]-trisodium salt) in powder form was purchased from GE Healthcare (Little Chalfont, Buckinghamshire, UK). A 72 mM trityl solution was prepared at neutral pH. The solution was sterilized by filtration through a 0.22 μm syringe filter. Animal Preparation for Imaging and Trityl Injection: Mice were weighed and anesthetized with an isoflurane-medical air mixture (1.5 L/min) inside a vented chamber. To maintain hydration during imaging, 1 mL of saline was administered. The trityl solution was injected i.v. at a rate of 0.8 mL/h. The total injection volume varied between 160-300 μL per animal, adjusted based on imaging feedback aiming to achieve optimal signal coverage with minimum injection. Throughout the imaging procedure, the animals' physiological parameters were maintained within controlled ranges: Breathing rate: 90-110 breaths/min; Core body temperature: 38.1° C. These parameters were regulated by adjusting anesthesia levels and the heating system power. Imaging was commenced at the start of the spin probe injection and continued in a sequence of 4-6 acquisitions, each lasting 7.5 minutes. For analysis, later images in the sequence acquired after the end spin-probe injection were used to minimize the influence of arterial blood oxygenation on observed oxygen levels. EPR Imaging System and Parameters: A 720 MHz EPR oxygen imager, developed at the University of Chicago, with a 26 mT permanent magnet (Jansen NMR Technology Co. Ningbo, CN) was used. The system was equipped with a (d=25 mm, L=20 mm) resonator. The animal was positioned within the resonator to ensure complete tumor coverage. Imaging parameters:
Pulse sequence: Inversion recovery spin echo (IRESE) [67]
π/2 and π pulse durations: 60 ns
Phase cycling: 16-step scheme
Spin-echo delay: 400 ns
Projection scheme: 208 projections with equal solid angle spacing, 53 baselines
Gradient strength: 0.75 G/cm
Time delays: 8 logarithmically spaced delays (410 ns-10 μs)
Repetition time: 35 μs
Image resolution: 64×64×64 (FOV: 4.236 cm)

EPR image reconstruction was performed using filtered back projection, and image analysis was carried out using the ArbuzGUI MATLAB toolbox [68]. For tumor delineation, T2-weighted 9.4T MRI image of the mice located in the same bed were taken. EPR and MRI images were registered using anatomical landmarks and fiducials embedded in the mouse bed. Tumor segmentation was transferred from MRI to EPR image coordinate system.

Metastasis Colonization Assays: luciferase-expressing BM1 cells (BM1-1uc; 1×10⁵) were injected into the left ventricle of the heart to enable systemic distribution and metastasis. Metastatic burden was quantified using the IVIS Imaging System (Xenogen, PerkinElmer) following a standard luciferin bioluminescence assay. D-luciferin (Gold Biotechnology, Cat. No. LUCK-1G) was prepared at 15 mg/mL in PBS (pH 7.8) and administered intraperitoneally at a dose of 150 mg/kg body weight. Imaging was performed 10 minutes post-luciferin injection under isoflurane anesthesia, with identical settings across all mice. Data were analyzed using Living Image Software (PerkinElmer).

Immunohistochemistry: tissue sections were processed using the Leica Bond RX automated stainer. Following deparaffinization, rehydration, and antigen retrieval (20 minutes heat treatment using AR9961, AR9640, or AR9661, Leica Biosystems), the following antibodies were applied: Anti-CD3 (Abcam, Clone SP162, 1:100, 60 minutes), Anti-CD4 (Sysy-HistoSure, Clone 78H9D2, 1:200, 60 minutes), Anti-CD8 (eBioscience, Clone 4SM15, 1:800, 60 minutes), and Anti-B220 (BD Pharmingen, Clone RA3-6B2, 1:800, 60 minutes). Detection was performed using the Bond Polymer Refine Detection Kit (DS9800, Leica Biosystems) or the ImmPress anti-rat IgG Polymer-HRP Kit (MP-7444, Vector Laboratories) with DAB staining. For F4/80 staining, antigen retrieval was conducted in DAKO buffer (S1699) at 97° C. for 20 minutes, followed by incubation with Anti-F4/80 (AbD Serotec, 1:200, 60 minutes), biotinylated anti-rat IgG (10 μg/mL), and detection with the VECTASTAIN Elite ABC kit (PK-6100) and DAB. All sections were counterstained with hematoxylin, washed, and mounted with cover glasses. Tissue sections were imaged using an Olympus VS200 microscope and analyzed with QuPath software in a blinded manner.

Cytokine Profiling: Flash-frozen tumors were lysed in 1X Buffer A using gentleMACS™ M Tubes. The buffer A contained 50 mM Tris-HCl (pH 7.4), 150 mM NaCl, 0.5% NP-40, 1 mM EDTA, and was supplemented with protease and phosphatase inhibitors, PMSF, and water. Lysates were normalized to 2.0 μg/μL. Cytokine levels were quantified using the LEGENDplex™ Mouse Cytokine Release Syndrome Panel (13-plex) and analyzed by flow cytometry.

Bulk-RNA Sequencing and Data Analysis: RNA sample quality assessment, library construction, and sequencing were performed at the University of Chicago Genomics Facility following standard protocols. All samples were sequenced across two runs using a NovaSeq 6000 platform, generating paired-end 100-base pair reads as described [69]. For each sample, raw FASTQ files from two flow cells were merged prior to downstream analysis. RNA-seq data processing was conducted using a local Galaxy 20.05 instance [59]. Quality and adapter trimming of raw reads were performed with Trim Galore! (v0.6.3). Trimmed reads were aligned to the human reference genome (UCSC hg19) using RNA STAR (v2.7.5b) [70]. Mapped reads were quantified at the gene level with featureCounts (v1.6.4) to produce per-gene read counts. Differential expression analysis between experimental conditions was performed using DESeq2 (v1.22.1) [72]. DESeq2 normalized counts from RNA-seq for all samples were transformed to Z-scores and plotted by the Morpheus software (Broad Institute): software.broadinstitute.org/morpheus.

Assay for Transposase-Accessible Chromatin with sequencing (ATAC-seq): cells exposed to normoxia or hypoxia for the indicated durations were cryopreserved using CELLBANKER® 2 (AMSBIO). ATAC-seq library preparation and sequencing were performed by Azenta Life Sciences (South Plainfield, NJ, USA). Live cells were thawed, washed, and treated with DNase I (Life Technologies, Cat. #EN0521) to remove genomic DNA contamination. Cell viability and concentration were assessed using the Countess Automated Cell Counter (Thermo Fisher Scientific, Waltham, MA, USA). Following cell lysis and cytosol removal, nuclei were isolated and tagmented using Tn5 transposase (Illumina, Cat. #20034197) at 37° C. for 30 minutes. Tagmented DNA was purified using the MinElute PCR Purification Kit (Qiagen, Cat. #28004) and barcoded with the Nextera Index Kit v2 (Illumina, Cat. #FC-131-2001). Libraries were PCR-amplified, purified using SPRI beads, and sequenced on an Illumina HiSeq 4000 (2×150 bp paired-end configuration). Image analysis and base calling were conducted using HiSeq Control Software (HCS), and raw sequencing data (.bcl files) were converted to FASTQ format and demultiplexed using bcl2fastq v2.17, allowing for one mismatch in index sequence identification. Chromatin accessibility using the nf-core/ATAC-seq pipeline (v2.1.2) was analyzed with Nextflow and Singularity for standardized and reproducible execution [73-75]. Raw sequencing reads were assessed for quality, adapter contamination, and GC content distribution using FastQC. Low-quality reads and adapter sequences were removed before alignment to the GRCh38 reference genome. Peak calling was performed using MACS2 [76-77] in narrow peak mode to detect open chromatin regions with high resolution, incorporating control samples to refine peak calling and minimize background noise. Chromatin accessibility around key genomic regions was evaluated using deepTools [78], computing signal intensities at transcription start sites (TSS) and genomic features. The computeMatrix function quantified read coverage, followed by plotProfile and plotHeatmap visualizations for accessibility pattern analysis. Fragment size distribution and TSS enrichment scores were generated and stored for downstream analyses. Peak annotation was conducted using ChIPseeker [79], mapping peaks to promoters, exons, and introns. Differential chromatin accessibility was analyzed using DiffBind [80], contrasting hypoxia vs. normoxia with fold-change (0.4) and p-value (0.05) thresholds. TMM normalization was applied to adjust for sequencing depth variations, and annotated peak reports were aggregated and outputted in Excel format for further interpretation.

Proteomic quantification: BM1 Cells were washed three times with (PBS) and lysed directly in 1 mL of 8M urea supplemented with protease (Millipore Sigma, 539134) and phosphatase inhibitors (GoldBio, GB-450). Cells were lysed under either normoxic (21% (2) or hypoxic (1% O₂) conditions in a hypoxia chamber (Coy Laboratory Products, Inc, Grass Lake, MI, US). The protein lysate of interest was first treated with 5 mM dithiothreitol (DTT) for 30 minutes at 56° C. followed by the alkylation step by adding 15 mM iodoacetamide (IAA) and incubating for 30 minutes in the dark at room temperature. The alkylation reaction was quenched by 30 mM cysteine for 30 mins at room temperature followed by dilution with 100 mM ammonium bicarbonate (Sigma-Aldrich, Inc., St. Louis, MO) at a ratio of 1:3 (v:v). The sample was then digested by trypsin (Promega Corp., Madison, WI) for 16.5 hrs i. The digested peptides were then labeled by tandem mass tag (TMT) reagents (#90066, Thermo Fisher Scientific, Inc., Waltham, MA) following the manufacturer's protocol. Equal quantities of labeled peptides for all samples were mixed and off-line fractionated using the Pierce™ high-pH reversed-phase kit (#84868, Thermo Fisher Scientific, Inc., Waltham, MA). The mixed peptides were loaded onto a home-made silica column (18 cm length×3 μm ID) packed with C18 resin (Dr. Maisch GmbH, Beim Bruckle, Germany). HPLC-MS/MS was performed on an Orbitrap Exploris™ 480 mass spectrometer (Thermo Fisher Scientific, Inc., Waltham, MA) coupled with an EASY-nLC 1000 system (Thermo Fisher Scientific, Inc., Waltham, MA). Mobile phase A was 0.1% formic acid in water, and mobile phase B was 0.1% formic acid in acetonitrile (v/v). The eluting flow rate was 0.3 μL/min. Samples were separated and eluted with a gradient of 5% to 35% mobile phase B in mobile phase A over 60 min. Under the positive-ion mode, full-scan mass spectra were acquired over the m/z range from 300 to 1,400 using the Orbitrap mass analyzer with mass resolution of 60,000. MS/MS fragmentation was performed in a data-dependent mode, in which the 15 most intense ions were selected for MS/MS analysis at a resolution of 15,000 using collision mode of HCD. Other important parameters: isolation window, 2.0 m/z units; default charge, 2+; normalized collision energy, 30%; maximum IT, auto; AGC target, standard; dynamic exclusion, exclude after 2 times within 20 s. MS/MS spectra were searched against the reverse-concatenated non-redundant FASTA human database compiled from the UniProt database by ProteomeDiscoverer 2.5 with precursor and fragment ion tolerances of 10 ppm and 0.06 Da, respectively. Fixed modifications of TMT (+229.163 Da) on lysines and N-termini and carbamidomethylation (+57.021 Da) of cysteines were added while variable oxidation (+15.995 Da) of methionine was considered. Maximum missed cleavage site number was 2. TMT quantitation of each protein was performed by summing reporter ion intensities across all matching peptide-spectral-matches with corresponding TMT data.

Statistical analysis: Western blot, EMSA data are representative of at least 2-6 independent biological replicates. Individual points in graphs plotting data from RT-qPCR, invasion, and ChIP-qPCR assays represent independent biological replicates and are presented as mean±s.d. Statistical significance was determined using Student's t-tests. ChIP-seq was performed as two independent biological replicates. For animal experiments, tumour growth data are shown as mean±s.e.m. Statistical significance was determined using Student's t-tests. RNA-seq and gene signature cut-off analyses are described in the Methods and Tables 5-7. The 2-oxoglutarate turnover results were representative of at least four independent experiments. P-values for KMplot [81, 82] analyses were obtained from the Kmplot website.

Gene Set Enrichment Analysis: gene expression data from bulk RNA-seq and mass spectrometry were used for GSEA analysis (version 4.1.0) [83, 84]. To identify the most significantly enriched pathways, the gene lists from RNA-seq, ATAC-seq, ChIP-seq, and scRNA-seq were analyzed using Metascape [53].

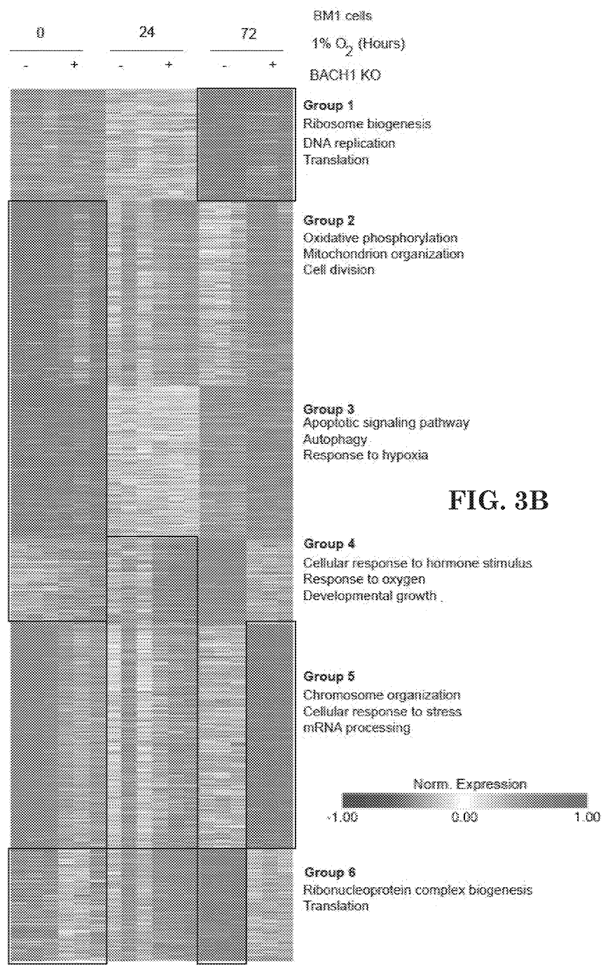

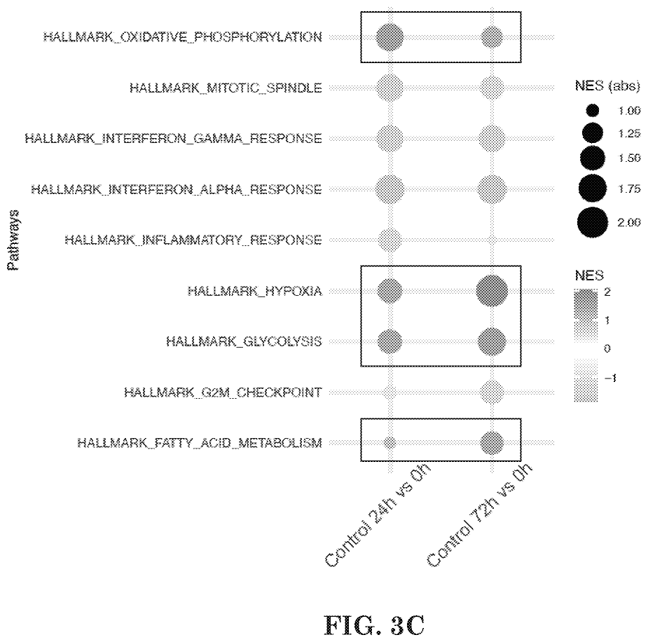

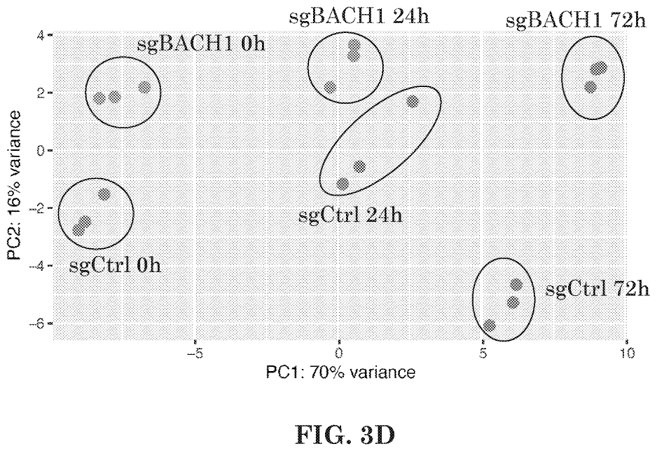

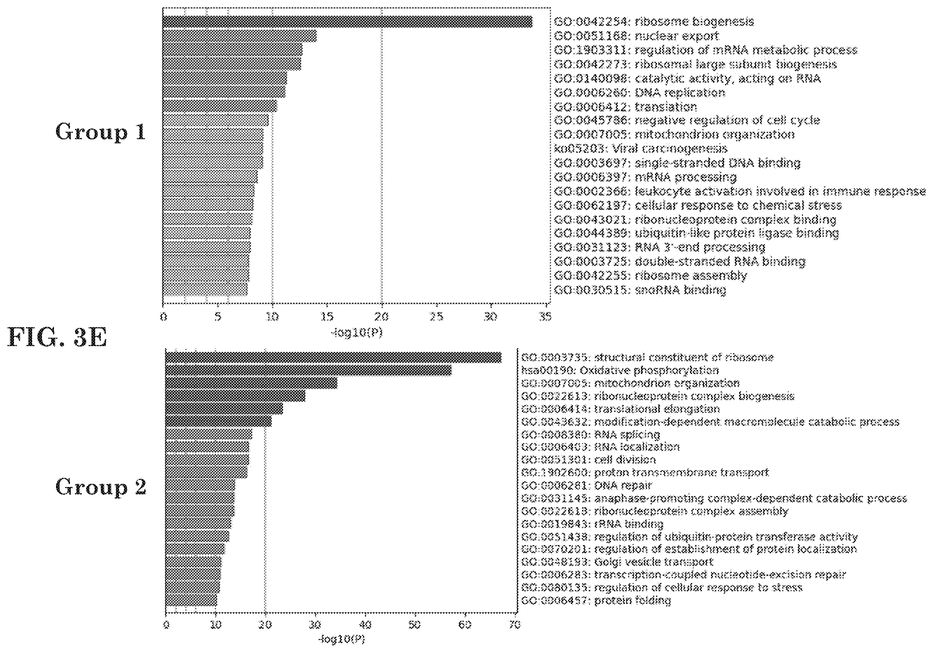

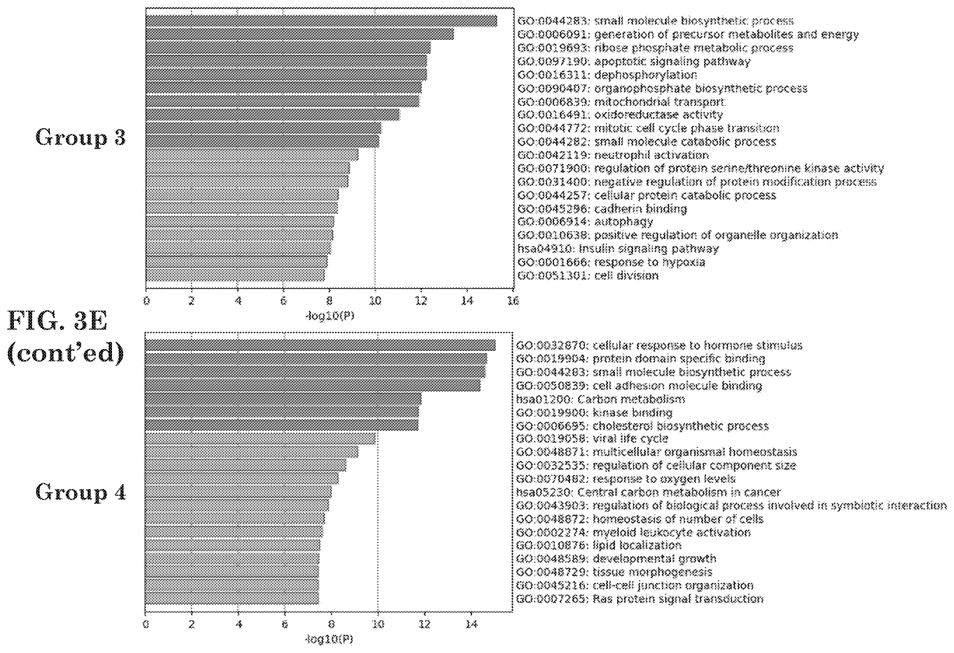

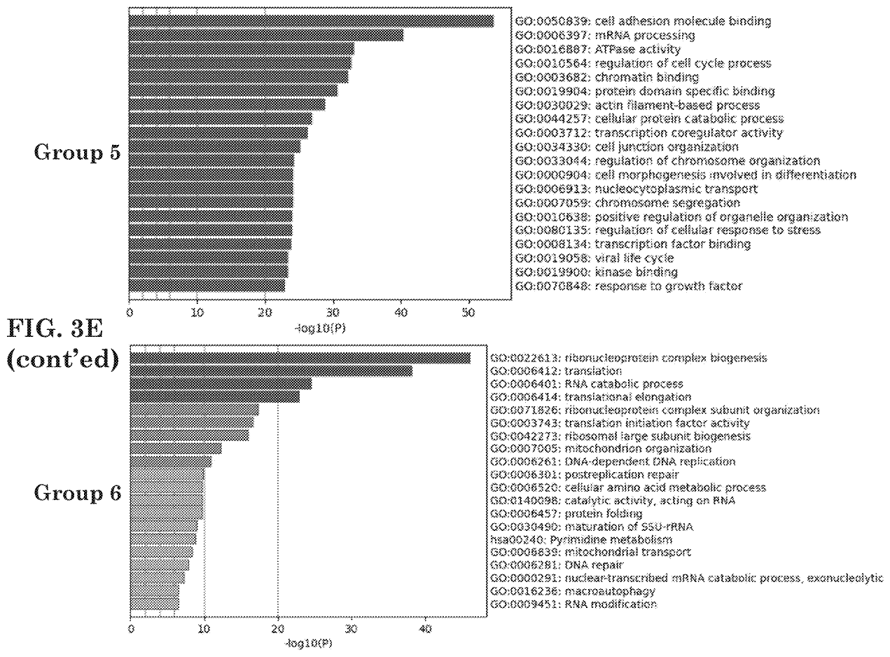

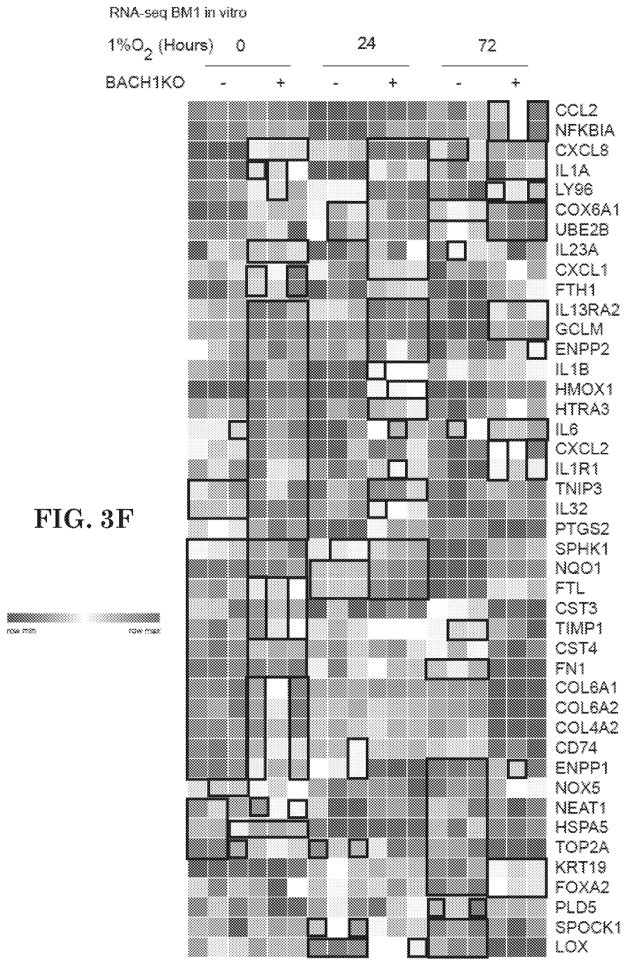

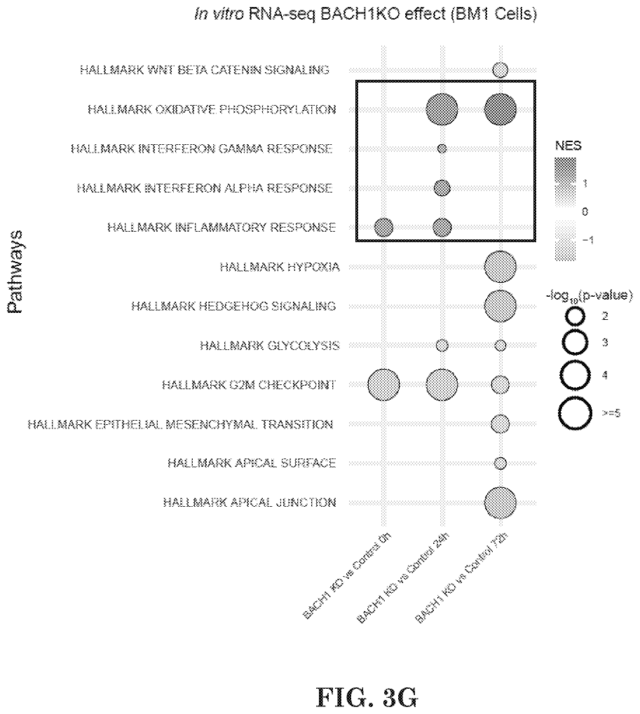

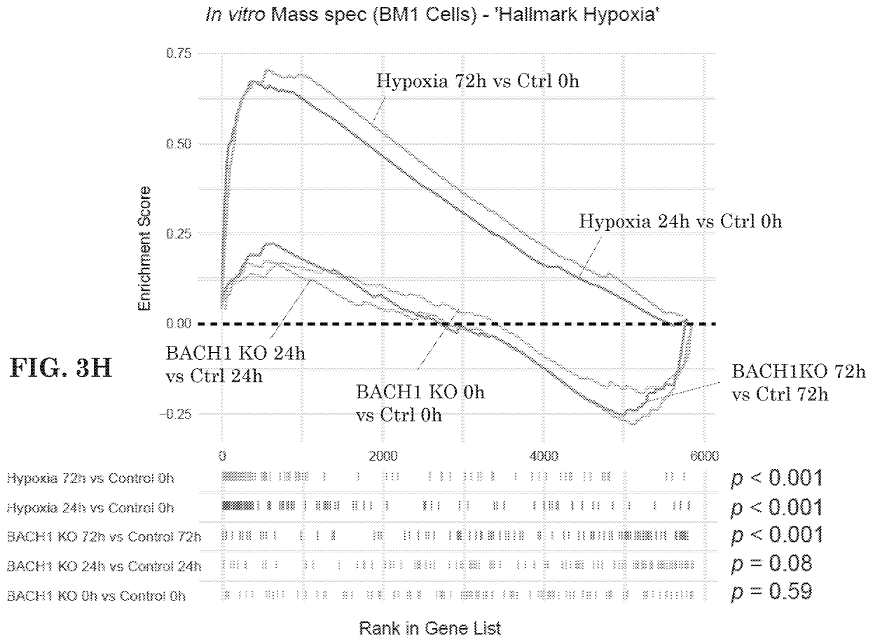

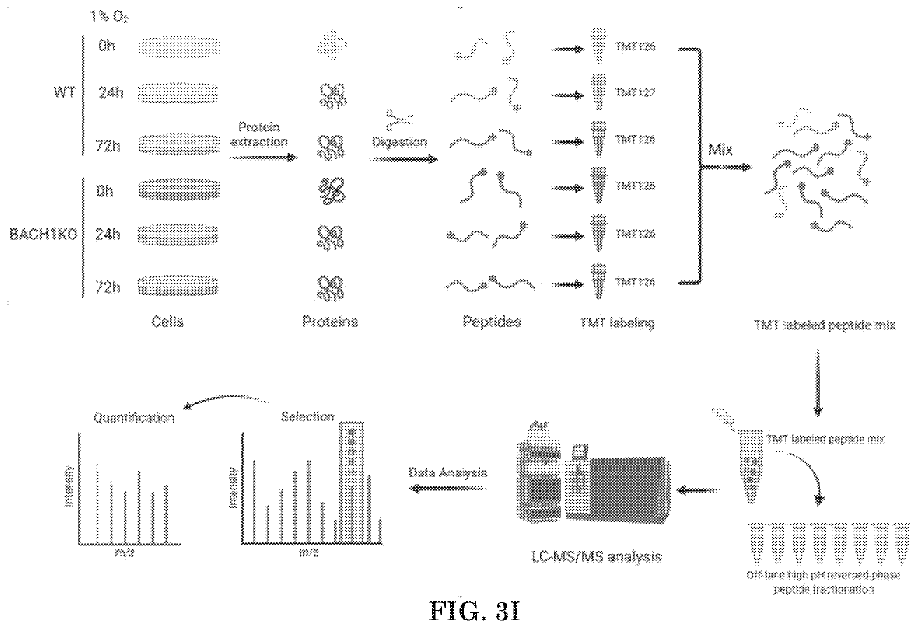

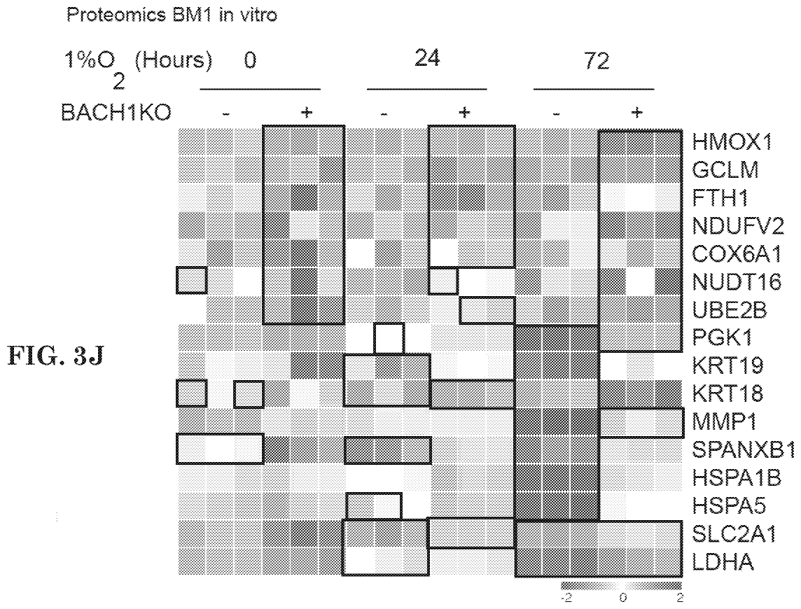

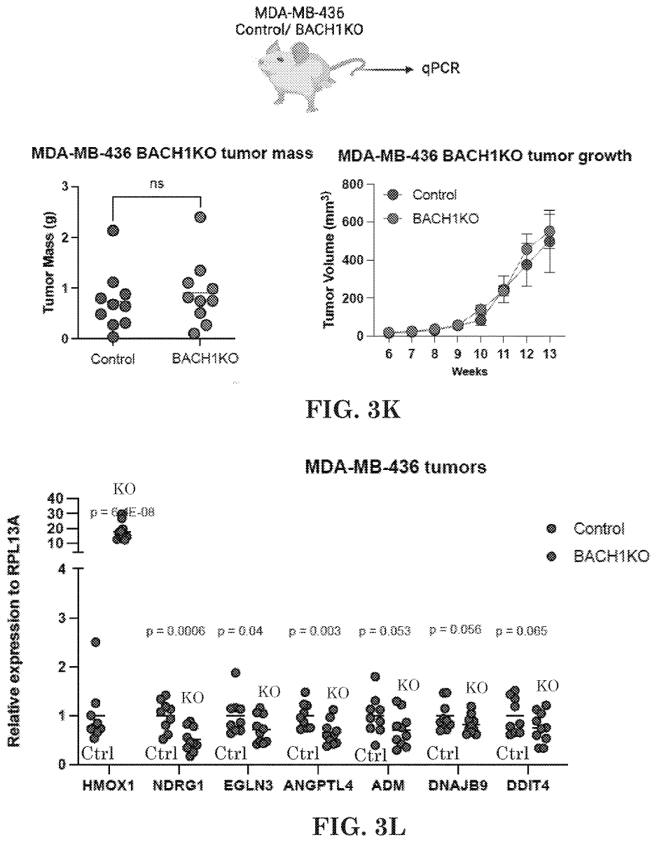

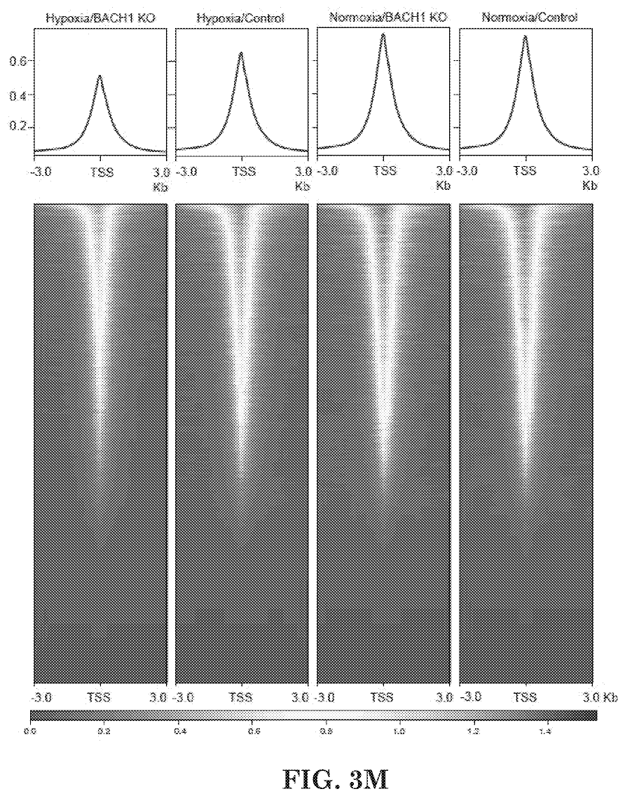

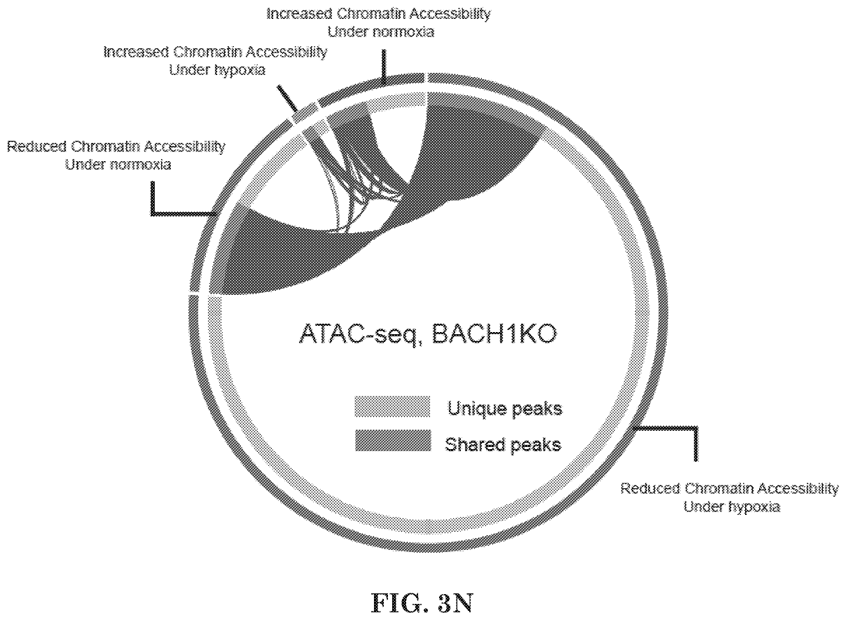

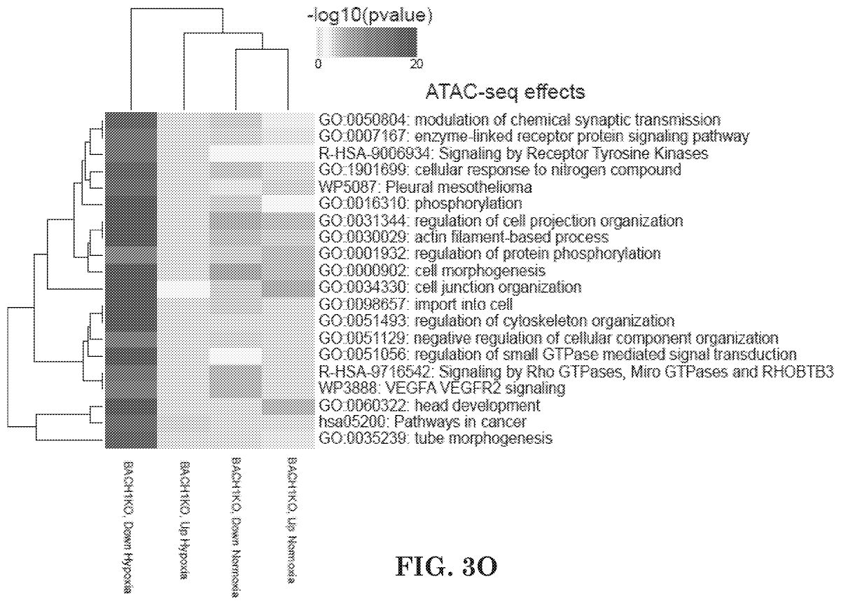

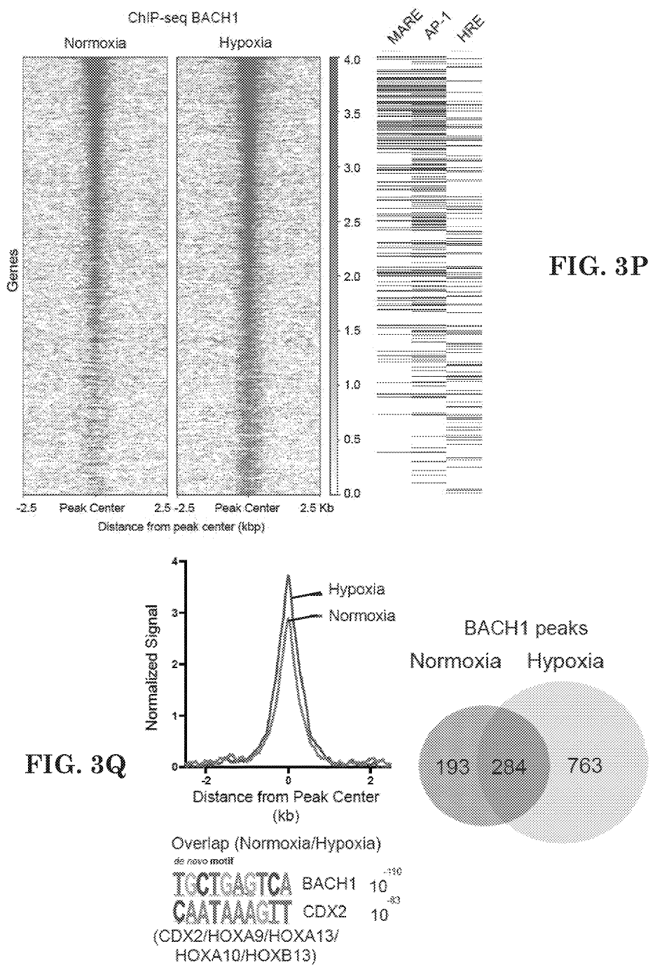

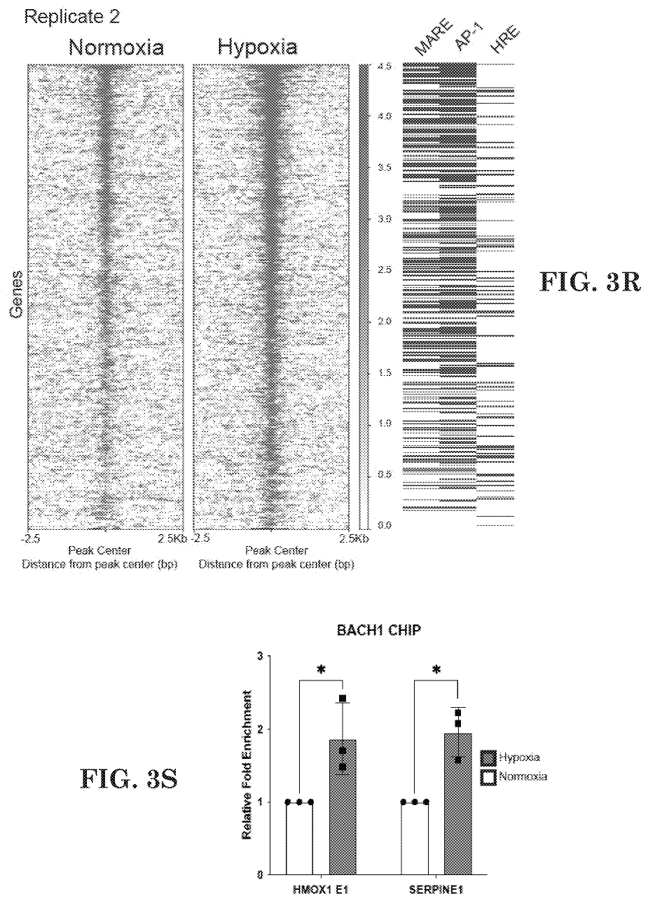

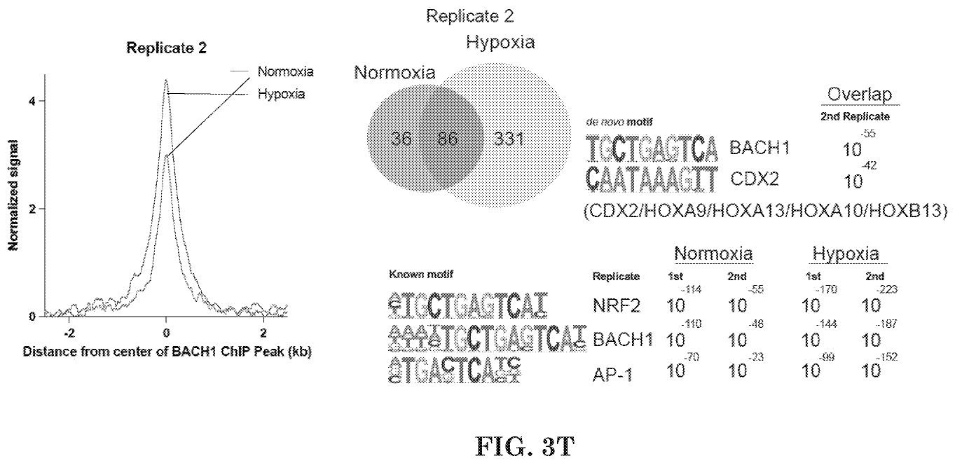

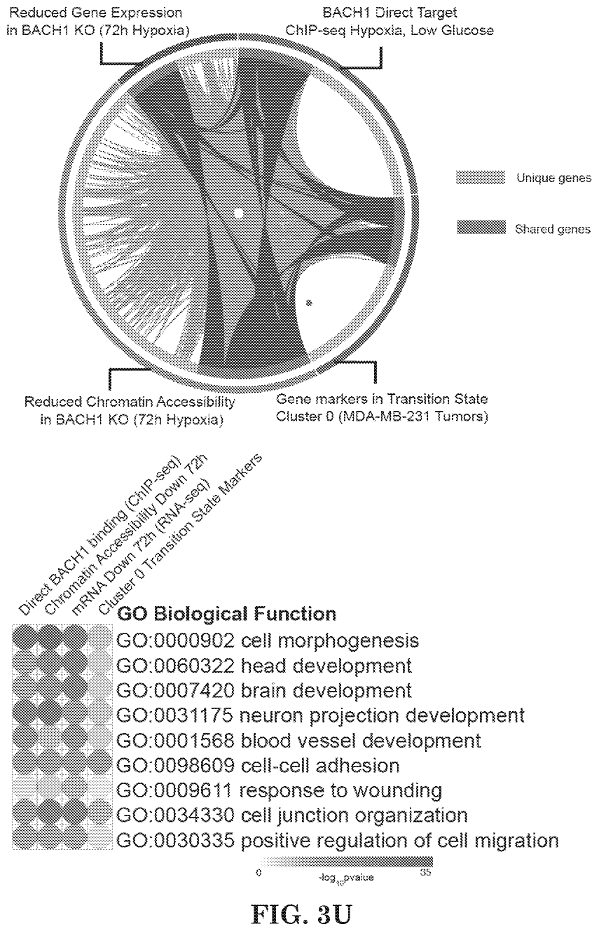

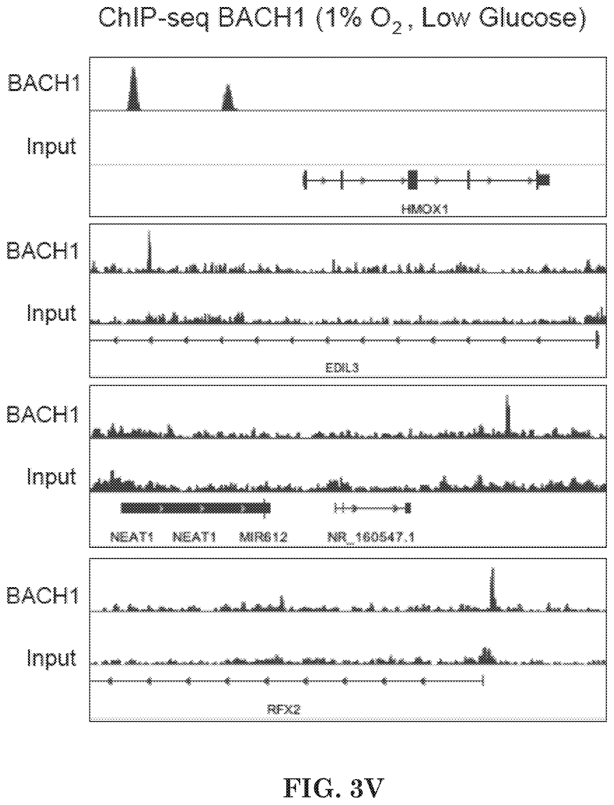

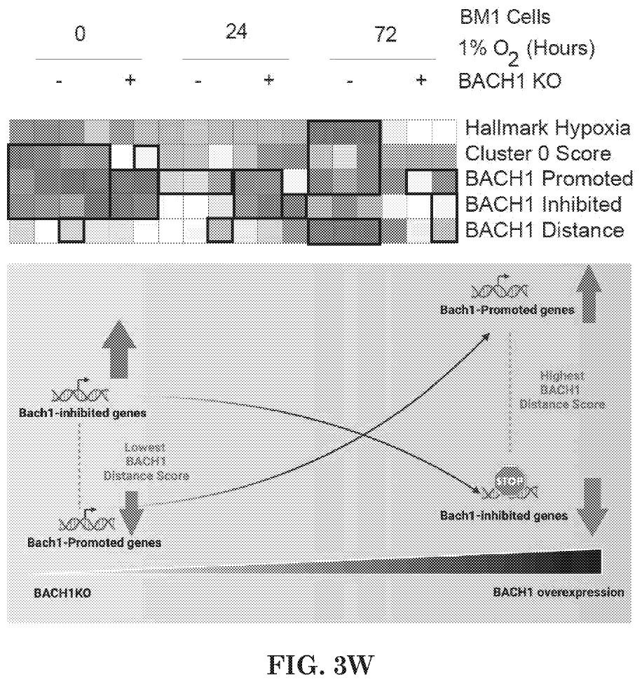

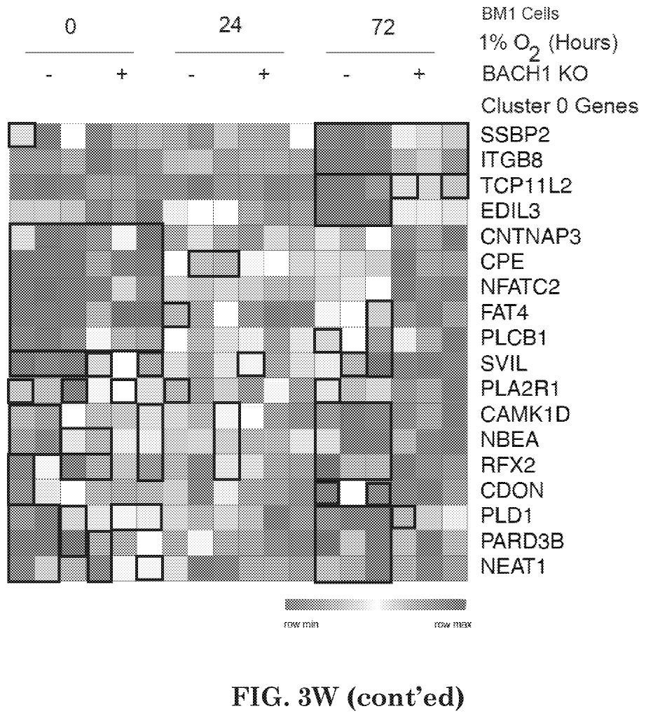

In vitro mass spectrometry for FIG. 3H: A total of 7497 proteins were detected. 6464 proteins were marked as master proteins. If more than one protein in a group has the same score, an equal number of PSMs, and an equal number of peptides, the protein with the longest sequence is designated as the master protein. Among these 6464 mater proteins, 5157 proteins are detected with at least two peptides. 4061 proteins can be quantified among all the TMT labeling channels across three replicates. Abundances for different TMT channels: For TMT-based quantification, the isotope labels are incorporated after peptide samples have been prepared. Each step before mixing the peptides labelled by different TMT tags can introduce errors for quantification. Therefore, normalizing the sample abundances for different TMT channels is crucial for accurate quantification. The total and median abundance are almost the same for all samples. This guarantees that the quantification results are accurate and not affected by experimental bias. Results from different replicates are consistent with ≥0.92 as R2, and nearly 1 as slopes for all least squares fit between replicates.

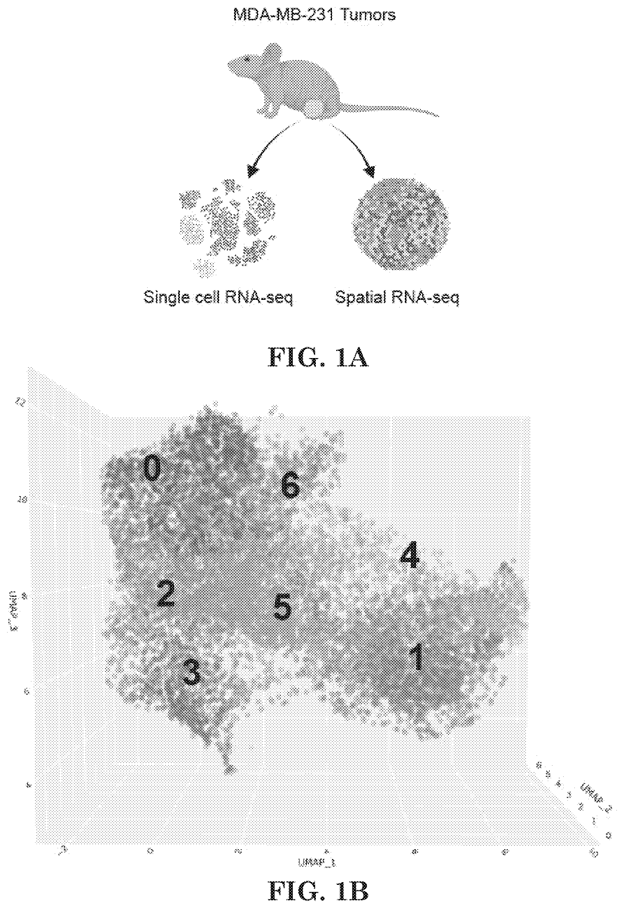

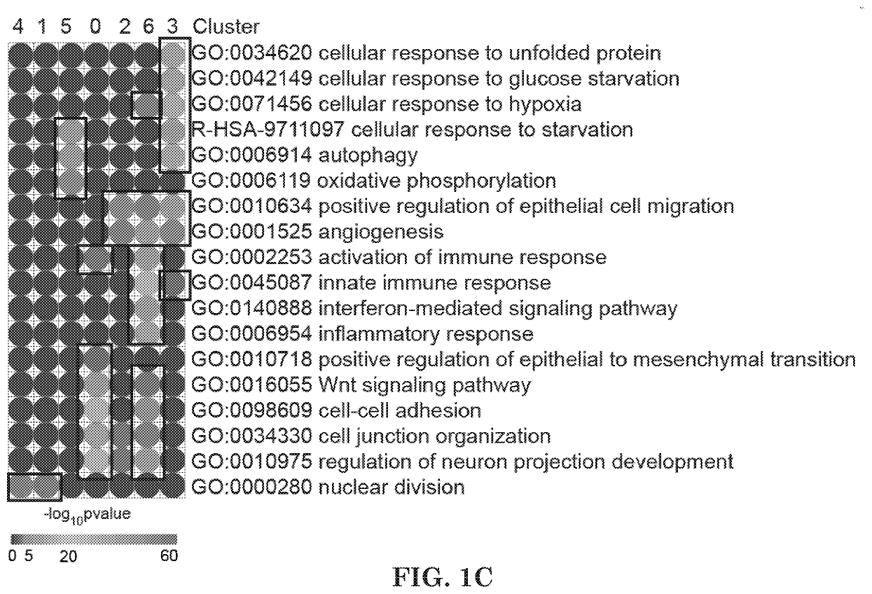

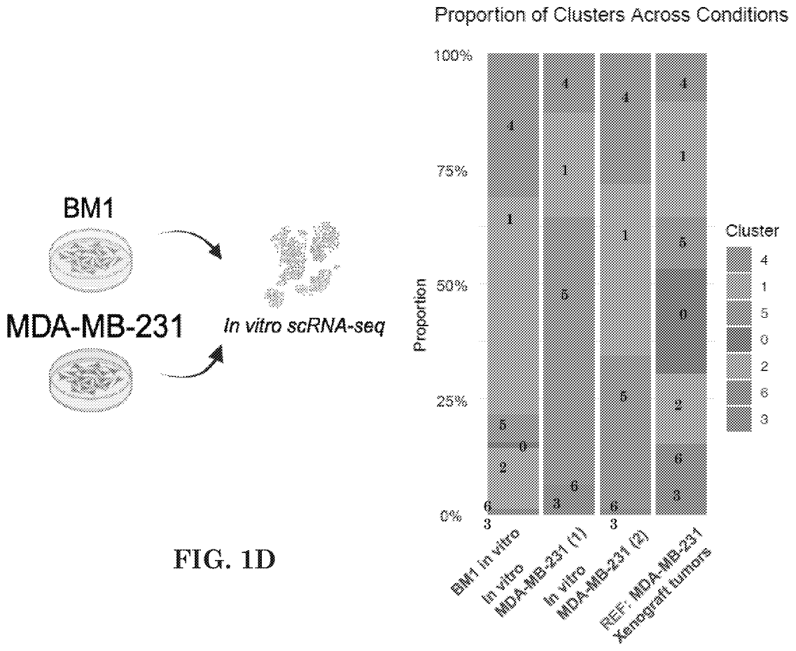

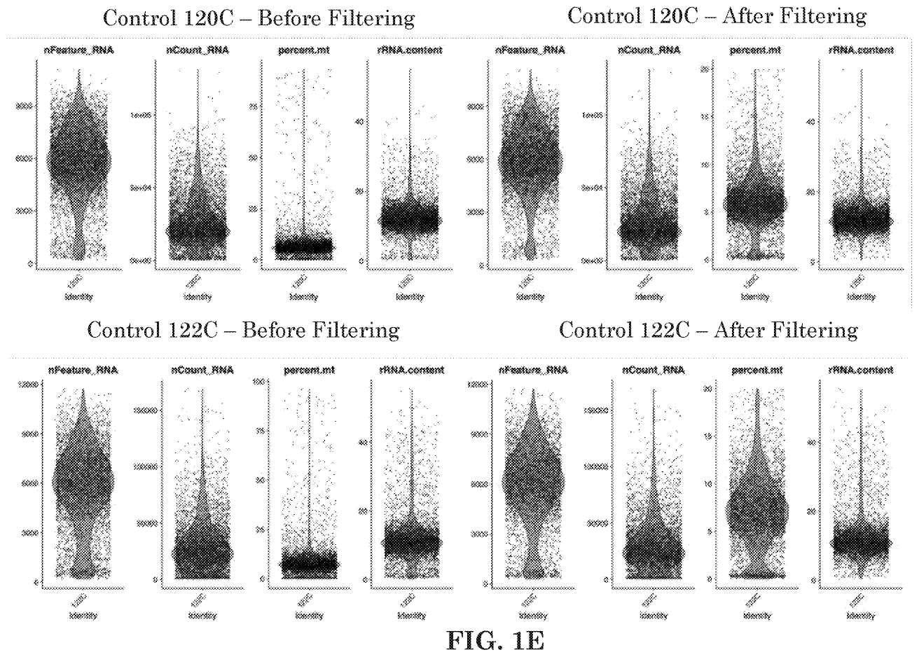

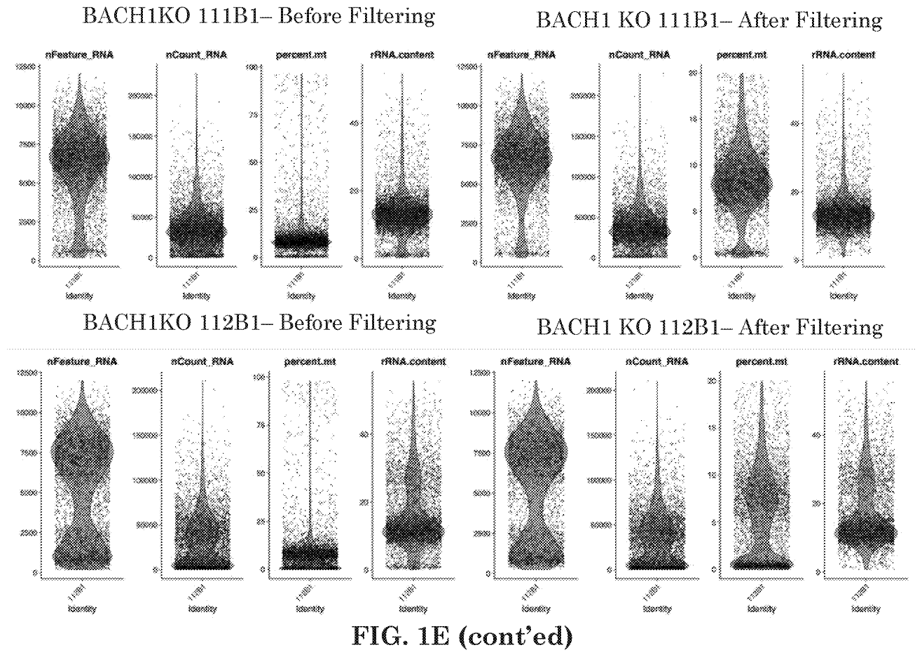

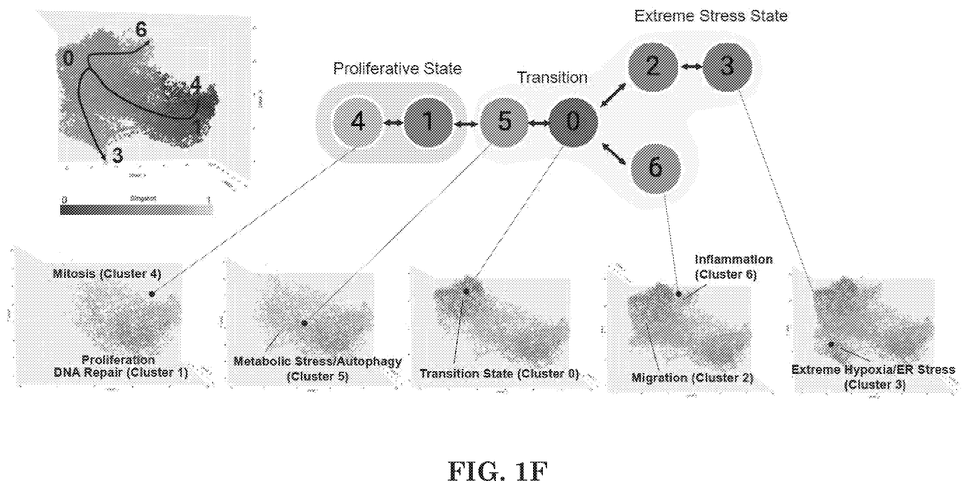

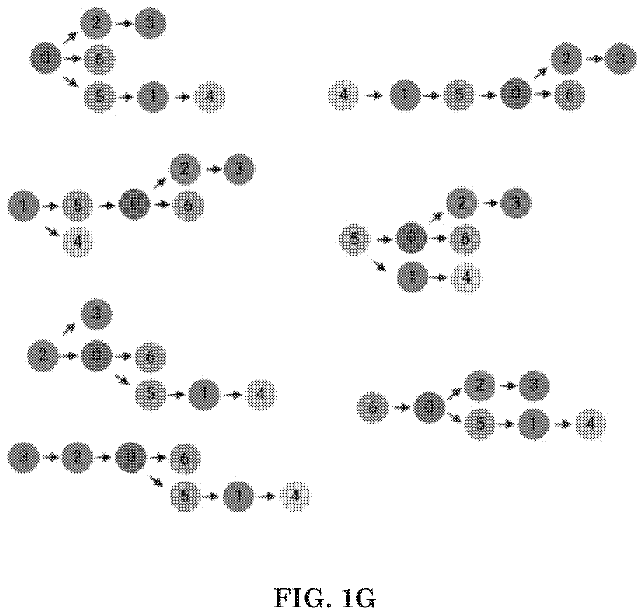

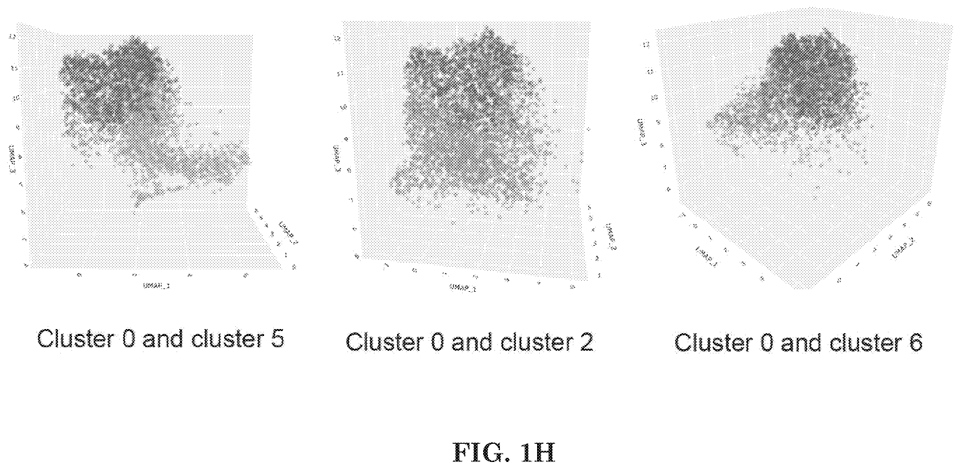

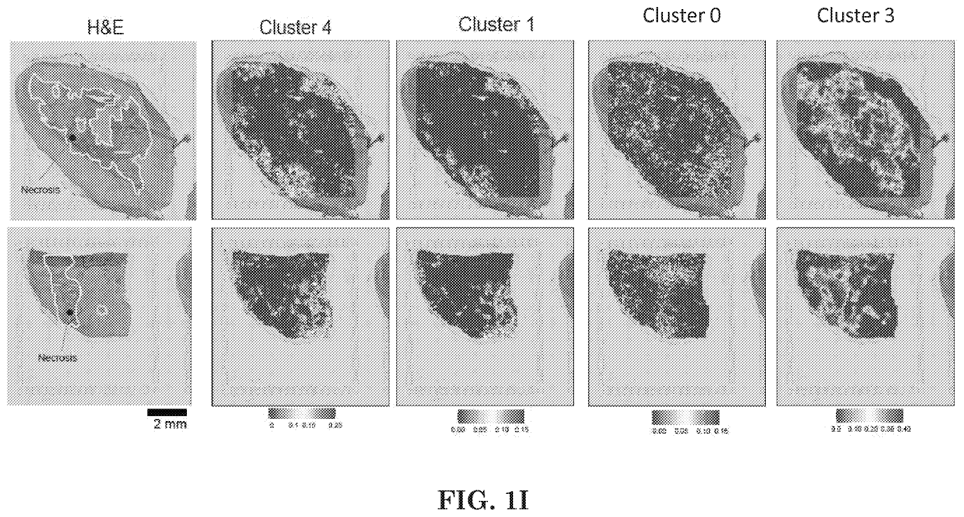

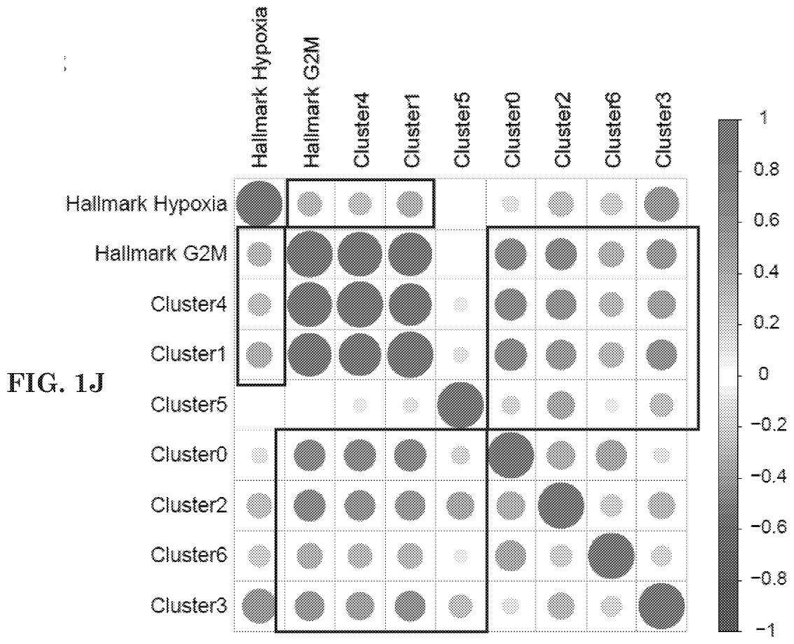

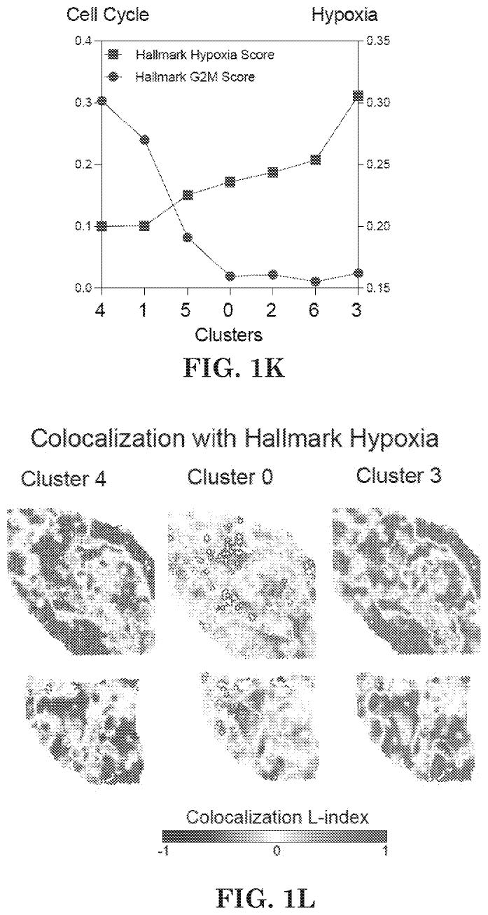

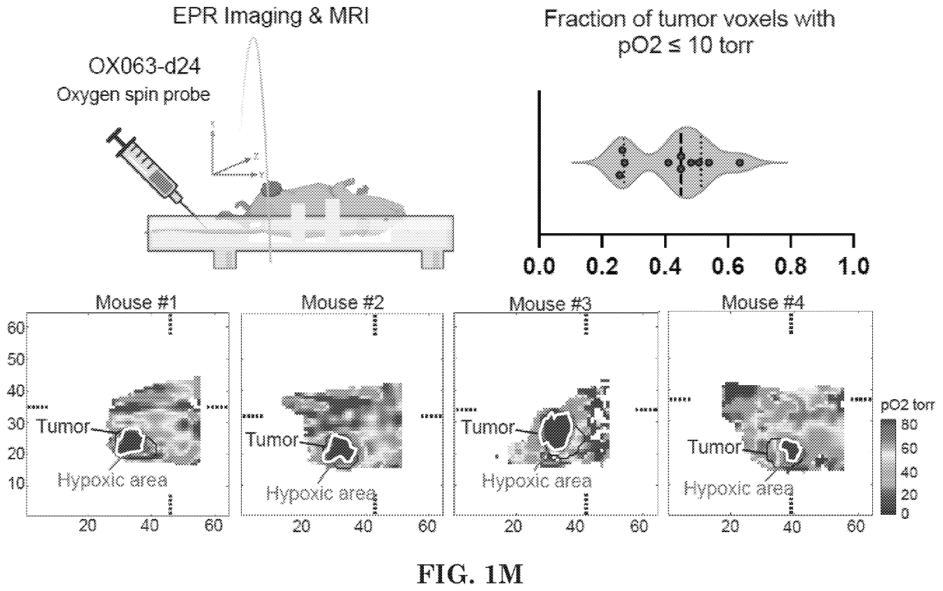

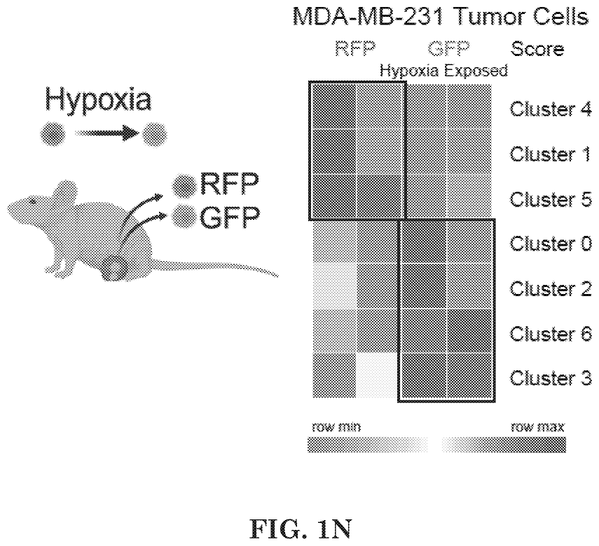

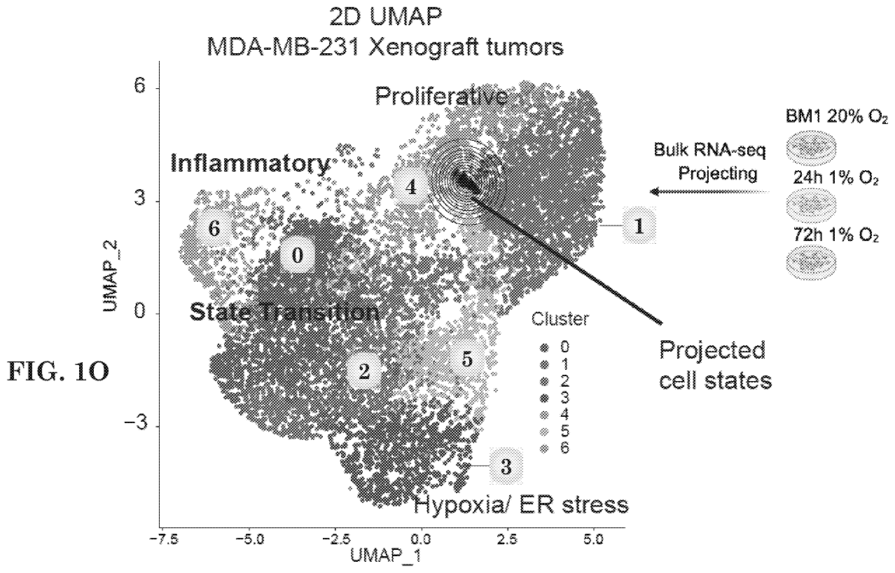

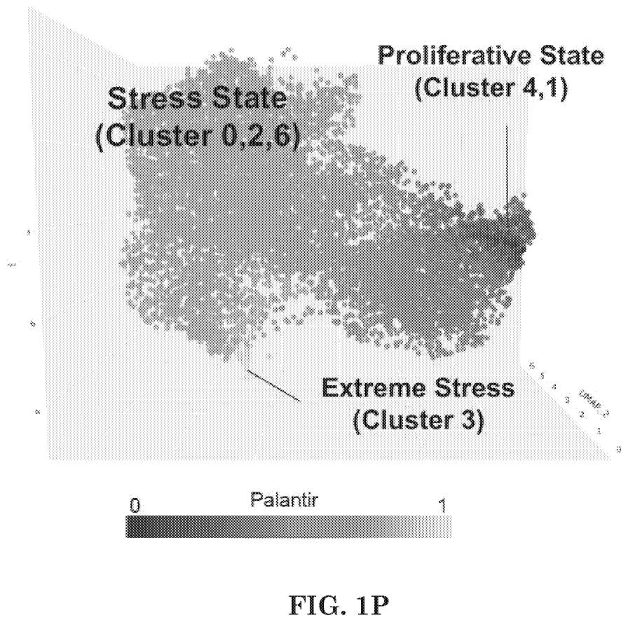

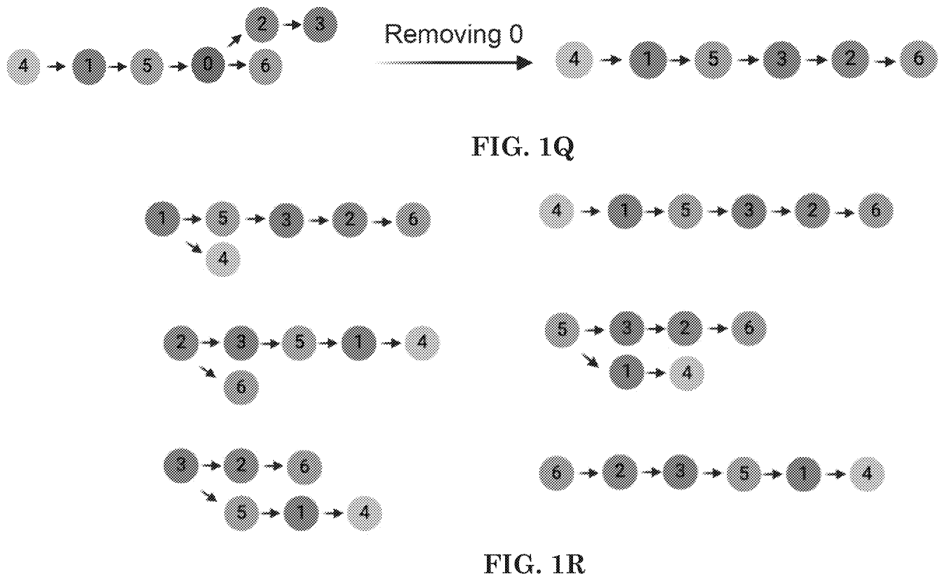

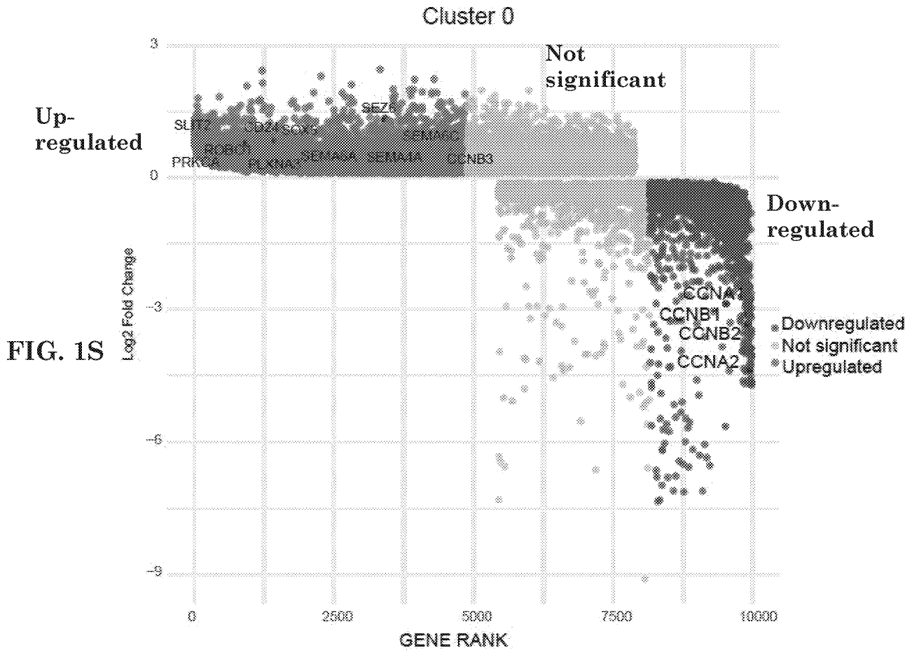

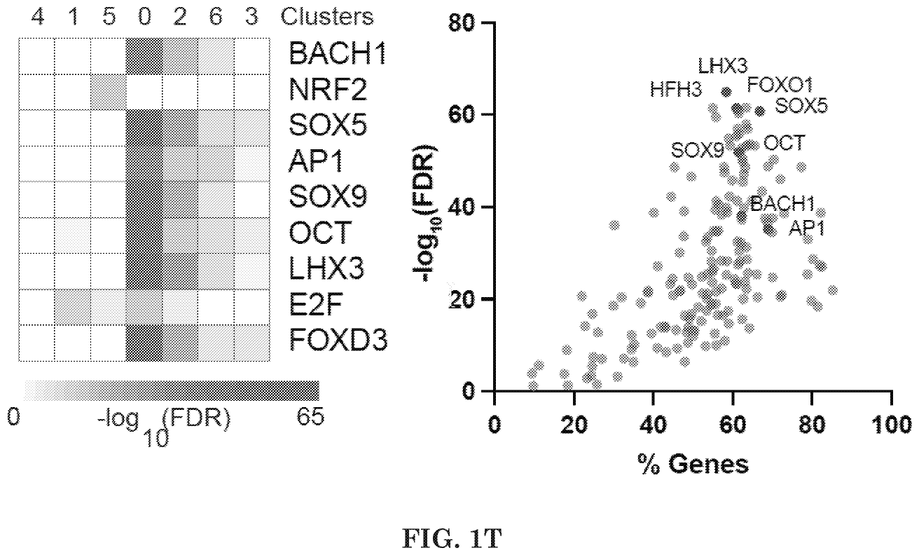

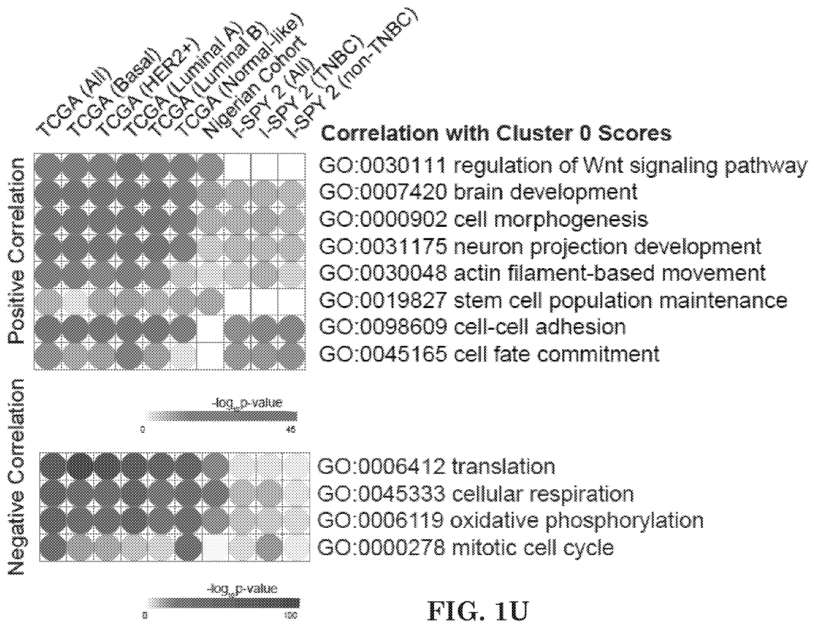

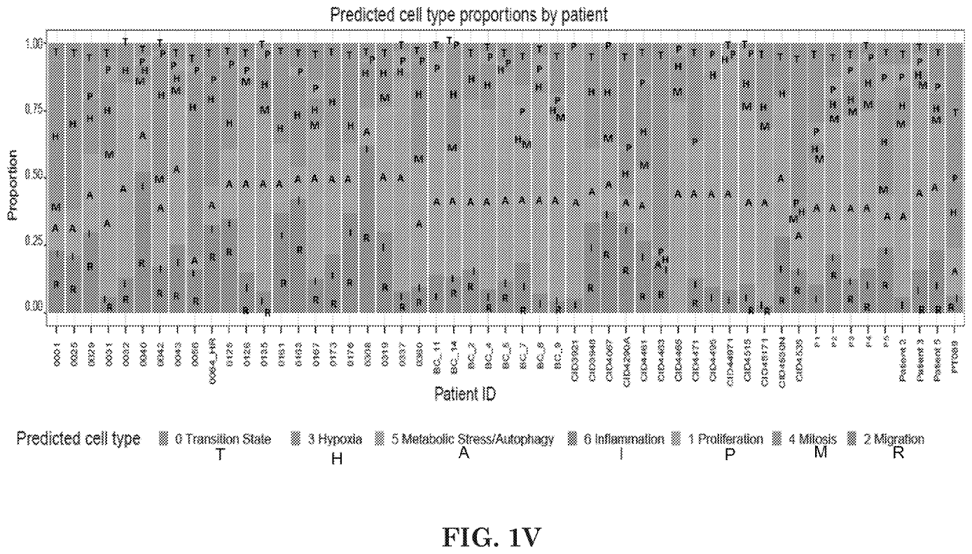

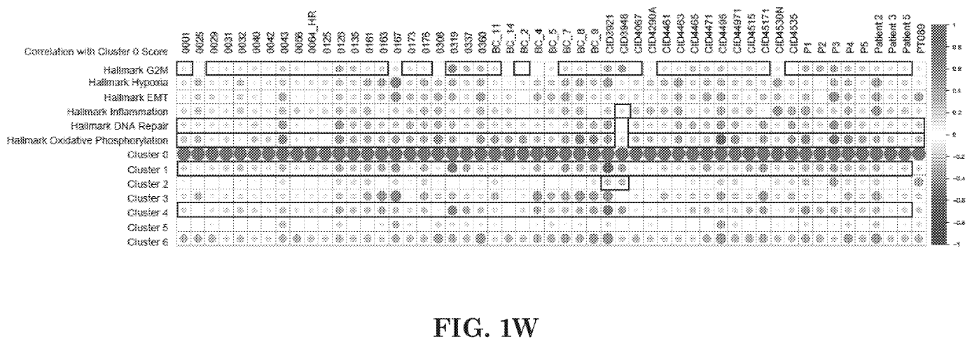

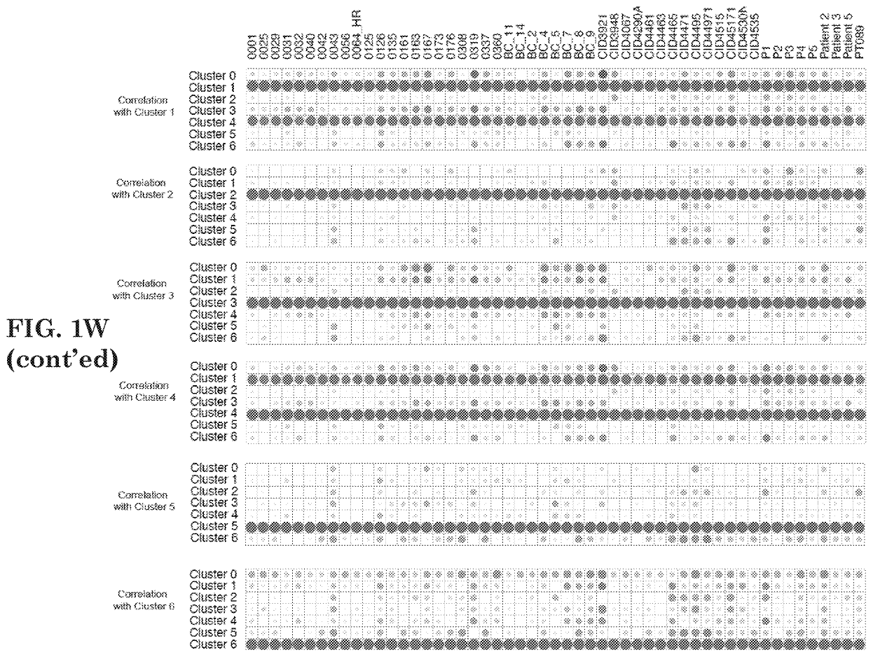

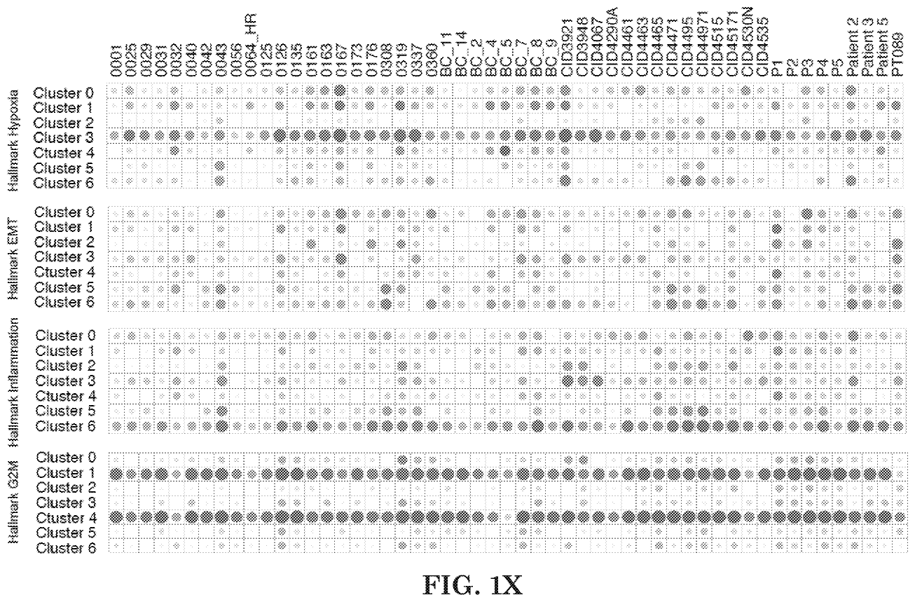

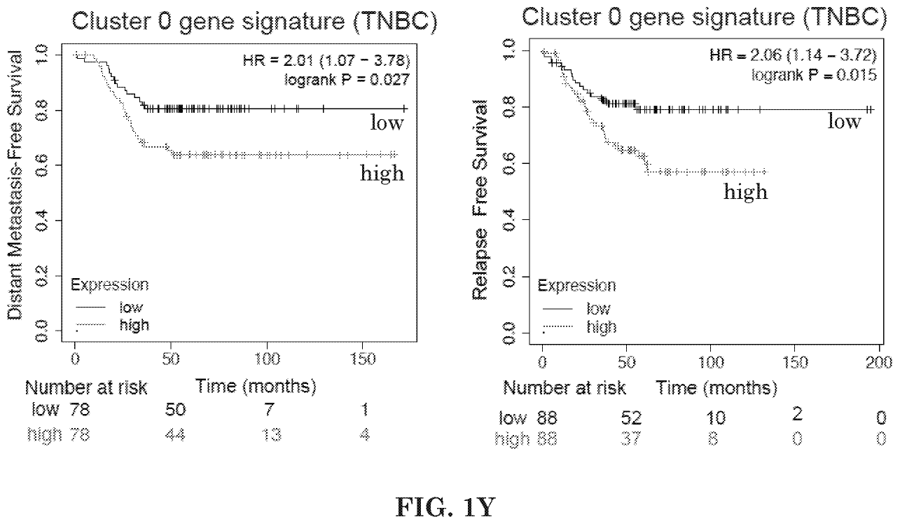

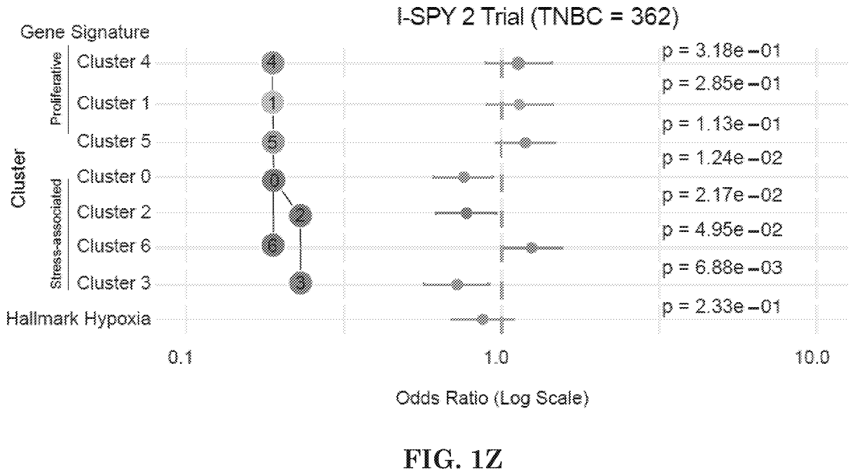

BMI BACH1 overexpression and shBACHI sc-RNAseq: 10⁶ BM1 cells were injected orthotopically into the mammary fat pad of athymic nude mice (Charles River). The mice were sacrificed four weeks after tumor cell implantation. BMI cells from the primary tumors were isolated through tumor dissociation and dead cell removal following the manufacturer's protocols (Miltenyi Biotec, 130-095-929, 130-090-101). Suspensions of single cells were processed for sequencing at the University of Chicago Genomics Facility following standard protocols. Cells were processed on an ICELL8 cx instrument (Takara Bio, 640188) using SMART-Seq chemistry for single-cell full-length transcriptome analysis. The library for each independent replicate was sequenced by an Illumina NovaSeq 6000 in two separate runs, with each run using one lane of a S1 flow cell to generate paired-end 100 bp reads. Sequencing reads from the runs were combined before downstream analyses of each independent replicate. The raw reads were processed by the Cogent NGS Analysis Pipeline (Takara Bio) for quality check, mapping to the human genome (UCSC hg38 with GENCODE annotations) and counting of reads per gene. For each replicate, the samples were processed for quality check using the following manufacturer's recommended parameters: cells with fewer than 10000 total reads or fewer than 300 genes detected were removed; genes with fewer than 100 total reads across all samples or expressed in fewer than 3 cells were removed; outlier cells with greater or less than 3 mean absolute deviation from the median were removed; cells with more than 20% mitochondrial reads or 10% intergenic reads were removed (see FIG. 1E). Gene expression data from three biological replicates were combined, and the batch effect was corrected via the “BatchCorrectedCounts” function in the CountClust package by specifying the replicate number as the variable to regress out [28]. Differential expression analysis was performed using the SCDE package to counter dropout and amplification biases [29]. The gene expression log 2 (fold-change) of BACH1 overexpression tumor cells over control tumor cells was determined from the “scde.expression.difference” function using the “maximum likelihood estimate” result. Genes with significant differential expression were determined with a cutoff of +/−0.5 on false discovery rate (FDR)-corrected Z-scores

MDA-MB-231 and 4T1 Tumors Single-Cell RNA Sequencing: MDA-MB-231 (2×10⁶) and 4T1 (2×10⁴) cells were injected into athymic nude and BALB/c mice, respectively. Tumors of similar size were collected at experimental endpoints: 8 weeks for MDA-MB-231 tumors and 6 weeks for 4T1 tumors. MDA-MB-231 tumors were processed using the Chromium Next GEM Single Cell Gene Expression v3.1 chemistry, while 4T1 tumors were profiled using the Chromium GEM-X platform at the University of Chicago Genomics Facility. MDA-MB-231 Tumors: Pre-demultiplexed FASTQ files were processed using Cell Ranger v7.0.0 (10×Genomics). Given the human-in-mouse xenograft model, reads were aligned to both human (hg38, 2020-A) and mouse (mm10, 2020-A) reference genomes. Reads were filtered and quantified as UMI counts, generating barcoded count matrices. Low-quality cells, including those with ≥20% mitochondrial content, poor sequencing quality, or low cell counts, were removed. Cells expressing ≥200 genes and genes detected in ≥3 cells were retained. Doublets and triplets were removed using DoubletDecon [31]. The UMI count matrix was log-normalized using Seurat (5.0.0) (NormalizeData, FindVariableFeatures) [32], and scRICA was used for integration, leveraging the top 2,000 highly variable genes as anchors for pairwise sample integration via Canonical Correlation Analysis (CCA). To correct for technical variation, we applied linear transformation scaling, followed by dimensionality reduction with PCA and UMAP. Graph-based clustering was conducted at resolution=0.5 (FindNeighbors, FindClusters) [32]. Human-mouse mixed and unidentified cells (Clusters 7, 8, and 9) were removed. Species identity was confirmed by computing the human-to-mouse gene count ratio (≥0.75 per cell) using Cell Ranger mixed reference genome analysis. Differential expression analysis was conducted using FindMarkers ( ) (Seurat v4.1.1, Wilcoxon rank-sum test) to compare KO vs. Control, calculating fold-change and adjusted p-values. For 3D UMAP, we used n_components=3 in the scanpy.tl.umap function.

4T1 Tumors-scRNA-seq FASTQ files were processed using Cell Ranger v8.0.1 (10×Genomics), aligning reads to the mouse genome (mm10-3.0.0). Filtered reads were quantified as UMI counts, generating barcoded count matrices. QC filtering removed low-quality cells, ≥20% mitochondrial content, and doublets using DoubletDecon [31]. Cells expressing ≥100 genes and genes detected in ≥3 cells were retained. Data were log-normalized and integrated using scRICA and Seurat (5.0.1) (CCA-based integration, top 500 variable genes), followed by PCA, UMAP, and graph-based clustering (resolution=0.5). Cell Type Annotation: CellAssign (v0.99.2) [32] was used to classify clusters based on mouse immune and 4T1 tumor markers [33], leveraging a probabilistic model. The dataset was converted to AnnData (h5ad) format for Python processing, normalized for sequencing depth, and cell types were assigned based on marker gene expression probabilities. InferCNV for Tumor Cell Isolation: CNVs were inferred using inferCNV [34], comparing tumor vs. normal cells based on expression profiles. CNV correlation scores were computed, and cells with a correlation >0.35 were classified as 4T1 tumor cells, enabling intra-tumoral heterogeneity analysis. ProjecTILs [35] was used to subcluster T cells, aligning query cells to a reference T cell atlas. CellChat [36] was used to analyze cell-cell communication in 4T1 single-cell RNA-seq data, identifying key ligand-receptor interactions and signaling pathways involved in tumor-immune crosstalk.

MDA-MB-231 Spatial RNA Sequencing: fresh-frozen matched MDA-MB-231 tumors were processed for spatial transcriptomics using 10× Genomics Visium Spatial Gene Expression, with library preparation and sequencing at Northwestern University NUSeq Core Facility. Spatial transcriptomics data from SpaceRanger outputs (230614, 4 samples) were processed using Load10X_Spatial( ) separating mouse and human matrices based on gene expression, consistent with scRNA-seq processing. Quality control (QC) criteria included filtering for spatial features (nFeature_Spatial: 100-8,000), total transcript counts (nCount_Spatial: 1-50,000), and mitochondrial content (percent.mt≤20%). Data underwent SCTransform (SCT) normalization, merging via variable features, and batch correction with Harmony to generate an integrated spatial transcriptomics dataset [32]. Spatial transcriptomics data were visualized using SpatialFeaturePlot( ) mapping cell type prediction scores onto spatial transcriptomics spots [32]. Differential expression analysis was performed using FindMarkers( ) (Wilcoxon rank-sum test) to assess fold-change and adjusted p-values (KO vs. CONTROL). SEAGEL [37] was used to analyze spatial co-localization in spatial transcriptomics data, leveraging the L-index to measure the spatial association of gene signatures.

Patient Single Cell RNA-seq: the raw scRNA-seq data from 85 breast cancer patients were obtained through collaboration [38] and provided in filtered matrix format (barcode.tsv, genes.tsv, counts.mtx, metadata.tsv). For downstream analysis, we selected cancer epithelial cells, retaining only patients with ≥100 such cells, yielding a final cohort of 53 patients. As QC and doublet detection were preprocessed, we applied linear transformation scaling to remove unwanted variation before dimensionality reduction using PCA and UMAP. Graph-based clustering was conducted using FindNeighbors and FindClusters (resolution=0.8). Hallmark and MDA-MB-231 cell state cluster scores were computed per patient using established marker genes. To explore score relationships and dependencies, we generated correlation matrices with rcorr( ) and visualized significant correlations using corrplot( ) offering insights into patient-specific cellular interdependencies.

Trajectory Analysis: Slingshot v2.14.0 was used to infer cellular trajectories from the top 30 principal components (PCs), initiating at cluster 4. Slingshot constructs a minimum spanning tree (MST) in PC space to model branching lineages and fit principal curves. Each cell's pseudotime was assigned based on its projection onto a specific trajectory curve, with weights determined by its distance from the curve. To further refine trajectory inference, we applied CellRank [50, 51], which estimates transition probabilities using a pseudotime kernel computed from Palantir [52] with default parameters. The pseudotime kernel was built from Palantir-derived pseudotime values, capturing cell differentiation dynamics. CellRank estimated state transitions by integrating pseudotime values and gene expression trends, allowing us to infer the likelihood of cells transitioning from one state to another. Using Markov chain analysis, CellRank assigned transition probabilities based on transcriptional similarity, identifying terminal states as absorbing states with the highest probability of capturing cells at the endpoint of differentiation. Additionally, fate probabilities were computed to quantify the likelihood of each cell progressing toward a specific terminal state.

Cell State Reference Mapping: the ProjecTILs [35] package was utilized to project single-cell RNA-seq (scRNA-seq) data from our query dataset onto reference data. We ran Run.ProjecTILs with default parameters which included correction for batch effects and project the queried cells in the UMAP of the reference map. Specifically, to correct for batch effects, ProjecTILS employed the STACAS integration method, aligning the query dataset to the reference atlas while preserving biological variation. Principal component analysis (PCA) was performed on the reference atlas, and the resulting PCA rotation matrix was applied to the query dataset to project cells into the same low-dimensional space. The UMAP transformation from the reference atlas was subsequently applied to the query cells, embedding them within the reference landscape without altering its structure. Cell type assignment was performed using a nearest-neighbor classifier, predicting the most likely subtype for each query cell based on its proximity to annotated reference cells.

Flow Cytometry: All antibodies were used at a final concentration (Table 9) in a 100 μL staining volume, following titration for optimal performance and according to manufacturer instructions. Antibodies included PD1 (BUV395), CD86 (BUV496), CD69 (BUV563), Ly6G (BUV661), CD103 (BV421), I-A/I-E (Pacific Blue), CD19 (BV510), CD11b (BV570), CD206 (BV605), PDCA (BV650), Ki67 (BV711), CD4 (Spark 685), Ly6C (BV785), CD44 (FITC), CD45 (AF532), F4/80 (PerCP/Cy5.5), CD8a (PerCP ef710), CD80 (PE/Dazzle 594), CD62L (PE/Cy7), CD11c (AF647), Foxp3 (APC), CD3 (AF700), and NK1.1 (APC Cy7), from BioLegend, BD Biosciences, and Thermo Fisher. For intracellular markers (Foxp3, Ki67), cells were fixed and permeabilized, and antibodies diluted 1:200 in permeabilization buffer. The Zombie NIR viability dye was used at 1:500 dilution. Fc Block (BD Biosciences) was included as recommended. Flow cytometry cells and/or beads (Ultracomp eBeads Plus compensation beads, Invitrogen) were used for staining of positive and reference controls. Cells were analyzed on a Cytek Aurora spectral flow cytometer, and data were processed using FlowJo software (Tree Star). Gating of the populations was performed based on Fluorescence-minus-one (FMO) tubes. All antibodies and reagents were prepared in accordance with manufacturer protocols.

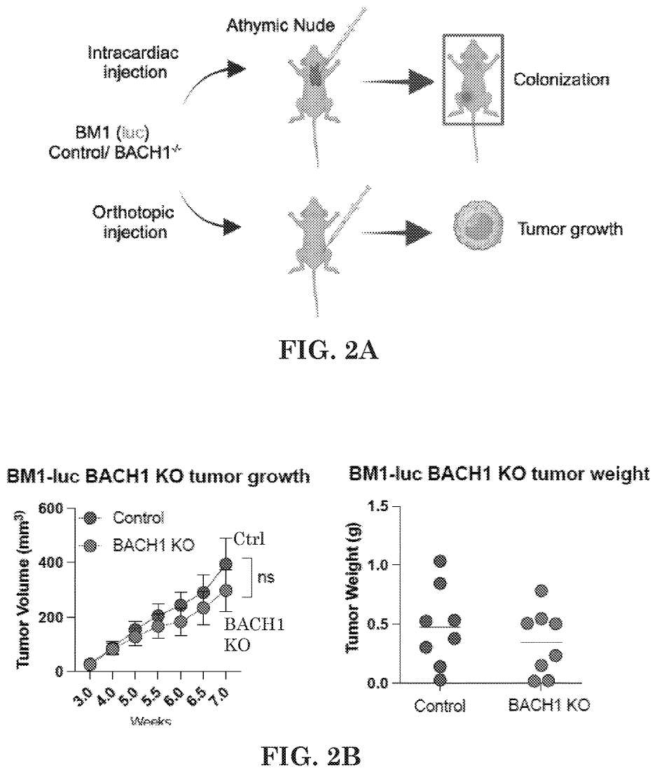

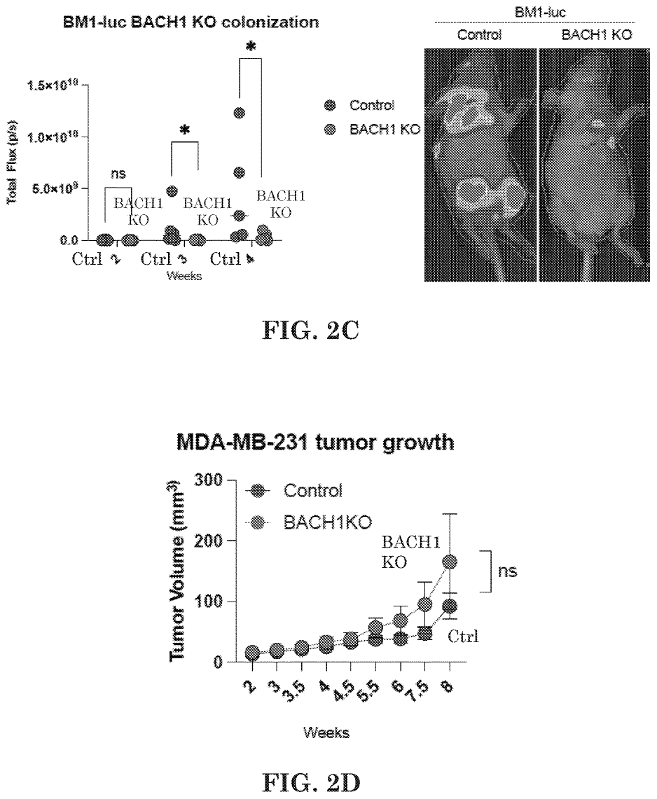

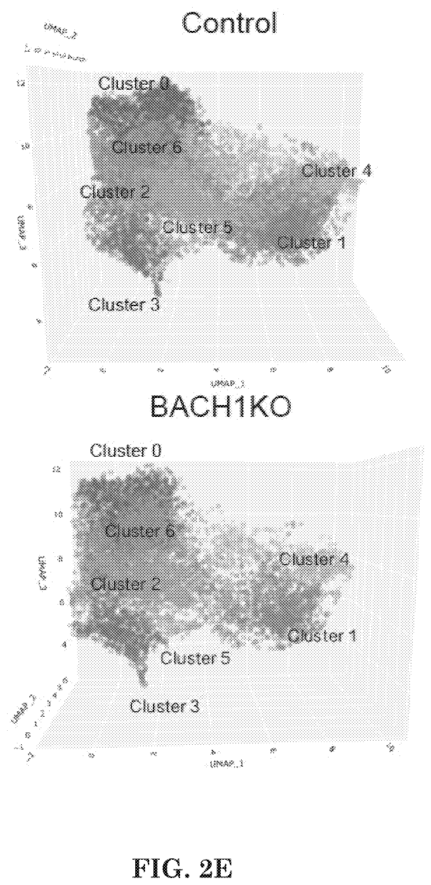

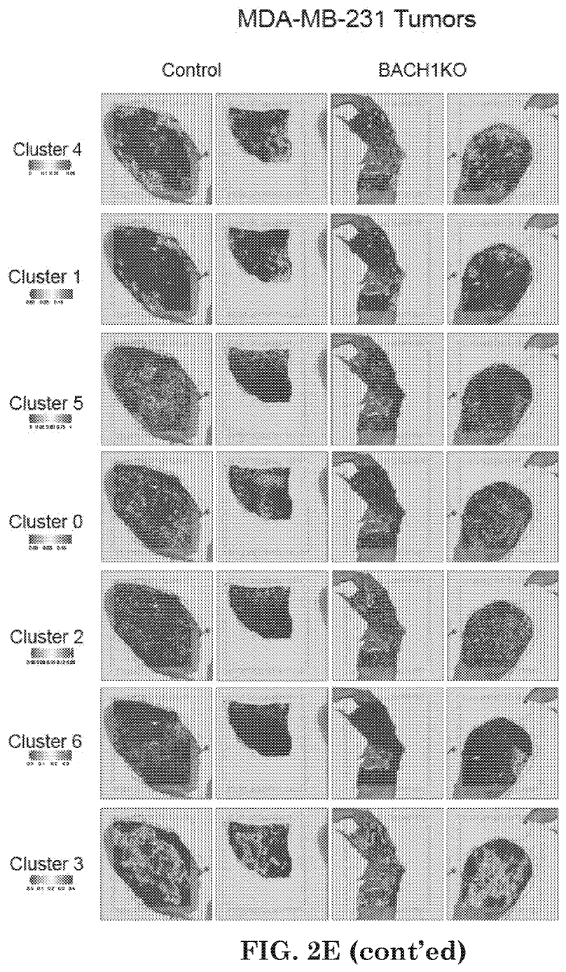

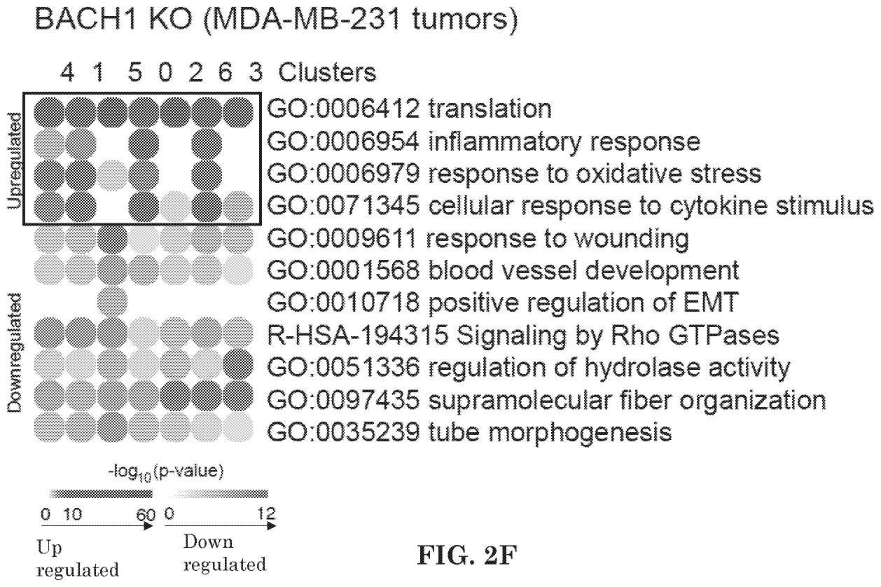

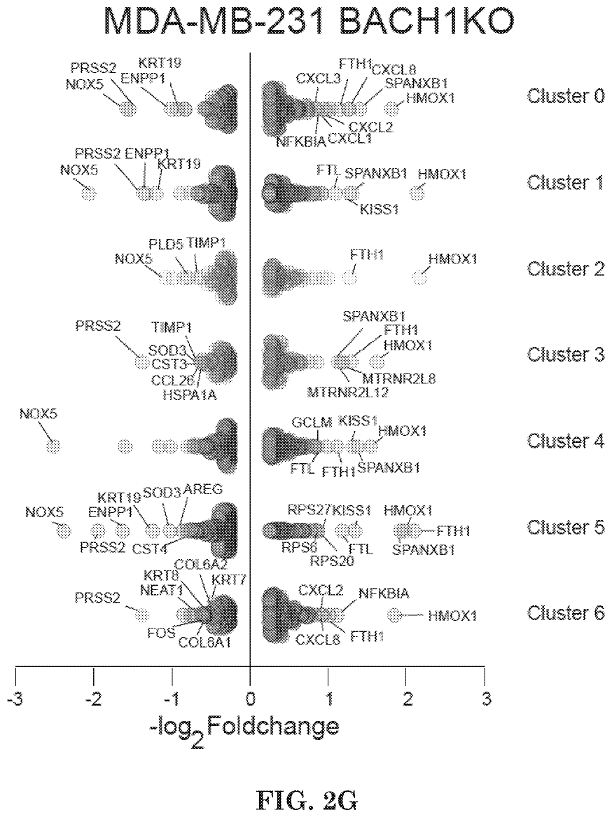

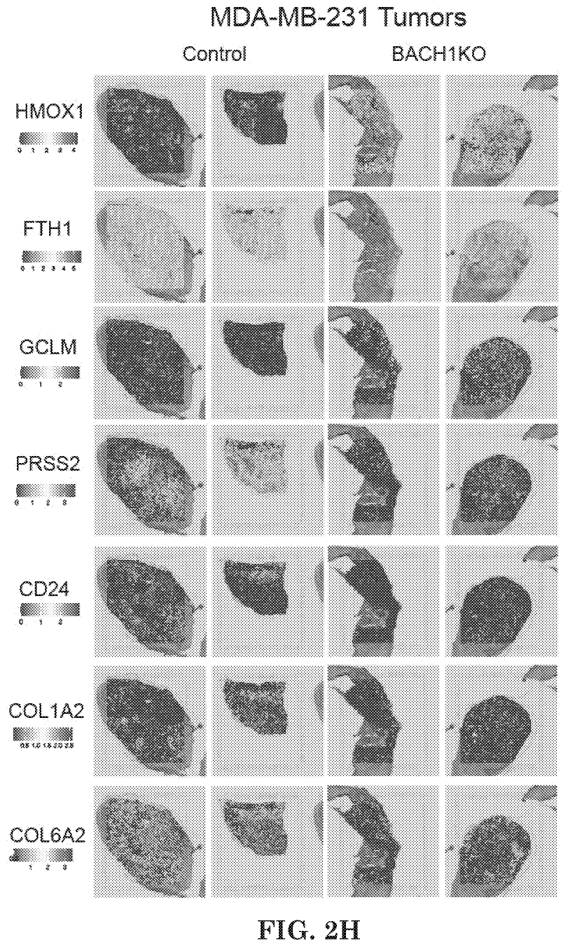

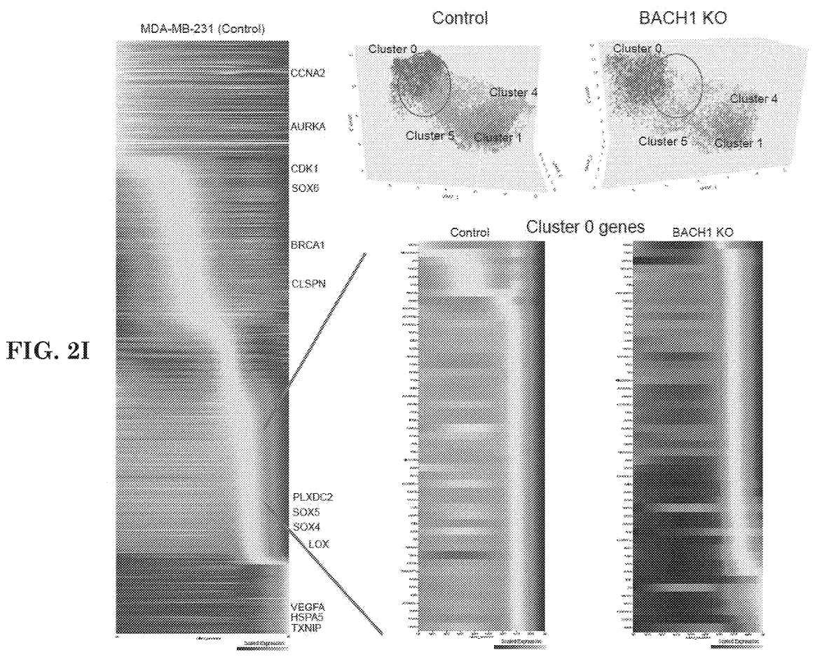

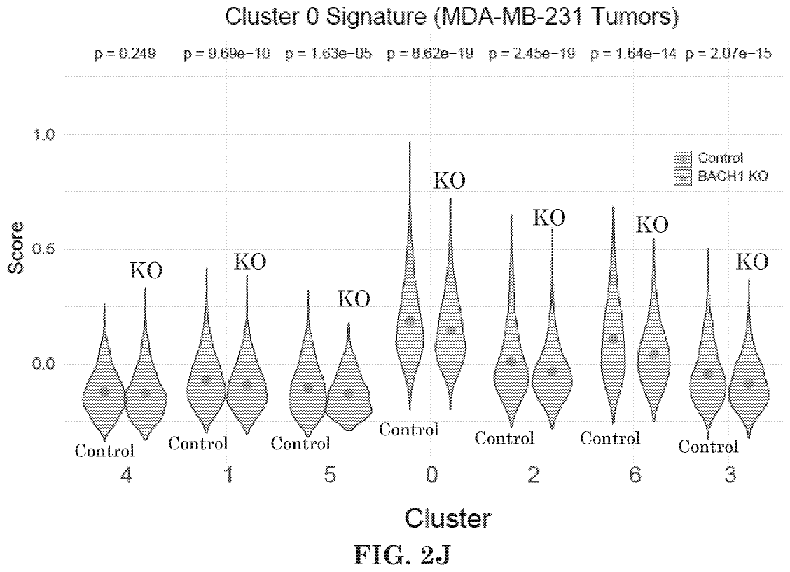

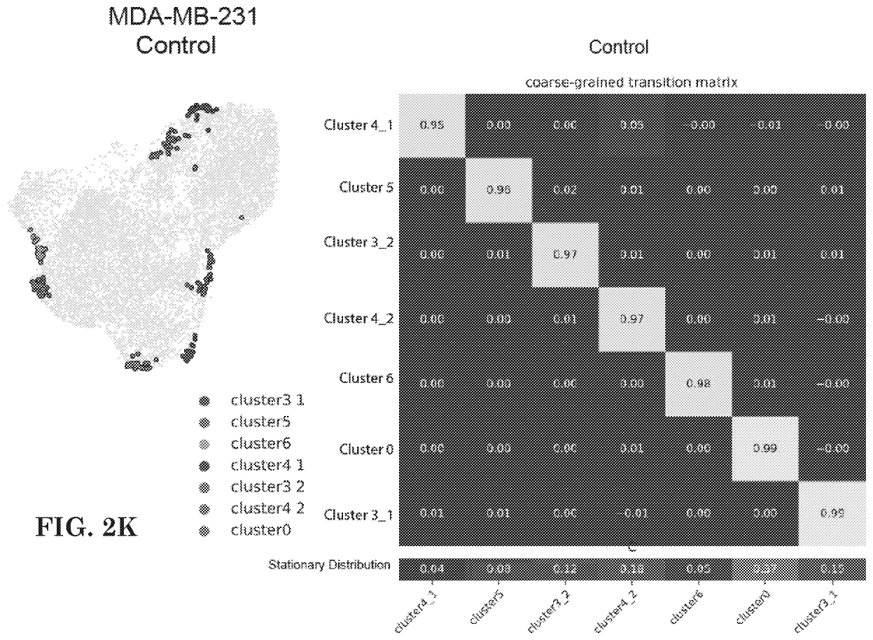

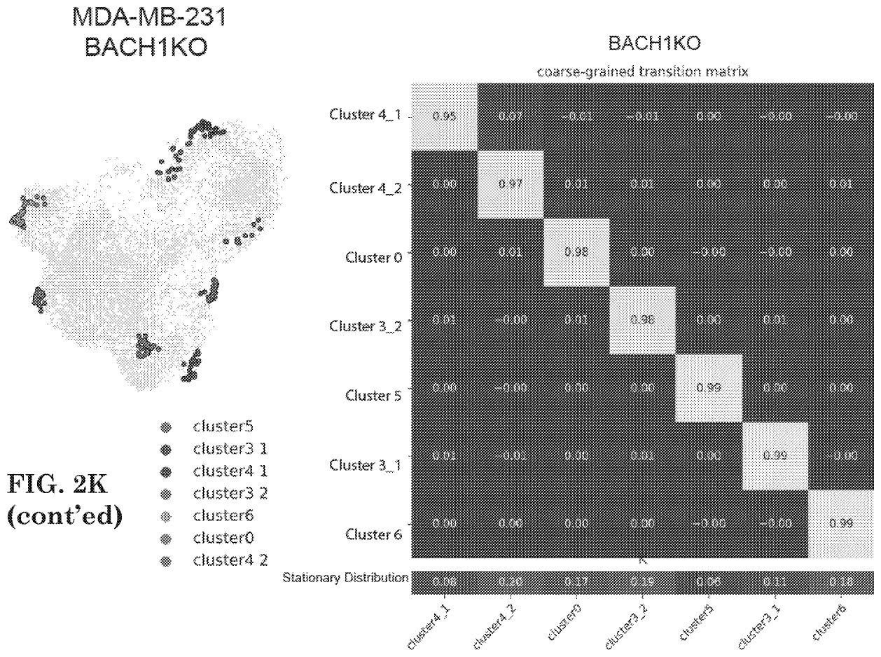

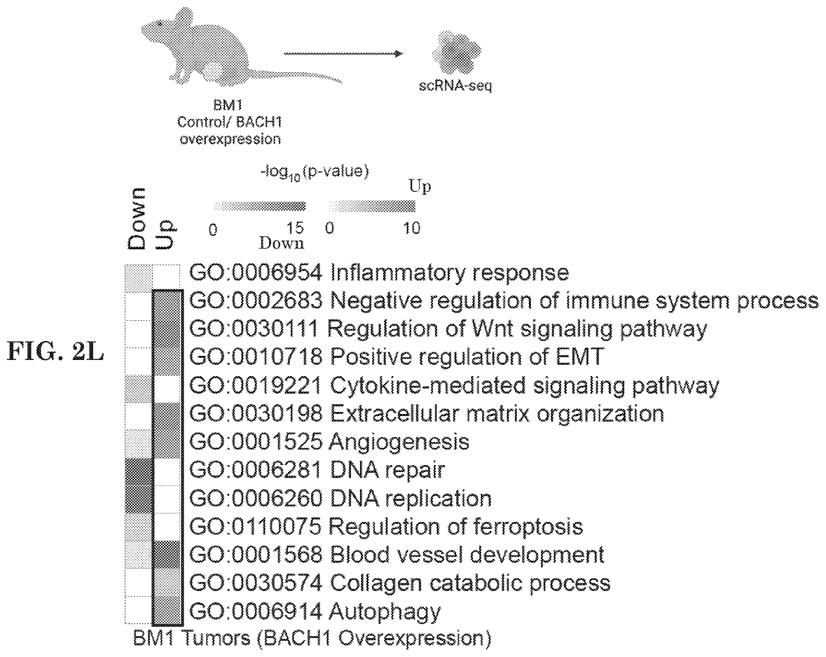

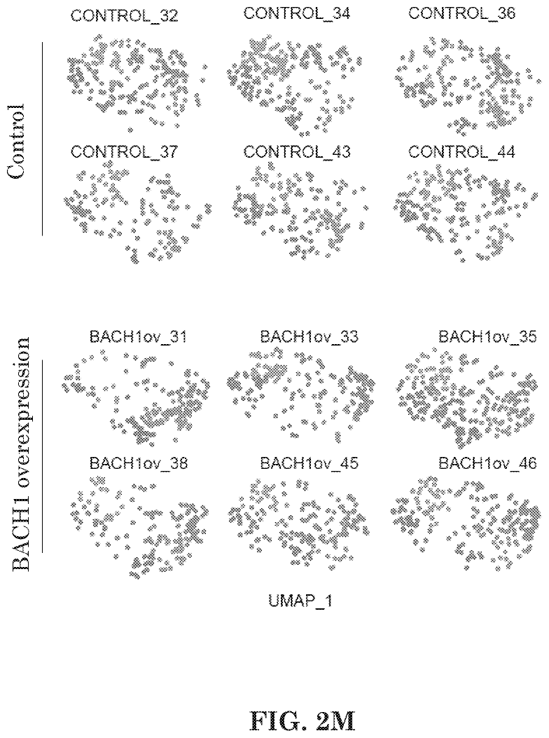

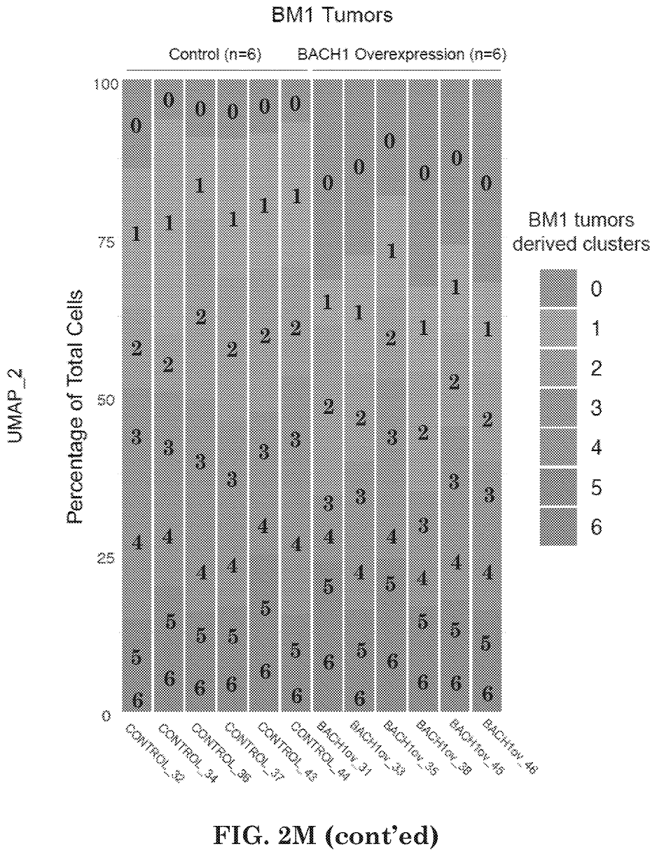

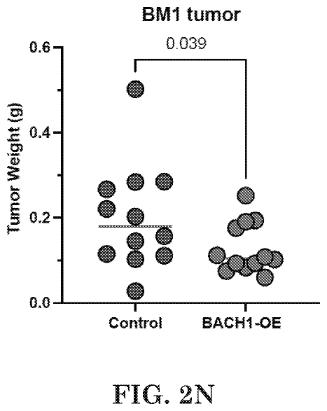

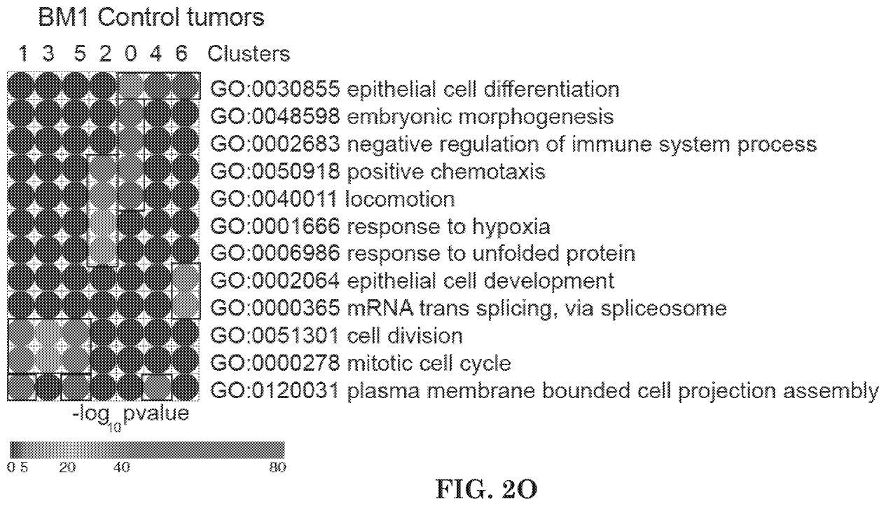

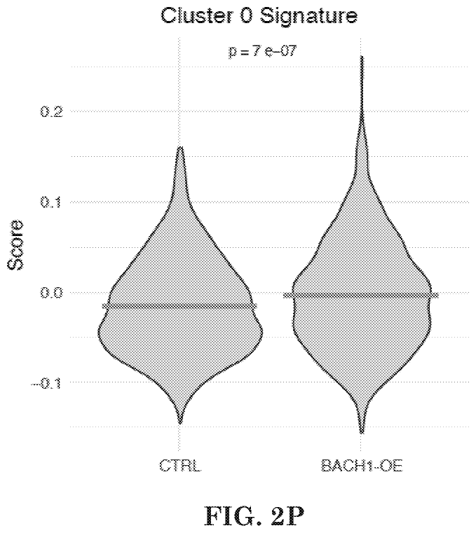

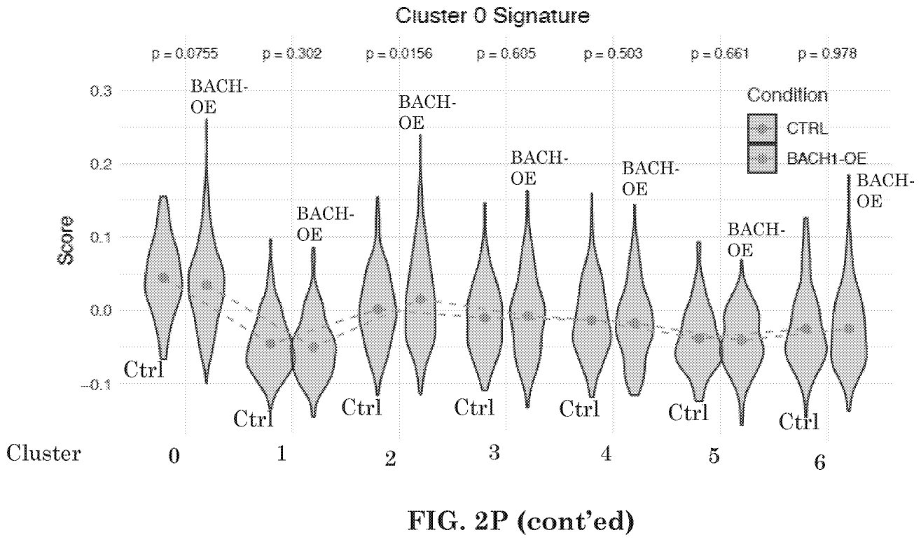

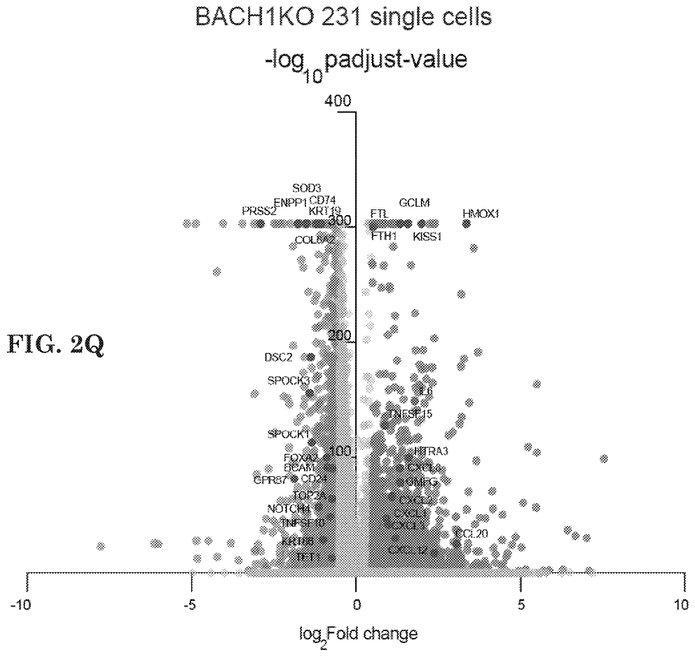

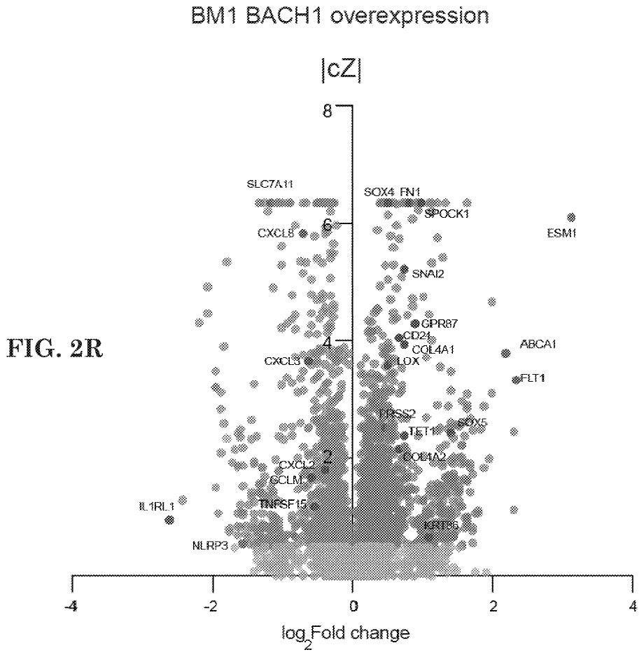

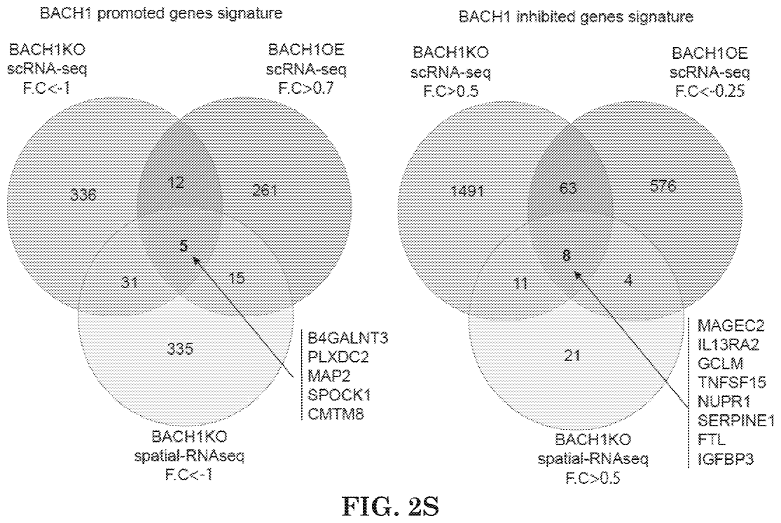

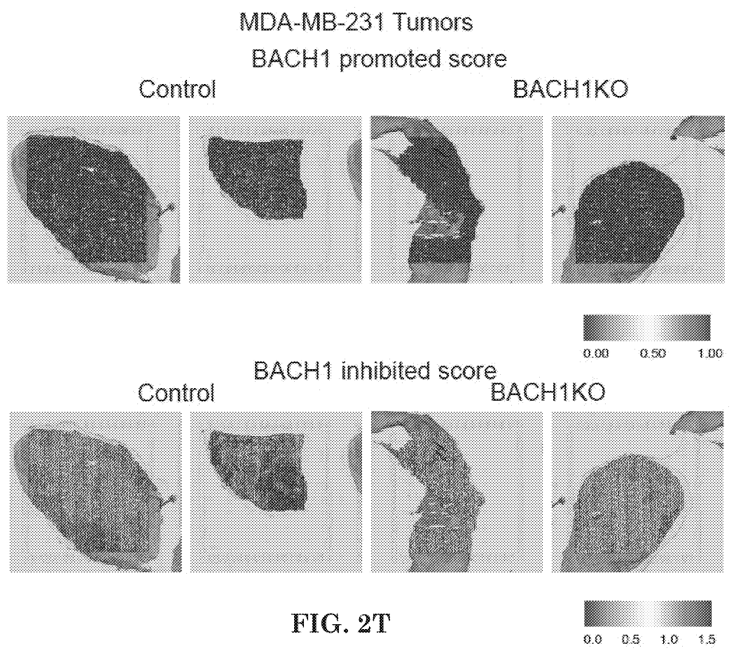

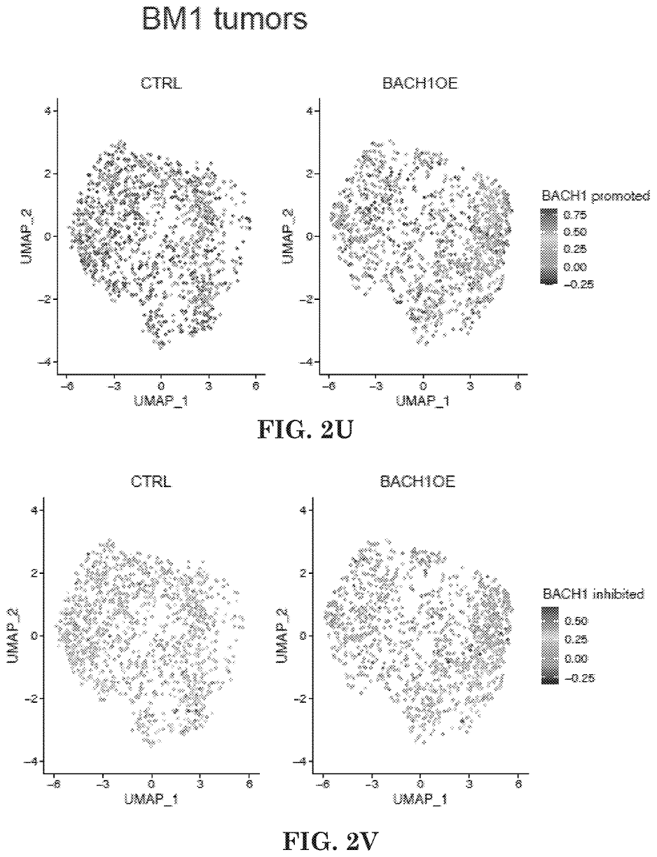

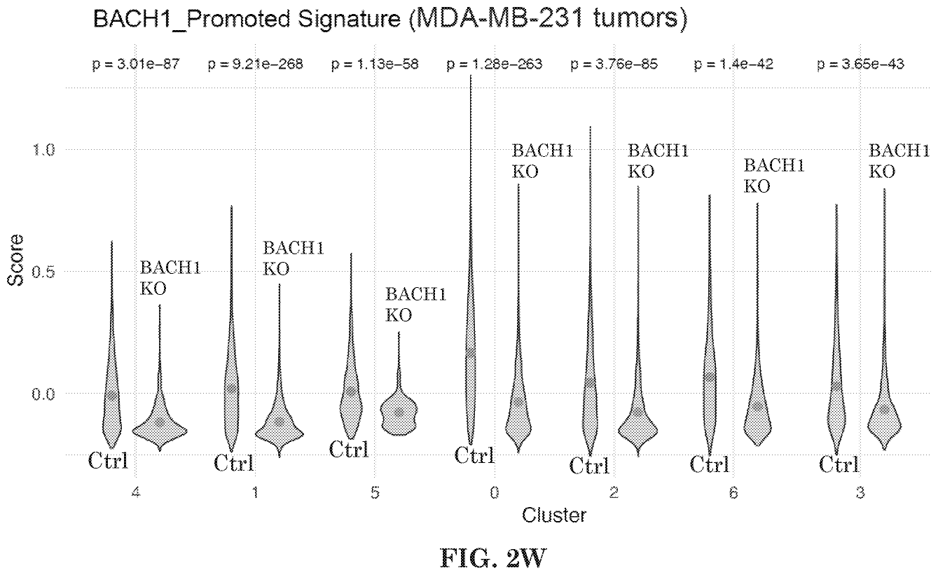

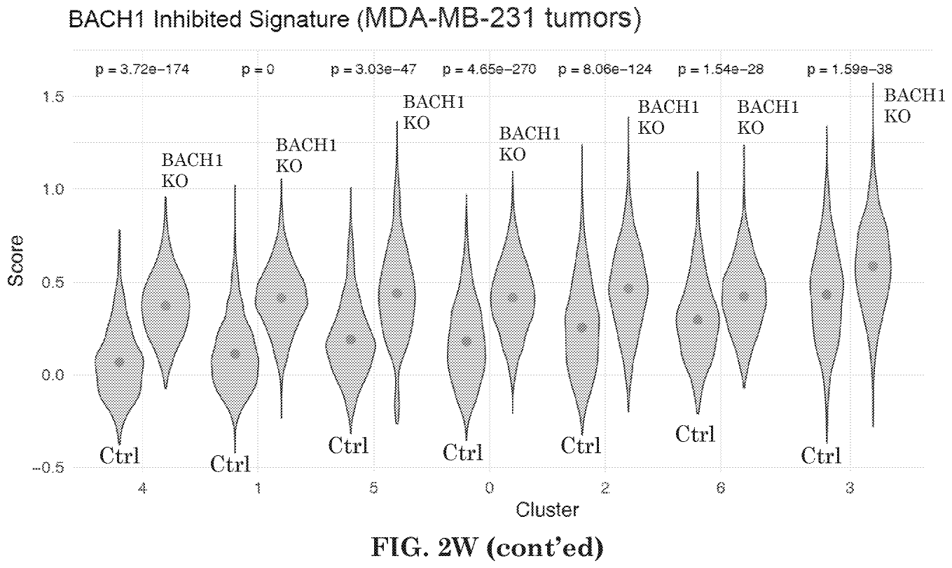

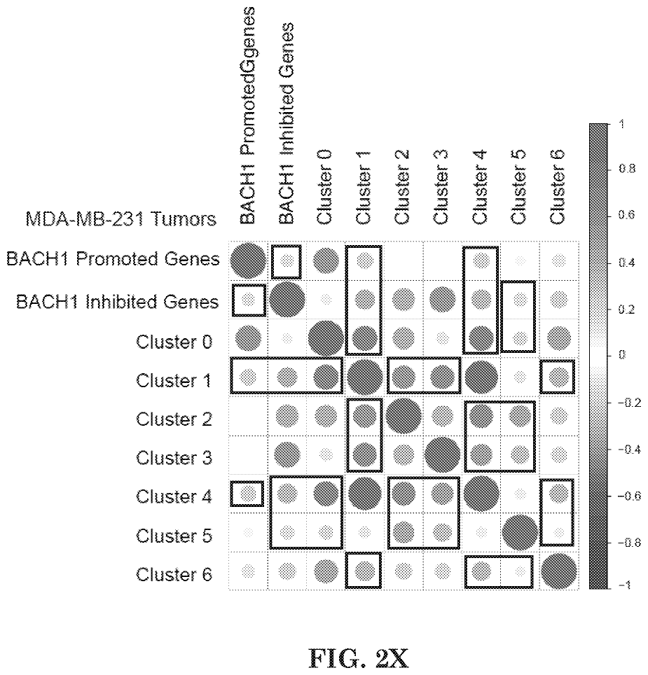

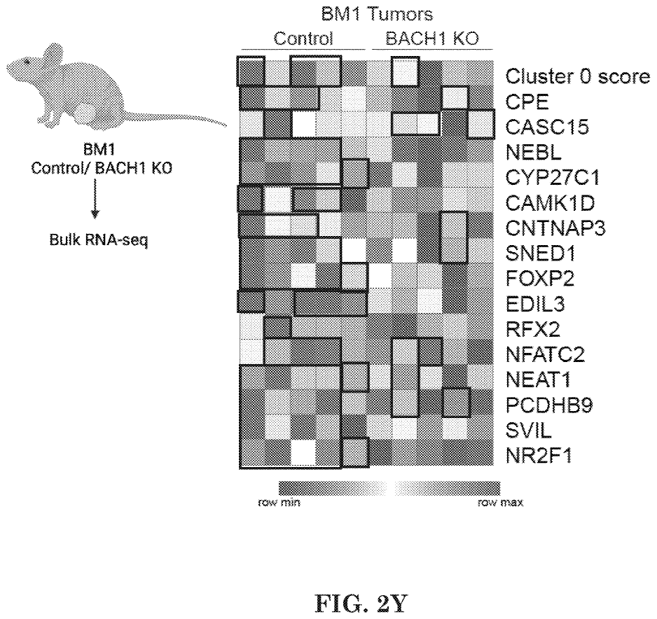

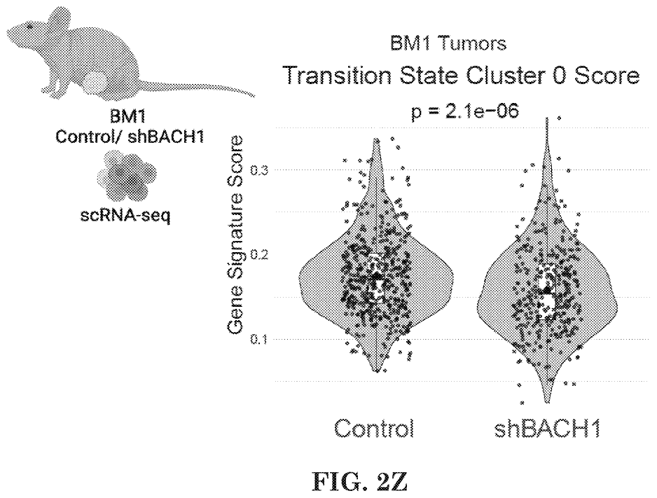

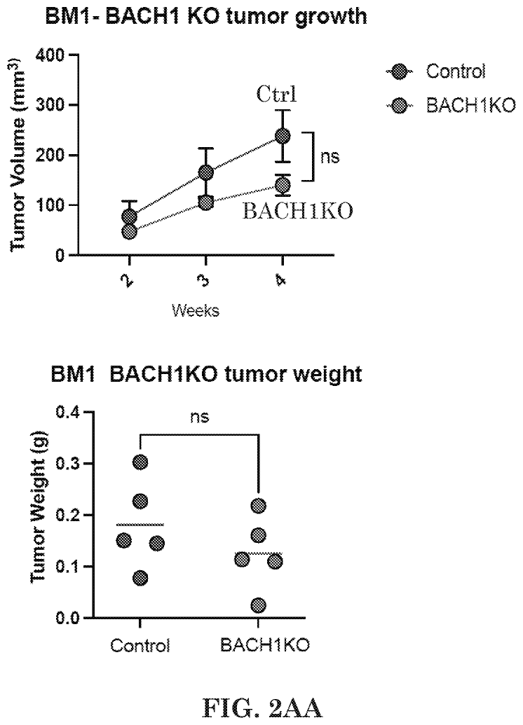

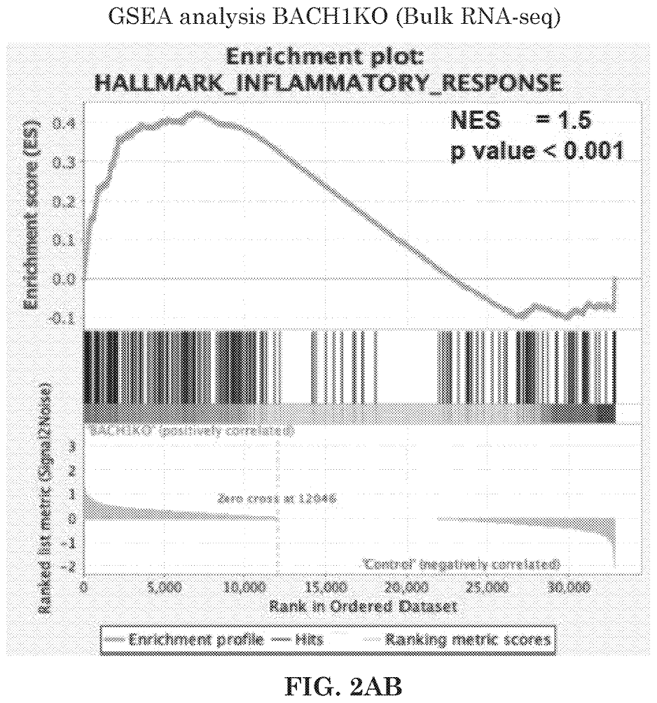

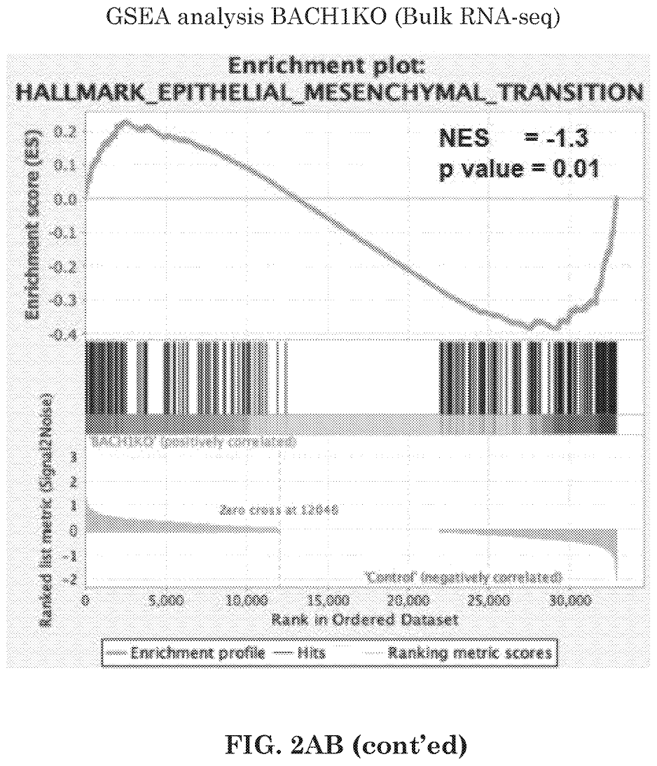

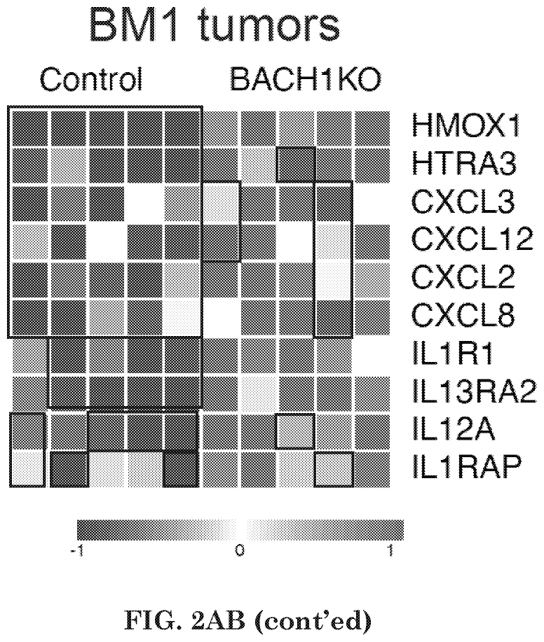

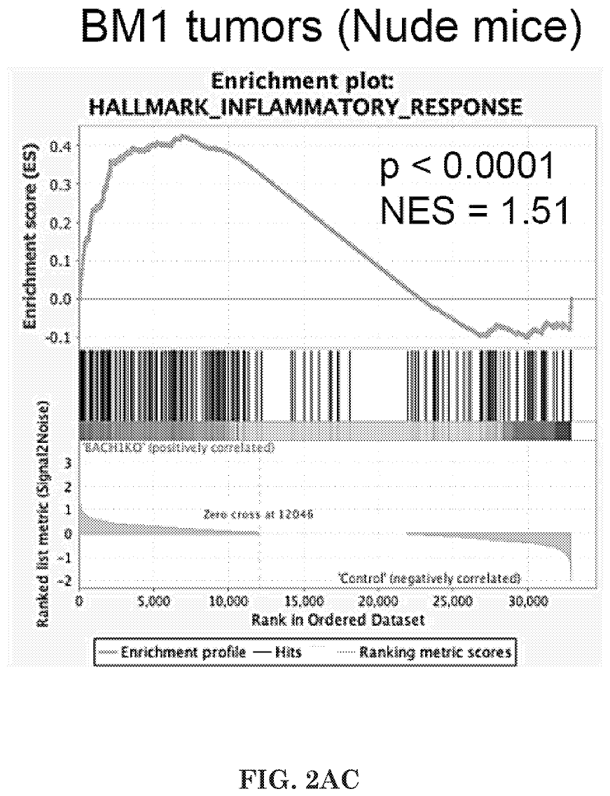

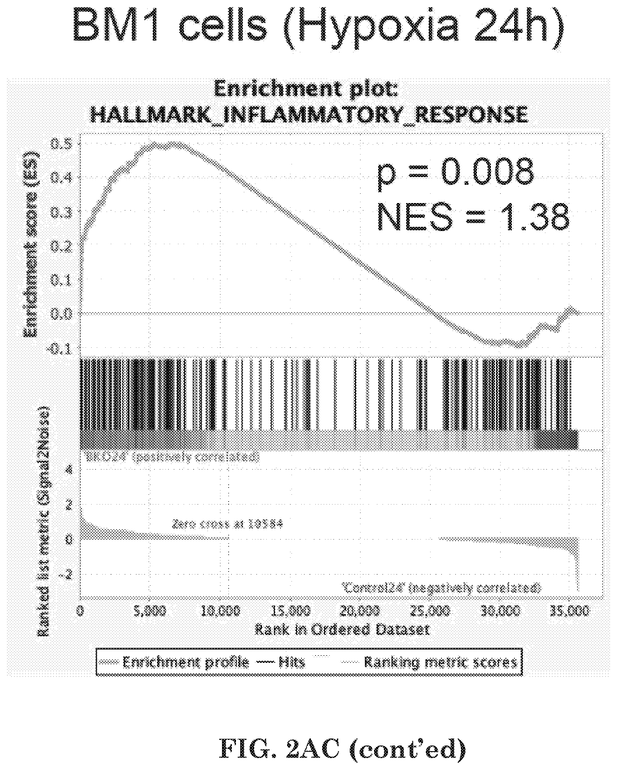

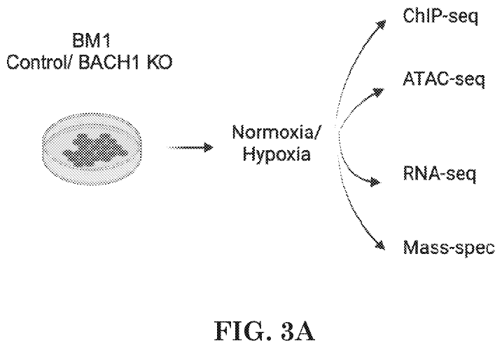

Gene Signature Calculation: BACH1 gene signature was constructed as described in (FIG. 2S), both hallmark gene signatures and BACH1-specific gene signatures provided in Tables 5-6. For scRNA-seq, the AddModuleScore( ) [32] function in Seurat was used to compute gene scores for each cell. The BACH1 Distance [27] Score was developed to quantify BACH1 transcriptional activity, evaluating the differential expression of BACH1-promoted and BACH1-inhibited genes derived from xenograft tumor models. For bulk RNA-seq, gene signature scores were computed as the mean log 2-transformed expression across the respective gene sets. The BACH1 Distance Score was defined as:

$$BACH 1 Distance Score = BACH {1}_{promoted} - BACH {1}_{inhibited}$$

A positive score indicates higher BACH1 transcriptional activity, while a negative score suggests reduced BACH1 activity.

Patient Outcome Analysis: the Cancer Genome Atlas Program (TCGA) results shown in this manuscript are based upon data generated by the TCGA Research Network: www.cancer.gov/tcga. Gene expression data from ISPY-2 were analyzed to assess the relationship between BACH1-associated gene signatures and pathologic complete response (pCR) across treatment arms. Z-scores for BACH1 mRNA expression (BmRNA), BACH1-promoted genes (B1 promote), BACH1-inhibited genes (B1 inhibit), and Tumor-Infiltrating Lymphocytes (TIL) as well as clusters gene signatures were computed. Patients were classified as high (Z>0) or low (Z≤0) for each score. Logistic regression was applied to assess the association between gene signatures and pathological complete response (pCR), modeling the logit transformation of P (pCR=1) as:

$$log ( \frac{P ( pCR = 1 )}{1 - P ( pCR = 1 )} ) = {β}_{0} + {β}_{1} \times Score$$

where score represents either a continuous z-score or a binary classification (high vs. low expression). The odds ratio (OR) was computed as:

$$OR = {e}^{{β}_{1}}$$

with confidence intervals:

$${CI}_{lower} = {e}^{{β}_{1} - 1.96 \times S E ( {β}_{1} )}$$

$${CI}_{upper} = {e}^{{β}_{1} + 1.96 \times S E ( {β}_{1} )}$$

For continuous scores, the OR reflects the change in pCR odds per unit increase, while for binary scores, it compares high vs. low expression groups. Forest plots visualized ORs and CIs (clipped to 0.1-10). For Kaplan-Meier analysis, KMPlot [81, 82] was used with gene sets derived from cell states or gene signatures.

Digital Histology Modeling: Breast cancer cases from TCGA were used for training and cross validation of gene expression signature prediction models. Slides and associated clinical data were accessed through the Genomic Data Commons Portal (portal.gdc.cancer.gov). Neoadjuvant datasets used for response prediction included a single arm phase II trial of durvalumab+neoadjuvant chemotherapy from Yale Cancer Center (NCT02489448) and prospectively consented patients receiving standard of care neoadjuvant chemotherapy at University of Chicago from 2006-2021. This analysis was reviewed by the University of Chicago IRB under approved protocol 22-0707. Slide images were extracted using the Slideflow pipeline with a tile size of 224 px per 224 μm to achieve an effective optical resolution of 10X, with filtering to remove tiles with >60% grayspace and additional Otsu thresholding and Gaussian blur based filtering to remove out of focus tiles/pen marks. No tumor region of interest annotations were performed in this analysis. Gene expression signature prediction models were trained in TCGA in a similar fashion to the previously published work [86], but updated to use the UNI [87] foundational model with the Bistro transformer architecture [88] for feature aggregation which was previously found to result in greater accuracy in signature prediction. A single model was trained to predict all cluster, TIL, and BACH1 signatures as a vector of outputs with a mean squared error loss function. Performance was estimated using five fold cross validation in TCGA, with a single model trained across the full dataset of TCGA for external predictions. Performance was assessed with Pearson correlation (versus true gene expression signature). Associations with pathologic complete response were assessed via AUC analysis, with confidence intervals generated via the Hanley and McNeil method [89]. An elastic net logistic regression model was trained will all signatures with an L1/L2 ratio of 0.5 using the University of Chicago dataset, and tested in the Yale dataset. UChicago and Yale patient data are presented in Table 8.

TABLE 1
Antibodies Used In Western Blot.

| | | | | Blocking |
|---|---|---|---|---|
| Antibody | Vendor Source | Identifier | Conc. | Conditions |
| BACH1 | Santa Cruz | RRID: AB_10608972, | 1:1000 | 5% Milk |
| | Biotechnology | Clone: N/A | | in TBS-T |
| GAPDH | Santa Cruz | RRID: AB_627679, | 1:1000 | 5% Milk |
| | Biotechnology | Clone: N/A | | in TBS-T |
| HIF1A | Novus | RRID: AB_3158237, | 1:1000 | 5% Milk |
| | | Clone: N/A | | in TBS |
| Tubulin | Thermo Fisher | RRID: AB_2210198, | 1:1000 | 5% Milk |
| | Scientific | Clone TU-02 | | in TBS-T |

TABLE 2
qPCR Primers.

| Primer | Sequence | SEQ ID NO |
|---|---|---|
| Mouse Bach1 Forward | CCATGACATCCGCAGAAGGAGT | 1 |
| Mouse Bach1 Reverse | GCGTTGACAGAATGTGGTCTCG | 2 |
| Human BACH1 Forward | CACCGAAGGAGACAGTGAATCC | 3 |
| Human BACH1 Reverse | GCTGTTCTGGAGTAAGCTTGTGC | 4 |
| Mouse Rpl13a Forward | CTGCTCTCAAGGTTGTTCGGCT | 5 |
| Mouse Rpl13a Reverse | CCTTCCGTTTCTCCTCCAGAGT | 6 |
| Mouse HMOX1 Forward | CACTCTGGAGATGACACCTGAG | 7 |
| Mouse HMOX1 Reverse | GTGTTCCTCTGTCAGCATCACC | 8 |
| Human EGLN3 Forward | GAACAGGTTATGTTCGCCACGTG | 9 |
| Human EGLN3 Reverse | CCCTCTGGAAATATCCGCAGGA | 10 |
| Human HMOX1 Forward | CCAGGCAGAGAATGCTGAGTTC | 11 |
| Human HMOX1 Reverse | AAGACTGGGCTCTCCTTGTTGC | 12 |
| Human NDRG1 Forward | ATCACCCAGCACTTTGCCGTCT | 13 |
| Human NDRG1 Reverse | GACTCCAGGAAGCATTTCAGCC | 14 |
| Human ANGPTL4 Forward | GATGGCTCAGTGGACTTCAACC | 15 |
| Human ANGPTL4 Reverse | TGCTATGCACCTTCTCCAGACC | 16 |
| Human ADM Forward | GACATGAAGGGTGCCTCTCGAA | 17 |
| Human ADM Reverse | CCTGGAAGTTGTTCATGCTCTGG | 18 |
| Human DNAJB9 Forward | GCCATGAAGTACCACCCTGACA | 19 |
| Human DNAJB9 Reverse | TCGTCTATTAGCATCTGAGAGTGT | 20 |
| Human DDIT4 Forward | GTTTGACCGCTCCACGAGCCT | 21 |
| Human DDIT4 Reverse | GCACACAAGTGTTCATCCTCAGG | 22 |
| Human HIF1A Forward | TATGAGCCAGAAGAACTTTTAGGC | 23 |
| Human HIF1A Reverse | CACCTCTTTTGGCAAGCATCCTG | 24 |
| Human EGLN1 Forward | TGAGCAGCATGGACGACCTGAT | 25 |
| Human EGLN1 Reverse | CGTACATAACCCGTTCCATTGCC | 26 |
| Human EGLN2 Forward | CTGTCTGGTATTTTGATGCCAAGG | 27 |
| Human EGLN2 Reverse | CGGCTGTGATACAGGTACTTGG | 28 |
| eGFP Forward | AGTCCGCCCTGAGCAAAGA | 29 |
| eGFP Reverse | TCCAGCAGGACCATGTGATC | 30 |
| Human CA9 Forward | GTGCCTATGAGCAGTTGCTGTC | 31 |
| Human CA9 Reverse | AAGTAGCGGCTGAAGTCAGAGG | 32 |
| Human P4HTM Forward | TGGTGGATGACTCCAGAGAGCA | 33 |
| Human P4HTM Reverse | ACTCTGCCTTGTGGCTCCTCAT | 34 |
| Human P4HA1 Forward | GCCAAAGCTCTGTTACGTCTCC | 35 |
| Human P4HA1 Reverse | CAAAGCAGTCCTCAGCCGTTAG | 36 |
| Human VHL Forward | GACACACGATGGGCTTCTGGTT | 37 |
| Human VHL Reverse | ACAACCTGGAGGCATCGCTCTT | 38 |
| Human FBXO22 Forward | TTCGTGTTGAGTAACCTGGCGG | 39 |
| Human FBXO22 Reverse | GAGATCCAGGTTACGCTCCGAT | 40 |
| Human FBXL17 Forward | AGAGACTGTGGATGTCGGATGG | 41 |
| Human FBXL17 Reverse | AGCTGTTCCACCGTCACTTCGT | 42 |
| Mouse exogenous BACH1 | GCGAGACAGATACAGAGGGCGA | 43 |
| Forward | | |
| Mouse exogenous BACH1 | CTGAGCGGCGATCCTGTTCTTG | 44 |
| Reverse | | |
| Mouse Cd74 Forward | GCTGGATGAAGCAGTGGCTCTT | 45 |
| Mouse Cd74 Reverse | GATGTGGCTGACTTCTTCCTGG | 46 |
| Mouse Cd80 Forward | CCTCAAGTTTCCATGTCCAAGGC | 47 |
| Mouse Cd80 Reverse | GAGGAGAGTTGTAACGGCAAGG | 48 |
| Mouse Cd86 Forward | ACGTATTGGAAGGAGATTACAGCT | 49 |
| Mouse Cd86 Reverse | TCTGTCAGCGTTACTATCCCGC | 50 |
| Mouse H2Ab1 Forward | AGGCTCAGTGATATGGTGCCCA | 51 |
| Mouse H2Ab1 Reverse | AGCCAAGTCCAGCAGTTCAGGA | 52 |
| Human HIF2A Forward | CTGTGTCTGAGAAGAGTAACTTCC | 53 |
| Human HIF2A Reverse | TTGCCATAGGCTGAGGACTCCT | 54 |

TABLE 3
CHIP qPCR Primers.

| | | SEQ |
|---|---|---|
| Primer | Sequence | ID NO |
| HS-CHIPBACH1- | AGTCGCGATTTCCTCATCCC | 55 |
| HMOX1 E1-Forward | | |
| HS-CHIPBACH1- | TTCCCTTTGTTTCCGCGAGT | 56 |
| HMOX1 E1-Reverse | | |
| HS-CHIPHIF1A- | GCTCTCGCTTCAGTCAGTCG | 57 |
| BACH1-Forward | | |
| HS-CHIPHIF1A- | TTCACTGGACCTGTCCGCC | 58 |
| BACH1-Reverse | | |
| HS-CHIPHIF1A- | CCGGTGACAGCCGATCC | 59 |
| PDK1-Forward | | |
| HS-CHIPHIF1A- | AGAAGCCACAGCCAGTACG | 60 |
| PDK1-Reverse | | |
| HS-CHIPBACH1- | ATGACTCAGCTAGTTGGACCG | 61 |
| SERPINE1-Forward | | |
| HS-CHIPBACH1- | CTCACATGGACCACAGCGAT | 62 |
| SERPINE1-Reverse | | |

TABLE 4
Guide RNA Sequences (CRISPR).

| gRNA | Sequence | SEQ ID NO |
|---|---|---|
| Mouse Bach1 | GCGGTTCCGAGCCCACCGCT | 63 |
| Human BACH1 | CTCAAGAATCGTAGGCCAGG | 64 |
| Mouse Hif1a | AGTGCACCCTAACAAGCCGG | 65 |

TABLE 5A
MDA-MB-231 Tumor Derived Gene Signatures of Cluster 0.
P value adjusted <0.05; percentage of cells expressing the
gene/feature in the cluster where the gene is detected (pct.
1) >0.25. MDA-MB-2231 control tumor with fold change (F.C) >1.
Cluster 0

| | | | | | |
|---|---|---|---|---|---|
| ADGRB3 | SOX5 | SSBP2 | SNED1 | HMCN1 | SCART1 |
| PLXDC2 | ITGB8 | GRIK2 | TCP11L2 | PARD3B | PLA2R1 |
| CPE | CYP27C1 | CNTNAP3 | FOXP2 | AC090015.1 | PCDHB9 |
| PLCB1 | AC233296.1 | TGFB2 | SPACA6 | AC098829.1 | MIR181A1HG |
| CSGALNACT1 | CAMK1D | AC079921.1 | PTPRK | LSAMP | SVIL |
| CASC15 | PCDH9 | PRKN | EDIL3 | CDON | MALRD1 |
| LRP1B | TCIM | MPP7 | RFX2 | AL050309.1 | NR2F1 |
| CEBPD | CNTNAP3B | EREG | NFATC2 | PCDHGA10 | CPEB2 |
| LINC02163 | AC109927.1 | FAT4 | ENOX1 | NEAT1 | AC060765.1 |
| NEBL | AC119674.1 | NBEA | PLD1 | EYS | TNFSF10 |
| | | | | | Z83843.1 |
| | | | | | AKAP6 |

TABLE 5B
MDA-MB-231 Tumor Derived Gene Signatures of Cluster 1.
P value adjusted <0.05; percentage of cells expressing the
gene/feature in the cluster where the gene is detected (pct.
1) >0.25. MDA-MB-2231 control tumor with fold change (F.C) >1.
Cluster 1

AC011447.3
AC073529.1
ADCY3
ADGRV1
ANKS1B
ANLN
AP001347.1
APOC1
ARHGAP11A
ARHGAP11B
ARHGAP19
ARPIN
ASF1B
ASPM
ATAD2
ATAD5
ATG16L2
ATRNL1
AURKB
BARD1
BCL2
BIRC5
BLM
BRCA1
BRCA2
BRI3BP
BRIP1
BUB1
BUB1B
C17orf53
C18orf54
C19orf48
C1orf112
C21orf58
C4orf46
C5orf34
C9orf40
CAMK4
CASP8AP2
CCDC15
CCDC150
CCDC34
CCHCR1
CCNA2
CCNE1
CCNE2
CCNF
CCP110
CDC25A
CDC25C
CDC45
CDC6
CDC7
CDCA2
CDCA3
CDCA4
CDCA5
CDCA7
CDCA8
CDK1
CDK2
CDT1
CENPE
CENPF
CENPH
CENPI
CENPJ
CENPK
CENPM
CENPN
CENPO
CENPP
CENPQ
CENPS
CENPU
CENPW
CEP128
CEP152
CEP55
CEP78
CEP85
CHAF1A
CHAF1B
CHEK1
CHEK2
CHTF18
CIP2A
CIT
CKAP2
CKAP2L
CKS1B
CLN6
CLSPN
CMC2
DBF4B
DCK
DCLRE1B
DDIAS
DEK
DEPDC1
DEPDC1B
DHFR
DIAPH3
DLGAP5
DNA2
DNAJC9
DNMT1
DNMT3B
DONSON
DPF1
DSCC1
DSN1
DTL
DTYMK
DUT
E2F1
E2F8
ECT2
EMP2
ERCC6L
ESCO2
ESPL1
EXO1
EXOSC8
EXOSC9
EZH2
FADS1
FAM111A
FAM111B
FAM161A
FANCA
FANCB
FANCD2
FANCG
FANCI
FANCL
FBXO43
FBXO5
FEN1
FIGNL1
FKBP5
FOXM1
FOXRED2
GEN1
GGCT
GGH
GINS1
GINS2
GINS4
GMNN
GPAT2
GPR137C
GTSE1
H2AFX
HAT1
HAUS1
HAUS2
HAUS5
HAUS8
HELLPAR
HELLS
HIRIP3
HIST1H1E
HIST1H2AH
HIST1H3B
HIST1H4C
HIST2H2AC
HJURP
HMGB2
HMMR
HMSD
HS6ST3
ICMT
IGFLR1
IL11
INCENP
IPO9
IQGAP3
KIF11
KIF14
KIF15
KIF18A
KIF18B
KIF20B
KIF22
KIF23
KIF24
KIF2C
KIF4A
KIFC1
KLHL23
KNL1
KNTC1
LIG1
LIN52
LIN54
LIN9
LINC01224
LINC01478
LINC01572
LMNB1
LMNB2
LRP8
LRR1
LRRC45
LRRCC1
MAD2L1
MASTL
MBOAT1
MCM10
MCM2
MCM3
MCM4
MCM5
MCM6
MCM7
MCM8
MELK
MGME1
MIR924HG
MIS18A
MIS18BP1
MKI67
MMD
MMS22L
MND1
MNS1
MPHOSPH9
MRE11
MSH2
MTBP
MTFR2
MTHFD1
MXD3
MYBL1
MYBL2
MYH15
MYO19
NASP
NCAPD2
NCAPD3
NCAPG
NCAPG2
NCAPH
NCAPH2
NDC1
NDC80
NEIL3
NEMP1
NR2C2AP
NRGN
NRM
NSD2
NUDT8
NUF2
NUP155
NUP85
NUSAP1
OIP5
ORC1
ORC6
PAQR4
PARP1
PARPBP
PBK
PBX3
PCLAF
PCNA
PCNT
PHF19
PHGDH
PKMYT1
PKN3
PLEKHA6
PLK4
POC1A
POGLUT3
POLA1
POLA2
POLD1
POLD3
POLE
POLE2
POLH
POLQ
POLR3K
PRC1
PRIM1
PRIM2
PRKDC
PRR11
PRRG4
PRTFDC1
PSMC3IP
PXMP2
RACGAP1
RAD18
RAD51
RAD51AP1
RAD51C
RAD54B
RAD54L
RAPGEF5
RARB
RBBP8
RBL1
RECQL4
RFC2
RFC3
RFC4
RFC5
RFWD3
RHNO1
RMI1
RMI2
RNASEH2A
RPA1
RPA2
RPA3
RRM1
RRM2
RTKN2
RTTN
RUSC1
SAC3D1
SAE1
SASS6
SCML2
SFN
SGO1
SGO2
SHCBP1
SHMT1
SKA1
SKA3
SKP2
SLC16A4
SLC25A10
SLF1
SMC2
SMC4
SNED1
SPAG5
SPATA5
SPC24
SPC25
SPDL1
STIL
SULT1B1
SUV39H2
TACC3
TBC1D31
TCF19
TEDC1
TEDC2
TEX30
TFDP1
THOP1
TICRR
TIMELESS
TK1
TLCD4
TMEM106C
TMEM97
TMPO
TONSL
TOP2A
TOP3A
TOPBP1
TPX2
TRAIP
TRIP13
TTF2
TTK
TUBA1B
TUBG1
TYMS
TYMSOS
UBE2C
UBE2T
UBR7
UHRF1
UNG
USP1
VRK1
WDHD1
WDR34
WDR62
WDR76
XRCC2
XRCC3
YEATS4
Z94721.1
Z94721.3
ZGRF1
ZNF367
ZNF519
ZNF724
ZNF93
ZWILCH
ZWINT

TABLE 5C
MDA-MB-231 Tumor Derived Gene Signatures of Cluster 2.
P value adjusted <0.05; percentage of cells expressing the
gene/feature in the cluster where the gene is detected (pct.
1) >0.25. MDA-MB-2231 control tumor with fold change (F.C) >1.
Cluster 2

AC006058.1
AC010210.1
AC010343.3
AC016745.1
AC023157.3
AC044810.2
AC060765.1
AC068633.1
AC116049.2
AC116345.1
ADAMTS9-AS2
ADTRP
AGBL4
AGMO
AL050309.1
AL354740.1
ARHGAP22
BMP2
BTBD8
CCDC9B
CFAP299
CNIH3
DMD
DUXAP8
EPCAM-DT
FAP
GPR173
IQSEC2
ITGB1-DT
KCNJ15
KCNN2
LDAH
LINC01376
LINC01605
LINC02163
LINC02377
LINC02577
LINC02649
MIR100HG
MSR1
NLRP1
PLA2R1
PRKN
RAD51-AS1
RIN2
RPS6KA2
SH3TC2-DT
SLC22A15
SLC22A3
SLC2A3
TET1
THSD4
TMEM163
TNFSF15
TXNIP
WFDC10B
ZNF41
ZNF514

TABLE 5D
MDA-MB-231 Tumor Derived Gene Signatures of Cluster 3.
P value adjusted <0.05; percentage of cells expressing the
gene/feature in the cluster where the gene is detected (pct.
1) >0.25. MDA-MB-2231 control tumor with fold change (F.C) >1.
Cluster 3

ABCA1
ABHD17B
AC097534.2
AC105402.3
AC107021.1
ADM
AKAP17A
AL118516.1
AL158152.1
AL731577.2
ANG
ANKRD37
AP5Z1
ARRDC3
ATG14
ATP6V1H
AVPI1
BAG3
BCL7A
BNIP3
C15orf48
C1orf52
CA9
CACYBP
CCL26
CCL28
CCN3
CCNT2
CEBPG
CEP95
CHORDC1
CITED2
CLEC2B
CNNM2
COG3
CREBRF
CTH
CTR9
CWF19L2
DDIT3
DDIT4
DEDD2
DENND4C
DEPP1
DIPK2A
DNAJB1
DNAJB4
DSEL
EAF1
ECD
EDRF1
EGLN3
EIF2AK3
ENO2
EPB41L4A-AS1
EPG5
ERO1A
ERO1B
EXOC1
F3
FAM13A
FAM160A1
FAM210A
FAM3C
FBXL2
FEM1C
FILIP1L
FLCN
FNBP1
FTL
GADD45A
GADD45B
GAS5
GGA2
GNAI1
GULP1
HARS2
HERC3
HIF1A-AS3
HILPDA
HK2
HMOX1
HSP90AA1
HSPA1A
HSPA1B
HSPA5
HSPA6
HSPB1
HSPH1
IARS
IGFBP3
IKZF5
INSIG2
ITPR1
KDM3A
KHDC4
KLHL24
KMT5B
LAMTOR3
LIMCH1
LINC01578
LMTK2
LOX
MANBA
MAPKAPK5-AS1
MBIP
MINDY3
MITF
MKNK2
MT1X
MTREX
MTRNR2L12
MTRNR2L8
MTUS1
MXI1
MYLIP
NDRG1
NECAP1
NFIL3
NMT2
NOG
ORC4
OSER1
P4HA1
P4HA2
PAG1
PAM
PDK1
PDK4
PFKFB3
PFKFB4
PLAG1
PLCG2
PLEKHF2
PLIN2
PLOD2
PPP1R15A
PPP1R3B
PRKAB2
PRPF18
PRPF39
PSAP
RAB30
RALA
RBM7
RGS4
RIOK3
RIT1
RLF
RNASE4
RNMT
RRAGB
RRAGD
RSRC2
RSRP1
SAT2
SEL1L
SERPINH1
SLC2A1
SLC2A3
SLC3A2
SLC5A3
SNAP23
SNAPC1
SNHG12
SNHG15
SNHG17
SNHG8
SNIP1
SPTY2D1
STAM
STX3
TAF1D
TAF7
THAP9-AS1
TICAM2
TMEM158
TMEM159
TMEM167B
TMEM265
TMEM45A
TOM1
TOX
TPD52
TRA2A
TRAF6
TSPYL2
UPP1
USPL1
VEGFA
VGLL3
WDR66
WSB1
YPEL2
ZCCHC8
ZDHHC7
ZFAND2A
ZFAS1
ZFPM2-AS1
ZNF10
ZNF14
ZNF160
ZNF317
ZNF330
ZNF395
ZNF410
ZNF420
ZNF581
ZNF704
ZNF830
ZNF844

TABLE 5E
MDA-MB-231 Tumor Derived Gene Signatures of Cluster 4.
P value adjusted <0.05; percentage of cells expressing the
gene/feature in the cluster where the gene is detected (pct.
1) >0.25. MDA-MB-2231 control tumor with fold change (F.C) >1.
Cluster 4

AC073529.1
AC240274.1
ADGRE5
ANLN
ANP32E
APOLD1
ARHGAP11A
ARHGAP11B
ARHGAP19
ARL6IP1
ASPM
ATP8B3
AURKA
AURKB
BIRC5
BORA
BUB1
BUB1B
BUB3
C2orf69
CBR3
CCDC85C
CCNA1
CCNA2
CCNB1
CCNB2
CCNF
CD3EAP
CD82
CDC20
CDC25B
CDC25C
CDCA2
CDCA3
CDCA8
CDK1
CDKN2C
CDKN3
CENPA
CENPE
CENPF
CENPI
CENPN
CENPW
CEP55
CEP70
CEP97
CIP2A
CIT
CKAP2
CKAP2L
CKAP5
CKS1B
CKS2
CRNDE
DBF4
DBF4B
DCK
DDIAS
DDX39A
DEPDC1
DEPDC1B
DLGAP5
DTYMK
EMC9
ENPP1
EPB41L4B
ERCC6L
ESAM
EVI2A
FAM110A
FAM83D
FAM89A
FBXO43
FOPNL
FOXM1
G2E3
GAS2L3
GEN1
GOT1
GTSE1
HELLPAR
HJURP
HMGB2
HMGB3
HMGN2
HMMR
HYLS1
INCENP
IQGAP3
KIF11
KIF14
KIF15
KIF18A
KIF18B
KIF20A
KIF20B
KIF22
KIF23
KIF2C
KIF4A
KIF5B
KIFC1
KNL1
KNSTRN
KPNA2
LAPTM5
LBR
LMNB1
LMNB2
LYAR
MAD2L1
MIS18BP1
MKI67
MXD3
MZT1
NCAPD2
NCAPG
NCAPH
NDC80
NEIL3
NEK2
NLGN1
NMU
NOX5
NUDCD2
NUF2
NUSAP1
OIP5
P2RY2
PARPBP
PBK
PCSK9
PHF19
PHF7
PIF1
PIMREG
PLK1
POC1A
PRC1
PRR11
PSRC1
PTTG1
RACGAP1
RAD21
RANGAP1
RCCD1
REEP4
RHNO1
RNF26
RNFT2
SAPCD2
SCLT1
SGO1
SGO2
SHCBP1
SKA1
SKA2
SKA3
SMC4
SMTN
SNHG4
SOGA1
SPA17
SPAG5
SPC24
SPC25
SPDL1
STARD8
STIL
SUN2
TACC3
TESMIN
TMEM19
TMEM71
TMPO
TNFAIP8L1
TOP2A
TPX2
TRAIP
TRIP13
TRMU
TROAP
TTK
TUBA1B
TUBA1C
TYMSOS
UBALD2
UBE2C
UBE2S
USP13
WDR62
WRAP53
ZNF699
ZWINT

TABLE 5F
MDA-MB-231 Tumor Derived Gene Signatures of Cluster 5.
P value adjusted <0.05; percentage of cells expressing the
gene/feature in the cluster where the gene is detected (pct.
1) >0.25. MDA-MB-2231 control tumor with fold change (F.C) >1.
Cluster 5

ACD
CD74
CST2
CST4
ICAM2
KRT81
MAGEA6
MYL9
SHISA4
SPDEF

TABLE 5G
MDA-MB-231 Tumor Derived Gene Signatures of Cluster 6.
P value adjusted <0.05; percentage of cells expressing the
gene/feature in the cluster where the gene is detected (pct.
1) >0.25. MDA-MB-2231 control tumor with fold change (F.C) >1.
Cluster 6

ABCC3
AC005920.2
AC090001.1
ACSL3
AIM2
AKR1C1
AL021707.1
AL133330.1
AL512603.2
ANGPTL2
AP002784.1
APOL1
APOL2
APOL6
ATP2B4
ATP9A
B4GALT5
BAMBI
C5orf56
CAVIN2
CCDC6
CCDC68
CCN3
CCND3
CFAP54
CHST15
CLIC3
CNKSR3
COL6A1
CPEB3
CRIP2
CXXC4
DDX58
DDX60
DDX60L
DHX58
DMD
DSP
EPAS1
EPSTI1
ETV3
FLVCR2
GALNT3
GALNT7
GRB10
GTPBP1
HAPLN3
HERC5
HERC6
HES4
HIP1R
HLA-B
HOMER1
HS6ST3
ICAM1
IFI27
IFI44
IFI44L
IFI6
IFIH1
IFIT1
IFIT2
IFIT3
IFIT5
IFITM1
IGFBP6
IL15
IL32
IL6
ISG15
ISG20
JAK2
KAZN
KDM6A
KLF2
KLHL3
LIMS2
LINC-PINT
LIPA
LY6E
MAP4K4
MAPRE2
MFSD14A
MUC1
MX1
MX2
N4BP1
NEAT1
NEBL
NEDD1
NNMT
NOCT
NTN4
OAS1
OAS2
OAS3
OASL
OLFML2A
PANX1
PARP14
PARP9
PCSK1N
PDK3
PHLDA3
PIK3R3
PLAAT4
PLAUR
PLEKHA4
PMAIP1
PPM1K
PPP1R13B
PTGER4
RAPGEF2
RASGRP3
RFK
RHEBL1
RIPK1
RIPK4
RNF149
RP2
RPS6KC1
S100A2
SAMD9
SAMD9L
SERINC2
SLC45A4
SLCO3A1
SOX5
SPAG9
ST8SIA4
STK24
SYNM
TCF7L1
TDRD7
TENT5A
THEMIS2
TINAGL1
TMEM62
TNFSF10
TOGARAM2
TRANK1
TRIM26
TTC28
TULP2
UBN2
WARS
WNT9A
XKR8
ZC3HAV1
ZFP36L2
ZNF704
ZNFX1
ZNRF2

TABLE 6
BACH1 Gene Signatures. Construction Shown In FIG. 2S.

| BACH1-promoted gene signature | BACH1-inhibited gene signature |
|---|---|
| B4GALNT3 | FTL |
| CMTM8 | GCLM |
| MAP2 | IGFBP3 |
| PLXDC2 | IL13RA2 |
| SPOCK1 | MAGEC2 |
| | NUPR1 |
| | SERPINE1 |
| | TNFSF15 |

TABLE 7
BACH1 Gene Signatures Used In Experiments. Full BACH1 promoted-
genes: Overlapped between MDA-MB-231 BACH1 KO vs Control
in both spatial (F.C <−0.7) and scRNA-seq (F.C <−0.7)
datasets, p.adjust value <0.05. Full BACH1 inhibited-
genes: Overlapped between MDA-MB-231 BACH1 KO vs Control
in both spatial (F.C >0.3) and scRNA-seq (F.C >0.5)
datasets, p.adjust value <0.05.

| Full BACH1-promoted gene | | | Full BACH1-inhibited gene |
|---|---|---|---|
| signature (99 genes) | | | signature (33 genes) |
| AC011893.1 | GNAO1 | RIMKLA | ABCA3 |
| AC068985.1 | GPR87 | RNF43 | AC005224.3 |
| AC090809.1 | IGFL2-AS1 | SCARA3 | AC064869.1 |
| ADAMTS15 | ITIH4 | SERPINA1 | AL590681.1 |
| AL590652.1 | KLHDC8B | SH3BP1 | CREB3L2 |
| ARHGAP25 | KRT86 | SLC38A5 | CT55 |
| ARHGAP28 | LINC00261 | SOD3 | EEF1A1 |
| ARHGAP40 | LINC00526 | SPINT1 | ESM1 |
| ARMH1 | LINC01088 | SPOCK1 | FBXO30 |
| B4GALNT3 | LINC02562 | SPOCK3 | FTH1 |
| BCAM | LONRF2 | SSTR2 | FTL |
| C5orf46 | LRRC75B | ST6GAL1 | GCLM |
| CASP1 | MANEAL | STK32A | GNAI1 |
| CCDC149 | MANSC1 | SYTL2 | HMOX1 |
| CD24 | MAOB | TCIM | ICAM5 |
| CD96 | MAP2 | TET1 | IGFBP3 |
| CEMIP | MPP7 | TFF2 | IL13RA2 |
| CENPV | ONECUT2 | TIAM1 | KISS1 |
| CHDH | PDE8B | TIMP4 | LINC02303 |
| CHRDL1 | PDGFD | TINAGL1 | MAGEC2 |
| CMTM8 | PLB1 | TMC5 | NEDD4 |
| COL6A2 | PLCH1 | TMEM38A | NKX2-1 |
| COLGALT2 | PLCL2 | TMLHE | NUPR1 |
| CST5 | PLD5 | TNFSF10 | PCK2 |
| DNALI1 | PLLP | TOGARAM2 | PLCG2 |
| DSC2 | PLXDC2 | TRBV30 | PTPRN2 |
| DUOX1 | PPARGC1A | TRPV4 | RGS20 |
| EEF1A2 | PRSS2 | TSPAN8 | SERPINE1 |
| ENPP4 | RARRES2 | URB1-AS1 | SLC6A8 |
| EWSAT1 | RASL10A | VSNL1 | SLITRK1 |
| EYA4 | RAVER2 | WFS1 | TNFSF15 |
| FIBCD1 | RBM24 | ZNF467 | TPT1 |
| GJB2 | RHPN1 | ZNF726 | XAGE1B |

TABLE 8
Patient Data Sheet. TMN staging system was used to grade the cancer (T:
tumor size/extent; N: lymph node involvement; M: distant metastasis).
HER2: growth hormone protein biomarker. HR: hormone receptor biomarker
indicating responsiveness to estrogen/progesterone. are biomarkers.
Grade reflects how aggressive or abnormal the cancer cells appear. TAB:
summary of grouping of tumor staging or assessment information.

| | | Grouped by Cohort | | | | | | |
|---|---|---|---|---|---|---|---|---|
| | | Missing | Overall | | UChicago | | Yale | |
| n | | | 191 | | 136 | | 55 | |
| Age, mean | | 0 | 51.9 | (11.9) | 52.7 | (12.5) | 50.1 | (10.0) |
| (SD) | | | | | | | | |
| Tab | T1 | 0 | 46 | (24.1) | 28 | (20.6) | 18 | (32.7) |
| | T2 | | 103 | (53.9) | 74 | (54.4) | 29 | (52.7) |
| | T3 | | 35 | (18.3) | 27 | (19.9) | 8 | (14.5) |
| | T4 | | 7 | (3.7) | 7 | (5.1) | | |
| N, n (%) | N0 | 0 | 82 | (42.9) | 53 | (39.0) | 29 | (52.7) |
| | N1 | | 86 | (45.0) | 64 | (47.1) | 22 | (40.0) |
| | N2 | | 15 | (7.9) | 14 | (10.3) | 1 | (1.8) |
| | N3 | | 7 | (3.7) | 4 | (2.9) | 3 | (5.5) |
| | Unknown | | 1 | (0.5) | 1 | (0.7) | | |
| M, n (%) | M0 | 0 | 188 | (98.4) | 133 | (97.8) | 55 | (100.0) |
| | M1 | | 2 | (1.0) | 2 | (1.5) | | |
| | Unknown | | 1 | (0.5) | 1 | (0.7) | | |
| HER2, n (%) | Negative | 0 | 191 | (100.0) | 136 | (100.0) | 55 | (100.0) |
| HR, n (%) | Negative | 0 | 126 | (66.0) | 71 | (52.2) | 55 | (100.0) |
| | Positive | | 65 | (34.0) | 65 | (47.8) | | |
| arm, n (%) | Other | 0 | 25 | (13.1) | 25 | (18.4) | | |
| | TC | | 6 | (3.1) | 6 | (4.4) | | |
| | Tax-AC | | 105 | (55.0) | 105 | (77.2) | | |
| | Durvalumab + | | 55 | (28.8) | | | 55 | (100.0) |
| | nab-Paclitaxel | | | | | | | |
| Grade, n (%) | 1 | 0 | 3 | (1.6) | 3 | (2.2) | | |
| | 2 | | 20 | (10.5) | 20 | (14.7) | | |
| | 3 | | 113 | (59.2) | 113 | (83.1) | | |
| | Unknown | | 55 | (28.8) | | | 55 | (100.0) |
| pCR, n (%) | 0.0 | 0 | 118 | (61.8) | 88 | (64.7) | 30 | (54.5) |
| | 1.0 | | 73 | (38.2) | 48 | (35.3) | 25 | (45.5) |

TABLE 9
Antibodies and concentrations used in flow cytometry.

| Antibody | Fluorophore | Concentrations |
|---|---|---|
| PD1 | BUV395 | 1:200 |
| CD86 | BUV 496 | 1:200 |
| CD69 | BUV 563 | 1:200 |
| Ly6G | BUV 661 | 1:200 |
| CD103 | BV421 | 1:200 |
| I-A/I-E | Pacific Blue | 1:400 |
| CD19 | BV510 | 1:200 |
| CD11b | BV570 | 1:200 |
| CD206 | BV605 | 1:200 |
| PDCA | BV650 | 1:200 |
| Ki67 | BV711 | 1:200 |
| CD4 | Spark 685 | 1:200 |
| Ly6C | BV785 | 1:200 |
| CD44 | FITC | 1:200 |
| CD45 | AF532 | 1:200 |
| F4/80 | PerCP/Cy5.5 | 1:200 |
| CD8a | PerCP ef710 | 1:200 |
| CD80 | PE/Dazzle 594 | 1:200 |
| CD62L | PE/Cy7 | 1:200 |
| CD11c | AF 647 | 1:200 |
| Foxp3 | APC | 1:150 |
| CD3 | AF 700 | 1:200 |
| Viability | Zombie NIR | 1:400 |
| NK1.1 | APC Cy7 | 1:200 |
| Fc Block | NA | 1:200 |

### Example 1: Phenotypic Plasticity was Associated with a Hypoxia Gradient in TNBC Tumors

To understand the impact of environmental stress on tumor cell states, the range of cancer cell heterogeneity was initially evaluated within a xenograft tumor generated by injection of the human TNBC cell line MDA-MB-231 into nude mice (FIG. 1A). Single-cell RNA sequencing (scRNA-seq) analysis of the human transcripts in xenograft tumors revealed considerable phenotypic diversity (FIG. 1B). Uniform Manifold Approximation and Projection (UMAP) clustering delineated seven distinct clusters specific to human cancer cells, comprising two proliferative clusters and five stress-associated clusters (FIG. 1B and FIG. 1C). Analysis of single cell RNA-seq from cultured MDA-MB-231 or BM1 cells indicates that the injected cells predominantly reside in proliferative and metabolic stress states (FIG. 1D, FIG. 1E). Modeling of the inferred cellular trajectories predicts that tumor cells transition from an initial proliferative (#4) or DNA damage (#1) state through an intermediate metabolic stress state (#5), eventually reaching a transition state (#0). From this state, they branch into either an inflammation (#6) or migration (#2) phenotype, with migration culminating in a high-stress state (#3) (FIG. 1F, FIG. 1G).

Three-dimensional UMAP resolution identifies Cluster 0 as a central nodule that interfaces with other clusters, consistent with predicted branch points (FIG. 1H). Supporting these assignments, spatial transcriptomic mapping of cluster gene signatures shows the localization of key clusters to distinct tumor regions. Specifically, proliferative cells (Clusters 4, 1) are mainly located at the periphery, cells under high stress (Cluster 3) are found in the center adjacent to the necrotic region, and cells associated with the transition state (Cluster 0) are largely situated at the intersection between these two states (FIG. 1I). Gene signatures for the individual clusters confirm these relative relationships among the various cell states (FIG. 1J).

Multiple lines of evidence suggest that most of the cell clusters are subject to a gradient of hypoxic stress that drives gene expression changes across different cell states. First, examination of hypoxia-induced genes using the Hallmark Hypoxia score revealed an increasing hypoxia response from metabolic stress Cluster 5 to high stress Cluster 3 (FIG. 1K). Correlations between single cell-based cluster gene signatures and the Hallmark Hypoxia signature further support this relationship (FIG. 1J). Second, spatial analysis of tumor cells showed a progressive increase in co-localization of cells expressing both the Hallmark Hypoxia and cluster gene signatures: lowest for Cluster 4, intermediate for Cluster 0, and highest for Cluster 3 (FIG. 1L). Third, electron paramagnetic resonance (EPR) analysis of MDA-MB-231 tumors in live mice directly confirmed the presence of low oxygen tension, indicating that ~50% of tumor cells are exposed to hypoxic stress with a similar distribution to that defined by spatial transcriptomics (FIG. 1M). Fourth, analysis of MDA-MB-231 tumor cells expressing GFP upon activation of a hypoxia response element (HRE) [7] demonstrated that all cell state trajectories originating from Cluster 0 had undergone hypoxic exposure during tumor growth (FIG. 1N). In contrast, neither proliferating (Clusters 4, 1) nor metabolically stressed (Cluster 5) cells exhibited significant HIF-induced hypoxic responses (FIG. 1N). Notably, hypoxia alone did not shift cultured cells out of a proliferative state, even after 72 hours of exposure, highlighting the importance of the in vivo context (FIG. 1O). Lastly, a UMAP depicting expression of the most variable genes in MDA-MB-231 tumors, ordered across inferred Palantir pseudotime, predicted a transition across distinct transcriptional programs from proliferation to a robust hypoxia response (FIG. 1P). These results suggest that hypoxic stress is a driving force behind phenotypic transitions among tumor cell states, culminating in the most hypoxic state (Cluster 3).

To assess the impact of a hypoxic stress gradient on cell fates associated with metastasis and inflammation, we conducted a detailed characterization of several cell clusters. One cell state in particular, Cluster 0, possesses attributes indicative of metastatic initiating cells [8-10]. These characteristics include phenotypic plasticity, pro-invasive and stem-like properties, drug resistance, and immune evasion [8-10]. As a key branch point in the cell trajectory that interacts with multiple stress cell states (FIG. 1H), Cluster 0 exhibits phenotypic plasticity, enabling the cells to generate diverse transcriptional states. Bioinformatic removal of Cluster 0 results in a simplified single trajectory of cell state transitions and a loss of branching, underscoring the importance of this cell state in generating multiple cell fates (FIG. 1Q and FIG. 1R).

Gene signatures for Cluster 0 include numerous features of pre-metastatic cells, such as positive regulation of epithelial-mesenchymal transition (EMT), cell adhesion, Wnt signaling pathway and neuronal development (FIG. 1C). While the role of partial EMT in facilitating invasive cancer cell migration is well-established, recent studies indicate that enhanced cell-cell adhesion can also lead to collective migration of tumor cells, promoting metastatic colonization more effectively than single cells [8-10]. Additionally, neuronal development genes have been linked to metastasis and reduced survival across multiple tumor types including Robo/SLIT2, SEMA family genes, and PLXNA3 that are expressed in Cluster 0 (FIG. 1S) [11]. Neural genes such as SEZ6 in Cluster 0 are hallmarks of the most aggressive and drug-resistant small cell neuroendocrine tumors in prostate, lung, and other cancers, and are promoters of metastatic migration [12, 13].

Promoter motif analysis revealed that stem-like factors (e.g., OCT1, SOX5, SOX9) act as upstream regulators of genes associated with tumor cell stress states, and these factors appear to be particularly elevated in Cluster 0 (FIG. 1T). Moreover, Cluster 0 is enriched with markers associated with highly invasive or basal-like lineages, such as CD2414, and PRKCA15 (FIG. 1S). Clinical correlates of the Cluster 0 gene signature across three datasets similarly shows a positive correlation with Wnt signaling, stem cell maintenance, cell-cell adhesion, and neuronal development genes and a negative correlation with translation, respiration, and cell cycle genes (FIG. 1U).

The survival of metastatic cells is also influenced by their interactions with the host immune system and their ability to evade immune detection [16]. Cluster 6 expresses genes related to the inflammatory response. A detailed analysis of genes in Cluster 6 indicates that multiple interferon pathways are activated (FIG. 1C). While interferons are known to regulate both innate and adaptive immune responses and can act as anti-cancer agents, they also have the potential to facilitate immune escape by the adaptive induction of negative feedback mechanisms that suppress the antitumor immune response [17]. Consequently, the anti-cancer efficacy of this cell state depends on the level of immune activation and the surrounding environmental context. As mouse interferons do not bind to human interferon receptors [11], an immune-competent model is necessary to understand this cell state (see Examples 5 and 6).

Projection of scRNA-seq data from human breast tumors onto MDA-MB-231 cluster annotations confirmed the clinical relevance of the identified cell states and highlighted the prevalence of phenotypic stress in breast cancer. Tumor cells from patient samples predominantly mapped to stress clusters linked to hypoxia (FIG. 1V). Gene signature correlations between patient cell clusters matched our MDA-MB-231 findings (FIG. 1W and FIG. 1X). For instance, Cluster 0 in patient tumor samples negatively correlated with proliferative clusters and positively with stress clusters, mirroring hallmark signatures found in preclinical xenograft tumors (FIG. 1W and FIG. 1X). Analyses of multiple TNBC datasets indicated that patients with high expression of the Cluster 0 gene signature had lower overall survival and distant metastasis-free survival rates, consistent with a metastatic-initiating and drug-resistant phenotype (FIG. 1Y). Using data from the I-SPY 2 adaptive clinical trial that includes patients with early stage, high-risk TNBC [18], we observed overall drug resistance in tumors with signatures from high-stress clusters (0, 2, and 3), compared to tumors from more proliferative or inflammatory clusters (4, 1, 5, and 6) (FIG. 1Z). These clinical data suggest that TNBC patients with tumors exhibiting high-stress cluster signatures face worse survival and treatment outcomes, highlighting the potential of these gene signatures as predictive and prognostic biomarkers.

### Example 2: BACH1 was a Regulator of Phenotypic Plasticity in TNBC

Having established the clinical significance of these stress-linked phenotypic states, we aimed to identify potential regulatory factors by analyzing predicted upstream transcription factors for these clusters. Among the top predicted transcription factors were developmental factors and a stress response protein, BACH1 (FIG. 1U). In contrast to stem cell factors such as SOX5 and SOX9 [19], BACH1 is not essential for embryonic development under non-stressed conditions [20]. BACH1 is known to reprogram cancer cell metabolism towards aerobic glycolysis, a hallmark of tumor cells, and to promote epithelial-mesenchymal transition (EMT), invasion, and metastasis by binding to AP-1-like motifs [21-24]. Additionally, BACH1 has been implicated in the regulation of oxidative stress by competing with NRF2 to bind to the MARE promoter sequence, thereby suppressing the expression of antioxidant genes [21]. However, its role in regulating in vivo transcriptional landscapes under cellular stress conditions remains poorly understood. Therefore, whether BACH1 acts as a key mediator of these stress-associated phenotypic states in TNBC was investigated.

Initially, it was confirmed that BACH1 promotes metastasis but does not significantly affect growth in several tumor models (FIGS. 2A-2C). ScRNA sequencing analysis of control versus BACH1 knockout (BACH1 KO) MDA-MB-231 tumors revealed comparable UMAP clusters and growth properties (FIG. 2D and FIG. 2E). Spatial transcriptomic analysis supported these findings (FIG. 2E). However, loss of BACH1 did alter expression of genes primarily related to stress, inflammation and metastatic progression (FIG. 2F). As expected [25], BACH1 KO resulted in the upregulation of genes involved in oxidative stress and ferroptosis such as HMOX1, GCLM, FTL and FTH1 (FIG. 2G and FIG. 2H). Additionally, the loss of BACH1 triggered ribosome biogenesis (e.g. RPS27, RPS6, RPS20) and inflammatory responses (FIG. 2G). However, given the lack of a complete immune system in this xenograft model, it is important to evaluate the BACH1-mediated inflammatory response in syngeneic models (see below Examples 5 and 6). Genes implicated in metastasis or immune evasion, particularly in breast cancer (e.g. PRSS2; CD24; KRT7, 8, & 19; COL6A1; ENPP1), were among those reduced by BACH1 KO (FIG. 2G and FIG. 2H).

3D-UMAP analysis showed that loss of BACH1 reduces connectivity between Cluster 0 and Clusters 4, 1, and 5 (FIG. 2I). Consistent with this observation, pseudotime state analyses revealed a more direct trajectory of Cluster 0 gene expression in BACH1 KO tumors, with a significant reduction in transcriptional heterogeneity (FIG. 2I). Specifically, BACH1 KO reduces the Cluster 0 gene signature across stress clusters 0, 2, 6, 3 (FIG. 2J). Bioinformatic analyses showing stationary distributions that represent the proportion of each cluster in steady state show a significant decrease for Cluster 0 in BACH1 KO tumors (FIG. 2K). Supporting these findings, overexpression of BACH1 (BACH-OE) in BM1 tumors selectively increased the population of cells associated with the Cluster 0 gene signature (FIGS. 2L-2P). Conversely, the cell population associated with proliferative clusters decreased slightly, along with a modest reduction in tumor growth (FIG. 2H; FIG. 2N and FIG. 2O). Consistent with the BACH1 KO findings, overexpression of BACH1 in BM1 tumors inhibited the inflammatory response, ferroptosis and DNA replication/repair pathways, while increasing EMT, Wnt signaling and blood vessel development (FIG. 2L, FIG. 2Q, and FIG. 2R).

To investigate the BACH1 transcriptional activity program, gene signatures designed to reflect both upregulated and downregulated BACH1 activity were generated. The BACH1 cancer cell-intrinsic gene signatures were constructed by integrating BACH1 overexpression and KO models utilizing both single-cell and spatial transcriptomic RNA-seq data (FIGS. 2S-2U). The BACH1-promoted score reflects the expression of genes induced by BACH1. Conversely, the BACH1-inhibited score measures the expression of genes, such as canonical NRF2 target genes, that are repressed by BACH1 and induced upon its loss.

An elevated BACH1-promoted gene signature in control versus KO tumor cells was observed across all clusters, with the highest expression levels of BACH1 activity in transition Cluster 0 (FIG. 2W). Correlation analysis of BACH1 gene signatures and individual clusters suggests that BACH1-promoted activity plays a more significant regulatory role in stress-associated clusters than in proliferation-associated clusters (FIG. 2X). In control cells, BACH1-inhibited genes had lowest expression in the proliferative clusters (1, 4) and highest in the extreme stress Cluster 3 (FIG. 2X). Knockout of BACH1 elevated expression of these BACH1-inhibited genes across all clusters, with the biggest increase in the proliferative cluster (FIG. 2W). These results suggest that BACH1 plays a key role in suppressing oxidative stress response genes such as canonical NRF2 targets (e.g. GCLM and FTL) under proliferative conditions but is less effective under extreme stress conditions.

These findings were further supported by the analysis of the BM1 TNBC xenograft model. Although tumor growth was not significantly affected, expression of the Cluster 0 gene signature was reduced in BACH1 KO tumors analyzed by bulk RNA-seq and in BACH1 shRNA tumors analyzed by scRNA-seq (FIGS. 2Y-2AA). The loss of BACH1 expression also led to a reduction in EMT markers and an upregulation of inflammatory genes (e.g. CXCL8, IL12, CXCL12, IL13RA2) and oxidative stress genes (e.g. HMOX1) in BM1 tumors (FIG. 2AB). Taken together, these results identify BACH1 as a crucial regulator of genes that define phenotypic state transitions.

### Example 3: BACH1 Regulated Cell State Transition Genes in Hypoxia-Dependent Manner

Given that BACH1's regulation of tumor state transitions is associated with hypoxic stress, TNBC cells were exposed to hypoxia in vitro to evaluate the direct impact of BACH1 on the cellular hypoxic response (FIG. 3A). Initially, we analyzed BMI cells exposed to hypoxia (1% oxygen) for 0, 24 and 72 hours in the presence or absence of BACH1. Clustering and GSEA analysis showed that genes downregulated under hypoxic conditions were primarily associated with translation, DNA replication and interferon response (FIGS. 3B-3D). In contrast, genes upregulated under hypoxia included those involved in metabolism, developmental growth and the hypoxia response, reflecting critical adaptive and stress-response mechanisms (FIGS. 3B-3E).

The most dramatic changes in gene expression due to BACH1 occurred at 72 hours (FIG. 3D and FIG. 3E). Under prolonged hypoxia, BACH1 repressed genes involved in oxidative stress and ferroptosis (e.g. HMOX1, GCLM, FLH1, FTL), cytokine and inflammatory response (IL1A-B; CXCL1, 2, & 8; IL13RA2) and oxidative phosphorylation (COX6A1, UBE2B), consistent with our TNBC tumor data (FIG. 3F). In contrast, BACH1 induced genes implicated in metastasis or immune evasion (e.g. KRT19, COL6A1, LOX, ENPP1) (FIG. 3F) as observed in murine tumors. GSEA analysis of RNA-seq data confirmed that BACH1 promoted numerous processes under hypoxia, including those related to pathways included in the Hallmark Hypoxia, Hedgehog signaling, G2M checkpoint, apical junction, Wnt and EMT gene signatures (FIG. 3G). GSEA based on mass spectrometry analysis of TMT-labeled protein from cells grown under hypoxia validated BACH1 induction of the Hallmark Hypoxia gene signature and other genes observed in RNA-seq (FIGS. 3H-3J). Similarly, we observed that BACH1 KO decreased hypoxia-induced genes, including NDRG1, EGLN3, and ANGPTL4, in the MDA-MB-436 tumor model, although tumor growth remained unaltered (FIG. 3K and FIG. 3L).

Prolonged hypoxia, a hallmark of the tumor microenvironment, is well-documented to induce widespread chromatin remodeling, enabling cells to adapt to low oxygen conditions [26]. Given its emerging role as a transcriptional and epigenetic regulator, we hypothesized that BACH1 regulation of the hypoxia response and Cluster 0 gene signature could occur either via direct binding and/or indirectly through modulation of chromatin accessibility. To test this, we assessed promoter accessibility using ATAC-seq in BMI cells grown under normoxic or hypoxic conditions for 72 hours. Hypoxia led to a significant reduction in overall chromatin accessibility, a change that was further exacerbated by the loss of BACH1 (FIGS. 3M-3O). Surprisingly, BACH1 loss also reduced chromatin accessibility without a significant difference in transcriptional output under normoxia, suggesting that the BACH1-regulated regions are poised to respond to stresses such as hypoxia (FIG. 3N).

BACH1 binding to DNA increased from normoxia to hypoxia, as assessed by ChIP-seq (FIGS. 3P-3T). Notably, BACH1 primarily bound to sites with AP-1-like motifs, where it can promote gene transcription, as well as the canonical MARE motif, where it generally functions as a transcriptional repressor [21]. Interestingly, BACH1 also bound to additional motifs generally associated with the HOX family that plays a role in development [26]. Many of these genes also contained HRE sites in addition to BACH1 binding sites, consistent with a role in the hypoxia response.

Network analysis revealed a significant overlap between genes whose expression and chromatin accessibility are reduced by BACH1 loss after prolonged hypoxia, genes directly bound by BACH1, and genes highly expressed in Cluster 0, the transition state in xenograft tumors (FIG. 3U and FIG. 3V). These genes are primarily involved in processes characteristic of Cluster 0 tumor cells, such as brain and blood vessel development, cell-cell adhesion, and migration (FIG. 3U). Supporting this observation, a 0 to 72-hour hypoxia treatment time course revealed that maintaining or promoting Cluster 0 gene expression under hypoxia for 72 hours is dependent on BACH1 (FIG. 3W). Applying our in vivo-derived BACH1-promoted and inhibited gene signatures, we noted that, under prolonged hypoxia, BACH1 decreases its repressive control over canonical target genes (e.g., HMOX1, FTL, GCLM) and shifts toward promoting gene expression (FIG. 3W and FIG. 3F). This transition is captured by the BACH1 ‘distance’ score (FIG. 3W) [27]. These findings highlight a functional shift in BACH1 during prolonged hypoxia towards transcriptional activation and confirm BACH1's role as a driver of cell state transition genes under hypoxia in both TNBC cells and tumors.

### Example 4: BACH1 Loss Restores T Cell Infiltration and Activation

Hypoxic stress exacerbates immune suppression by altering the tumor microenvironment and driving immune checkpoint expression, promoting immune evasion, metastatic spread and therapy resistance [39-41]. As a component of its response to hypoxia, BACH1 suppresses inflammatory genes across multiple TNBC models (FIG. 2AC, FIG. 2F, and FIG. 2L). Therefore, it was investigated whether BACH1 directly promotes immune evasion using immunocompetent syngeneic tumor models.

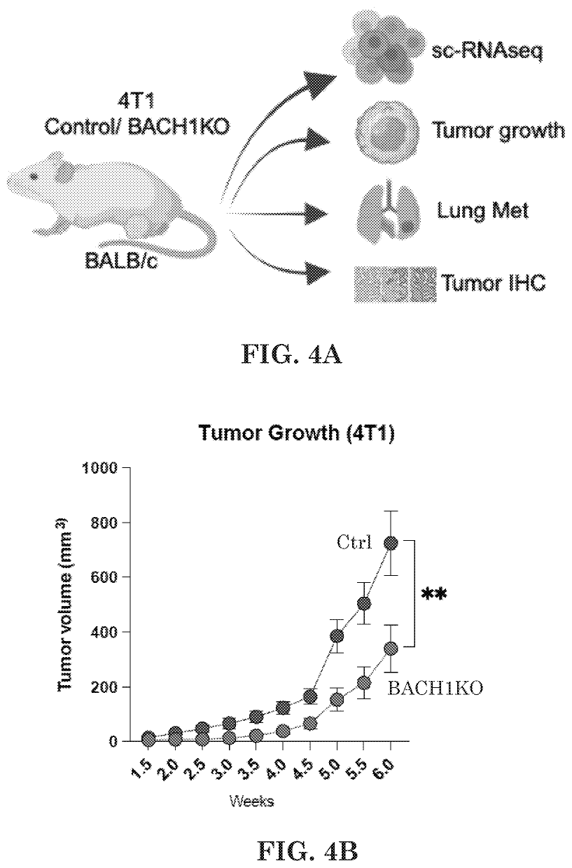

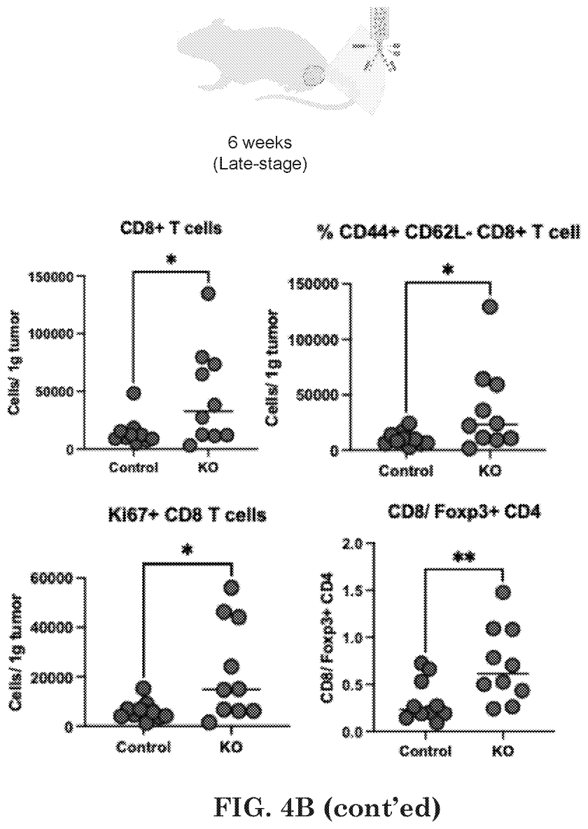

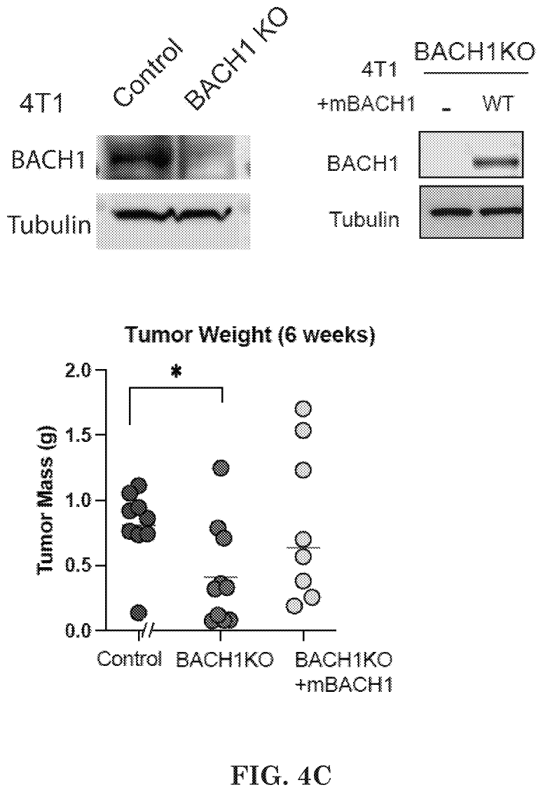

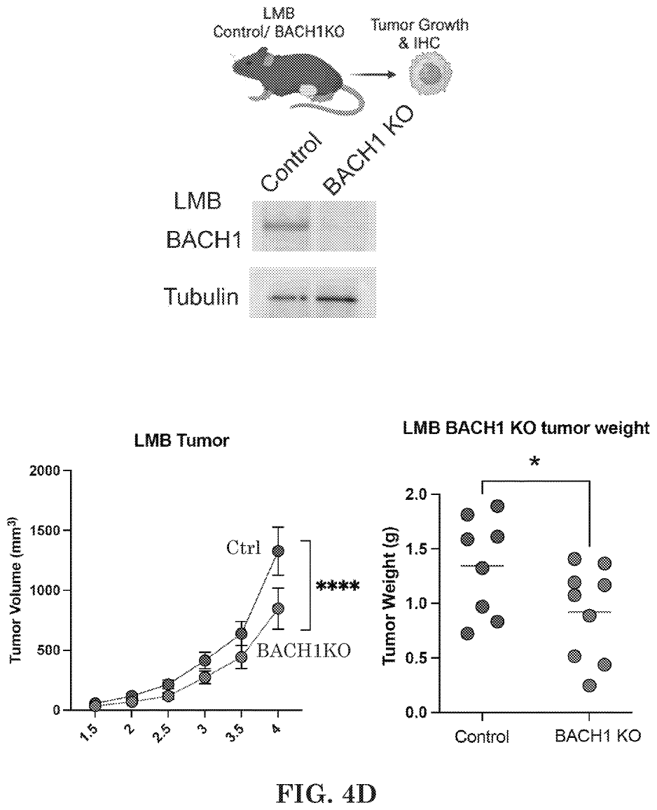

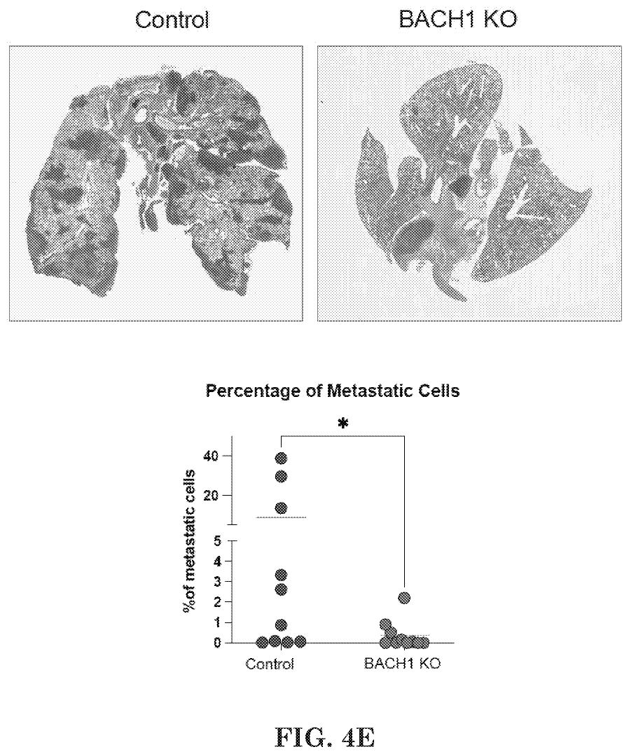

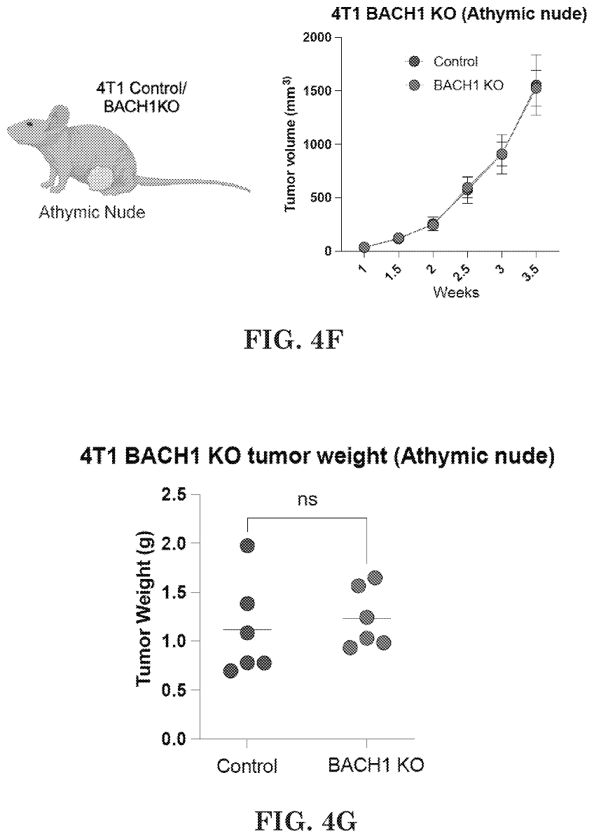

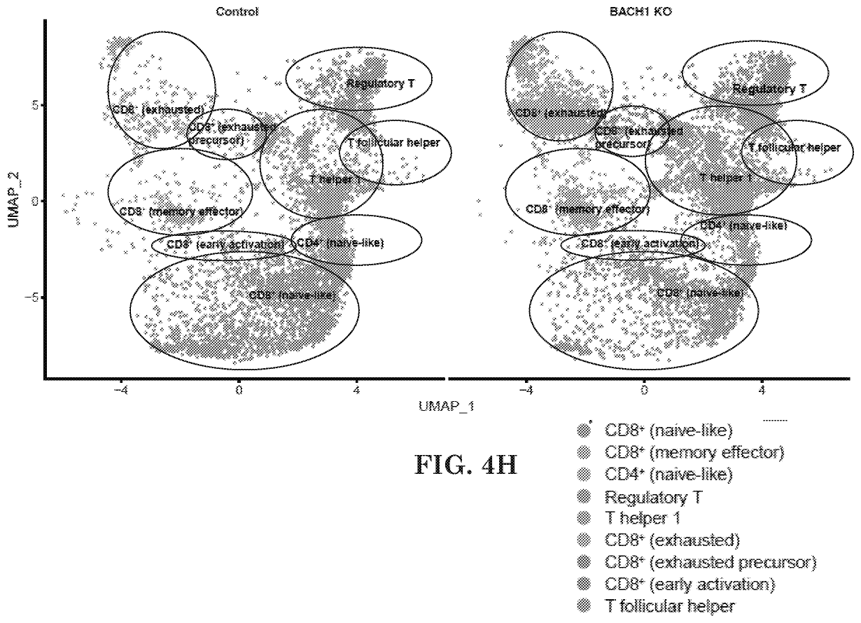

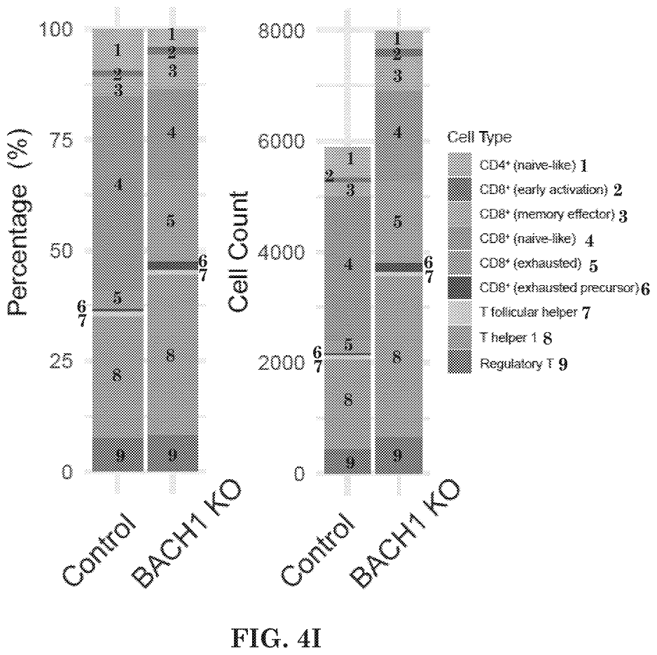

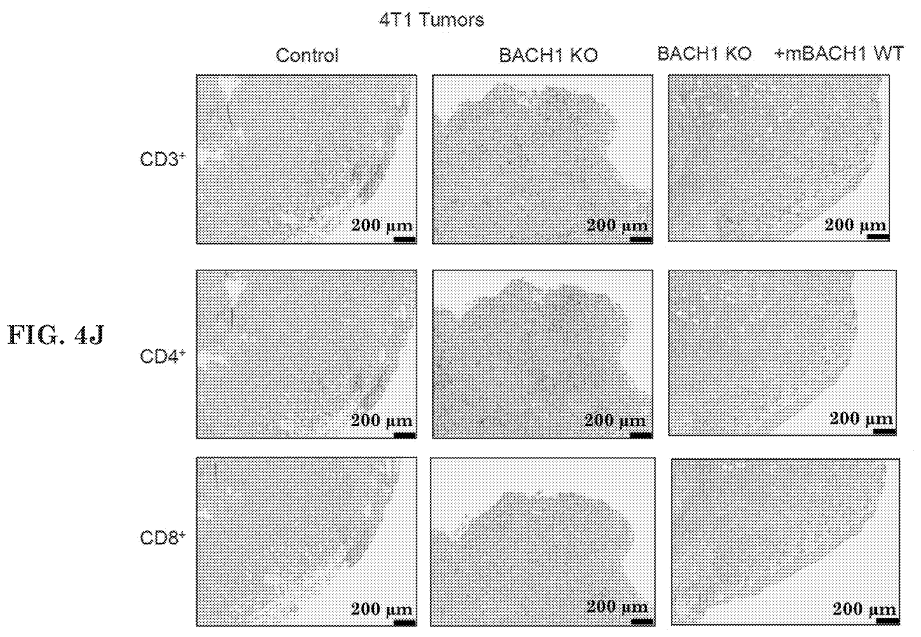

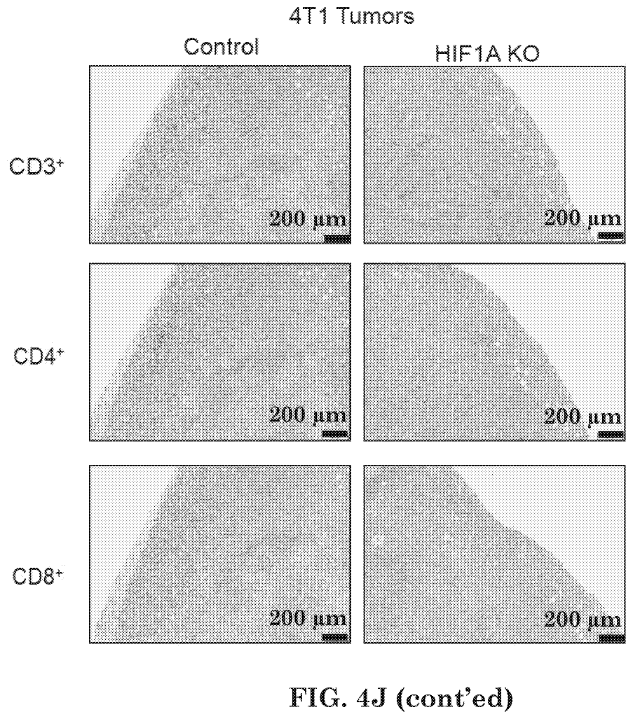

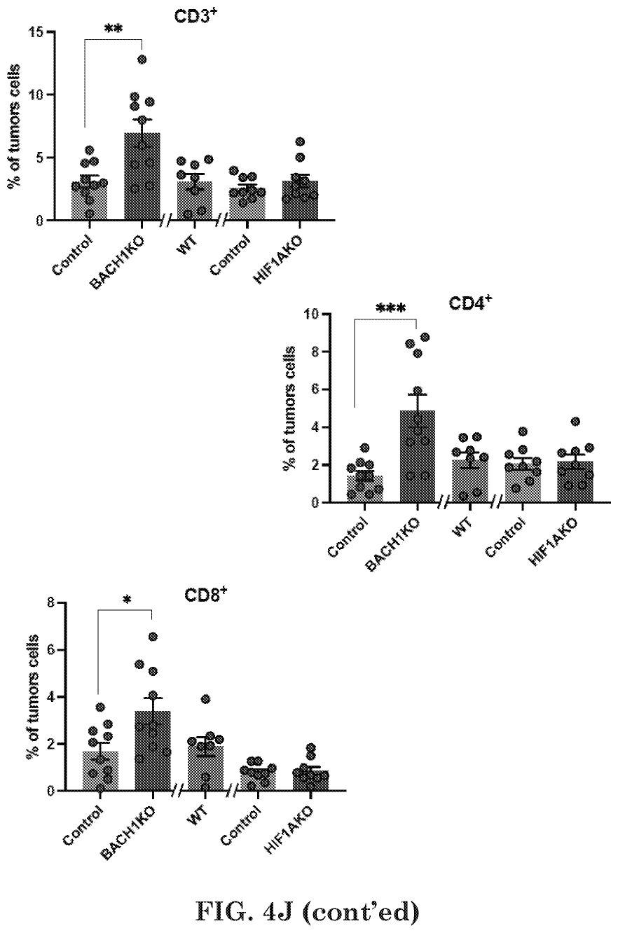

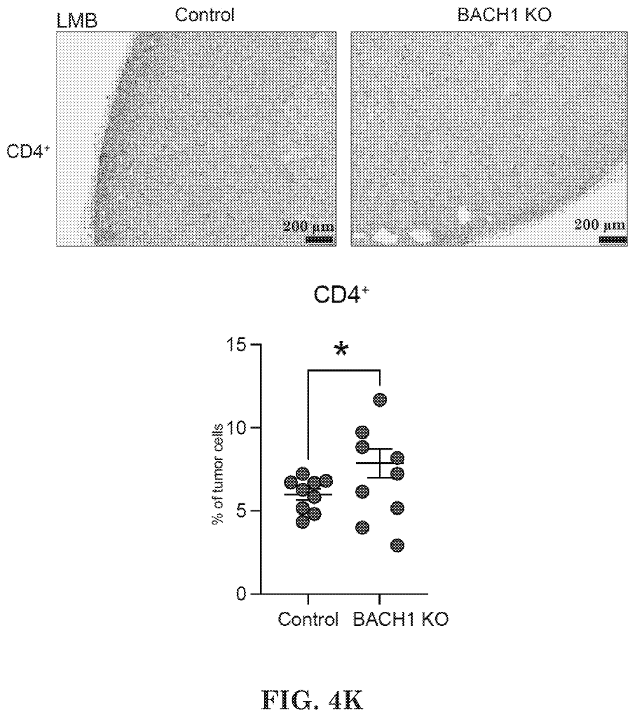

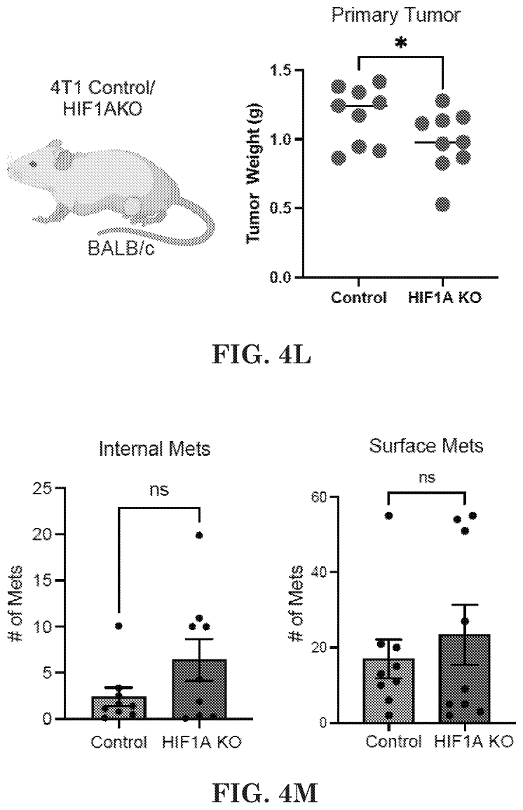

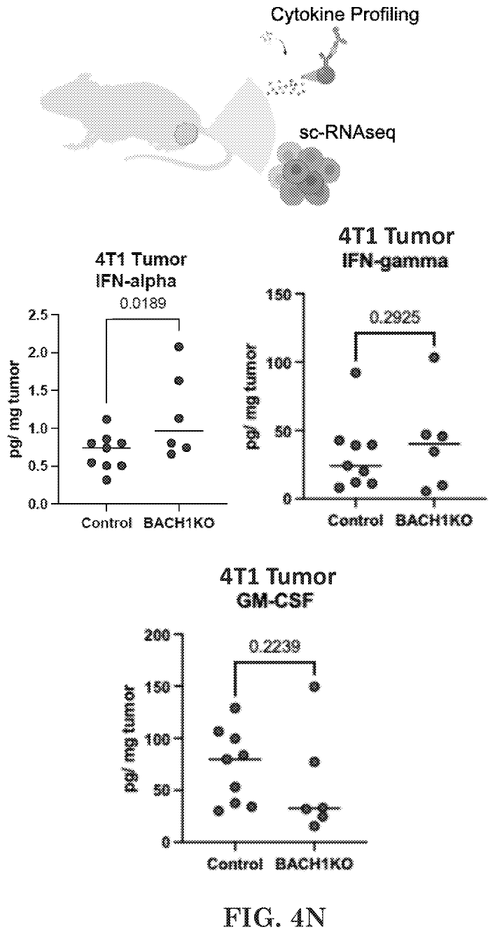

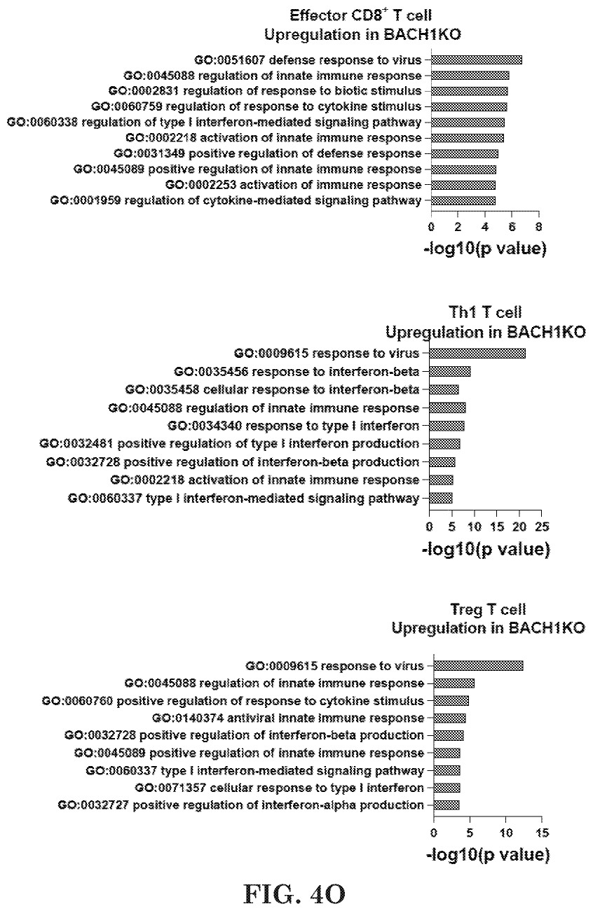

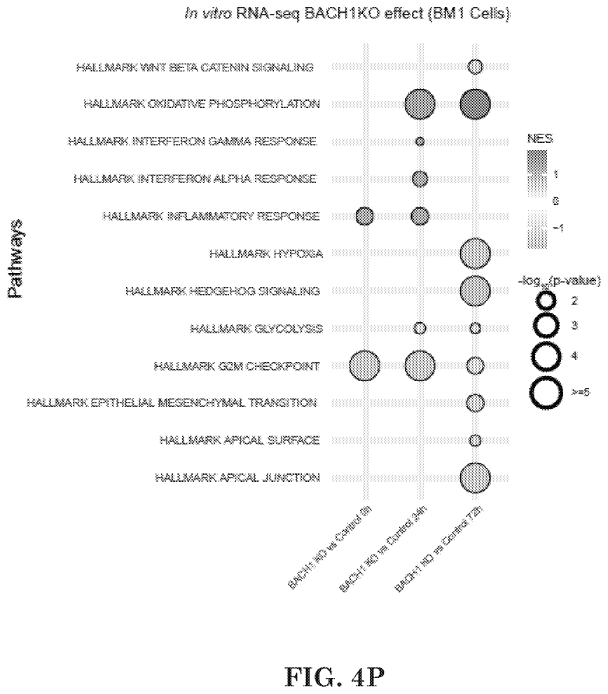

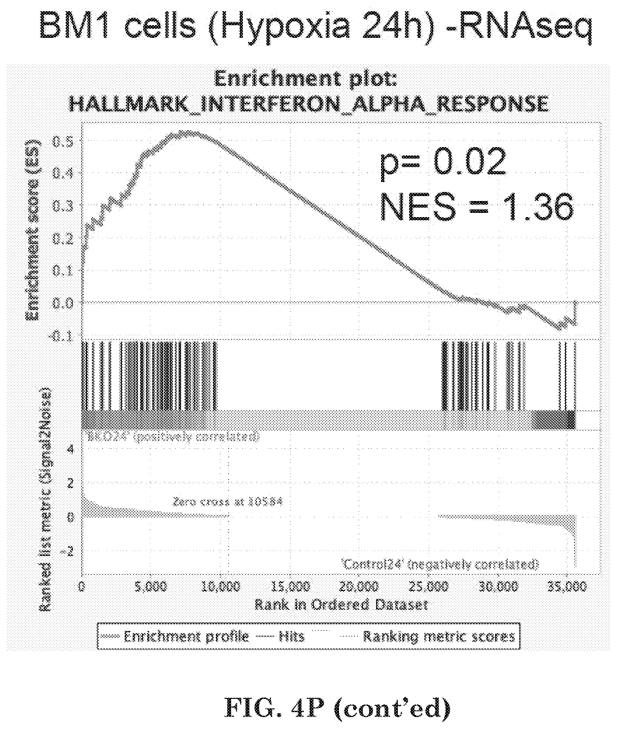

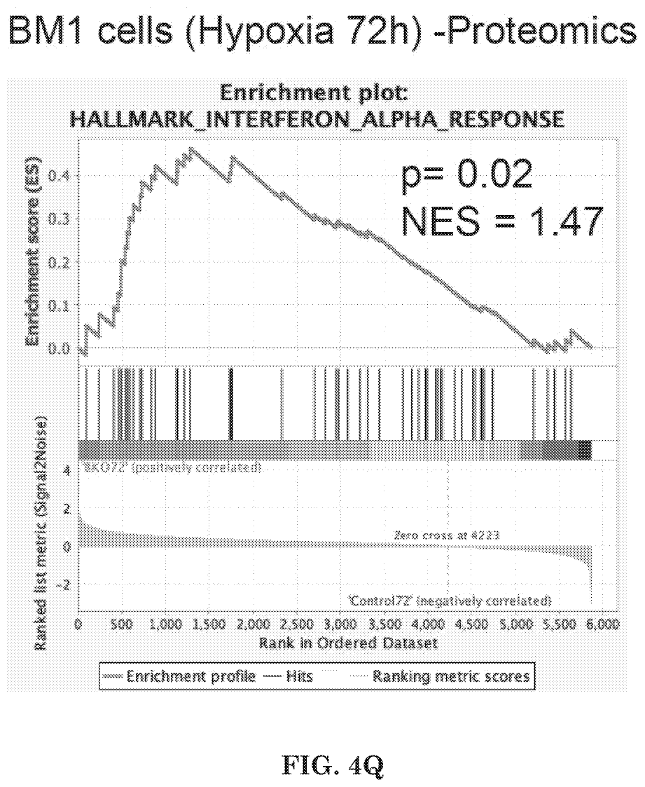

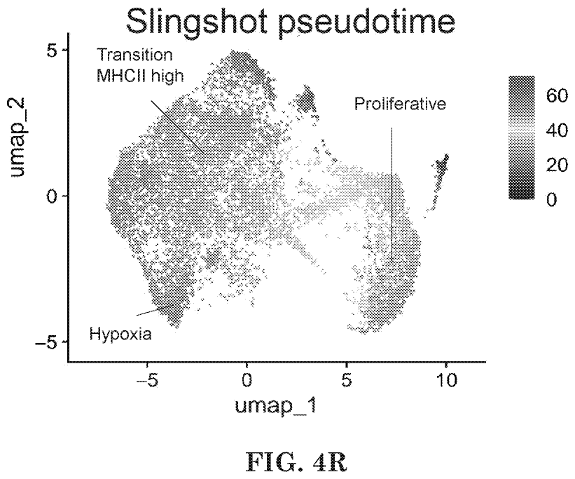

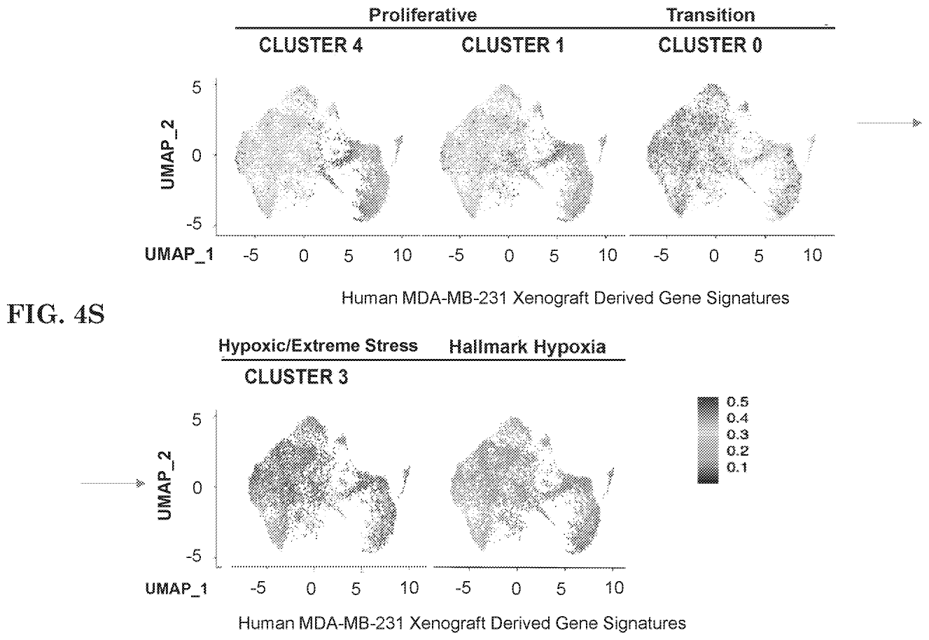

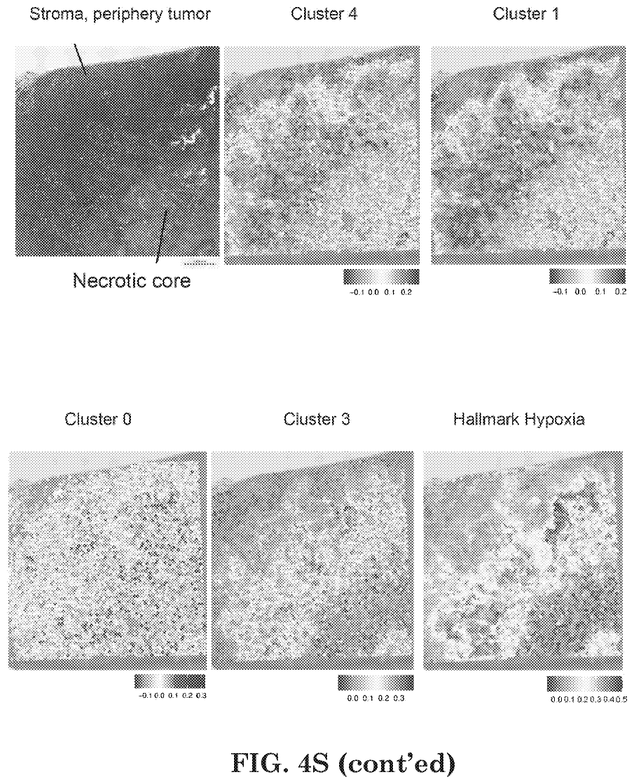

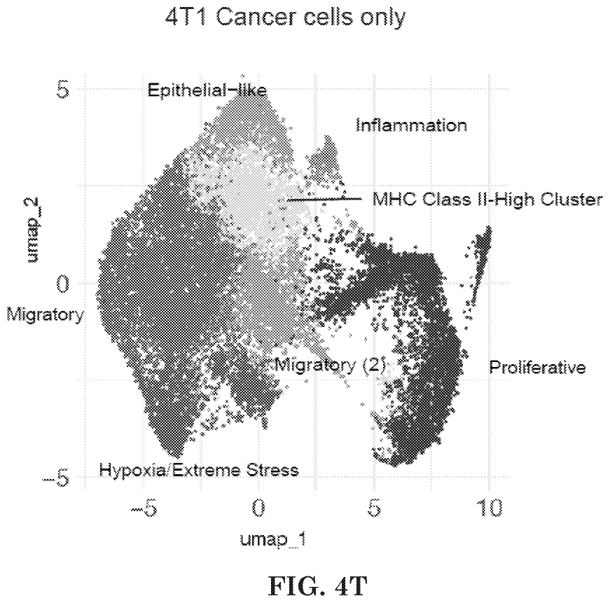

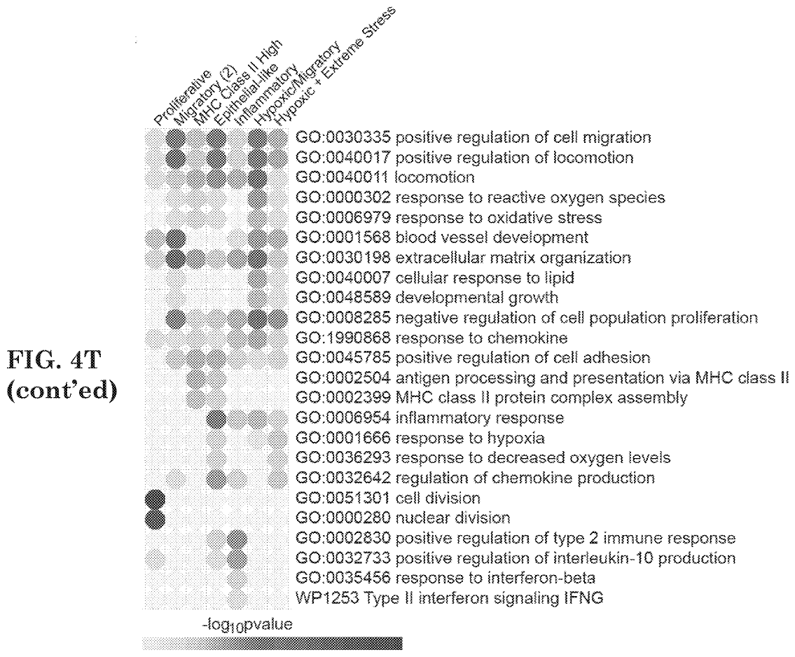

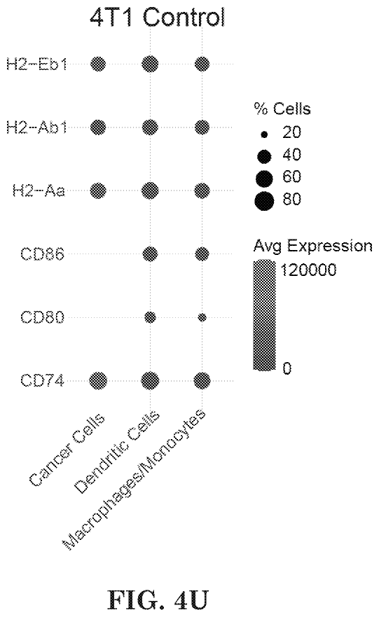

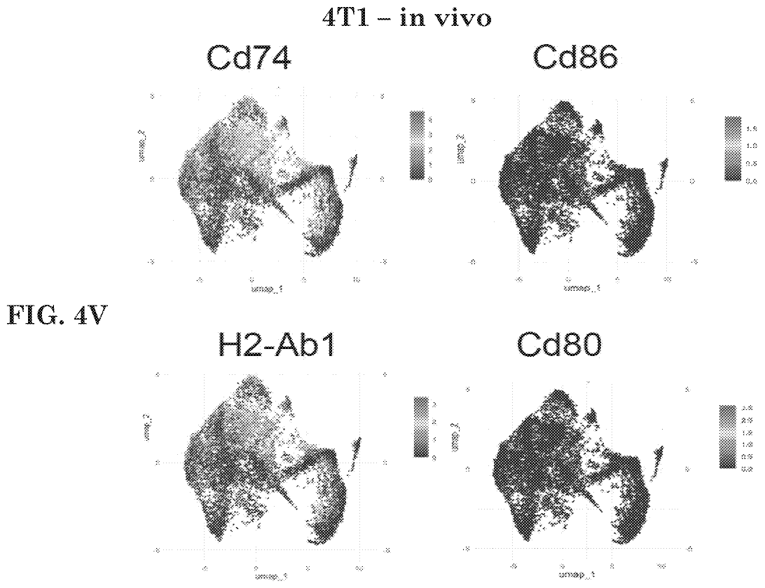

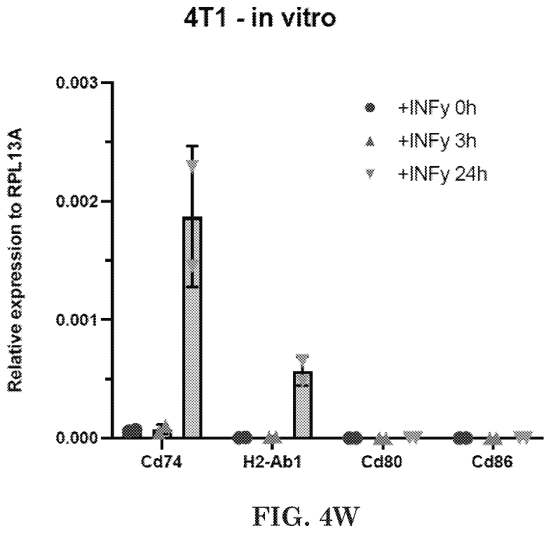

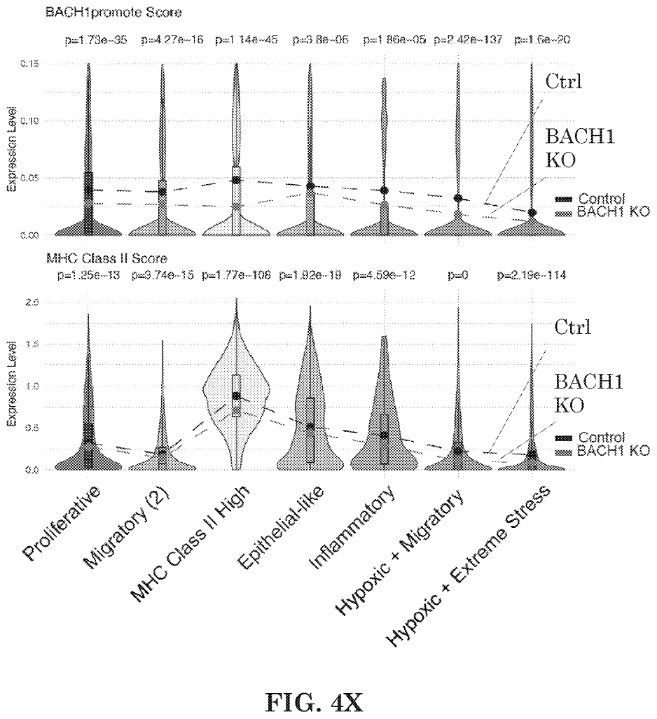

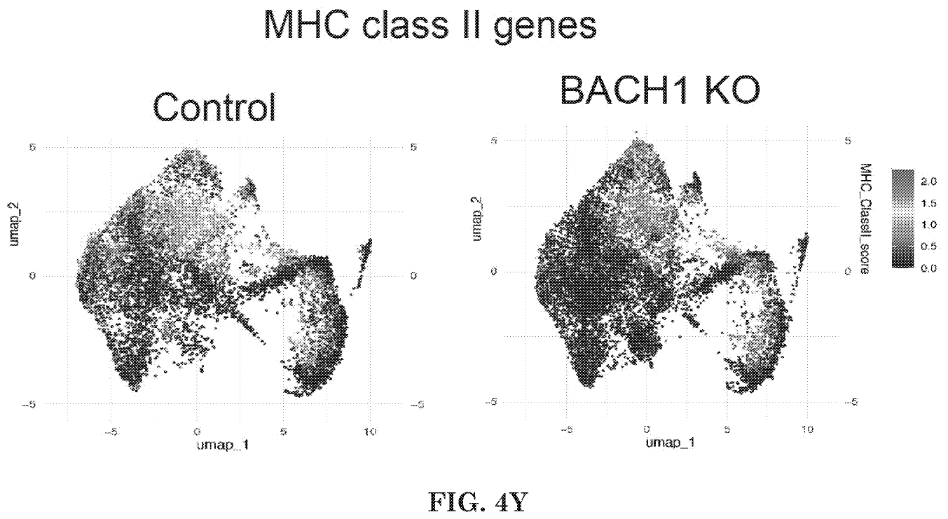

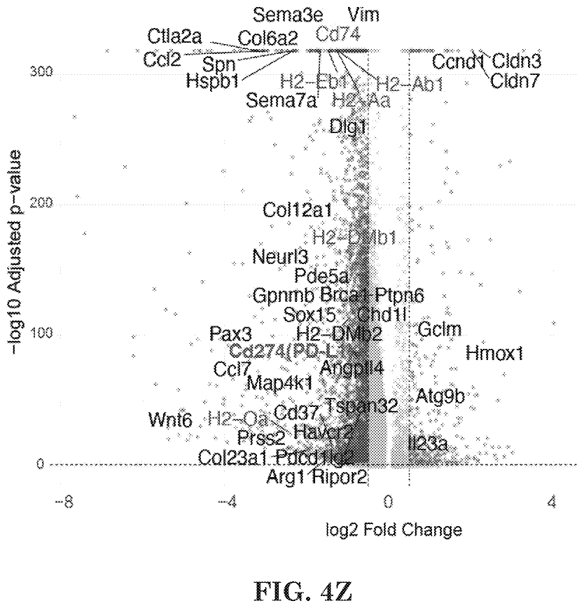

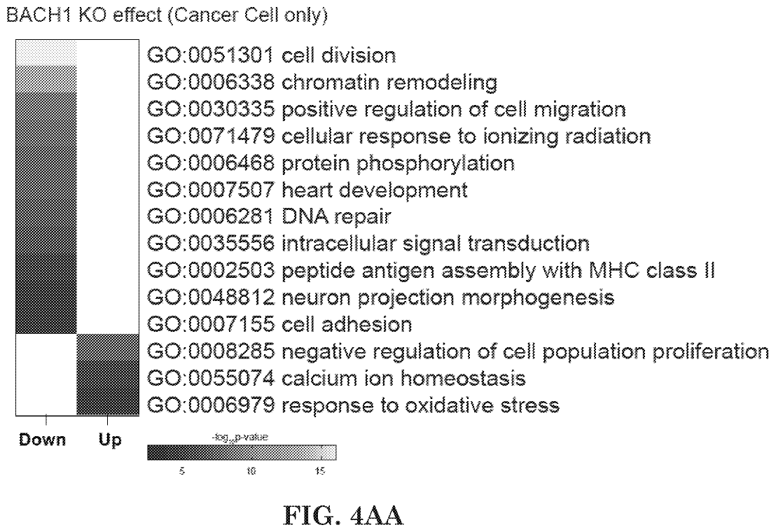

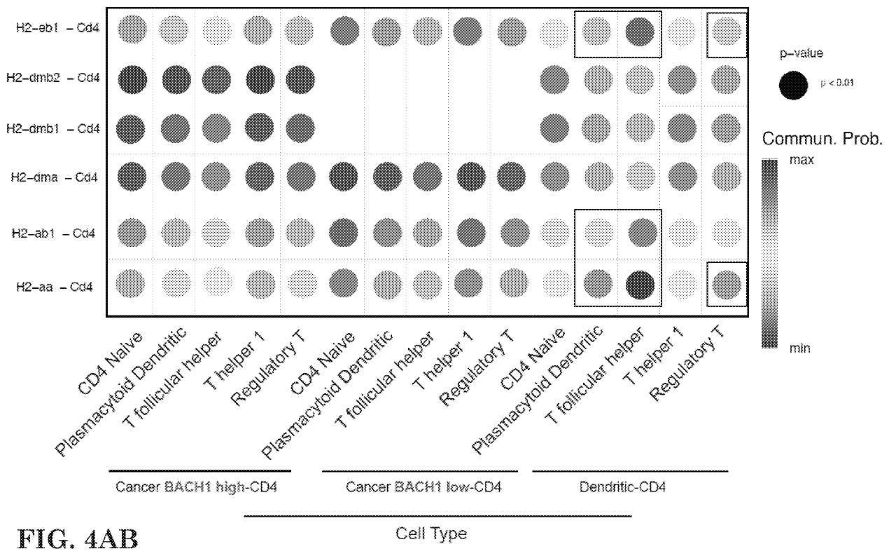

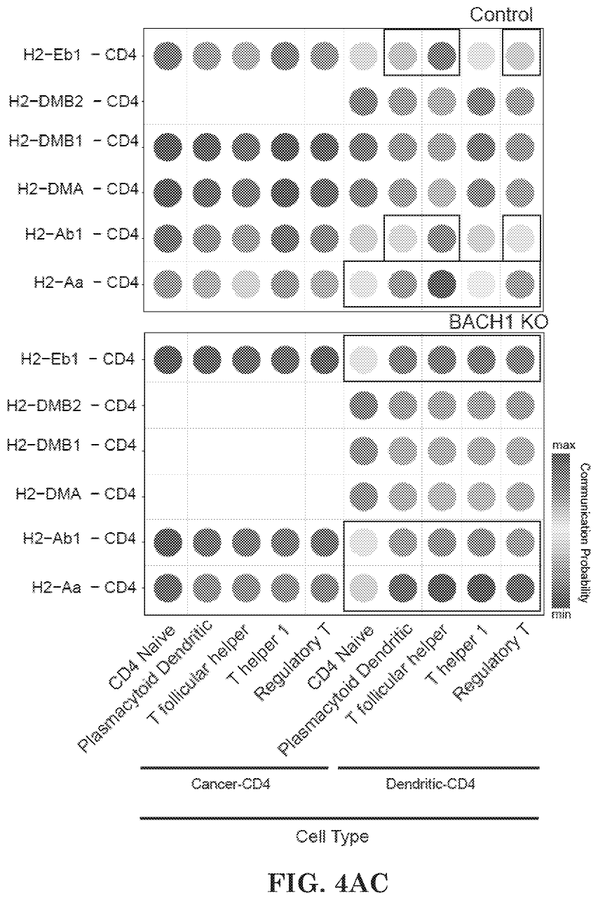

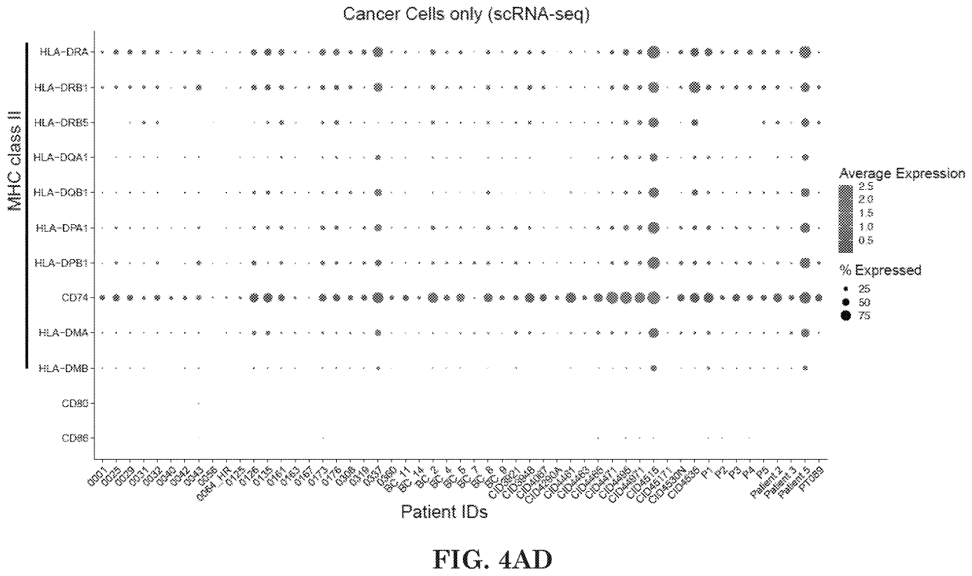

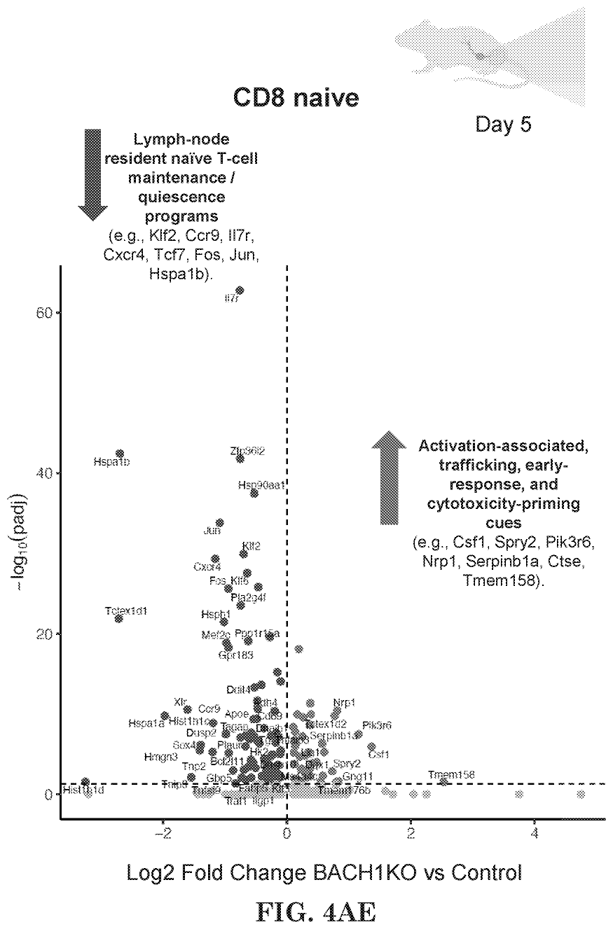

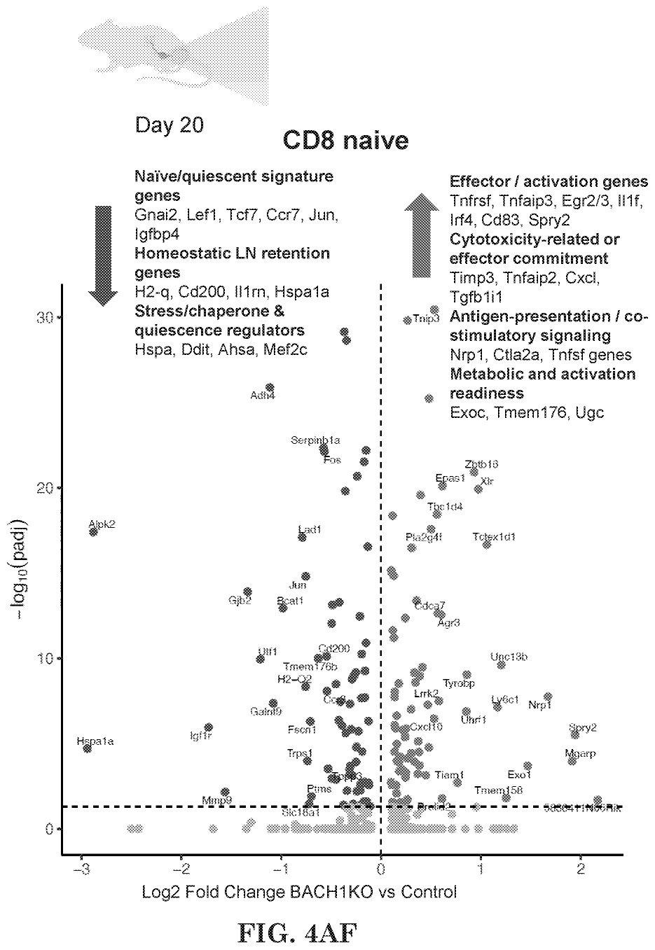

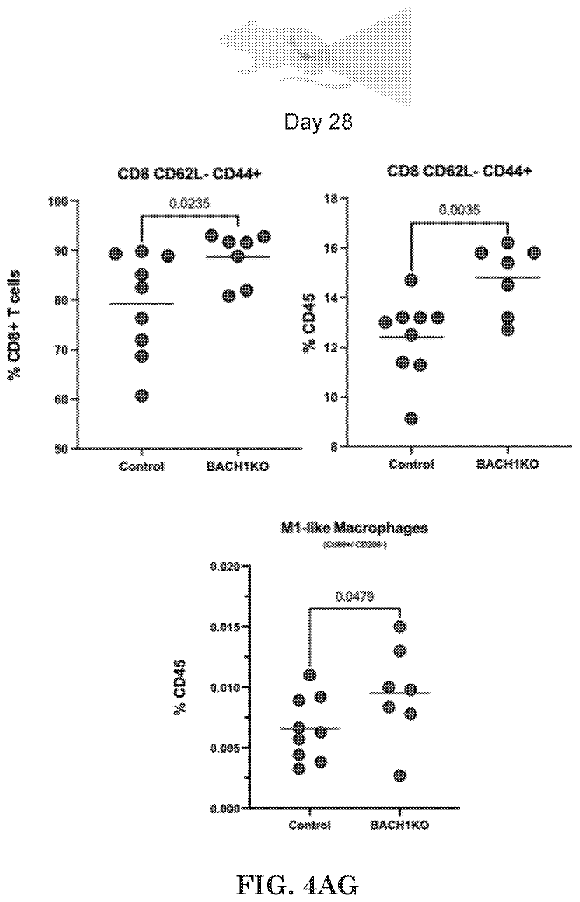

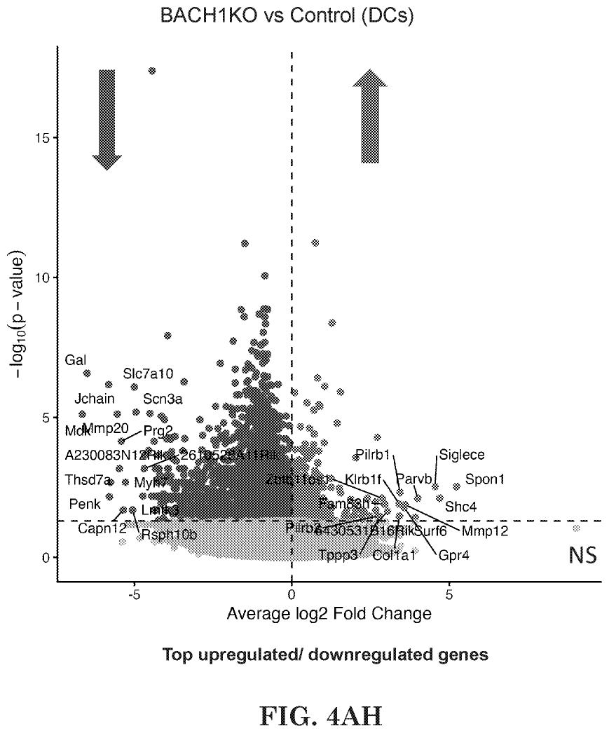

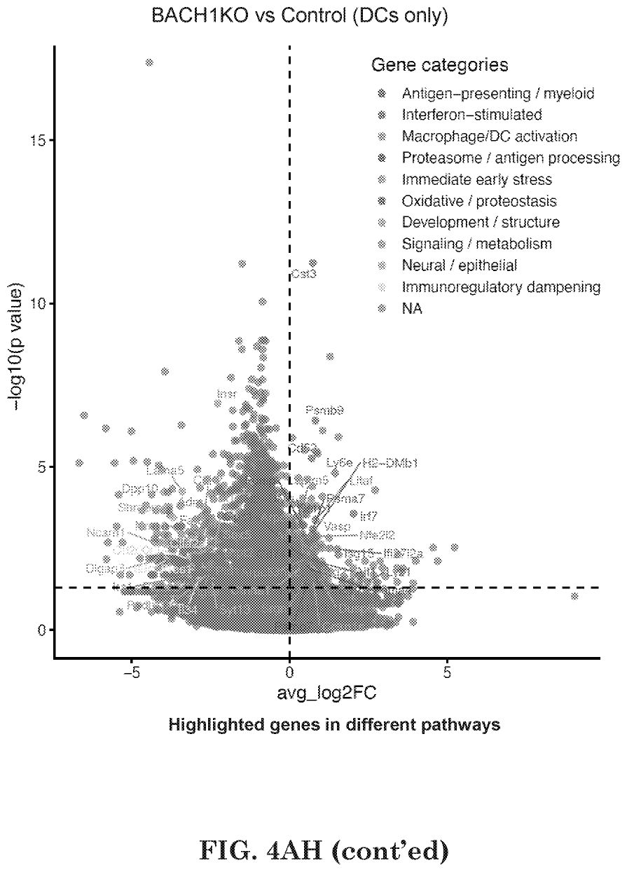

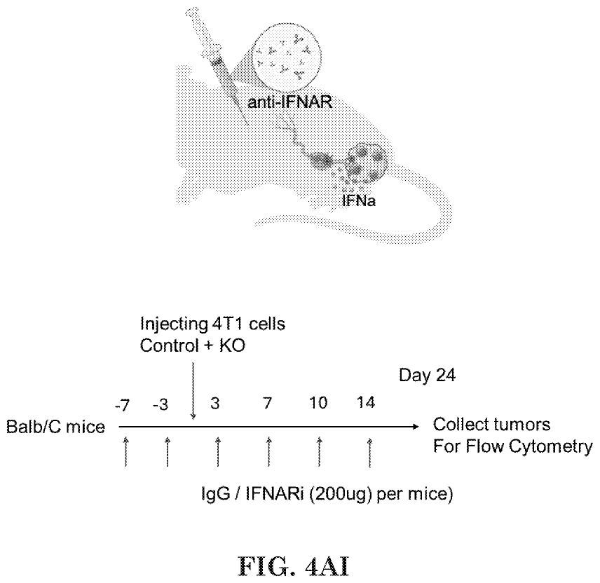

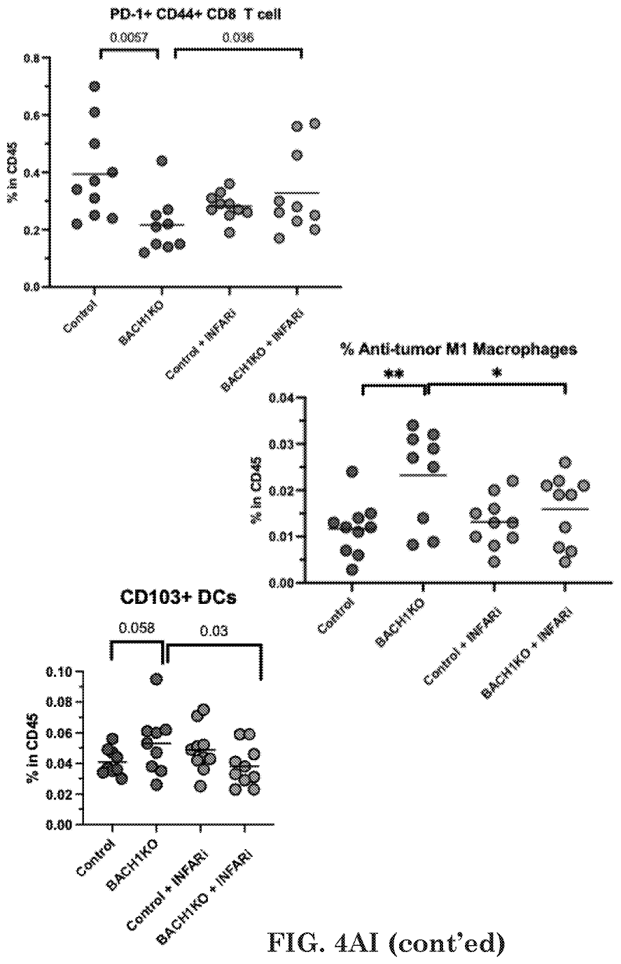

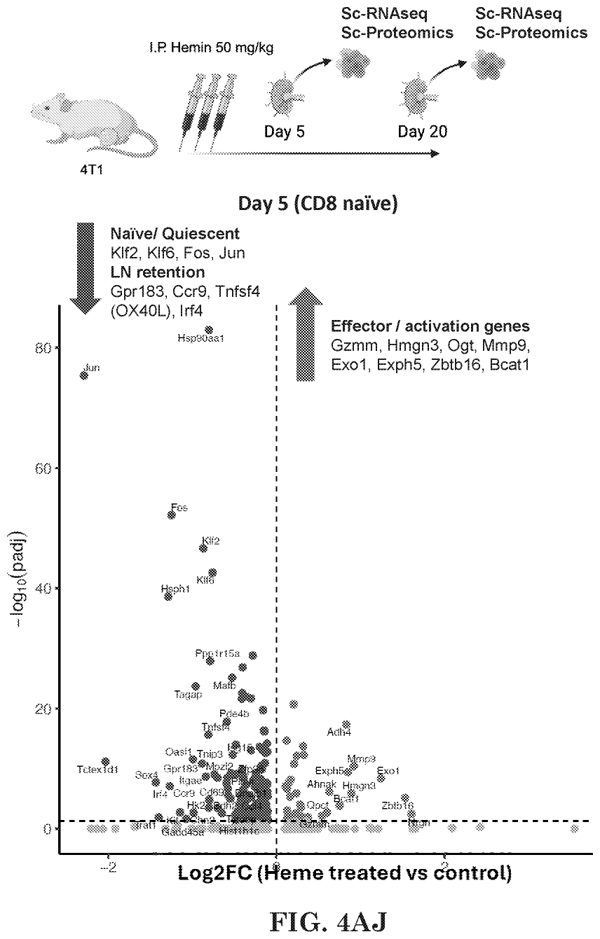

BACH1 deletion in 4T1 and LMB tumors significantly reduced tumor growth, particularly in the 4T1 model, where knockout tumors also demonstrated reduced lung metastases (FIGS. 4A-4E, 4G, and 4K), consistent with BACH1's role in promoting metastasis [23]. Importantly, the growth of 4T1 BACH1 KO tumors remained comparable to controls in nude mice lacking thymus-matured T cells (FIGS. 4F-4G). Furthermore, in xenograft models with impaired T cell involvement, BACH1 deletion had no significant impact on tumor growth (FIGS. 3K, 2D, 2AA, 4S, 41, and 4N), indicating BACH1's effect is at least partially mediated through the adaptive immune system.

Single-cell RNA sequencing of 4T1 tumors revealed expansion of activated and exhausted T cell states in BACH1-deficient tumors, with reduced naïve populations (FIG. 4H and FIG. 4I). Immunohistochemical (IHC) analysis confirmed greater infiltration of CD3⁺, CD4⁺, and CD8⁺ T cells in BACH1KO 4T1 and CD4⁺ T cells in BACH1KO LMB tumors (FIG. 4J, FIG. 4D, and FIG. 4K). Re-expressing WT BACH1 in BACH1 KO 4T1 tumor cells restored T cell exclusion, confirming BACH1's role in limiting T cell infiltration (FIG. 4J). In contrast to BACH1, HIF1A loss did not significantly affect immune infiltration (FIG. 4J) nor decrease internal lung metastases in 4T1 tumors despite reduced tumor growth [42] (FIG. 4L and FIG. 4M). These findings indicate that BACH1, rather than HIF1A, is the primary regulator of immune evasion and lung metastasis under hypoxic stress in the 4T1 model.

Furthermore, BACH1 deletion in 4T1 tumors significantly increased activated CD8+ T cells, in which the CD8+/Foxp3+CD4 ratio was significantly elevated, indicating enhanced cytotoxic T cell activity and a reduced regulatory T cell presence within the tumor microenvironment (FIG. 4B). Single-cell suspensions were prepared from lymph nodes of control and BACH1 KO mice bearing 4T1 tumors at 5 days and 20 days post-inoculation and expression profiles were analyzed by single-cell RNA sequencing (FIG. 4AE and FIG. 4AF). Activated CD8+ T cells were confirmed in the lymph nodes of knockout tumor-bearing mice through gene expression differentiation analysis (FIG. 4AE). This suggests enhanced priming and activation of cytotoxic T cells early in the tumor response within the lymphatic compartment. Assessment of gene programs associated with T cell quiescence and activation showed that naïve CD8⁺ T cells in BACH1KO draining lymph nodes exhibited downregulation of quiescence-associated genes and upregulation of activation and effector gene programs, indicating enhanced T cell priming and functional activation in the knockout context (FIG. 4AF). Flow cytometry analysis at 28 days post-inoculation of single-cell suspensions from lymph nodes revealed that KO tumors showed increased infiltration of activated CD8⁺ T cells and M−1 like macrophages compared to controls (FIG. 4AG).

To understand how BACH1 regulates infiltration of T lymphocytes, cytokine profiling were conducted on late-stage (6 weeks) 4T1 tumors from control mice. Tumor lysates were assessed for IFNα levels. Type I interferon pathway activity was evaluated across different T cell populations from sc-RNA-seq. In the analysis of tumor secreted cytokines, an elevated Type I interferon-a (IFN-α) was observed in BACH1 knockout (KO) tumors (FIG. 4N). IFN-α serves as a key activator of naïve CD8+ T cells, coordinating with antigen recognition and co-stimulation to drive their clonal expansion, effector differentiation, and IFN-γ production [85]. Supporting this mechanism, scRNA-seq analysis revealed enhanced innate immune signaling and Type I interferon pathways across various T cell states following BACH1 loss (FIG. 4O). Increased IFN-α signaling pathways were also detected by RNAseq and proteomics analysis of BM1 cells after BACH1 knockout in vitro, supporting a role for BACH1 as a tumor-intrinsic suppressor of inflammatory responses (FIG. 4P and FIG. 4Q). Analysis revealed significant shifts in dendritic cell gene expression profiles of 4T1 tumors at 6 weeks, with upregulation and downregulation of key immune-related transcripts, and volcano plots illustrating functional categories such as antigen presentation, activation, and cytokine signaling (FIG. 4AH). These changes indicate altered dendritic cell activity and functionality in the tumor microenvironment. To further characterize the role of IFNα in mediating the effects of BACH1 on lymph nodes, Balb/c mice were pretreated with control IgG or anti-IFNAR1 antibody (200 μg per mouse) on days-7 and -3, followed by injection of 4T1 tumor cells on day 0. Antibody treatments continued on days 3, 7, 10, and 14. On day 24 post-tumor inoculation, tumors were harvested and analyzed by flow cytometry (FIG. 4AI). In 4T1 tumor-bearing lymph nodes from knockout mice, there was an increased frequency of M1-like macrophages and CD103⁺ dendritic cells, along with a reduction in exhausted CD8+ T cells. These immunological changes were reversed upon pre-treatment with IFNAR1-blocking antibodies, restoring a more suppressed phenotype (FIG. 4AI). Additionally, short-course hemin treatment to mimic BACH1KO activated CD8 naïve T cells in lymph node as soon as 5 day post-treatment (FIG. 4AJ). Particularly, naïve CD8⁺ T cells in BACH1KO draining lymph nodes exhibited downregulation of quiescence-associated genes and upregulation of activation and effector gene programs, indicating enhanced T cell priming and functional activation in the knockout context (FIG. 4AJ).

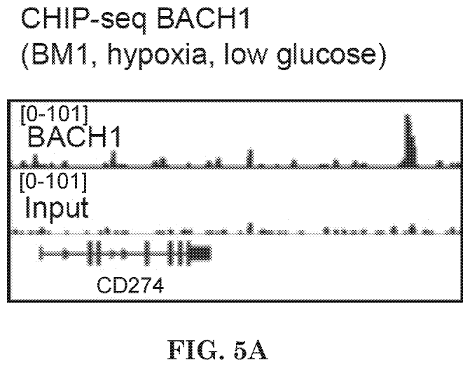

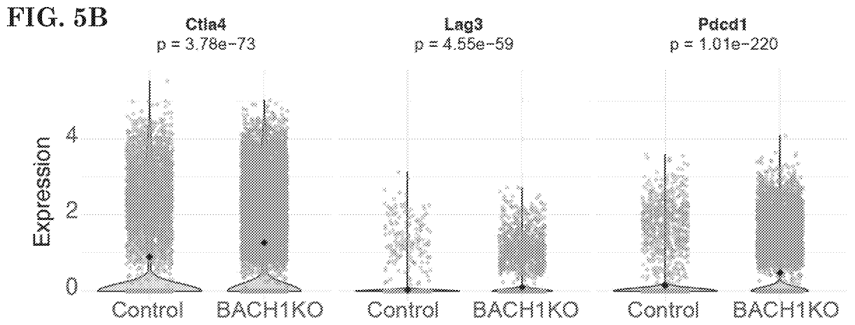

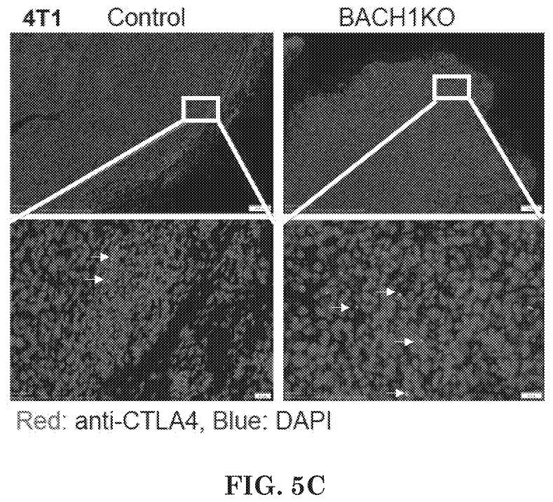

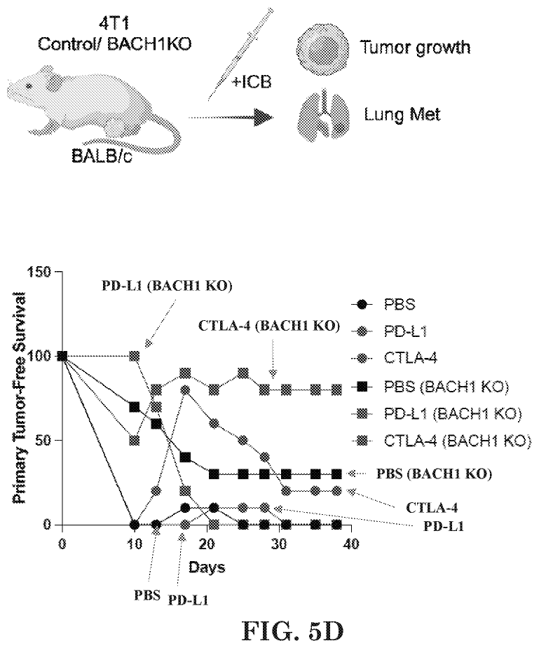

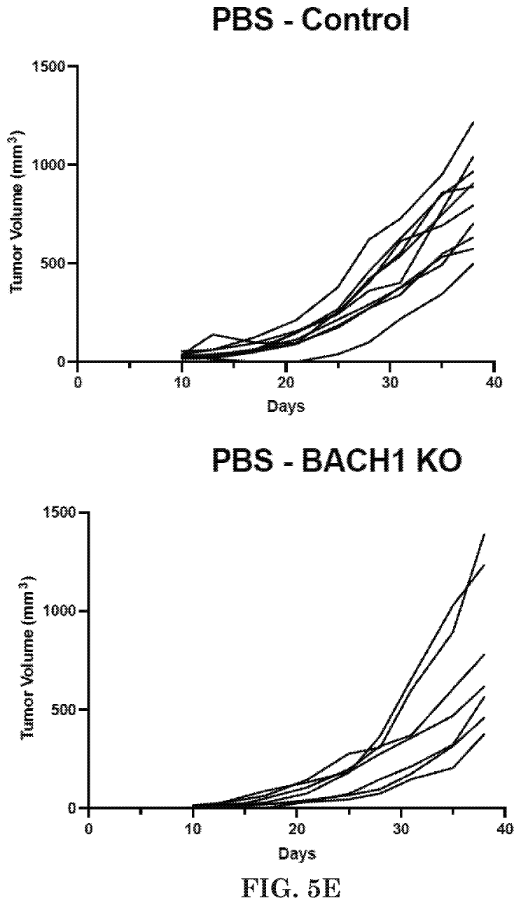

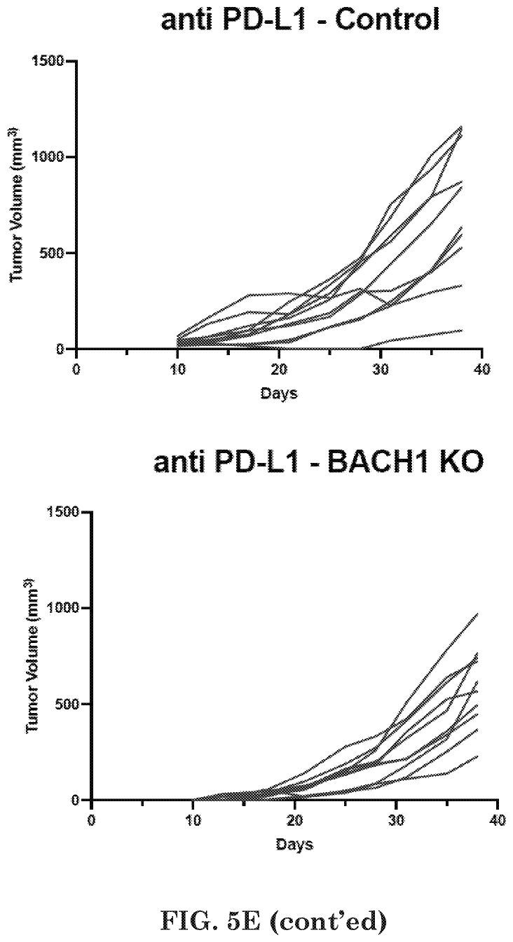

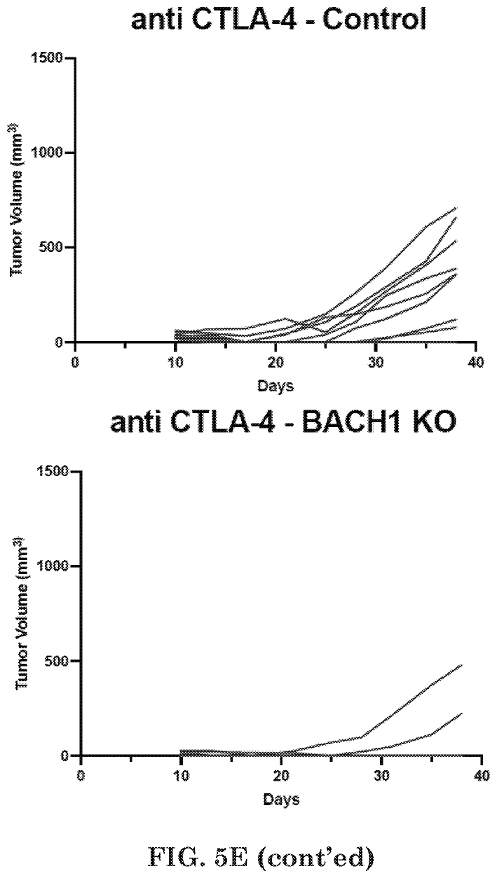

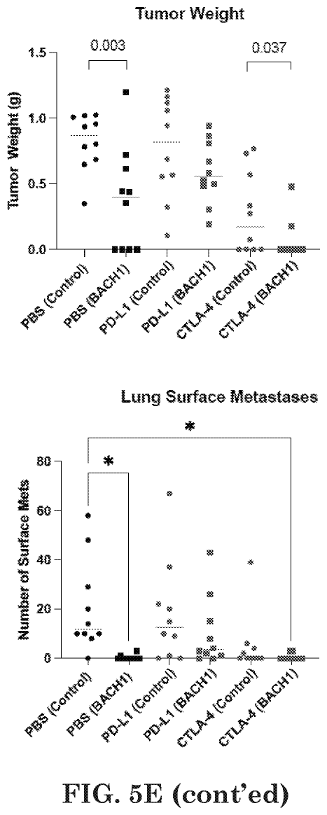

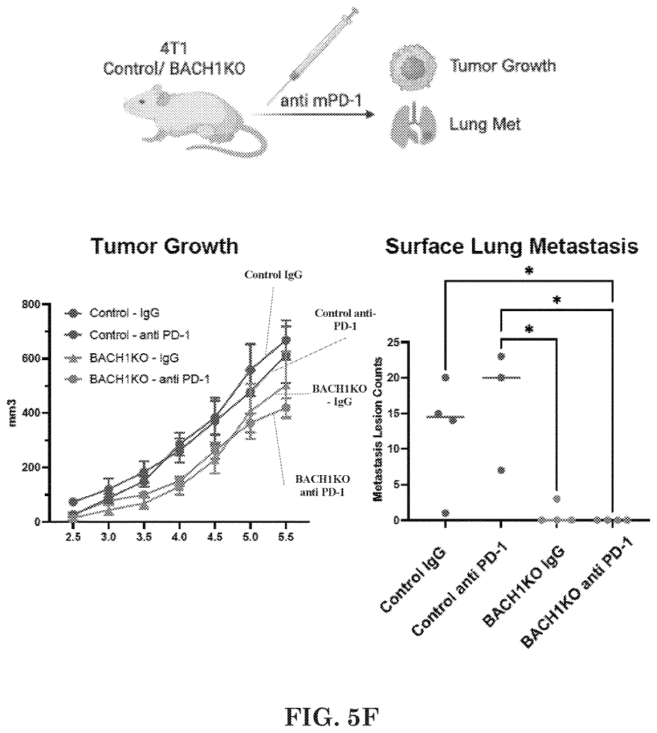

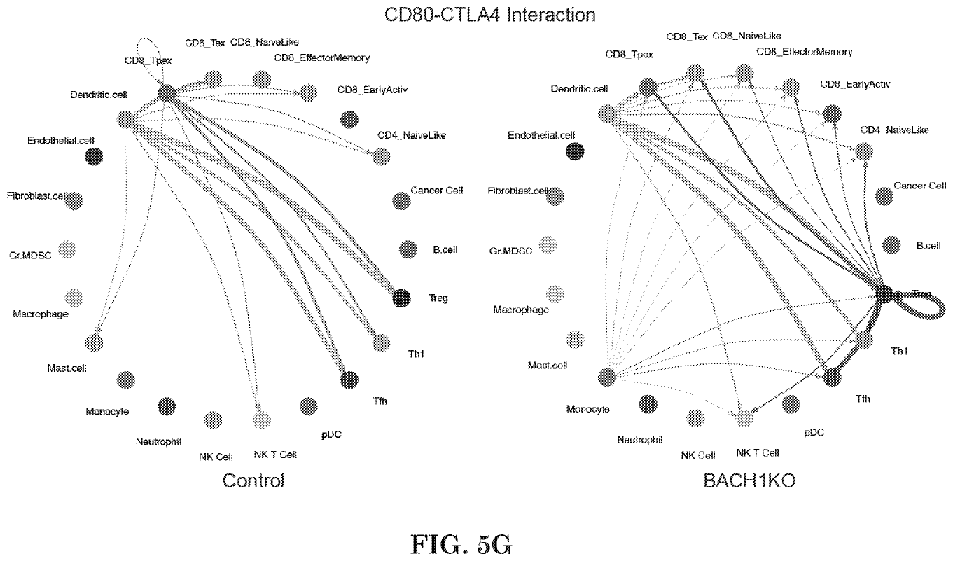

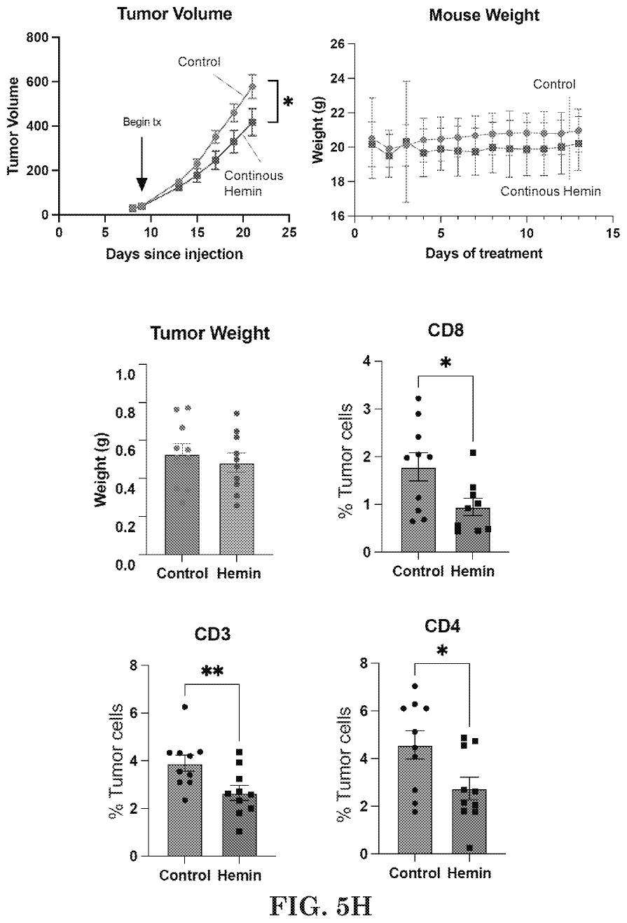

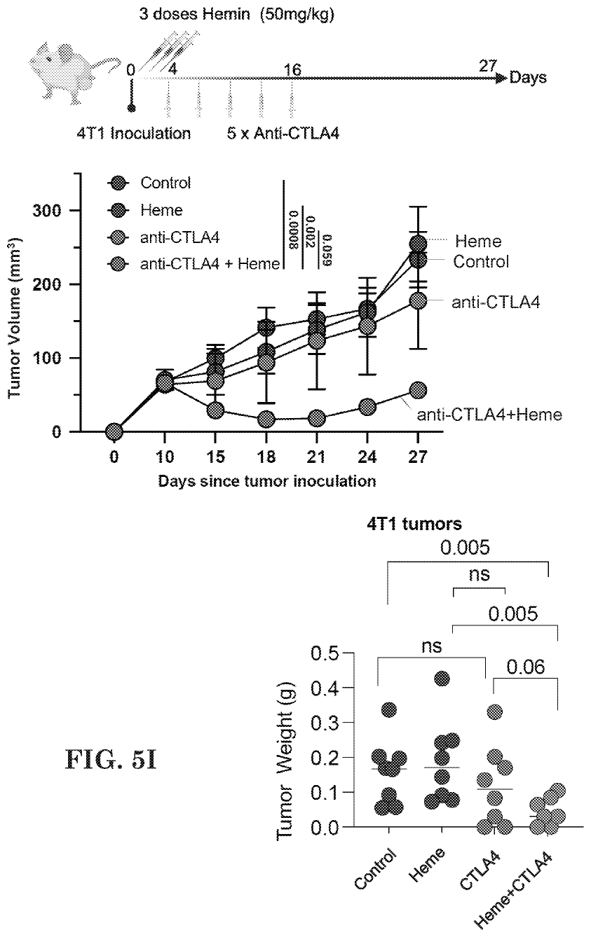

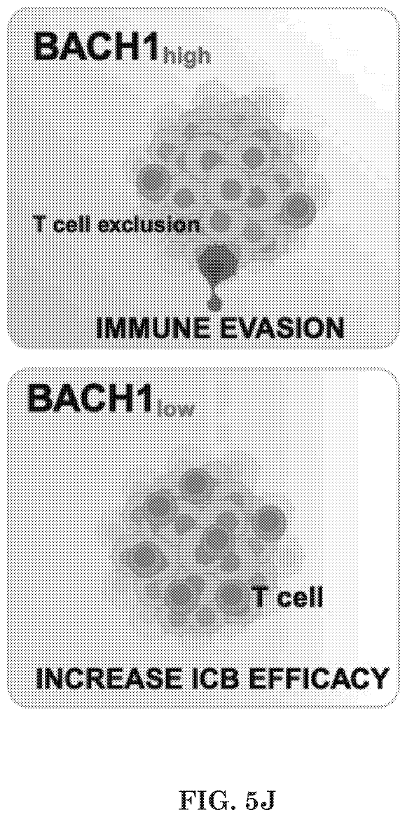

To further understand the role of cancer intrinsic-BACH1 in regulating immune evasion, single cell transcriptomic analyses of 4T1 cancer cells was conducted. As in MDA-MB-231 tumors, 4T1 tumors exhibit a similar pseudotime trajectory corresponding to a gradient of hypoxic stress (FIGS. 4R-5T). Spatial mapping of MDA-MB-231 cluster gene signatures onto 4T1 tumors demonstrated comparable patterns with proliferative cells at the tumor periphery, the most hypoxic cells near the necrotic center and transition cells in between (FIG. 4S). Notably, we observed an additional MHC class II+-rich cluster in 4T1 tumors that is devoid of co-stimulatory molecules (Cd80, Cd86) (FIGS. 4T-4V). These MHC-II-expressing 4T1 tumor cells were previously demonstrated to promote T cell tolerance, cancer cell plasticity and enhanced lymph node metastasis [45]. One potential explanation for this phenotype is the lack of induction of the co-stimulators Cd80/Cd86 by IFN-γ in 4T1 cells in vitro despite robust induction of MHC class II-related genes including H2-Ab1 and CD74 (FIG. 4W). Consistent with demonstrated plasticity, MHC-II high cells corresponded to the highest BACH1 activity cluster based on the BACH1 signature generated above (FIG. 4X). Genetic loss of BACH1 significantly decreased MHC-II expression particularly in stress clusters. (FIGS. 4X-4AA).

Although MHC-II typically facilitates CD4⁺ T cell activation by antigen-presenting cells (APCs), macrophages, and B cells, it can also induce tolerance in the absence of co-stimulatory molecules by competing for interaction with CD4⁺ T cells [43, 44]. Consistent with this, cellChat modeling predicted strong MHC-II-mediated engagement of CD4⁺ T cells by cancer cells in BACH1-high compared with BACH1 low control tumors (FIG. 4AB) whereas BACH1 knockout shifted CD4⁺ interactions toward APCs (FIG. 4AC). Importantly, this MHC-II⁺ cell state was observed in primary human breast tumors. ScRNA-seq analysis of 53 breast tumors revealed that 81% contained MHC-II⁺ tumor cells lacking CD80/CD86, highlighting the clinical relevance of BACH1-driven immune evasion though MHC-II in breast tumors (FIG. 4AD).

Together, these findings reveal that BACH1 drives immune evasion through multiple mechanisms including suppressing Type I interferon signaling, inhibiting lymphocyte infiltration, and promoting MHC class II-mediated immunosuppression. By promoting both the emergence and expression of MHC-II⁺ across multiple cell states, BACH1 enables tumors to evade T cell-mediated immune response while preserving the potential to adapt to hypoxic stress, establishing BACH1 as a key therapeutic target in non-T cell-inflammed TNBC.

### Example 5: BACH1 Inhibition Increases Sensitivity to CTLA4 Immune Checkpoint Blockade Therapy

Given BACH's role in sustaining an immune-suppressive and transcriptionally plastic tumor state, it was investigated whether BACH1 loss improves responsiveness to immune checkpoint blockade. It was first noted that BACH1 enhances PD-L1 expression by directly binding to the CD274 (PD-L1) gene locus, suggesting that PD-1/PD-L1 might not be the most effective therapeutic targets (FIG. 4Z and FIG. 5A). In contrast, CTLA4 was the most abundantly expressed immune checkpoint receptor among tumor-infiltrating T cells in both control and BACH1 KO 4T1 tumors (FIG. 5B and FIG. 5C).

Strikingly, dual targeting of BACH1 and CTLA4 in 4T1 tumors robustly reduces tumor growth and lung metastasis in mice, outcomes not achieved with PD-1/PD-L1 blockade alone or in combination with BACH1 loss (FIGS. 5D-5F). Notably, BACH1 inhibition alone significantly reduced metastatic burden, even in the absence of effective checkpoint blockade (FIG. 5D and FIG. 5E). The enhanced efficacy of CTLA4 targeting in BACH1KO tumors is likely driven by increased CD80-CTLA4 engagement demonstrated through cell-cell interaction analysis and the accumulation of activated but exhausted T cells (FIG. 4H, FIG. 4I, and FIG. 5G).

Given previous findings that hemin binds BACH1 and promotes its degradation in TNBC tumors [23], it was tested whether hemin treatment could mimic genetic BACH1 deletion. Continuous 50 mg/kg hemin administration resulted in diminished T cell infiltration, potentially due to adverse effects of chronic hemin administration on T cells (FIG. 5H). To avoid this outcome, we used a limited pretreatment strategy in which tumors received only three doses of hemin prior to anti-CTLA4 therapy. This sequential regimen recapitulated the therapeutic benefit of BACH1 deletion, demonstrating synergy between hemin and CTLA4 treatment and significantly reducing tumor growth compared to anti-CTLA4 treatment alone (FIG. 5I). Targeting BACH1 not only reprograms the tumor epigenetic and immunologic landscape but also sensitizes otherwise unresponsive tumors to CTLA4 blockade, offering a compelling therapeutic strategy for high-risk, immune-excluded breast cancers (FIGS. 5E and 5J).

### Example 6: BACH1 Activity Predicts a Non-T Cell-Inflammed Tumor Status and Poor Patient Outcome

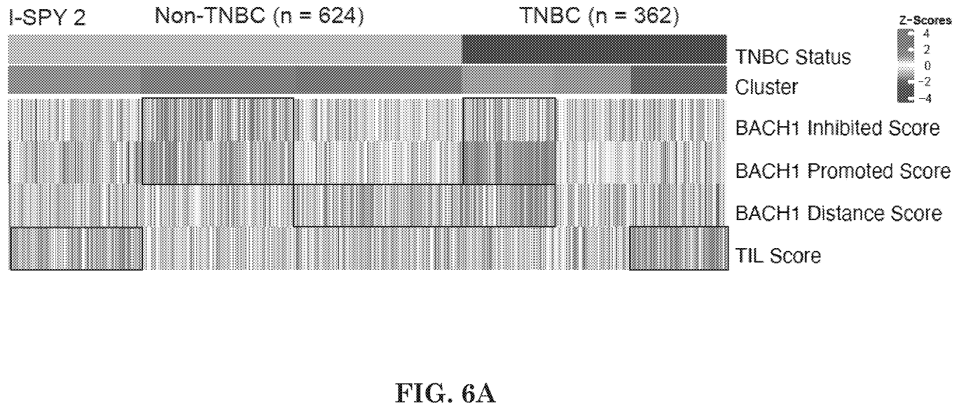

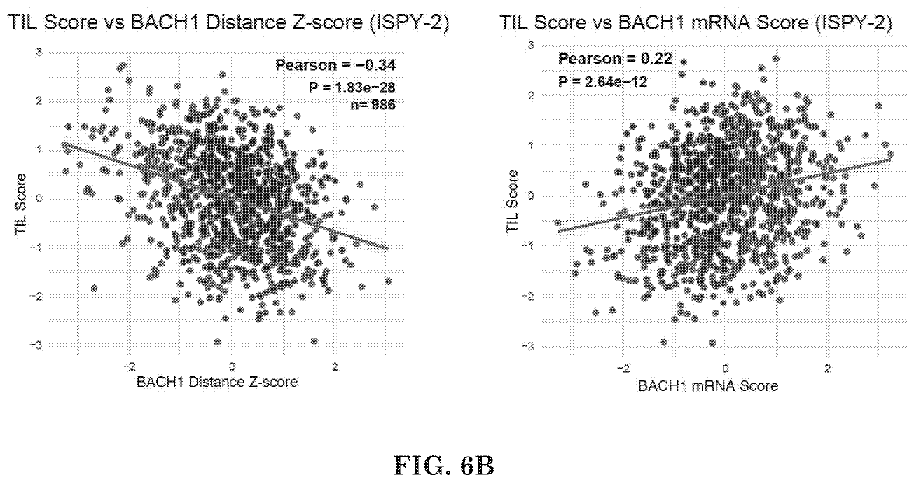

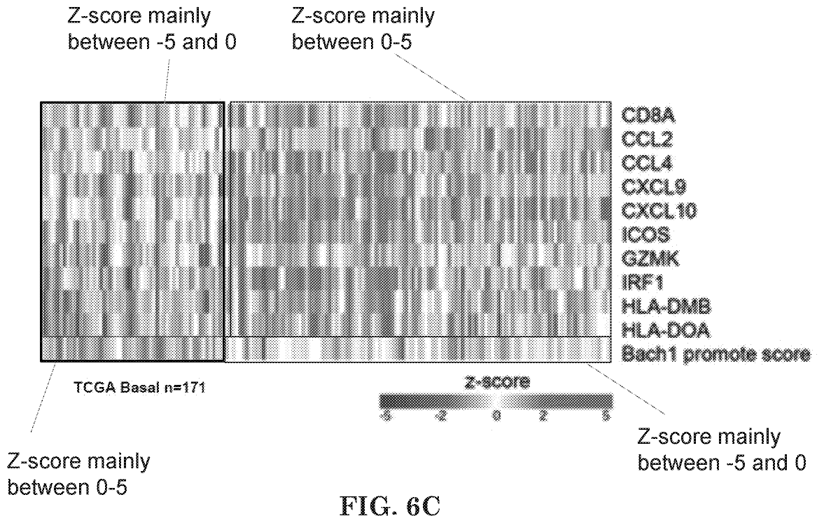

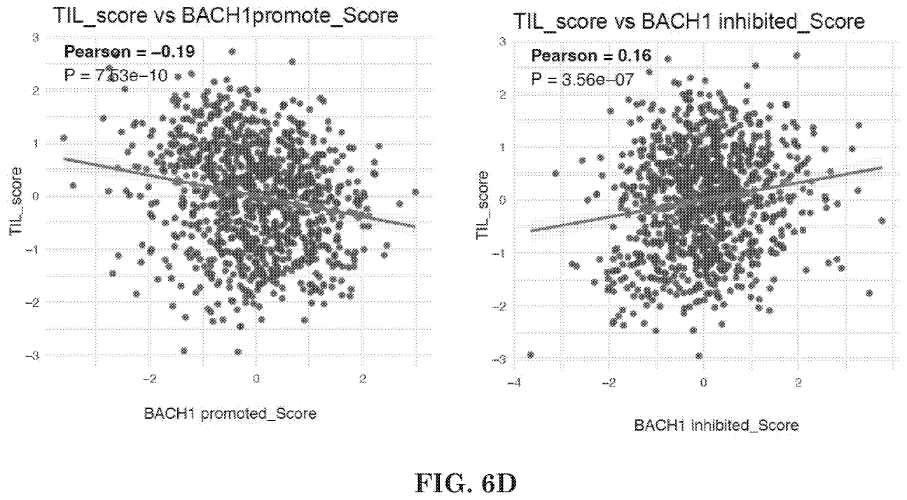

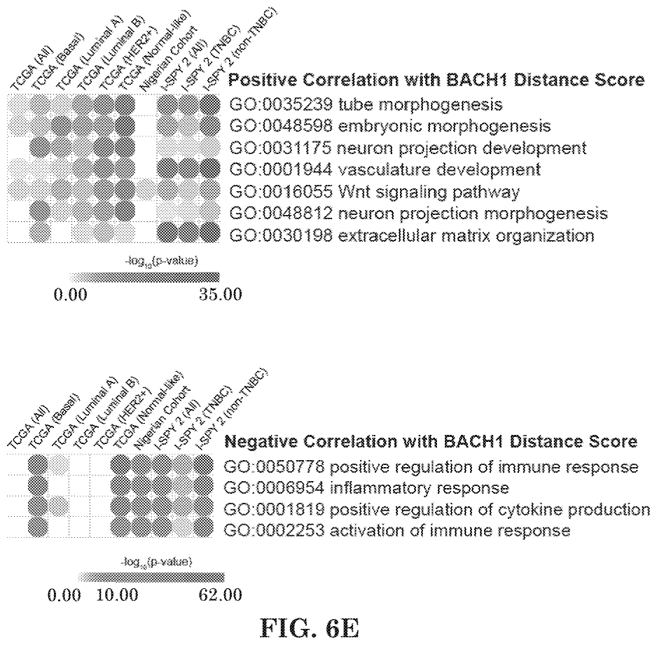

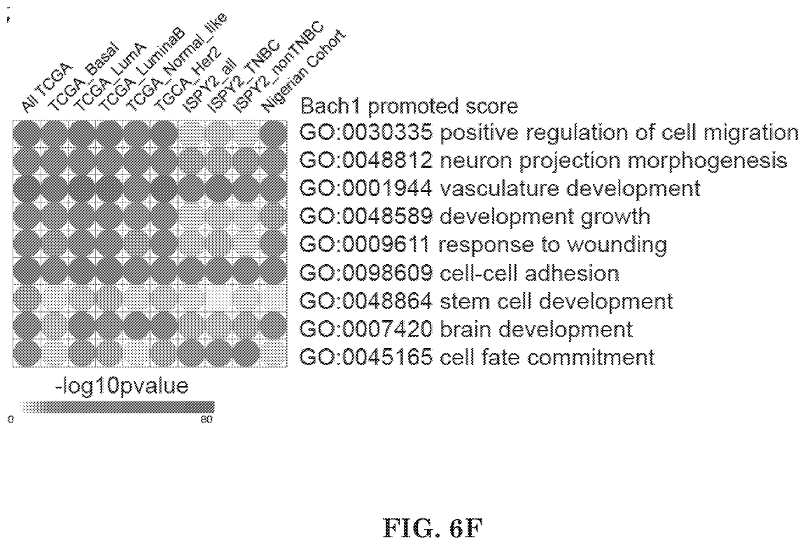

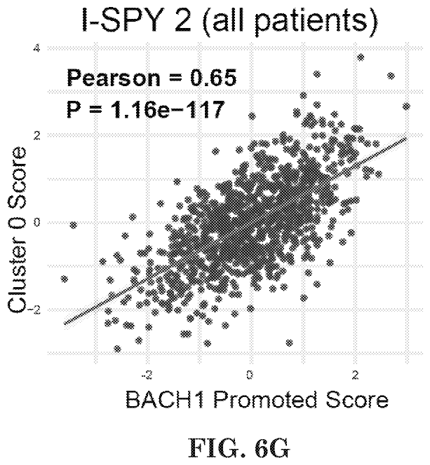

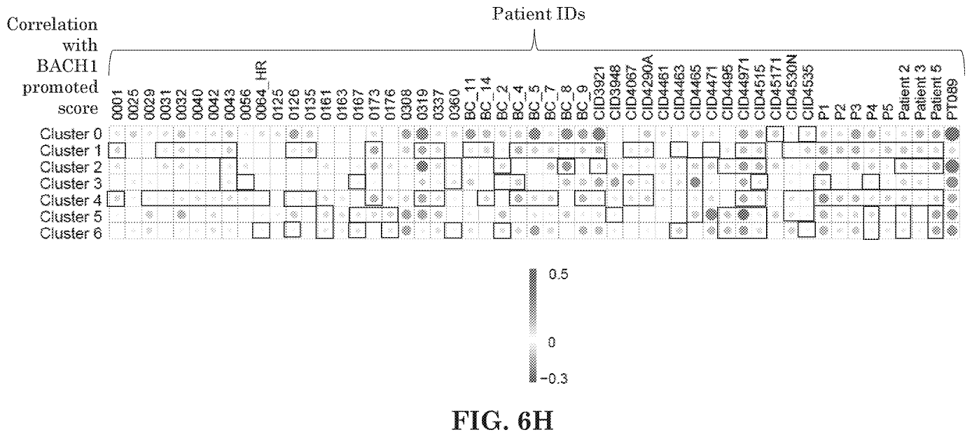

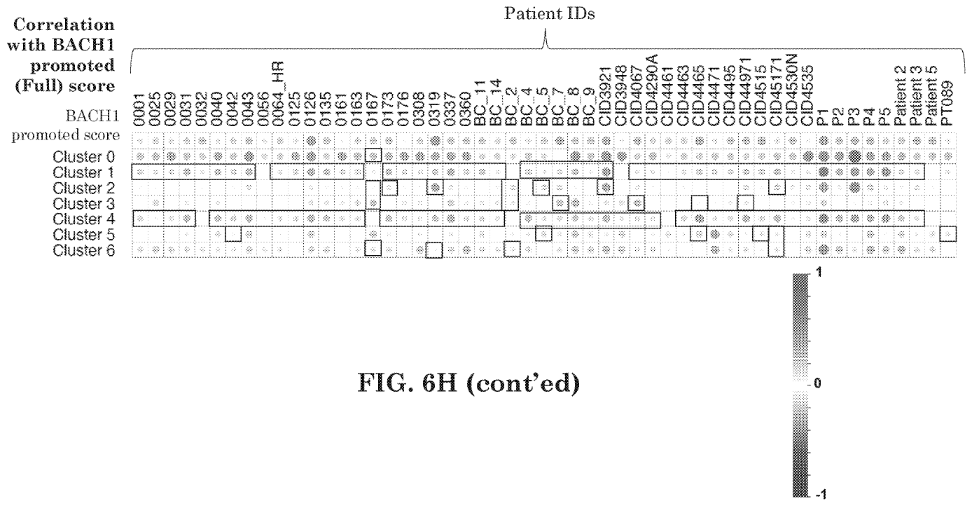

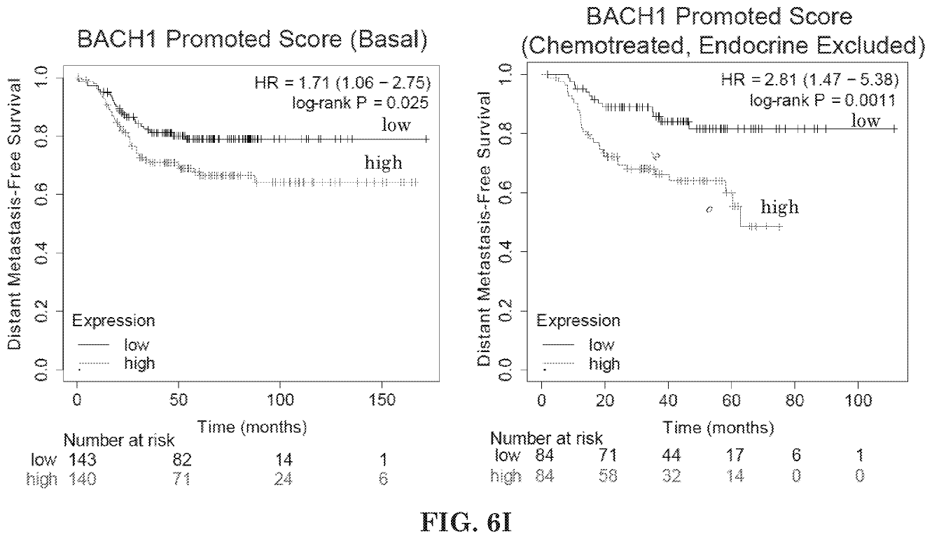

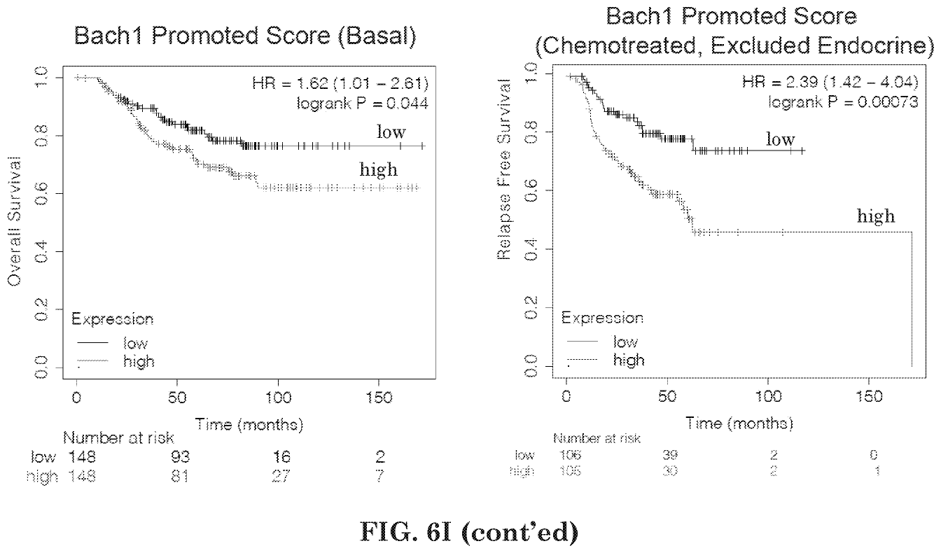

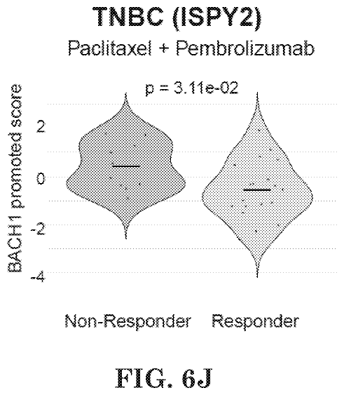

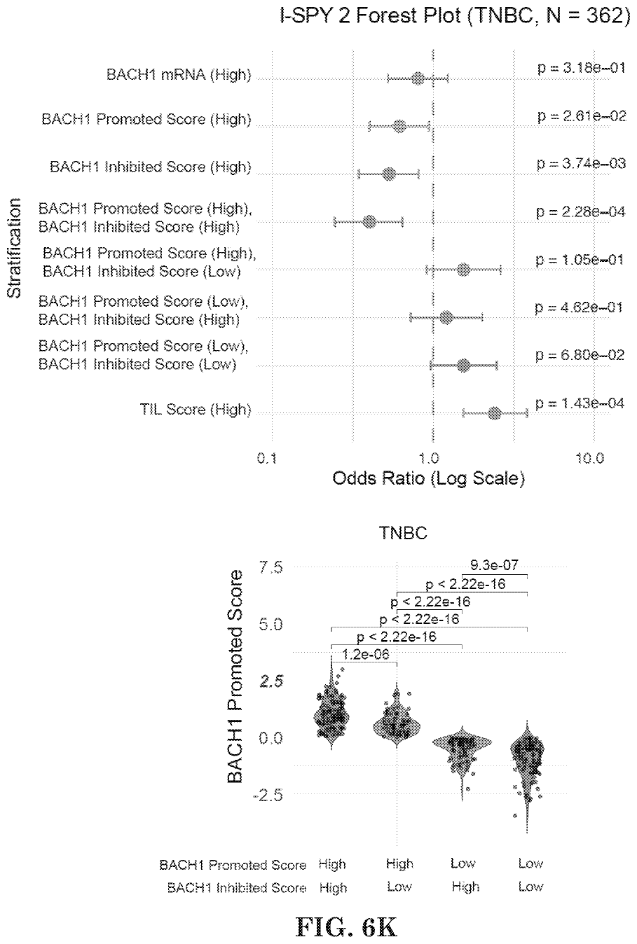

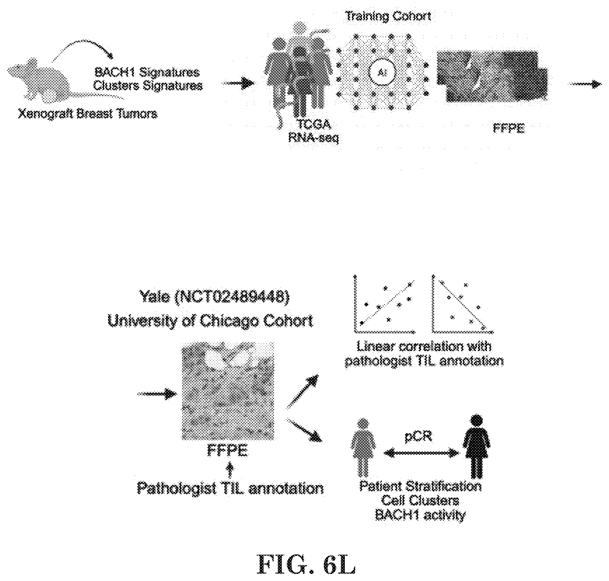

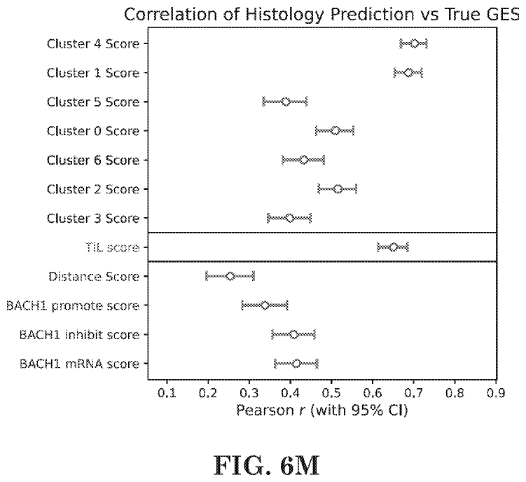

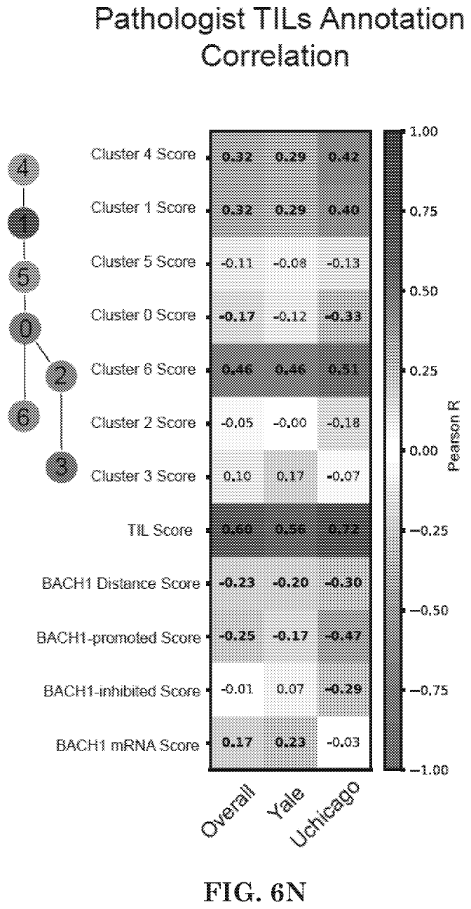

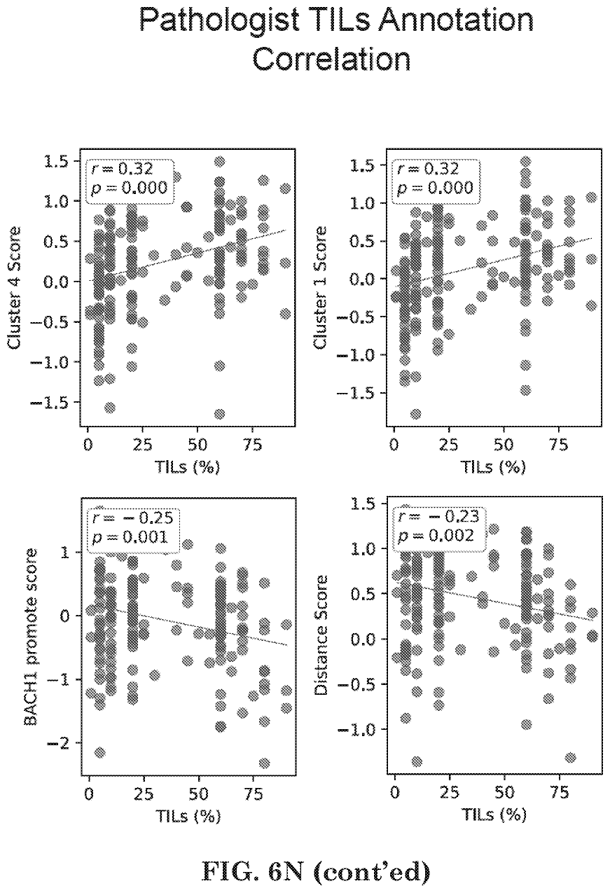

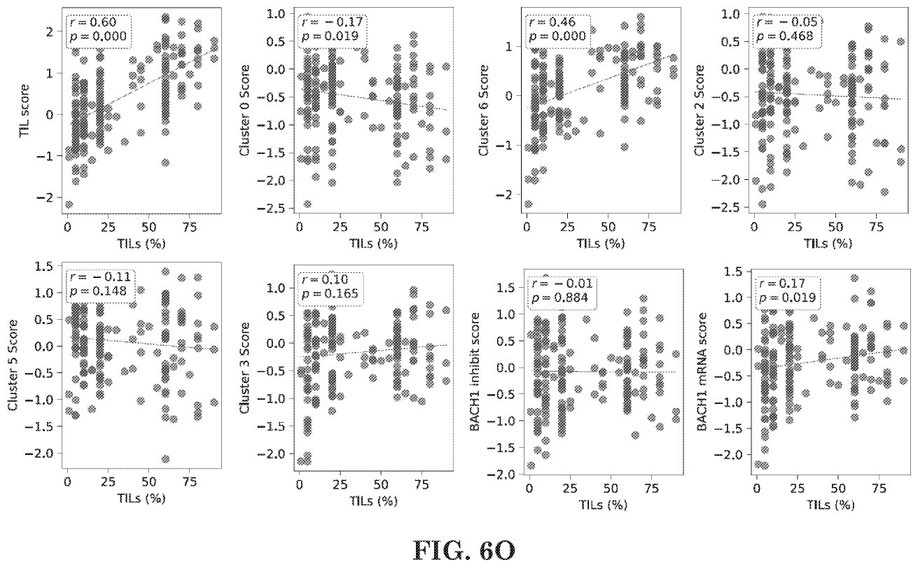

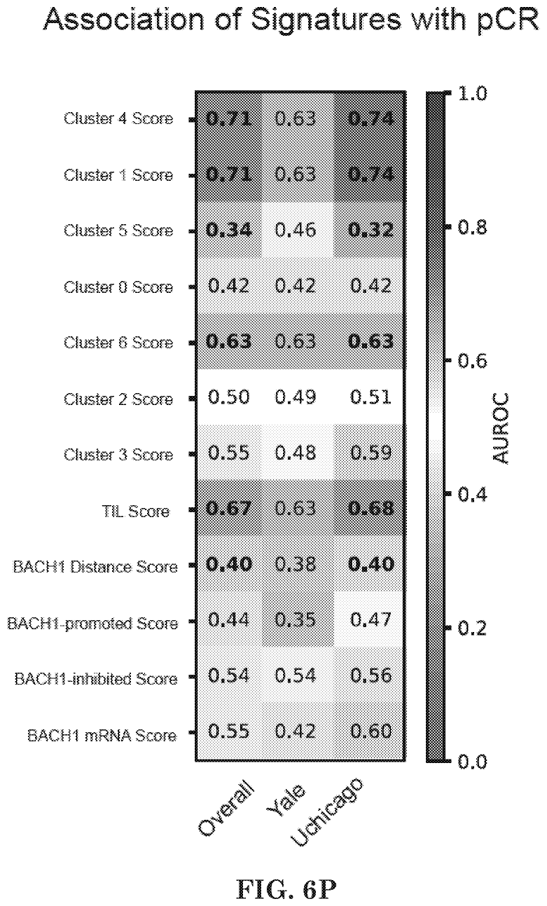

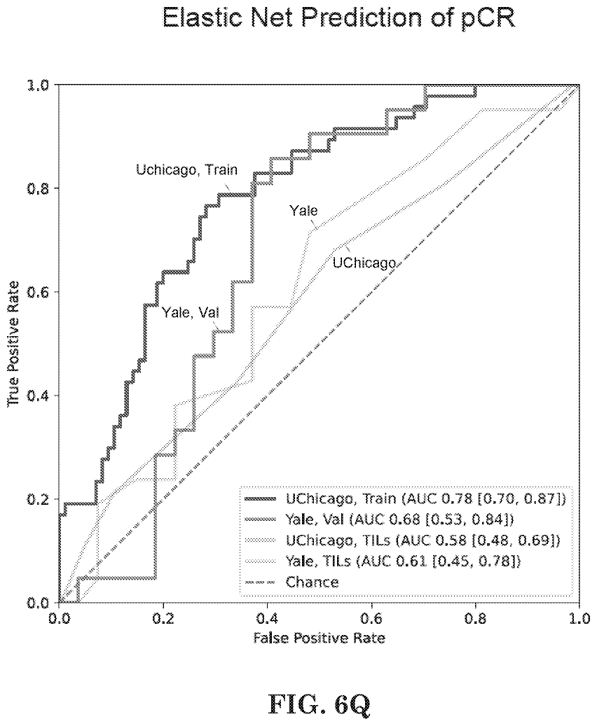

To assess clinical relevance, in vivo-derived BACH1 gene signatures were applied to patient datasets, including TCGA, a Nigerian cohort and high-risk TNBC and non-TNBC patients from the I-SPY 2 trial. Approximately one-third of the I-SPY2 patients had high BACH1 distance scores, a reflection of BACH1 activity as measured by the difference between BACH1 promoted and inhibited gene scores (FIG. 6A). Tumors with high BACH1 activity scores exhibited reduced Tumor-Infiltrating Lymphocycte (TIL)-associated gene expression, indicating a non-T cell-inflammed phenotype not predicted by BACH1 transcripts alone (FIG. 6B, FIG. 6C, and FIG. 6D). Across multiple cohorts, BACH1 activity correlated with genes related to cell adhesion, migration, neuronal and stem cell development characteristic of transition cluster 0 (FIG. 6E, FIG. 6F, and FIG. 6G). Similarly, analysis of scRNA-seq data from patient breast epithelial cancer cells showed BACH1 activity most strongly associated with Cluster 0 (FIG. 6H). These findings support BACH1's regulatory role in promoting Cluster 0 and mediating cell state transitions in patient tissue. Consistent with this observation, BACH1 activity correlated with poor distant metastasis-free and overall survival, especially in basal-like subtypes and patients treated with chemotherapy (FIG. 6I). Similarly, BACH1 activity was significantly elevated in tumors from I-SPY2 TNBC patients who did not respond to treatment with paclitaxel and pembrolizumab (anti-PD-1) immunotherapy (FIG. 6J). Patients in the I-SPY2 TNBC cohort with the highest BACH1 promoted score also had high BACH1 inhibited scores (FIG. 6K). Odds ratio analysis reveals that these patients were most likely to have less responsive tumors to all arms of neoadjuvant therapy and don't achieve a complete pathological response (FIG. 6K).

To enable broader clinical application of these findings, artificial intelligence was leveraged to analyze tissue slides and train digital histology models to predict single cell-derived cluster, TIL, and BACH1-related gene signatures (FIG. 6L). These histology-based signatures were applied to two independent sets of breast cancer patients receiving neoadjuvant therapy: (1) a University of Chicago (UChicago) cohort treated with standard anthracycline/taxane-based chemotherapy; and (2) a Yale cohort of TNBC patients receiving durvalumab (anti PD-L1) plus chemotherapy from Yale (FIG. 6L). Overall, proliferative clusters 1 and 4, as well as the TIL score, were most accurately predicted (correlation coefficients 0.65-0.70 from predicted histology versus true gene signature), although all signatures could be detected (FIG. 6M). In both cohorts, predicted BACH1 activity scores were inversely correlated with pathologist-assigned TIL scores from diagnostic core biopsies, reinforcing the non-T cell-inflammed phenotype associated with BACH1-driven programs (FIG. 6N and FIG. 6O).

It was found that the proliferation (clusters 1 and 4) and TIL scores were associated with improved response rates in both cohorts (AUROC>0.5), achieving statistical significance in the larger UChicago group (FIG. 6P). Notably, the BACH1 distance score was negatively associated with response (AUROC<0.5), reaching statistical significance in the combined cohorts (FIG. 6P). To enhance predictive power, 10 digital histology signatures were combined, including BACH1 activity and cluster signatures, into an elastic net logistic regression model that was applied to the UChicago cohort and further tested in the Yale cohort (FIG. 6Q). This integrated model significantly outperformed all individual signatures as well as pathologist-derived TIL scores, achieving superior accuracy in predicting treatment response for both cohorts (U Chicago, AUC 0.78; Yale, AUC 0.68).

In summary, these results support BACH1 as a key driver of tumor plasticity and immune exclusion in TNBC. Integrating BACH1 activity with cluster signatures offers a clinically relevant approach to stratify patients with immune-cold tumors who may benefit from BACH1-targeted therapies or rational immunotherapy combinations.

The Examples examined the impact of environmental stress, particularly hypoxia, on the ability to populate and stabilize these states, resulting in phenotypic plasticity largely independent of genetic drivers. Notably, cell clusters linked to stress-induced cell states are the dominant populations in patient breast tumors. The findings implicate BACH1 as a principal regulator of these stress-mediated cell state transitions, but it is not required to generate these states. Specifically, a cell state (Cluster 0) was identified to exhibit a pro-metastatic phenotype whose heterogeneity and plasticity were regulated by BACH1 and correlate with poor survival outcomes.

Targeting cancer cell-intrinsic BACH1 reprograms the tumor immune microenvironment, shifting it toward a more activated and immune-responsive state, while simultaneously sensitizing tumors to anti-CTLA-4 therapy. Importantly, this dual mechanism presents a promising strategy to overcome immune-cold phenotypes and enhance therapeutic efficacy. Moreover, the selective effects of BACH1 on immune modulation highlight its potential as a biomarker for patient stratification in future CTLA-4-based immunotherapy trials. With the emergence of next-generation CTLA-4 inhibitors designed to reduce toxicity in clinical settings, co-targeting BACH1 may pave the way for safer and more effective treatments, offering new hope for patients with TNBC and other immune-refractory malignancies. These results underscore the transformative potential of BACH1 as a therapeutic target, with implications for improving outcomes in TNBC and other malignancies that rely on immune evasion for progression and metastasis.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification, and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth herein.

### REFERENCES

1. Igarashi et al. The transcription factor BACH1 at the crossroads of cancer biology: From epithelial-mesenchymal transition to ferroptosis. J Biol Chem 297, 101032 (2021).
2. Vranic et al. PD-L1 testing by immunohistochemistry in immuno-oncology. Biomol Biomed. 23, 15-25 (2023).
3. Kassardjian et al. Expression of immune checkpoint regulators, cytotoxic T lymphocyte antigen 4 (CTLA-4) and programmed death-ligand 1 (PD-L1), in female breast carcinomas. PLOS One 13, e0195958 (2018).
4. Zenke-Kawasaki et al. Heme induces ubiquitination and degradation of the transcription factor Bach1. Mol Cell Biol. 27, 6962-71 (2007).
5. Johnstone et al. Functional and molecular characterisation of EO771.LMB tumours, a new C57BL/6-mouse-derived model of spontaneously metastatic mammary cancer. Dis Model Mech. 8, 237-51 (2015).
6. Zhu, Q. et al. Single cell multi-omics reveal intra-cell-line heterogeneity across human cancer cell lines. Nat Commun 14, 8170 (2023).
7. Godet, I. et al. Fate-mapping post-hypoxic tumor cells reveals a ROS-resistant phenotype that promotes metastasis. Nat Commun 10, 4862 (2019).
8. Massague, J. & Ganesh, K. Metastasis-Initiating Cells and Ecosystems. Cancer Discov 11, 971-994 (2021).
9. Ganesh, K. & Massague, J. Targeting metastatic cancer. Nat Med 27, 34-44 (2021).
10. Gerstberger, S., Jiang, Q. & Ganesh, K. Metastasis. Cell 186, 1564-1579 (2023).
11. Wang, Y. et al. Interferon stimulated immune profile changes in a humanized mouse model of HBV infection. Nat Commun 14, 7393 (2023).
12. Klughammer, J. et al. A multi-modal single-cell and spatial expression map of metastatic breast cancer biopsies across clinicopathological features. Nat Med 30, 3236-3249 (2024).
13. Park, J. W. et al. Reprogramming normal human epithelial tissues to a common, lethal neuroendocrine cancer lineage. Science 362, 91-95 (2018).
14. Liberzon, A. et al. The Molecular Signatures Database (MSigDB) hallmark gene set collection. Cell Syst 1, 417-425 (2015).
15. Hirsila, M., Koivunen, P., Gunzler, V., Kivirikko, K. I. & Myllyharju, J. Characterization of the human prolyl 4-hydroxylases that modify the hypoxia-inducible factor. J Biol Chem 278, 30772-30780 (2003).
16. Haynes, N. M., Chadwick, T. B. & Parker, B. S. The complexity of immune evasion mechanisms throughout the metastatic cascade. Nat Immunol 25, 1793-1808 (2024).
17. Zhang, X. et al. Double-edged effects of interferons on the regulation of cancer-immunity cycle. Oncoimmunology 10, 1929005 (2021).
18. Wolf, D. M. et al. Redefining breast cancer subtypes to guide treatment prioritization and maximize response: Predictive biomarkers across 10 cancer therapies. Cancer Cell 40, 609-623 e606 (2022).
19. Akiyama, H., Chaboissier, M. C., Martin, J. F., Schedl, A. & de Crombrugghe, B. The transcription factor Sox9 has essential roles in successive steps of the chondrocyte differentiation pathway and is required for expression of Sox5 and Sox6. Genes Dev 16, 2813-2828 (2002).
20. Ota, K., Brydun, A., Itoh-Nakadai, A., Sun, J. & Igarashi, K. Bach1 deficiency and accompanying overexpression of heme oxygenase-1 do not influence aging or tumorigenesis in mice. Oxid Med Cell Longev 2014, 757901 (2014).
21. Yun, J. et al. Signaling pathway for RKIP and Let-7 regulates and predicts metastatic breast cancer. EMBO J 30, 4500-4514 (2011).
22. Igarashi, K., Nishizawa, H., Saiki, Y. & Matsumoto, M. The transcription factor BACH1 at the crossroads of cancer biology: From epithelial-mesenchymal transition to ferroptosis. J Biol Chem 297, 101032 (2021).
23. Lee, J. et al. Effective breast cancer combination therapy targeting BACH1 and mitochondrial metabolism. Nature 568, 254-258 (2019).
24. Sato, M. et al. BACH1 Promotes Pancreatic Cancer Metastasis by Repressing Epithelial Genes and Enhancing Epithelial-Mesenchymal Transition. Cancer Res 80, 1279-1292 (2020).
25. Nishizawa, H. et al. Ferroptosis is controlled by the coordinated transcriptional regulation of glutathione and labile iron metabolism by the transcription factor BACH1. J Biol Chem 295, 69-82 (2020).
26. Hur, H., Lee, J. Y., Yun, H. J., Park, B. W. & Kim, M. H. Analysis of HOX gene expression patterns in human breast cancer. Mol Biotechnol 56, 64-71 (2014).
27. Di Giovannantonio, M. et al. Defining hypoxia in cancer: A landmark evaluation of hypoxia gene expression signatures. Cell Genom 5, 100764 (2025).
28. Dey, K. K., Hsiao, C. J. & Stephens, M. Visualizing the structure of RNA-seq expression data using grade of membership models. PLOS Genet 13, e1006599 (2017).
29. Kharchenko, P. V., Silberstein, L. & Scadden, D. T. Bayesian approach to single-cell differential expression analysis. Nat Methods 11, 740-742 (2014).
30 Lignitto, L. et al. Nrf2 Activation Promotes Lung Cancer Metastasis by Inhibiting the Degradation of Bach1. Cell 178, 316-329 e318 (2019).
31. DePasquale, E. A. K. et al. DoubletDecon: Deconvoluting Doublets from Single-Cell RNA-Sequencing Data. Cell Rep 29, 1718-1727 e1718 (2019).
32. Hao, Y. et al. Integrated analysis of multimodal single-cell data. Cell 184, 3573-3587 e3529 (2021).
33. Li, J. et al. Non-cell-autonomous cancer progression from chromosomal instability. Nature 620, 1080-1088 (2023).
34. Slyper, M. et al. Author Correction: A single-cell and single-nucleus RNA-Seq toolbox for fresh and frozen human tumors. Nat Med 26, 1307 (2020).
35. Andreatta, M. et al. Interpretation of T cell states from single-cell transcriptomics data using reference atlases. Nat Commun 12, 2965 (2021).
36. Jin, S. et al. Inference and analysis of cell-cell communication using CellChat. Nat Commun 12, 1088 (2021).
37. Wang, L., Liu, C., Gao, Y., Zhang, X. H. & Liu, Z. Unravelling spatial gene associations with SEAGAL: a Python package for spatial transcriptomics data analysis and visualization. Bioinformatics 39 (2023).
38. Xu, L. et al. A comprehensive single-cell breast tumor atlas defines epithelial and immune heterogeneity and interactions predicting anti-PD-1 therapy response. Cell Rep Med 5, 101511 (2024).
39. Ma, S. et al. Hypoxia induces HIF1alpha-dependent epigenetic vulnerability in triple negative breast cancer to confer immune effector dysfunction and resistance to anti-PD-1 immunotherapy. Nat Commun 13, 4118 (2022).
40. Scharping, N. E. et al. Mitochondrial stress induced by continuous stimulation under hypoxia rapidly drives T cell exhaustion. Nat Immunol 22, 205-215 (2021).
41. Barsoum, I. B., Smallwood, C. A., Siemens, D. R. & Graham, C. H. A mechanism of hypoxia-mediated escape from adaptive immunity in cancer cells. Cancer Res 74, 665-674 (2014).
42. Qiao, Z. et al. Direct inhibition of tumor hypoxia response with synthetic transcriptional repressors. Nat Chem Biol 21, 247-255 (2025).
43. Wosen, J. E., Mukhopadhyay, D., Macaubas, C. & Mellins, E. D. Epithelial MHC Class II Expression and Its Role in Antigen Presentation in the Gastrointestinal and Respiratory Tracts. Front Immunol 9, 2144 (2018).
44. Alegre, M. L., Frauwirth, K. A. & Thompson, C. B. T-cell regulation by CD28 and CTLA-4. Nat Rev Immunol 1, 220-228 (2001).
45. Lei, P. J. et al. Cancer cell plasticity and MHC-II-mediated immune tolerance promote breast cancer metastasis to lymph nodes. J Exp Med 220 (2023).
46. Liu, Z. et al. CD74 interacts with CD44 and enhances tumorigenesis and metastasis via RHOA-mediated cofilin phosphorylation in human breast cancer cells. Oncotarget 7, 68303-68313 (2016).
47. Zhang, H. et al. Recent research and clinical progress of CTLA-4-based immunotherapy for breast cancer. Front Oncol 13, 1256360 (2023).
48. Schmid, P. et al. Atezolizumab and Nab-Paclitaxel in Advanced Triple-Negative Breast Cancer. N Engl J Med 379, 2108-2121 (2018).
49. Iglesia, M. D. et al. Differential chromatin accessibility and transcriptional dynamics define breast cancer subtypes and their lineages. Nat Cancer 5, 1713-1736 (2024).
50 Lange, M. et al. CellRank for directed single-cell fate mapping. Nat Methods 19, 159-170 (2022).
51. Weiler, P., Lange, M., Klein, M., Pe′er, D. & Theis, F. CellRank 2: unified fate mapping in multiview single-cell data. Nat Methods 21, 1196-1205 (2024).
52. Setty, M. et al. Characterization of cell fate probabilities in single-cell data with Palantir. Nat Biotechnol 37, 451-460 (2019).
53. Zhou, Y. et al. Metascape provides a biologist-oriented resource for the analysis of systems-level datasets. Nat Commun 10, 1523 (2019).
54. Kang, Y. et al. A multigenic program mediating breast cancer metastasis to bone. Cancer Cell 3, 537-549 (2003).
55. Matossian, M. D. et al. In-depth characterization of a new patient-derived xenograft model for metaplastic breast carcinoma to identify viable biologic targets and patterns of matrix evolution within rare tumor types. Clin Transl Oncol 24, 127-144 (2022).
56. Sachs, N. et al. A Living Biobank of Breast Cancer Organoids Captures Disease Heterogeneity. Cell 172, 373-386 e310 (2018).
57. Wan, Y. et al. Nonmonotone invasion landscape by noise-aware control of metastasis activator levels. Nat Chem Biol 19, 887-899 (2023).
58. Yesilkanal, A. E. et al. Limited inhibition of multiple nodes in a driver network blocks metastasis. Elife 10 (2021).
59. Afgan, E. et al. The Galaxy platform for accessible, reproducible and collaborative biomedical analyses: 2018 update. Nucleic Acids Res 46, W537-W544 (2018).
60. Li, H. Aligning sequence reads, clone sequences and assembly contigs with BWA-MEM. arXiv (2013).
61. Robinson, J. T. et al. Integrative genomics viewer. Nat Biotechnol 29, 24-26 (2011).
62. Li, H. et al. The Sequence Alignment/Map format and SAMtools. Bioinformatics 25, 2078-2079 (2009).
63. Heinz, S. et al. Simple combinations of lineage-determining transcription factors prime cis-regulatory elements required for macrophage and B cell identities. Mol Cell 38, 576-589 (2010).
64. Ramirez, F., Dundar, F., Diehl, S., Gruning, B. A. & Manke, T. deepTools: a flexible platform for exploring deep-sequencing data. Nucleic Acids Res 42, W187-191 (2014).
65. Jiang, Z. et al. TYRO3 induces anti-PD-1/PD-L1 therapy resistance by limiting innate immunity and tumoral ferroptosis. J Clin Invest 131 (2021).
66. Epel, B. & Halpern, H. J. Imaging. Emagres 6, 149-160 (2017).
67. Epel, B., Bowman, M. K., Mailer, C. & Halpern, H. J. Absolute oxygen Rle imaging in vivo with pulse electron paramagnetic resonance. Magnet Reson Med 72, 362-368 (2014).
68. Epel, B. & Halpern, H. J. EPR Oxygen Imaging Workflow with MATLAB Image Registration Toolbox. Applied Magnetic Resonance 52, 1311-1319 (2021).
69. Nguyen, L. C. et al. Cannabidiol inhibits SARS-COV-2 replication through induction of the host ER stress and innate immune responses. Sci Adv 8, eabi6110 (2022).
70. Dobin, A. et al. STAR: ultrafast universal RNA-seq aligner. Bioinformatics 29, 15-21 (2013).
71. Liao, Y., Smyth, G. K. & Shi, W. featureCounts: an efficient general purpose program for assigning sequence reads to genomic features. Bioinformatics 30, 923-930 (2014).
72. Love, M. I., Huber, W. & Anders, S. Moderated estimation of fold change and dispersion for RNA-seq data with DESeq2. Genome Biol 15, 550 (2014).
73. Ewels, P. A. et al. The nf-core framework for community-curated bioinformatics pipelines. Nat Biotechnol 38, 276-278 (2020).
74. Di Tommaso, P. et al. Nextflow enables reproducible computational workflows. Nat Biotechnol 35, 316-319 (2017).
75. Kurtzer, G. M., Sochat, V. & Bauer, M. W. Singularity: Scientific containers for mobility of compute. PLOS One 12, e0177459 (2017).
76. Zhang, Y. et al. Model-based analysis of ChIP-Seq (MACS). Genome Biol 9, R137 (2008).
77. Liu, T. Use model-based Analysis of ChIP-Seq (MACS) to analyze short reads generated by sequencing protein-DNA interactions in embryonic stem cells. Methods Mol Biol 1150, 81-95 (2014).
78. Ramirez, F. et al. deepTools2: a next generation web server for deep-sequencing data analysis. Nucleic Acids Res 44, W160-165 (2016).
79. Yu, G., Wang, L. G. & He, Q. Y. ChIPseeker: an R/Bioconductor package for ChIP peak annotation, comparison and visualization. Bioinformatics 31, 2382-2383 (2015).
80. Ross-Innes, C. S. et al. Differential oestrogen receptor binding is associated with clinical outcome in breast cancer. Nature 481, 389-393 (2012).
81. Gyorffy, B. Survival analysis across the entire transcriptome identifies biomarkers with the highest prognostic power in breast cancer. Comput Struct Biotechnol J 19, 4101-4109 (2021).
82. Gyorffy, B. Integrated analysis of public datasets for the discovery and validation of survival-associated genes in solid tumors. Innovation (Camb) 5, 100625 (2024).
83. Wu, M. C. & Lin, X. Prior biological knowledge-based approaches for the analysis of genome-wide expression profiles using gene sets and pathways. Stat Methods Med Res 18, 577-593 (2009).
84. Subramanian, A. et al. Gene set enrichment analysis: a knowledge-based approach for interpreting genome-wide expression profiles. Proc Natl Acad Sci USA 102, 15545-15550 (2005).
85. Holicek, P. et al. Type I interferon and cancer. Immunol Rev 321, 115-127 (2024). doi.org:10.1111/imr.13272.
86. Howard, F. M. et al. Generative adversarial networks accurately reconstruct pan-cancer histology from pathologic, genomic, and radiographic latent features. Sci Adv 10, eadq0856 (2024). doi.org: 10.1126/sciadv.adq0856.
87. Chen, R. J. et al. Towards a general-purpose foundation model for computational pathology. Nat Med 30, 850-862 (2024). doi.org: 10.1038/s41591-024-02857-3.
88. Wagner, S. J. et al. Transformer-based biomarker prediction from colorectal cancer histology: A large-scale multicentric study. Cancer Cell 41, 1650-1661 e1654 (2023). doi.org: 10.1016/j.ccell.2023.08.002.
89. Hanley, J. A. & McNeil, B. J. The meaning and use of the area under a receiver operating characteristic (ROC) curve. Radiology 143, 29-36 (1982). doi.org: 10.1148/radiology.143.1.7063747.

1. A method of treating breast cancer in a subject, comprising administering a therapeutically effective amount of a transcription factor BTB and CNC Homology 1 (BACH1) inhibitor to the subject.

2. The method of claim 1, further comprising administering a therapeutically effective amount of an anti-cancer agent.

3. The method of claim 2, wherein the therapeutically effective amount of the BACH1 inhibitor is administered prior to or concurrent with the therapeutically effective amount of the inhibitor of the anti-cancer agent.

4. The method of claim 3, wherein the anti-cancer agent is an inhibitor of an immune checkpoint protein.

5. The method of claim 4, wherein the immune checkpoint protein is CTLA4, PD-1, PD-L1, LAG-3, TIM-3, TIGIT, PD-1H, CD278, CD39/CD73, or CD276.

6. The method of claim 5, wherein the CTLA4 inhibitor is an anti-CTLA4 antibody.

7. The method of claim 6, wherein the anti-CTLA4 antibody is ipilimumab, tremelimumab, botensilimab, or a combination thereof.

8. The method of claim 1, wherein the breast cancer is triple-negative breast cancer.

9. The method of claim 2, wherein a higher therapeutic efficacy of the anti-cancer agent is achieved compared to administration of the anti-cancer agent alone.

10. A pharmaceutical composition, comprising:
a) a therapeutically effective amount of a BACH1 inhibitor;
b) a therapeutically effective amount of an anti-cancer agent; and
c) a pharmaceutically acceptable carrier, solvent, adjuvant, diluent, or a combination thereof.

11. The pharmaceutical composition of claim 10, wherein the anti-cancer agent is an inhibitor of an immune checkpoint protein.

12. The pharmaceutical composition of claim 11, wherein the immune checkpoint protein is CTLA4, PD-1, PD-L1, LAG-3, TIM-3, TIGIT, PD-1H, CD278, CD39/CD73, or CD276.

13. The pharmaceutical composition of claim 12, wherein the CTLA4 inhibitor is an anti-CTLA4 antibody.

14. The pharmaceutical composition of claim 13, wherein the anti-CTLA4 antibody is ipilimumab, tremelimumab, botensilimab, or a combination thereof.

15. The pharmaceutical composition of claim 10, wherein the BACH1 inhibitor comprises hemin.

16. A method of treating cancer in a subject, comprising:
a) taking a sample from a cancer tumor of the subject;
b) detecting a level of expression and/or activity of BACH1 in the tumor sample;
c) detecting a level of expression of an immune checkpoint protein in the tumor sample; and
d) administering a therapeutically effective amount of a BACH1 inhibitor and a therapeutically effective amount of an inhibitor of an immune checkpoint protein to the subject, if the level of expression and/or activity of BACH1 in the tumor sample is greater than in cancer subjects that are responsive to the inhibitor of the immune checkpoint protein alone without the BACH1 inhibitor and if the level of expression of the immune checkpoint protein in the tumor sample is scored as high.

17. The method of claim 16, wherein detecting the level of expression of BACH1 or immune checkpoint protein comprises single cell RNA sequencing, Northern blot, serial analysis of gene expression (SAGE), microarray analysis, quantitative reverse transcription polymerase chain reaction (RT-qPCR), bulk RNA sequencing (bulk RNAseq), Western Blot, immunofluorescence, immunohistochemistry, in situ hybridization, or a combination thereof.

18. The method of claim 16, wherein the therapeutically effective amount of the BACH1 inhibitor is administered prior to or concurrent with the therapeutically effective amount of the inhibitor of the immune checkpoint protein.

19. The method of claim 16, wherein detecting the level of activity of BACH1 comprises detecting expression of one or more genes that are promoted and/or inhibited by BACH1.

20. The method of claim 16, wherein the immune checkpoint protein is CTLA4, PD-1, PD-L1, LAG-3, TIM-3, TIGIT, PD-1H, CD278, CD39/CD73, or CD276.

21. The method of claim 20, wherein the inhibitor of the immune checkpoint protein is an anti-CTLA4 antibody.

22. The method of claim 21, wherein the anti-CTLA4 antibody is ipilimumab, tremelimumab, botensilimab, or a combination thereof.

23. The method of claim 16, wherein the cancer is a breast cancer.

24. The method of claim 16, wherein the BACH1 inhibitor comprises hemin.
